(19) United States
(12) Patent Application Publication
(10) Pub. No.: US 2025/0326861 A1
(43) Pub. Date: Oct. 23, 2025

(54) VECTORIZED LANADELUMAB AND ADMINISTRATION THEREOF

(71) Applicant: REGENXBIO INC., Rockville, MD (US)

(72) Inventors: Ye Liu, Clarksville, MD (US); Joseph Bruder, Gaithersburg, MD (US); Devin McDougald, Boston, MA (US); Subha Karumuthil-Melethil, Germantown, MD (US); Jennifer M. Egley, Gaithersburg, MD (US); Andrew Mercer, Poolesville, MD (US)

(21) Appl. No.: 18/026,117

(22) PCT Filed: Sep. 15, 2021
(86) PCT No.: PCT/US21/50564

§ 371 Date: Mar. 13, 2023

Related U.S. Application Data
(60) Provisional application No. 63/078,879, filed on Sep. 15, 2020.
(60) Provisional application No. 63/106,843, filed on Oct. 28, 2020.

(51) Int. Cl. C07K 16/40; A61K 9/00; A61K 39/00; A61K 48/00; A61P 37/06; C12N 15/86

(52) U.S. Cl. CPC C07K 16/40; A61K 9/0019; A61K 48/0058; A61P 37/06; C12N 15/86; A61K 2039/505; C07K 2317/21; C07K 2317/51; C07K 2317/515; C07K 2317/55; C07K 2317/622; C07K 2317/76; C07K 2319/02; C07K 2319/50; C12N 2750/14122; C12N 2750/14143; C12N 2830/008; C12N 2830/50

(57) ABSTRACT

Compositions and methods are described for the delivery of a fully human post-translationally modified therapeutic monoclonal antibody that binds to plasma kallekrein (pKal) to a human subject diagnosed with a disease or condition indicated for treatment with an anti-pKal antibody. Compositions and methods are also described with liver specific promoter combinations to enhance gene expression in liver cells. Such diseases include hereditary angioedema, as well as ocular indications, such as diabetic retinopathy and diabetic macular edema. Dosing of viral vectors encoding the anti-pKal antibody to achieve therapeutically effective serum levels is provided.

### 1. INTRODUCTION

Compositions and methods are described for the delivery of a fully human post-translationally modified (HuPTM) therapeutic monoclonal antibody (“mAb”) that binds to pKal or the HuPTM antigen-binding fragment of a therapeutic mAb that binds to pKal—e.g., a fully human-glycosylated (HuGly) Fab of the therapeutic mAb—to a human subject diagnosed with a disease or condition indicated for treatment with the therapeutic mAb. Such diseases include hereditary angioedema, as well as ocular indications, such as diabetic retinopathy and diabetic macular edema. Dosing of viral vectors encoding the anti-pKal antibody to achieve therapeutically effective serum levels is provided herein.

### 2. BACKGROUND OF THE INVENTION

Therapeutic mAbs have been shown to be effective in treating a number of diseases and conditions. However, because these agents are effective for only a short period of time, repeated injections for long durations are often required, thereby creating considerable treatment burden for patients. Lanadelumab is a therapeutic antibody that binds to the plasma kallikrein protein (“pKal”) and may be used for treatment of hereditary angioedema as well as ocular indications, such as diabetic retinopathy and diabetic macular edema. Currently, lanadelumab, as approved for the treatment of hereditary angioedema, is dosed by subcutaneous injection every two weeks. There is a need for more effective treatments that reduce the treatment burden on patients suffering from hereditary angioedema, or ocular indications such as diabetic retinopathy and diabetic macular edema.

### 3. SUMMARY OF THE INVENTION

Therapeutic antibodies delivered by gene therapy have several advantages over injected or infused therapeutic antibodies that dissipate over time resulting in peak and trough levels. Sustained expression of the transgene product antibody, as opposed to injecting an antibody repeatedly, allows for a more consistent level of antibody to be present at the site of action, and is less risky and more convenient for patients, since fewer injections need to be made. Furthermore, antibodies expressed from transgenes are post-translationally modified in a different manner than those that are directly injected because of the different microenvironment present during and after translation. Without being bound by any particular theory, this results in antibodies that have different diffusion, bioactivity, distribution, affinity, pharmacokinetic, and immunogenicity characteristics, such that the antibodies delivered to the site of action are “biobetters” in comparison with directly injected antibodies. Accordingly, provided herein are compositions and methods for anti-pKal gene therapy, particularly recombinant AAV gene therapy, designed to target the liver or in alternate embodiments the muscle, or the liver and the muscle, and generate a depot of transgenes for expression of anti-pKal antibodies, particularly lanadelumab, or an antigen binding fragment thereof, that result in a therapeutic or prophylactic serum levels of the antibody within 20 days, 30 days, 40 days, 50 days, 60 days, or 90 days of administration of the rAAV composition. Serum levels include 1.5 to 35 μg/ml antibody for an anti-pKal antibody, particularly, lanadelumab or an antigen binding fragment thereof.

Compositions and methods are described for the systemic delivery of an anti pKal HuPTM mAb or an anti-pKal HuPTM antigen-binding fragment of a therapeutic mAb (for example, a fully human-glycosylated Fab (HuGlyFab) of a therapeutic mAb, to a patient (human subject) diagnosed with hereditary angioedema or other condition indicated for treatment with the therapeutic anti-pKal mAb. Such antigen-binding fragments of therapeutic mAbs include a Fab, F(ab′)₂, or scFv (single-chain variable fragment) (collectively referred to herein as “antigen-binding fragment”). “HuPTM Fab” as used herein may include other antigen binding fragments of a mAb. In an alternative embodiment, full-length mAbs can be used. Delivery may be advantageously accomplished via gene therapy—e.g., by administering a viral vector or other DNA expression construct encoding a therapeutic anti-pKal mAb or its antigen-binding fragment (or a hyperglycosylated derivative of either) diagnosed with a condition indicated for treatment with the therapeutic anti-pKal mAb—to create a permanent depot in liver, or in alternative embodiments, muscle, of the patient that continuously supplies the HuPTM mAb or antigen-binding fragment of the therapeutic mAb, e.g., a human-glycosylated transgene product, or peptide to the circulation of the subject where the mAb or antigen-binding fragment thereof or peptide exerts its therapeutic or prophylactive effect.

Provided are gene therapy vectors, particularly rAAV gene therapy vectors, which when administered to a human subject result in expression of an anti-pKal antibody to achieve a maximum or steady state serum concentration (for example, 20, 30, 40, 50, 60 or 90 days after administration) of 1.5 μg/ml to 35 μg/ml (or, 1.5 μg/ml to 15 μg/ml, or 5 μg/ml to 20 μg/ml, or 10 μg/ml to 35 μg/ml) anti-pKal antibody (including lanadelumab). In certain embodiments, the antibody binds to its target, for example, in an antibody binding assay (e.g. enzyme-linked immunosorbent assay (ELISA) binding assay or surface plasmon resonance (SPR)—based real-time kinetics assay), preferably in the picomolar or nanomolar range, and/or exhibits biological activity in an appropriate assay. Dosages include 1E11 to 1E14 vector genomes per kilogram body weight (vg/kg) administered, particularly, parenterally, including intravenously. Dosages result in sufficient copy number of viral genomes incorporated into liver cells, for example, from at least 10, 20, 50, 60 or 80 vector genome copies (or vector genomes, vg) per diploid genome (vg/dg) in liver tissue and up to 100, 150, 200, 500 or 100 vg/dg in liver tissue by 30, 60, 90 or 100 days or one year after administration. Dosages result in sufficient copy number of viral genomes incorporated into muscle or liver and muscle cells, for example, from at least 10, 20, 50, 60 or 80 vector genome copies (or vector genomes, vg) per diploid genome (vg/dg) in muscle or liver and muscle tissue and up to 100, 150, 200, 500 or 100 vg/dg in muscle or liver and muscle tissue by 30, 60, 90 or 100 days or one year after administration. In certain embodiments, the administration is a single administration. The dosage achieves the therapeutic or prophylactive serum levels of the anti-pKal antibody while minimizing or avoiding adverse effects such as transaminitis and/or the presence of anti-drug antibodies.

The recombinant vector used for delivering the transgene includes non-replicating recombinant adeno-associated virus vectors (“rAAV”). In embodiments, the AAV type has a tropism for liver and/or muscle cells, for example AAV8 subtype of AAV. However, other viral vectors may be used, including but not limited to lentiviral vectors; vaccinia viral vectors, or non-viral expression vectors referred to as “naked DNA” constructs. Expression of the transgene can be controlled by constitutive or tissue-specific expression control elements, particularly elements that are liver and/or muscle specific control elements (such as dual muscle-liver promoter elements), for example one or more elements of Table 1 or one or more lements of SEQ ID Nos 163-293 (liver enhancer elements).

In certain embodiments, the HuPTM mAb or HuPTM antigen-binding fragment encoded by the transgene can include, but is not limited to, a full-length or an antigen-binding fragment of a therapeutic antibody that binds to pKal, particularly lanadelumab, see, for example FIG. 3.

Gene therapy constructs for the therapeutic antibodies are designed such that both the heavy and light chains are expressed. The coding sequences for the heavy and light chains can be engineered in a single construct in which the heavy and light chains are separated by a cleavable linker or IRES so that separate heavy and light chain polypeptides are expressed. In particular embodiments, the linker is a Furin T2A linker (SEQ ID NOS: 103 or 104). In certain embodiments, the coding sequences encode for a Fab or F(ab′)₂ or an scFv, including an scFv-Fc construct. In certain embodiments the full length heavy and light chains of the antibody are expressed. In other embodiments, the constructs express an scFv in which the heavy and light chain variable domains are connected via a flexible, non-cleavable linker. In certain embodiments, the construct expresses, from the N-terminus, NH₂-V_{L}-linker-V_{H}—COOH or NH₂-V_{H}-linker-V_{L}—COOH. In certain embodiments, the scFv is linked to an Fc domain and the construct expresses, from the N-terminus, NH₂-V_{L}-linker-V_{H}-optionally a linker-Fc domain (including all or a portion of the hinge)-COOH or NH₂-V_{H}-linker-V_{L}-linker-Fc domain (including the hinge)-COOH.

In addition, antibodies expressed from transgenes in vivo are not likely to contain degradation products associated with antibodies produced by recombinant technologies, such as protein aggregation and protein oxidation. Aggregation is an issue associated with protein production and storage due to high protein concentration, surface interaction with manufacturing equipment and containers, and purification with certain buffer systems. These conditions, which promote aggregation, do not exist in transgene expression in gene therapy. Oxidation, such as methionine, tryptophan, and histidine oxidation, is also associated with protein production and storage, and is caused by stressed cell culture conditions, metal and air contact, and impurities in buffers and excipients. The proteins expressed from transgenes in vivo may also oxidize in a stressed condition. However, humans, and many other organisms, are equipped with an antioxidation defense system, which not only reduces the oxidation stress, but sometimes also repairs and/or reverses the oxidation. Thus, proteins produced in vivo are not likely to be in an oxidized form. Both aggregation and oxidation could affect the potency, pharmacokinetics (clearance), and immunogenicity.

The production of HuPTM mAb or HuPTM Fab in liver and/or muscle cells of the human subject should result in a “biobetter” molecule for the treatment of disease accomplished via gene therapy—e.g., by administering a viral vector or other DNA expression construct encoding a full-length HuPTM mAb or HuPTM Fab of a therapeutic mAb to a patient (human subject) diagnosed with a disease indication for that mAb, to create a permanent depot in the subject that continuously supplies the human-glycosylated, sulfated transgene product produced by the subject's transduced cells. The cDNA construct for the HuPTMmAb or HuPTM Fab should include a signal peptide that ensures proper co- and post-translational processing (glycosylation and protein sulfation) by the transduced human cells.

As an alternative, or an additional treatment to gene therapy, the full-length HuPTM mAb or HuPTM Fab can be produced in human cell lines by recombinant DNA technology, and the glycoprotein can be administered to patients.

Combination therapies involving systemic delivery of the full-length HuPTM anti-pKal mAb or HuPTM anti-pKal Fab to the patient accompanied by administration of other available treatments are encompassed by the methods provided herein. The additional treatments may be administered before, concurrently or subsequent to the gene therapy treatment. Such additional treatments can include but are not limited to co-therapy with the therapeutic mAb.

Also provided are methods of manufacturing the viral vectors, particularly the AAV based viral vectors. In specific embodiments, provided are methods of producing recombinant AAVs comprising culturing a host cell containing an artificial genome comprising a cis expression cassette flanked by AAV ITRs, wherein the cis expression cassette comprises a transgene encoding a therapeutic antibody operably linked to expression control elements that will control expression of the transgene in human cells; a trans expression cassette lacking AAV ITRs, wherein the trans expression cassette encodes an AAV rep and capsid protein operably linked to expression control elements that drive expression of the AAV rep and capsid proteins in the host cell in culture and supply the rep and cap proteins in trans; sufficient adenovirus helper functions to permit replication and packaging of the artificial genome by the AAV capsid proteins; and recovering recombinant AAV encapsidating the artificial genome from the cell culture.

The inventors found that intravenous administration of an AAV8-based vector comprising an optimized expression cassette containing a liver-specific promoter or a muscle-specific promoter or a dual liver-muscle specific promoter and a codon optimized and CpG depleted transgene with a modified furin-T2A processing signal results in dose-dependent and sustained serum antibody concentrations in non-human primates. Accordingly, provided are compositions comprising rAAV vectors which comprise an optimized expression cassette containing a liver-specific promoter, or a muscle specific promoter or a dual muscle- and liver-specific promoter and a codon optimized and CpG depleted transgene with a modified furin-T2A processing signal that express a transgene, for example HuPTMmAb or HuPTM Fab or heavy and light chains of an anti-pKal therapeutic antibody, including lanadelumab. Methods of administration and manufacture are also provided. The liver specific promoters can comprise ApoE.hAAT (SEQ ID NO:21) regulatory sequence, an LMTP6 promoter (SEQ ID NO:14), a LSPX1 promoter (SEQ ID NO:9), a LSPX2 promoter (SEQ ID NO:10), a LTP1 promoter (SEQ ID NO:11), a LTP2 (SEQ ID NO:12) promoter, a liver specific cis-regulating element selected from sequences having SEQ ID Nos: 163-293), a CRE.hAAT, or a LTP3 (SEQ ID NO: 13) promoter.

### 3.1 Illustrative Embodiments

### Compositions of Matter

1. A pharmaceutical composition for treating hereditary angioedema, diabetic retinopathy or diabetic macular edema in a human subject in need thereof, comprising an adeno-associated virus (AAV) vector having:
(a) a viral capsid that has a tropism for liver and/or muscle cells; and
(b) an artificial genome comprising an expression cassette flanked by AAV inverted terminal repeats (ITRs), wherein the expression cassette comprises a transgene encoding a heavy chain variable region, a light chain variable region and an Fc domain of a substantially full-length or full-length anti-pKal mAb or an antigen binding fragment thereof, operably linked to one or more regulatory sequences that promote expression of the transgene in human liver and/or muscle cells;
wherein said AAV vector is formulated for administration to said human subject such that within 20 days after said administration, the anti-pKal mAb is present at a serum concentration of 1.5 μg/ml to 35 μg/ml in said human subject.

2. The pharmaceutical composition of paragraph 1 wherein the viral capsid is at least 95% identical to the amino acid sequence of an AAV3B, AAV5, AAV7 (SEQ ID NO:1), AAV8 (SEQ ID NO: 2), AAV9 (SEQ ID NO:3), AAVrh10 (SEQ ID NO:4), AAVrh46 (SEQ ID NO:5), AAVrh73 (SEQ ID NO:6), AAVS3 (SEQ ID NO:8), AAV-LK03 (SEQ ID NO:7), AAVrh8, AAV64R1, or AAVhu37.

3. The pharmaceutical composition of any of paragraphs 1 or 2, wherein the AAV capsid is AAV8 or AAVS3.

4. The pharmaceutical composition of any of paragraphs 1 to 3, wherein the regulatory sequence includes a regulatory sequence from Table 1.

5. The pharmaceutical composition of any of paragraphs 1 to 4, wherein the regulatory sequence comprises an ApoE.hAAT (SEQ ID NO:21) regulatory sequence, a LSPX1 promoter (SEQ ID NO: 9), a LSPX2 promoter (SEQ ID NO:10), a LTP1 promoter (SEQ ID NO:11), a LTP2 (SEQ ID NO: 12) promoter, an LMTP6 promoter (SEQ ID NO: 14), a CRE selected from SEQ ID Nos: 163-293, a CRE.hAAT, a LTP3 (SEQ ID NO:13) promoter or a dual liver- and muscle-specific promoter.

6. The pharmaceutical composition of any of paragraphs 1 to 5, wherein the transgene comprises a Furin/2A linker between the nucleotide sequences coding for the heavy and light chains of said mAb.

7. The pharmaceutical composition of paragraph 6, wherein said Furin 2A linker is a Furin/T2A linker having the amino acid sequence RKRR(GSG)APVKQTLNFDLLKLAGDVESNPGP (SEQ ID NOS: 103 or 104).

8. The pharmaceutical composition of any of paragraphs 1 to 7, wherein the transgene encodes a signal sequence at the N-terminus of the heavy chain and the light chain of said antigen-binding fragment, or at the N-terminus of the heavy chain variable region or the light chain variable region that directs secretion and post translational modification in said human liver and/or muscle cells.

9. The pharmaceutical composition of paragraph 8, wherein said signal sequence is MYRMQLLLLIALSLALVINS (SEQ ID NO: 50) or a signal sequence from Table 2.

10. The pharmaceutical composition of any of paragraphs 1 to 9, wherein transgene has the structure: signal sequence-Heavy chain-Furin site-2A site-signal sequence-Light chain-PolyA.

11. The pharmaceutical composition of any of paragraphs 1 to 10 which is administered at a dosage of 1E11 to 1E14 vg/kg.

12. The pharmaceutical composition of any of paragraphs 1 to 11 wherein said administration results in a 10-100 vector genome per decagram of liver or muscle tissue at 100 days after administration.

13. The pharmaceutical composition of any of paragraphs 1 to 12, wherein the anti-pKal antibody is lanadelumab or an antigen binding fragment thereof, such as an anti-pKal antibody comprising a lanadelumab light chain variable region SEQ ID NO: 318) and a lanadelumab heavy chain variable region (SEQ ID NO: 314).

14. The pharmaceutical composition of any of paragraphs 1 to 13 wherein said transgene has the nucleotide sequence of any one of SEQ ID NOs: 239 to 247 (TABLE 7).

15. The pharmaceutical composition of any of paragraphs 1 to 5, 8 to 9, or 11 to 13, wherein the anti-pKal antibody is an scFv or an scFv-Fc.

16. The pharmaceutical composition of paragraph 15, wherein the transgene encodes an scFv-Fc having an amino acid sequence of SEQ ID NO: 324 or 393.

17. The pharmaceutical composition of claim 15 or 16, wherein the transgene comprises a nucleotide sequence of any one of SEQ ID Nos: 308, 325, 332 or 333.

18. The pharmaceutical composition of any of paragraphs 1 to 17, wherein the anti-pKal antibody plasma levels are maintained for at least 3 months.

19. The pharmaceutical composition of paragraphs 1 to 18 wherein the anti-pKal antibody secreted into the plasma exhibits greater a greater than at least 40%, 45%, 50%, 55%, 60%, 65% or 70 reduction in pKal activity as measured by a kinetic enzymatic functional assay.

20. The pharmaceutical composition of paragraph 18 wherein the activity of the lanadelumab antibody is measured at 2 weeks, 3 weeks, 4 weeks, 5 weeks, 6 weeks, 7 weeks, 8 weeks, 9 weeks, 10 weeks, 11 weeks or 12 weeks after said administration.

21. A composition comprising an adeno-associated virus (AAV) vector having:
a. a viral AAV capsid, that is optionally at least 95% identical to the amino acid sequence of an AAV3B, AAV5, AAV7 (SEQ ID NO:1), AAV8 (SEQ ID NO:2), AAV9 (SEQ ID NO:3), AAVrh10 (SEQ ID NO:4), AAVrh46 (SEQ ID NO:5), AAVrh73 (SEQ ID NO:6), AAVS3 (SEQ ID NO: 8), AAV-LK03 (SEQ ID NO:7), AAVrh8, AAV64R1, or AAVhu37; and
b. an artificial genome comprising an expression cassette flanked by AAV inverted terminal repeats (ITRs), wherein the expression cassette comprises a transgene encoding a transgene encoding a heavy chain variable region, a light chain variable region and an Fc domain of a substantially full-length or full-length anti-pKal mAb or an antigen binding fragment thereof, operably linked to one or more regulatory sequences that promote expression of the transgene in human liver and/or muscle cells;
c. wherein the transgene encodes a signal sequence at the N-terminus of the heavy chain and the light chain of said mAb that directs secretion and post translational modification of said mAb in liver and/or muscle cells.

22. The composition of paragraph 21, wherein the anti-pKal antibody is lanadelumab or an antigen binding fragment thereof.

23. The composition of paragraphs 21 or 22 wherein said transgene has the nucleotide sequence of any one of SEQ ID NOs: 239 to 247 (TABLE 7).

24. The composition of any of paragraphs 21 to 23, wherein the transgene comprises a Furin/2A linker between the nucleotide sequences coding for the heavy and light chains of said mAb.

25. The composition of paragraph 234, wherein the nucleic acid encoding a Furin 2A linker is incorporated into the expression cassette in between the nucleotide sequences encoding the heavy and light chain sequences, resulting in a construct with the structure: Signal sequence-Heavy chain-Furin site-2A site-Signal sequence-Light chain-PolyA.

26. The composition of paragraphs 21 to 25, wherein said Furin 2A linker is a Furin/T2A linker having the amino acid sequence RKRR(GSG)APVKQTLNFDLLKLAGDVESNPGP (SEQ ID NOS: 103 or 104).

27. The composition of paragraph 21 or 22 wherein the transgene encodes an scFv or scFv-Fc.

28. The composition of paragraph 27, wherein the scFv or scFv-Fc has the heavy chain variable domain and the light chain variable domain of lanadelumab.

29. The composition of paragraph 28, wherein the transgene encodes an scFv-Fc having an amino acid sequence of SEQ ID NO: 324 or 393.

30. The composition of paragraph 28 or 29 which comprises a nucleotide sequence of any one of SEQ ID Nos: 308, 325, 332 or 333.

31. The composition of any one of paragraphs 22 to 30, wherein said signal sequence is MYRMQLLLLIALSLALVINS (SEQ ID NO:50) or a signal sequence from Tables 2 or 3.

### Method of Treatment

32. A method of treating hereditary angioedema in a human subject in need thereof, comprising intravenously or intramuscularly administering to the subject a dose of a composition comprising a recombinant AAV comprising a transgene encoding lanadelumab or an antigen binding protein comprising a heavy chain variable region, a light chain variable region and an Fc domain of lanadelumab or an antigen binding fragment thereof, operably linked to one or more regulatory sequences that control expression of the transgene in liver and/or muscle cells, in an amount sufficient to result in expression from the transgene and secretion of lanadelumab, or the antigen binding protein or the antigen binding fragment thereof into the bloodstream of the human subject to produce lanadelumab or the antigen binding protein or antigen binding fragment thereof, plasma levels of at least 1.5 μg/ml to 35 μg/ml lanadelumab or the antigen binding protein or antigen binding fragment thereof, in said subject, or of at least 5 μg/ml to 35 μg/ml lanadelumab or antigen binding protein or antigen binding fragment thereof, or of at least 1.5 μg/ml to 20 μg/ml lanadelumab or antigen binding protein or antigen binding fragment thereof, of at least 1.5 μg/ml to 10 μg/ml lanadelumab or antigen binding protein or antigen binding fragment thereof, or of at least 5 μg/ml to 20 μg/ml lanadelumab or antigen binding protein or antigen binding fragment thereof, within at least 20, 30, 40 or 60 days of said administering.

33. A method of treating diabetic retinopathy or diabetic macular edema in a human subject in need thereof, comprising intravenously or intramuscularly administering to the subject a dose of a composition comprising a recombinant AAV comprising a transgene encoding lanadelumab or an antigen binding protein comprising a heavy chain variable region, a light chain variable region and an Fc domain of lanadelumab or an antigen binding fragment thereof, operably linked to one or more regulatory sequences that control expression of the transgene in liver and/or muscle cells, in an amount sufficient to result in expression from the transgene and secretion of lanadelumab, or the antigen binding protein or the antigen binding fragment thereof into the bloodstream of the human subject to produce lanadelumab or the antigen binding protein or antigen binding fragment thereof, plasma levels of at least 1.5 μg/ml to 35 μg/ml lanadelumab or the antigen binding protein or antigen binding fragment thereof, in said subject, or of at least 5 μg/ml to 35 μg/ml lanadelumab or antigen binding protein or antigen binding fragment thereof, or of at least 1.5 μg/ml to 20 μg/ml lanadelumab or antigen binding protein or antigen binding fragment thereof, of at least 1.5 μg/ml to 10 μg/ml lanadelumab or antigen binding protein or antigen binding fragment thereof, or of at least 5 μg/ml to 20 μg/ml lanadelumab or antigen binding protein or antigen binding fragment thereof, within at least 20, 30, 40 or 60 days of said administering.

34. The method of paragraph 32 or 33 wherein the transgene encodes a full length or substantially full length lanadelumab.

35. The method of any of paragraphs 32 to 34, wherein the transgene comprises a Furin/2A linker between the nucleotide sequences coding for the heavy and light chains of said mAb.

36. The method of paragraph 35, wherein said Furin 2A linker is a Furin/T2A linker having the amino acid sequence RKRR(GSG)APVKQTLNFDLLKLAGDVESNPGP (SEQ ID NOS: 103 or 104).

37. The method of paragraphs 32 to 35 wherein said transgene has the nucleotide sequence of any one of SEQ ID NOs: 239-247 (TABLE 7).

38. The method of paragraph 32 or 33 wherein the transgene encodes an scFv or scFv-Fc having the heavy chain variable domain and light chain variable domain of lanadelumab.

39. The method of paragraph 32, 33 or 37 wherein the transgene encodes an scFv-Fc having an amino acid sequence of SEQ ID NO: 324 or 393 or has a nucleotide sequence of any one of SEQ ID Nos: 308, 325, 332 or 333.

40. The method of paragraphs 32 to 39 wherein the viral capsid is at least 95% identical to the amino acid sequence of an AAV3B, AAV5, AAV7 (SEQ ID NO:1), AAV8 capsid (SEQ ID NO:2), AAV9 (SEQ ID NO:3), AAVrh10 (SEQ ID NO:4), AAVrh46 capsid (SEQ ID NO:5), AAVrh73 (SEQ ID NO:6), AAVS3 (SEQ ID NO:8), AAV-LK03 (SEQ ID NO:7), AAVrh8, AAV64R1, or AAVhu37.

41. The method of any of paragraphs 32 to 39, wherein the AAV capsid is AAV8 or AAVS3.

42. The method of any of paragraphs 32 to 41, wherein the regulatory sequence includes a regulatory sequence from Table 1.

43. The method of any of paragraphs 32 to 43, wherein the regulator sequence comprises an ApoE.hAAT (SEQ ID NO:21) regulatory sequence, a LMTP6 promoter (SEQ ID NO; 14), a LSPX1 promoter (SEQ ID NO:9), a LSPX2 promoter (SEQ ID NO:10), a LTP1 promoter (SEQ ID NO: 11), a LTP2 (SEQ ID NO:12) promoter, a CRE selected from SEQ ID Nos: 163-293, a CRE.hAAT, or a LTP3 (SEQ ID NO: 13) promoter.

44. The method of any of paragraphs 32 to 44, wherein the transgene encodes a signal sequence at the N-terminus of the heavy chain and the light chain of said lanadelumab or at the N-terminus of an scFv or scFv-Fc that directs secretion and post translational modification in said human liver and/or muscle cells.

45. The method of paragraph 44, wherein said signal sequence is MYRMQLLLLIALSLALVINS (SEQ ID NO:50) or a signal sequence from Tables 2 or 3.

46. The method of any of paragraphs 32 to 37 and 40 to 45, wherein transgene has the structure: Signal sequence-Heavy chain-Furin site-2A site-Signal sequence-Light chain-PolyA.

47. The method of any of paragraphs 32 to 46, wherein the mAb is a hyperglycosylated mutant or wherein the Fc polypeptide of the mAb is glycosylated or aglycosylated.

48. The method of any of paragraphs 32 to 47 wherein the mAb contains an alpha 2,6-sialylated glycan.

49. The method of any of paragraphs 32 to 48 wherein the mAb is glycosylated but does not contain detectable NeuGc and/or α-Gal.

50. The method of any of paragraphs 32 to 49 wherein the mAb contains a tyrosine sulfation.

51. The method of any of paragraphs 32 to 50 in which production of said HuPTM form of said mAb or antigen-binding fragment thereof is confirmed by transducing human liver and/or muscle cells in culture with said recombinant nucleotide expression vector and expressing said mAb or antigen-binding fragment thereof.

52. The method of any of paragraphs 32 to 51 wherein the vector is administered at a dosage of 1E11 to 1E14 vg/kg.

53. The method of any of paragraphs 32 to 52 or the composition of any of paragraphs 1 to 20, wherein said administration results in a vector genome concentration of 10-100 vg/dg as measured in the liver or muscle at 100 days after administration.

54. The method of any of paragraphs 32 to 53 or the composition of any of paragraphs 1 to 20, wherein said administration achieves within 20, 30, 40, 50 or 60 days of said administration a Cₘᵢₙ serum level of at least 1.0 μg/ml, 1.1 μg/ml, 1.2 μg/ml, 1.3 μg/ml, 1.4 μg/ml, 1.5 μg/ml, 1.6 μg/ml, 1.7 μg/ml, 1.8 μg/ml or 1.9 μg/ml antibody or antigen binding fragment but no more than 200 μg/ml, 300 μg/ml or 400 μg/ml antibody or antigen binding fragment.

55. The method of any of paragraphs 32 to 54, or the composition of any one of paragraphs 1 to 20, wherein the anti-pKal antibody, or antigen binding fragment, plasma levels are maintained for at least 3 months.

56. The method of any of paragraphs 32 to 55, or the composition of any one of paragraphs 1 to 20, wherein the anti-pKal antibody or antigen binding fragment secreted into the plasma exhibits a greater than at least 40%, 45%, 50%, 55%, 60%, 65% or 70 reduction in pKal activity as measured by a kinetic enzymatic functional assay.

57. The method or composition of paragraph 56 wherein the activity of the lanadelumab antibody is measured at 2 weeks, 3 weeks, 4 weeks, 5 weeks, 6 weeks, 7 weeks, 8 weeks, 9 weeks, 10 weeks, 11 weeks or 12 weeks after said administration.

### Method of Manufacture

58. A method of producing recombinant AAVs comprising:
(a) culturing a host cell containing:
   (i) an artificial genome comprising a cis expression cassette flanked by AAV ITRs, wherein the cis expression cassette comprises comprising a transgene encoding a substantially full-length or full-length anti-pKal mAb, or scFv or scFv-Fc having the heavy and light chain variable domains thereof, or antigen binding fragment thereof, operably linked to one or more regulatory sequences that promote expression of the transgene in human liver and/or muscle cells;
   (ii) a trans expression cassette lacking AAV ITRs, wherein the trans expression cassette encodes an AAV rep and an AAV capsid protein operably linked to expression control elements that drive expression of the AAV rep and the AAV capsid protein in the host cell in culture and supply the AAV rep and the AAV capsid protein in trans, wherein the capsid has liver and/or muscle tropism;
   (iii) sufficient adenovirus helper functions to permit replication and packaging of the artificial genome by the AAV capsid protein; and
(b) recovering recombinant AAV encapsidating the artificial genome from the cell culture.

59. The method of paragraph 58, wherein the transgene encodes a substantially full-length or full-length mAb or antigen binding fragment that comprises the heavy and light chain variable domains of lanadelumab.8, wherein the AAV capsid protein is an AAV8, AAVrh46, AAVrh73, AAVS3, or AAV-LK03 capsid protein.

60. A host cell containing:
a. an artificial genome comprising a cis expression cassette flanked by AAV ITRs, wherein the cis expression cassette comprises comprising a transgene encoding a substantially full-length or full-length anti-pKal mAb, or antigen binding fragment thereof, or scFv or scFv-Fc having the heavy and light chain variable domains thereof operably linked to one or more regulatory sequences that promote expression of the transgene in human liver and/or muscle cells;
b. a trans expression cassette lacking AAV ITRs, wherein the trans expression cassette encodes an AAV rep and an AAV capsid protein operably linked to expression control elements that drive expression of the AAV rep and the AAV capsid protein in the host cell in culture and supply the AAV rep and the AAV capsid protein in trans, wherein the capsid has liver and/or muscle tropism;
c. sufficient adenovirus helper functions to permit replication and packaging of the artificial genome by the AAV capsid protein.

61. The host cell of paragraph 60 wherein the transgene encodes a substantially full-length or full-length mAb or antigen binding fragment that comprises the heavy and light chain variable domains of lanadelumab.

62. The host cell of paragraphs 60 or 61, wherein the AAV capsid protein is an AAV8, AAVrh46, AAVrh73, AAVS3, or AAV-LK03 capsid protein.

### Composition of Matter

63. A pharmaceutical composition comprising an adeno-associated virus (AAV) vector having:
(a) a viral capsid that has a tropism for liver and/or muscle cells; and
(b) an artificial genome comprising an expression cassette flanked by AAV inverted terminal repeats (ITRs), wherein the expression cassette comprises a transgene, operably linked to one or more regulatory sequences that promote expression of the transgene in human liver and/or muscle cells;
wherein the one or more regulatory elements are selected from an ApoE.hAAT (SEQ ID NO: 21) regulatory sequence, a LSPX1 promoter (SEQ ID NO:9), a LSPX2 promoter (SEQ ID NO: 10), a LTP1 promoter (SEQ ID NO:11), a LTP2 (SEQ ID NO:12) promoter, a LMTP6 promoter (SEQ ID NO: 14), a CRE selected from SEQ ID Nos: 163-293, a ApoE.hAAT, a LTP3 (SEQ ID NO:13) promoter or a dual liver- and muscle-specific promoter.
wherein said AAV vector is formulated for administration to said human subject.

64. The pharmaceutical composition of paragraph 63 wherein the viral capsid is at least 95% identical to the amino acid sequence of an AAV3B, AAV5, AAV7 (SEQ ID NO:1), AAV8 (SEQ ID NO: 2), AAV9 (SEQ ID NO:3), AAVrh10 (SEQ ID NO:4), AAVrh46 (SEQ ID NO:5), AAVrh73 (SEQ ID NO:6), AAVS3 (SEQ ID NO:8), AAV-LK03 (SEQ ID NO:7), AAVrh8, AAV64R1, or AAVhu37.

65. The pharmaceutical composition of any of paragraphs 63 or 64, wherein the AAV capsid is AAV8 or AAVS3.

66. A method of treatment comprising using the composition of any of paragraphs 63 to 65.

### 4. BRIEF DESCRIPTION OF THE DRAWINGS

FIG. 1. A schematic of an rAAV vector genome construct containing an expression cassette encoding the heavy and light chains of a therapeutic mAb separated by a Furin-2A linker, operably linked to a liver-specific enhancer and/or promoter, controlled by expression elements, flanked by the AAV ITRs.

FIGS. 2A and 2B Schematics of an rAAV vector genome construct containing an expression cassette encoding the heavy and light chains of a therapeutic mAb separated by a Furin-T2A linker, controlled by expression elements, flanked by the AAV ITRs. The transgene can comprise nucleotide sequences encoding the heavy and light chains of the Fab portion or the full-length heavy (CH1 plus hinge) and light chains with Fc regions. FIG. 2A depicts a constract with an ApoE enhancer as part of the promoter and FIG. 2B depicts a construct with a liver-specific cis-regulating element (CRE) as part of the promoter.

FIG. 3. The amino acid sequence of a transgene construct for the Fab region of lanadelumab, a therapeutic antibody to plasma kallikrein (pKal). Glycosylation sites are boldface. Glutamine glycosylation sites; asparaginal (N) glycosylation sites, non-consensus asparaginal (N) glycosylation sites; and tyrosine-O-sulfation sites (italics) are as indicated in the legend. Complementarity-determining regions (CDR) are underscored. The hinge region is highlighted in grey.

FIG. 4. Clustal Multiple Sequence Alignment of various capsids with liver and/or muscle tropism. Amino acid substitutions (shown in bold in the bottom rows) can be made to AAV8 capsids by “recruiting” amino acid residues from the corresponding position of other aligned AAV capsids. Sequence shown in gray=hypervariable regions. The amino acid sequences of the AAV capsids are assigned SEQ ID NOs as follows: AAV2 is SEQ ID NO:334; AAV7 is SEQ ID NO:1; AAV8 is SEQ ID NO:2; AAV9 is SEQ ID NO:3; AAVrh10 is SEQ ID NO:4; AAVrh46 (SEQ ID NO:5), AAVrh73 (SEQ ID NO:6), AAVS3 (SEQ ID NO:8), and AAV-LK03 (SEQ ID NO:7).

FIG. 5. Glycans that can be attached to HuGlyFab regions of full length mAbs or the antigen-binding domains. (Adapted from Bondt et al., 2014, Mol & Cell Proteomics 13.1:3029-3039).

FIG. 6. Clustal Multiple Sequence Alignment of constant heavy chain regions (CH2 and CH3) of IgG1 (SEQ ID NO: 141), IgG2 (SEQ ID NO: 142), and IgG4 (SEQ ID NO: 143). The hinge region, from residue 219 to residue 230 of the heavy chain, is shown in italics. The numbering of the amino acids is in EU-format.

FIGS. 7A-D. A. Schematic showing the genome configuration of recombinant AAV8 and AAV9 vectors for expression of lanadelumab. The expression cassette utilizes the CAG promoter (SEQ ID NO:36) to drive expression of a human antibody that binds to and inhibits for example, plasma kallikrein (pKal). A mutant IL2 signal sequence (mIL2, SEQ ID NO:50) targets the heavy and light chains for secretion and the furin-F2A sequence (SEQ ID NO:106) drives the cleavage of the polyprotein into heavy and light chain components. B. Transfection titration comparing CAG.L01 (SEQ ID NO:151; containing lanadelumab sequence L01 (SEQ ID NO:148)) and CAG.L02 (SEQ ID NO: 153; containing lanadelumab sequence L02 (SEQ ID NO:149) proviral plasmid constructions. Top panels demonstrate reporter transgene (eGFP) expression following transfection of different plasmid quantities (4 μg-nontransfected). Bottom left panel depicts lanadelumab expression in the cell lysate while the bottom right panel detects plasmid expressed lanadelumab secreted into the cell supernatant. C. Transfection titration comparing CAG.L02 and CAG.L03 proviral plasmid constructions. Panels depict different exposure lengths (30 seconds or 60 seconds) of expressed lanadelumab from CAG.L02 or CAG.L03 constructs secreted into the cell supernatant. D. Transfection titration comparing Lanadelumab Fab proviral plasmid constructions. Figure depicts levels of Lanadelumab Fab following transfection of different plasmid quantities. L01 construct (CAG.L01: SEQ ID NO:151) is driven by the CB promoter, while L02 (CAG. L02: SEQ ID NO:153) is driven by the CAG promoter (SEQ ID NO:36).

FIG. 8. The indicated AAV9 and AAV8 vectors (n=5 per group) were administered to NGS mice via either intravenous (IV) or intramuscular (IM) routes. IV administrations were into the tail vein and IM administrations were bilateral into the gastrocnemius muscles. Mice treated with vehicle were included as controls. Seven weeks post administration mice were sacrificed, and serum human antibody levels were determined by ELISA.

FIG. 9. A time course of antibody expression (lanadelumab serum levels) in NGS mice post-AAV9 administration (n=5 per group) is shown. AAV9 vectors (2e11 gc) were injected either IV or IM and serum antibody levels were determined by ELISA at day 7 (D7), day 21 (D21), day 35 (D35), and day 49 (D49).

FIG. 10 depicts the expression of the monoclonal antibody lanadelumab (Mab1) in C2C12 muscle cells upon transduction of the cells with different cis plasmids expressing lanadelumab under the control of different regulatory elements: CAG (SEQ ID NO: 128), LMTP6 (SEQ ID NO:14), and ApoE.hAAT (SEQ ID NO:21). For detection of antibody protein, following transduction, the cells were treated with FITC conjugated anti-Fc (IgG) antibody. DAPI staining is shown to confirm confluency and viability of the cells under all conditions tested.

FIGS. 11A and B. A Serum expression levels (μg/ml) of lanadelumab upon intravenous injection of C/57BL6 mice with 2.5×10¹² vg/kg of AAV8 vectors encoding a lanadelumab regulated by different liver-specific, liver-tandem and liver-muscle regulatory elements (see Table 1). CAG (SEQ ID NO:36) and TBG (SEQ ID NO:40) promoters were used as controls. Data from the blood draw at 1, 3, 5 and 7 weeks post injection are shown. LSPX1, liver-specific promoter 1 (SEQ ID NO: 9); LSXP2, liver-specific promoter 2 (SEQ ID NO:10); LTP1, liver-specific tandem promoter 1 (SEQ ID NO:11); LMTP6, liver and muscle dual-specific tandem promoter 6 (SEQ ID NO:14). Protein expression levels were quantified by ELISA from biweekly serum collections. N=5 mice per vector. Numbers on x-axis represent the weeks post vector administration. Data represent mean+SEM. B. Quantification of viral genomes in liver. C57Bl/6 mice were administrated intravenously with AAV8 vectors driven by different liver-specific promoters at equivalent doses (2.5×10¹² vg/kg). N=5 mice per group. Vector DNA was analyzed by ddPCR in mouse liver samples collected at 49 days post vector administration. Data represent mean+SEM.

FIGS. 12A and 12B. A. Route of administration and dose selection in Wistar rats. AAV8 vectors encoding vectorized lanadelumab driven by CAG promoters were injected intramuscularly at 1×10¹³ vg/kg (body weight) or intravenously at 1×10¹³ vg/kg and 1×10¹⁴ vg/kg into SD rats. Protein expression was quantified by ELISA from serum collected every three to seven days. N=3 rats per vector. Data represent mean+SEM. * indicates p<0.05, ** indicates p<0.01 with Welch's t test. B. AAV8 vectors encoding vectorized lanadelumab driven by CAG (SEQ ID NO:36) or ApoE.hAAT (SEQ ID NO:21) promoters were injected intravenously at 5×10¹³ vg/kg into Wistar and SD rats. Protein expression was quantified by ELISA from weekly serum collection. N=3 rats per vector. Data represent mean+SEM. P value: *, p<0.05; **, p<0.01. Serum antibody concentrations (mean and SEM) in animals of each group at each time point are presented in the table.

FIGS. 13A-13D. A. Serum anti-kallikrein (pKal) (lanadelumab) antibody concentration following AAV8 delivery. Animals received bilateral injections of 5×10¹⁰ vg/kg into the GA muscle. Serum was collected biweekly and vectorized antibody concentration was quantified with ELISA. B. Vector genome quantification from relevant tissues with digital droplet PCR (ddPCR). C. Comparison of vector gene expression from liver. Data represent relative fold gene expression as quantified by the ΔΔCT method. D. Comparison of AAV transgene expression from tissues using digital droplet PCR (ddPCR). Anti-pKal antibody mRNA copies were normalized to GAPDH mRNA copies across tissues. Data are represented as mean±SEM. Statistical significance was determined using a one-way ANOVA followed by Tukey's HSD post-test. * P<0.05, ** P<0.01.

FIG. 14: Antibody concentrations in the serum of wild type mice treated with AAV8.Lanadelumab vectors produced with different BV/Sf9 production systems compared to an HEK system. C57BL/6 mice were intravenously injected with vectors at a dose of 2.5×10¹² vg/kg.

FIGS. 15A-15F. A and B show the pKal titration curve and signal-to-noise ratios for indicated pKal concentrations. C. Two pKal concentrations (6.25 nM and 12.5 nM) were used to measure the suppressive range of lanadelumab (compared to non-specific human IgG control antibody) in an antibody-dose response. C57BL/6 mice (n=5) were administered 5×10¹⁰ vector genomes (vg) (2.5×10¹² vg/kg) of ApoE.hAAT.L02.AAV8 per mouse intravenously. Shown are the compiled enzyme activities and percent reductions in pKal activity for both mouse groups D and E. The slopes of enzymatic progressive activity curves and an AMC standard were used to calculate specific pKal enzymatic activity, where significantly less activity was recorded at day 49 compared to day −7. F. The percent reduction in enzymatic activity was calculated as day 49 activity divided by that of day −7. Vectorized anti-pKal antibody-containing IgG significantly reduced pKal activity. All results are a compilation of 2-5 mice per group. To determine significance of differences, Student's t-tests were used (paired, two-tailed), where *p<0.05, ** p<0.01, *** p<0.001.

FIGS. 16A-16L. Quantification of mouse paw volumes and paw swellings in carrageenan-induced paw edema mice treated with test articles. Bar charts show the paw volumes (A, C, E, G, I, and K) measured at 2 (A), 4 (C), 6 (E), 8 (G), 24 (I) and 48 (K) hours after carrageenan injection in C57BL/6 mice. Paw swelling difference (B, D, F, H, J, and L) was evaluated by calculating the difference of paw volumes measured at each time point and the baseline. N=10 mice per group. Data analysis was done with One-way ANOVA with Dunnett's post-hoc test for multiple comparisons. Data represent mean+S.DEM. P values: *, p<0.05; **, p<0.01; ***, p<0.001; ****, p<0.0001.

FIGS. 17A and 17B: Time course of mouse paw volumes measured in carrageenan-induced paw edema mice treated with test articles. Mouse paw volumes were measured before (baseline) and at different time points after 0.7% (A) or 1% (B) carrageenan injection. N=10 mice per group. Data represent mean±SEM.

FIG. 18: Time course of anti-pKal antibody concentrations (μg/mL) measured in naïve cynomolgus monkeys injected with increasing doses (1E12, 1E13, or 1E14 gc/kg) of AAV8.ApoE.hAAT.Lan vector. Serum levels were measured before (baseline) and at different time points after injection. N=1-3 animals per group. Data represent mean±SEM.

FIG. 19: A schematic of an rAAV vector genome construct containing an expression cassette encoding eGFP+10-basepair barcode, operably linked to a liver-specific CRE selected from SEQ ID Nos: 163-293 and a hAAT promoter, controlled by expression elements, flanked by the AAV ITRs.

FIG. 20: Lanadelumab scFv-Fc cis plasmid constructs.

FIGS. 21A and 21B: Determination of ligand binding parameters for human kallikrein for A. full length anti-pKal antibodies and B. scFv-Fc constructs VH-VL-Fc (SEQ ID NO: 324) and VL-VH-Fc (SEQ ID NO: 393).

FIG. 22: Relative production levels of various Lanadelumab scFv-Fc constructs in supernatant and cell lysates as determined by ELISA assay with human kallikrein.

FIGS. 23A and 23B: vector copy number/ug gDNA and LAN transcripts/ug RNA in the left lateral lobe of the liver from treated mice. A) and B) are the same graph just on different scales.

FIGS. 24A and 24B: (A) LAN antibody (or scFv-Fc) levels in serum at 14 days and 28 days post infection after administration of vehicle and AAV8-ApoEhAAT.HL-ScFv-Fc, AAV8-LMTP6-HL-ScFv-Fc, AAV8-ApoEhAAT-LH-ScFv-Fc, AAV8-LMTP6-LH-ScFv-Fc, AAV8-LMTP6-LANA, AAV9-LMTP6-LANA contructs; (B) LAN antibody levels in serum after iv administration of 1×10¹² GC.kg, 1×10¹³ GC/kg, or 1×10¹⁴ GC/kg of AAV8-ApoEhAAT-LANA.

### 5. DETAILED DESCRIPTION OF THE INVENTION

Compositions and methods are described for the systemic delivery: of a fully human post-translationally modified (HuPTM) therapeutic monoclonal antibody (mAb) or a HuPTM antigen-binding fragment of a therapeutic anti-pKal mAb (for example, a fully human-glycosylated Fab (HuGlyFab) of a therapeutic mAb) to a patient (human subject) diagnosed with a hereditary angioedema or other indication indicated for treatment with the therapeutic mAb. Delivery may be advantageously accomplished via gene therapy—e.g., by administering a viral vector or other DNA expression construct encoding a therapeutic mAb or its antigen-binding fragment (or a hyperglycosylated derivative of either) to a patient (human subject) diagnosed with a condition indicated for treatment with the therapeutic mAb—to create a permanent depot in a tissue or organ of the patient, particularly liver and/or muscle that continuously supplies the HuPTM mAb or antigen-binding fragment of the therapeutic mAb, e.g., a human-glycosylated transgene product, into the circulation of the subject to where the mAb or antigen-binding fragment there of exerts its therapeutic effect.

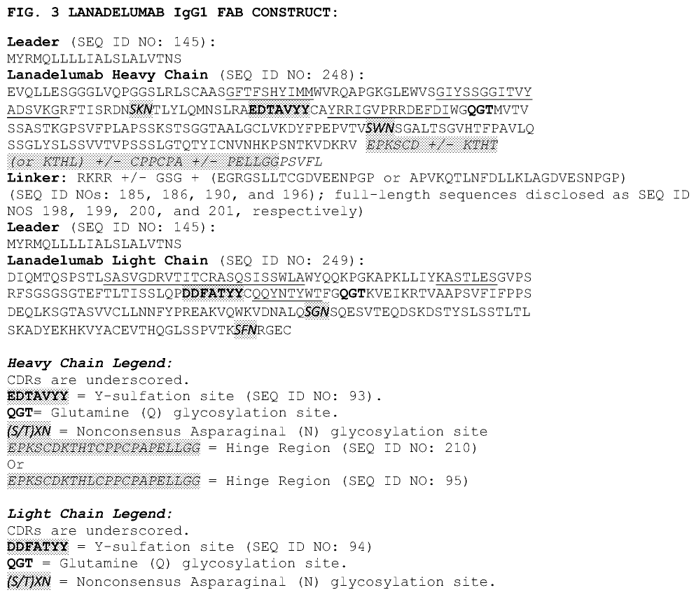

In certain embodiments, the HuPTM mAb or HuPTM antigen-binding fragment encoded by the transgene, but it not limited to, is a full-length or an antigen-binding fragment of a HuPTM mAb or HuPTM that binds pKal, particularly lanadelumab (see FIG. 3 for the heavy and light chain sequences of the Fab portion of lanadelumab) or an scFv-Fc having the heavy and light chain variable domains and Fc domain of lanadelumab (for example, VH-VL-Fc or VL-VH-Fc).

The compositions and methods provided herein systemically deliver anti-pKal antibodies, particularly, lanadelumab, from a depot of viral genomes, for example, in the subject's liver (or muscle) at a serum level that is therapeutically or prophylactically effective to treat or ameliorate the symptoms of hereditary angioedema or other indication that may be treated with an anti-pKal antibody. Identified herein are viral vectors for delivery of transgenes encoding the therapeutic anti-pKal antibodies to cells in the human subject, including, in embodiments, liver cells and/or muscle cells, and regulatory elements operably linked to the nucleotide sequence encoding the heavy and light chains of the anti-pKal antibody that promote the expression of the antibody in the cells, in embodiments, in the liver cells and/or in muscle cells. Such regulatory elements, including liver specific regulatory elements, muscle specific regulatory elements and dual liver specific and muscle specific regulatory elements, are provided in Table 1 herein. Accordingly, such viral vectors may be delivered to the human subject at appropriate dosages, for example 10E11 to 10E14 vg/kg, such that at least 20, 30, 40, 50 or 60 days after administration, the anti-pKal antibody or lanadelumab or antigen binding fragment thereof is present in the serum of said human subject at a level of at least 1.5 μg/ml to 35 μg/ml anti-pKal antibody or lanadelumab or antigen binding fragment thereof in said subject, or of at least 5 μg/ml to 35 μg/ml anti-pKal antibody or lanadelumab or antigen binding fragment thereof, or of at least 1.5 μg/ml to 20 μg/ml anti-pKal antibody or lanadelumab or antigen binding fragment thereof or of at least 1.5 μg/ml to 10 μg/ml anti-pKal antibody or lanadelumab or antigen binding fragment thereof or of at least 5 μg/ml to 20 μg/ml anti-pKal antibody or lanadelumab or antigen binding fragment thereof within at least 20, 30, 40, 50, or 60 days of said administering.

The HuPTM mAb or HuPTM antigen-binding fragment encoded by the transgene can include, but is not limited to, a full-length or an antigen-binding fragment of a therapeutic antibody that binds to pKal, including but not limited to, lanadelumab. The amino acid sequences of the heavy and light chain of antigen binding fragments of the foregoing are provided in Table 7, infra. Heavy chain variable domain having an amino acid sequence of SEQ ID NO: 248 and light chain variable domain having an amino acid sequence of SEQ ID NO: 249 (encoded by nucleotide sequence SEQ ID NO: 250 and 251, respectively) of The HuPTM mAb or HuPTM antigen-binding fragment encoded by the transgene can include, but is not limited to, a full-length or an antigen-binding fragment or scFv (including an scFv-Fc) of a therapeutic antibody or antigen-binding fragments engineered to contain additional glycosylation sites on the Fab domain (e.g., see Courtois et al., 2016, mAbs 8:99-112 which is incorporated by reference herein in its entirety for its description of derivatives of antibodies that are hyperglycosylated on the Fab domain of the full-length antibody).

The recombinant vector used for delivering the transgene includes non-replicating recombinant adeno-associated virus vectors (“rAAV”). rAAVs are particularly attractive vectors for a number of reasons-they can be modified to preferentially target a specific organ of choice; and there are hundreds of capsid serotypes to choose from to obtain the desired tissue specificity, and/or to avoid neutralization by pre-existing patient antibodies to some AAVs. The AAV types for use here in preferentially target the liver, i.e., have a tropism for liver cells and/or target muscle tissue, i.e., have a tropism for muscle cells. Such rAAVs include but are not limited to AAV based vectors comprising capsid components from one or more of AAV2, AAV3B, AAV-LK03, AAVS3, AAV7, AAV8, AAV9, AAV10, AAV11, AAVrh10, AAVrh46 or AAVrh73. In certain embodiments, AAV based vectors provided herein comprise capsids from one or more of AAV8, AAVrh46, AAVrh73, or AAVS3, or AAV-LK03 serotypes.

However, other viral vectors may be used, including but not limited to lentiviral vectors; vaccinia viral vectors, or non-viral expression vectors referred to as “naked DNA” constructs. Expression of the transgene can be controlled by constitutive or tissue-specific expression control elements.

Gene therapy constructs are designed such that both the heavy and light chains are expressed. In certain embodiments, the full length heavy and light chains of the antibody are expressed. In certain embodiments, the coding sequences encode for a Fab or F(ab′)₂ or an scFv or an scFv-Fc. The heavy and light chains should be expressed at about equal amounts, in other words, the heavy and light chains are expressed at approximately a 1:1 ratio of heavy chains to light chains. The coding sequences for the heavy and light chains can be engineered in a single construct in which the heavy and light chains are separated by a cleavable linker or IRES so that separate heavy and light chain polypeptides are expressed. In specific embodiments, the linker separating the heavy and light chains is a Furin-2A linker, for example a Furin-F2A linker RKRR(GSG)APVKQTLNFDLLKLAGDVESNPGP (SEQ ID NOS: 105 or 106) or a Furin-T2A linker RKRR(GSG)EGRGSLLTCGDVEENPGP (SEQ ID NOS: 103 or 104). In certain embodiments, the construct expresses, from the N-terminus to C-terminus, NH2-VL-linker-VH—COOH or NH2-VH-linker-VL-COOH. In other embodiments, the construct expresses, from the N-terminus to C-terminus, NH2-signal or localization sequence-VL-linker-VH—COOH or NH2-signal or localization sequence-VH-linker-VL-COOH. In other embodiments, the constructs express an scFv in which the heavy and light chain variable domains are connected via a flexible, non-cleavable linker or an scFv-Fc in which the Fc is connected to the scFv via a flexible, non-cleavable linker.

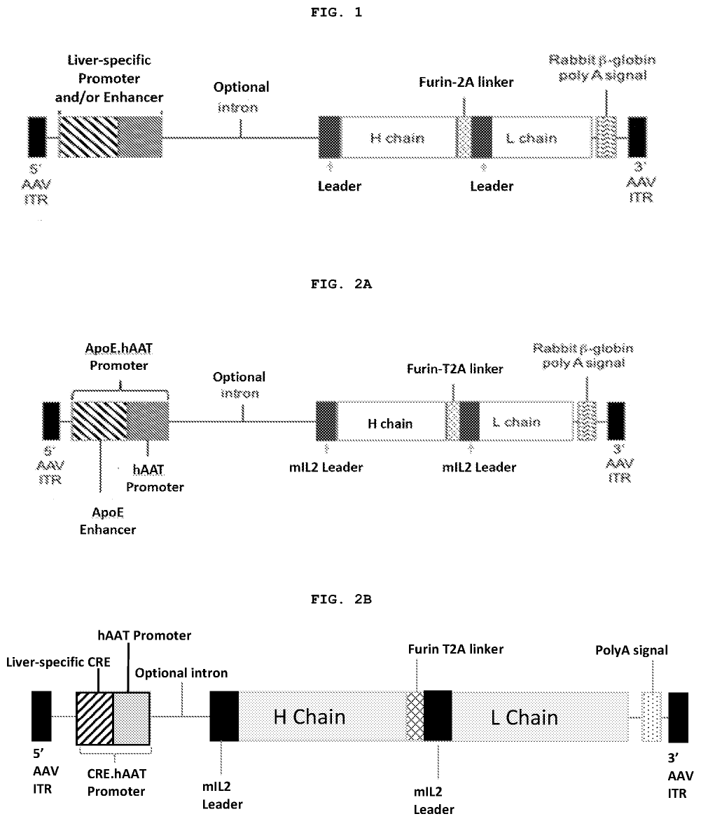

In certain embodiments, nucleic acids (e.g., polynucleotides) and nucleic acid sequences disclosed herein may be codon-optimized, for example, via any codon-optimization technique known to one of skill in the art (see, e.g., review by Quax et al., 2015, Mol Cell 59:149-161) and may also be optimized to reduce CpG dimers. Codon optimized sequences of the lanadelumab heavy and light chains are provided in Table 7 (SEQ ID NOs: 148-150) and of the scFv-Fcs in Table 14 (SEQ ID Nos: 323 and 392). Each heavy and light chain requires a signal sequence to ensure proper post-translation processing and secretion (unless expressed as an scFv or scFv-Fc, in which only the N-terminal of the construct requires a signal sequence sequence). Useful signal sequences for the expression of the heavy and light chains of the therapeutic antibodies in human cells are disclosed herein. Exemplary recombinant expression constructs are shown in FIGS. 1, 2 and 20.

The production of HuPTM mAb or HuPTM Fab (including an HuPTM scFv) should result in a “biobetter” molecule for the treatment of disease accomplished via gene therapy—e.g., by administering a viral vector or other DNA expression construct encoding a full-length HuPTM mAb or HuPTM Fab or other antigen binding fragment, such as an scFv, of a therapeutic mAb to a patient (human subject) diagnosed with a disease indication for that mAb, to create a permanent depot in the subject that continuously supplies the human-glycosylated, sulfated transgene product produced by the subject's transduced cells. The cDNA construct for the HuPTM mAb or HuPTM Fab or HuPTM scFv should include a signal peptide that ensures proper co- and post-translational processing (glycosylation and protein sulfation) by the transduced human cells.

Pharmaceutical compositions suitable for administration to human subjects comprise a suspension of the recombinant vector in a formulation buffer comprising a physiologically compatible aqueous buffer, a surfactant and optional excipients. Such formulation buffer can comprise one or more of a polysaccharide, a surfactant, polymer, or oil.

As an alternative, or an additional treatment to gene therapy, the full-length HuPTM mAb or HuPTM Fab or other antigen binding fragment thereof can be produced in human cell lines by recombinant DNA technology, and the glycoprotein can be administered to patients. Human cell lines that can be used for such recombinant glycoprotein production include but are not limited to human embryonic kidney 293 cells (HEK293), fibrosarcoma HT-1080, HKB-11, CAP, HuH-7, and retinal cell lines, PER.C6, or RPE to name a few (e.g., see Dumont et al., 2015, Crit. Rev. Biotechnol. 36(6):1110-1122, which is incorporated by reference in its entirety for a review of the human cell lines that could be used for the recombinant production of the HuPTM mAb, HuPTM Fab or HuPTM scFv product, e.g., HuPTM Fab glycoprotein). To ensure complete glycosylation, especially sialylation, and tyrosine-sulfation, the cell line used for production can be enhanced by engineering the host cells to co-express α-2,6-sialyltransferase (or both α-2,3- and α-2,6-sialyltransferases) and/or TPST-1 and TPST-2 enzymes responsible for tyrosine-O-sulfation in human cells.

It is not essential that every molecule produced either in the gene therapy or protein therapy approach be fully glycosylated and sulfated. Rather, the population of glycoproteins produced should have sufficient glycosylation (including 2,6-sialylation) and sulfation to demonstrate efficacy. The goal of gene therapy treatment of the invention is to slow or arrest the progression of disease.

Combination therapies involving delivery of the full-length HuPTM mAb or HuPTM Fab or antigen binding fragment thereof to the patient accompanied by administration of other available treatments are encompassed by the methods of the invention. The additional treatments may be administered before, concurrently or subsequent to the gene therapy treatment. Such additional treatments can include but are not limited to co-therapy with the therapeutic mAb.

Also provided are methods of manufacturing the viral vectors, particularly the AAV based viral vectors. In specific embodiments, provided are methods of producing recombinant AAVs comprising culturing a host cell containing an artificial genome comprising a cis expression cassette flanked by AAV ITRs, wherein the cis expression cassette comprises a transgene encoding a therapeutic antibody operably linked to expression control elements that will control expression of the transgene in human cells; a trans expression cassette lacking AAV ITRs, wherein the trans expression cassette encodes an AAV rep and capsid protein operably linked to expression control elements that drive expression of the AAV rep and capsid proteins in the host cell in culture and supply the rep and cap proteins in trans; sufficient adenovirus helper functions to permit replication and packaging of the artificial genome by the AAV capsid proteins; and recovering recombinant AAV encapsidating the artificial genome from the cell culture.

### 5.1 Constructs

Viral vectors or other DNA expression constructs encoding an anti-pKal HuPTM mAb or antigen-binding fragment thereof, particularly a HuGlyFab or a scFv-Fc, or a hyperglycosylated derivative of a HuPTM mAb antigen-binding fragment are provided herein. The viral vectors and other DNA expression constructs provided herein include any suitable method for delivery of a transgene to a target cell. The means of delivery of a transgene include viral vectors, liposomes, other lipid-containing complexes, other macromolecular complexes, synthetic modified mRNA, unmodified mRNA, small molecules, non-biologically active molecules (e.g., gold particles), polymerized molecules (e.g., dendrimers), naked DNA, plasmids, phages, transposons, cosmids, or episomes. In some embodiments, the vector is a targeted vector, e.g., a vector targeting liver cells or a vector that has a tropism for liver cells or a vector targeting muscle cells or a vector that has a tropism for muscle cells.

In some aspects, the disclosure provides for a nucleic acid for use, wherein the nucleic acid comprises a nucleotide sequence that encodes a HuPTM mAb or HuGlyFab or other antigen-binding fragment thereof, as a transgene described herein, operatively linked to an ubiquitous promoter, a liver-specific and/or muscle-specific promoter, or an inducible promoter, wherein the promoter is selected for expression in tissue targeted for expression of the transgene. Promoters may, for example, be a CB7/CAG promoter (SEQ ID NO:36) and associated upstream regulatory sequences, cytomegalovirus (CMV) promoter, EF-1 alpha promoter (SEQ ID NO:39), mU1a (SEQ ID NO:38), UB6 promoter, chicken beta-actin (CBA) promoter, and liver-specific promoters, such as TBG (Thyroxine-binding Globulin) promoter (SEQ ID NO:40), APOA2 promoter, any one of the sequences of SEQ ID Nos: 163-293, SERPINA1 (hAAT) promoter, ApoE.hAAT (SEQ ID NO:21), or muscle-specific promoters, such as a human desmin promoter, CK8 promoter (SEQ ID NO:37) or Pitx3 promoter, inducible promoters, such as a hypoxia-inducible promoter or a rapamycin-inducible promoter, or a combination thereof. In preferred embodiments, the promoter is a liver-specific promoter or a liver- and muscle-specific (dual) promoter. In preferred embodiments, the promoter is the liver-specific ApoE.hAAT (SEQ ID NO:21) promoter. In other preferred embodiments, the promoter is one, two, or three liver-specific cis-regulatory elements selected from the sequences in of SEQ ID Nos: 163-293 or a dual promoter comprising one of the cis-regulatory elements selected from the sequences of SEQ ID Nos: 163-293 and the hAAT promoter.

In some aspects herein, transgene expression is controlled by engineered nucleic acid regulatory elements that have more than one regulatory element (promoter or enhancer), including regulatory elements that are arranged in tandem (two or three copies) that promote liver-specific expression, or both liver-specific expression and muscle-specific expression. These regulatory elements include for the liver-specific expression, LSPX1 (SEQ ID NO:9), LSPX2 (SEQ ID NO:10), LTP1 (SEQ ID NO:11), LTP2 (SEQ ID NO:12), or LTP3 (SEQ ID NO:13), and for the liver and muscle expression, LMTP6 (SEQ ID NO:14), LMTP13 (SEQ ID NO:15), LMTP14 (SEQ ID NO:16), LMTP15 (SEQ ID NO:17), LMTP18 (SEQ ID NO:18), LMTP19 (SEQ ID NO:19), or LMTP20 (SEQ ID NO: 20), the sequences of which are provided in Table 1.

In certain embodiments, provided herein are recombinant vectors that comprise one or more nucleic acids (e.g., polynucleotides). The nucleic acids may comprise DNA, RNA, or a combination of DNA and RNA. In certain embodiments, the DNA comprises one or more of the sequences selected from the group consisting of promoter sequences, the sequence of the gene of interest (the transgene, e.g., the nucleotide sequences encoding the heavy and light chains of the HuPTMmAb or HuGlyFab or other antigen-binding fragment), untranslated regions, and termination sequences. In certain embodiments, viral vectors provided herein comprise a promoter operably linked to the gene of interest.

In certain embodiments, nucleic acids (e.g., polynucleotides) and nucleic acid sequences disclosed herein may be codon-optimized, for example, via any codon-optimization technique known to one of skill in the art (see, e.g., review by Quax et al., 2015, Mol Cell 59:149-161).

In a specific embodiment, the constructs described herein comprise the following components: (1) AAV2 inverted terminal repeats that flank the expression cassette; (2) one or more control elements, b) optionally, a chicken β-actin or other intron and c) a rabbit β-globin poly A signal; and (3) nucleic acid sequences coding for the heavy and light chains of a mAb or Fab, separated by a self-cleaving furin (F)/(F/T)2A linker (SEQ ID NOS: 103, 104, 105 or 106), ensuring expression of equal amounts of the heavy and the light chain polypeptides. An exemplary construct is shown in FIG. 1.

In a specific embodiment, the constructs described herein comprise the following components: (1) AAV2 inverted terminal repeats that flank the expression cassette; (2) ApoE.hAAT promoter, b) optionally, a chicken β-actin or other intron and c) a rabbit β-globin polyA signal; and (3) nucleic acid sequences coding for a full-length antibody comprising the heavy and light chain sequences using sequences that encode the Fab portion of the heavy chain, including the hinge region sequence, plus the Fc polypeptide of the heavy chain for the appropriate isotype and the light chain, wherein heavy and light chain nucleotide sequences are separated by a self-cleaving furin (F)/(F/T) 2A linker (SEQ ID NOS: 103, 104, 105 or 106), ensuring expression of equal amounts of the heavy and the light chain polypeptides. An exemplary construct is shown in FIG. 2A.

In a specific embodiment, the constructs described herein comprise the following components: (1) AAV2 inverted terminal repeats that flank the expression cassette; (2) liver-specific CRE.hAAT promoter, b) optionally, a chicken β-actin or other intron and c) a rabbit β-globin polyA signal; and (3) nucleic acid sequences coding for a full-length antibody comprising the heavy and light chain sequences using sequences that encode the Fab portion of the heavy chain, including the hinge region sequence, plus the Fc polypeptide of the heavy chain for the appropriate isotype and the light chain, wherein heavy and light chain nucleotide sequences are separated by a self-cleaving furin (F)/(F/T) 2A linker (SEQ ID NOS: 103, 104, 105 or 106), ensuring expression of equal amounts of the heavy and the light chain polypeptides. An exemplary construct is shown in FIG. 2B.

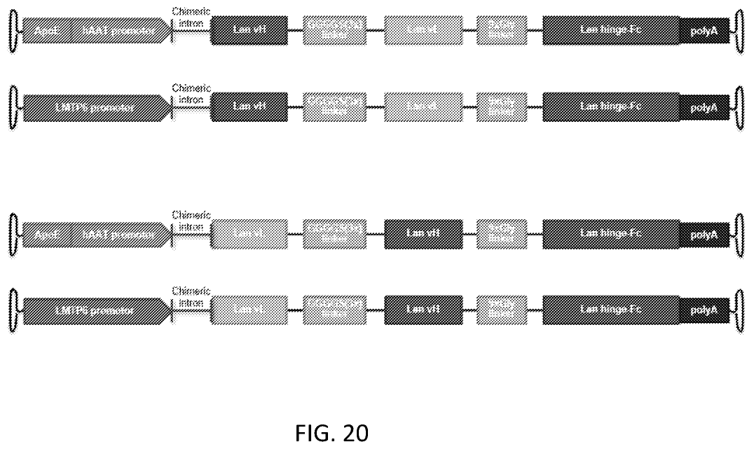

In other embodiments, exemplary constructs for the expression of scFv-Fcs are provided, for example as shown in FIG. 20 and in Table 14.

5.1.1 mRNA Vectors

In certain embodiments, as an alternative to DNA vectors, the vectors provided herein are modified mRNA encoding for the gene of interest (e.g., the transgene, for example, HuPTMmAb or HuGlyFab or other antigen binding fragment thereof). The synthesis of modified and unmodified mRNA for delivery of a transgene to retinal pigment epithelial cells is taught, for example, in Hansson et al., J. Biol. Chem., 2015, 290(9):5661-5672, which is incorporated by reference herein in its entirety.

In certain embodiments, provided herein is a modified mRNA encoding for a HuPTMmAb, HuPTM Fab, or HuPTM scFv.

### 5.1.2 Viral Vectors

Viral vectors include adenovirus, adeno-associated virus (AAV, e.g., AAV8, AAV9, AAVrh10, AAVS3), lentivirus, helper-dependent adenovirus, herpes simplex virus, poxvirus, hemagglutinin virus of Japan (HVJ), alphavirus, vaccinia virus, and retrovirus vectors. Retroviral vectors include murine leukemia virus (MLV) and human immunodeficiency virus (HIV)-based vectors. Alphavirus vectors include semliki forest virus (SFV) and sindbis virus (SIN). In certain embodiments, the viral vectors provided herein are recombinant viral vectors. In certain embodiments, the viral vectors provided herein are altered such that they are replication-deficient in humans. In certain embodiments, the viral vectors are hybrid vectors, e.g., an AAV vector placed into a “helpless” adenoviral vector. In certain embodiments, provided herein are viral vectors comprising a viral capsid from a first virus and viral envelope proteins from a second virus. In specific embodiments, the second virus is vesicular stomatitus virus (VSV). In more specific embodiments, the envelope protein is VSV-G protein.

In certain embodiments, the viral vectors provided herein are HIV based viral vectors. In certain embodiments, HIV-based vectors provided herein comprise at least two polynucleotides, wherein the gag and pol genes are from an HIV genome and the env gene is from another virus.

In certain embodiments, the viral vectors provided herein are herpes simplex virus-based viral vectors. In certain embodiments, herpes simplex virus-based vectors provided herein are modified such that they do not comprise one or more immediately early (IE) genes, rendering them non-cytotoxic.

In certain embodiments, the viral vectors provided herein are MLV based viral vectors. In certain embodiments, MLV-based vectors provided herein comprise up to 8 kb of heterologous DNA in place of the viral genes.

In certain embodiments, the viral vectors provided herein are lentivirus-based viral vectors. In certain embodiments, lentiviral vectors provided herein are derived from human lentiviruses. In certain embodiments, lentiviral vectors provided herein are derived from non-human lentiviruses. In certain embodiments, lentiviral vectors provided herein are packaged into a lentiviral capsid. In certain embodiments, lentiviral vectors provided herein comprise one or more of the following elements: long terminal repeats, a primer binding site, a polypurine tract, att sites, and an encapsidation site.

In certain embodiments, the viral vectors provided herein are alphavirus-based viral vectors. In certain embodiments, alphavirus vectors provided herein are recombinant, replication-defective alphaviruses. In certain embodiments, alphavirus replicons in the alphavirus vectors provided herein are targeted to specific cell types by displaying a functional heterologous ligand on their virion surface.

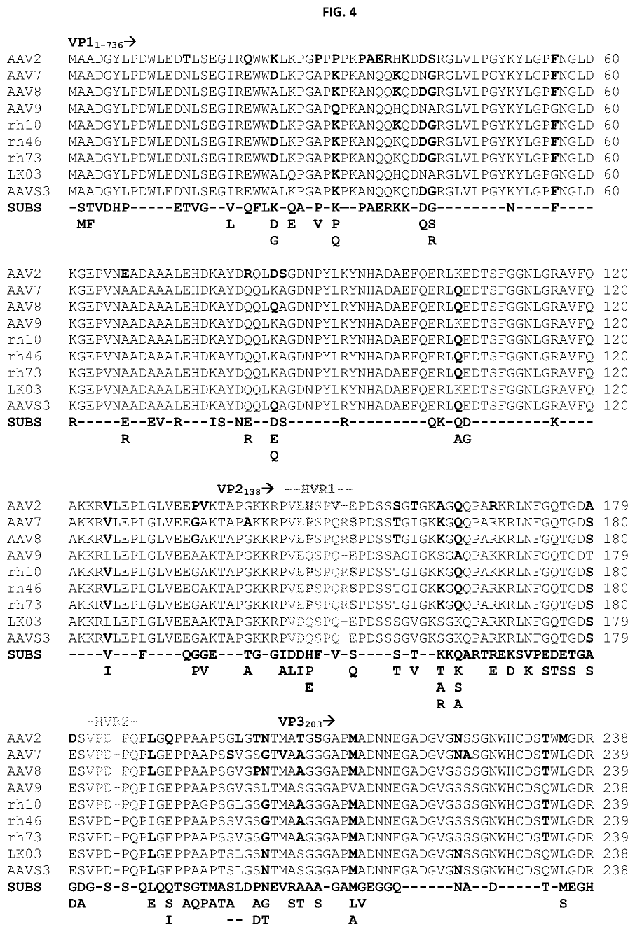

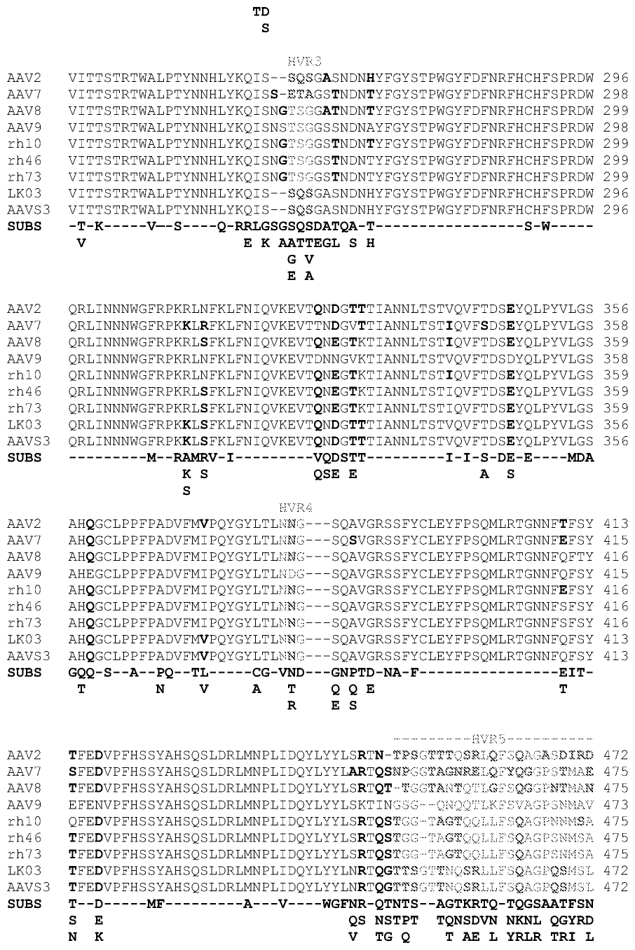

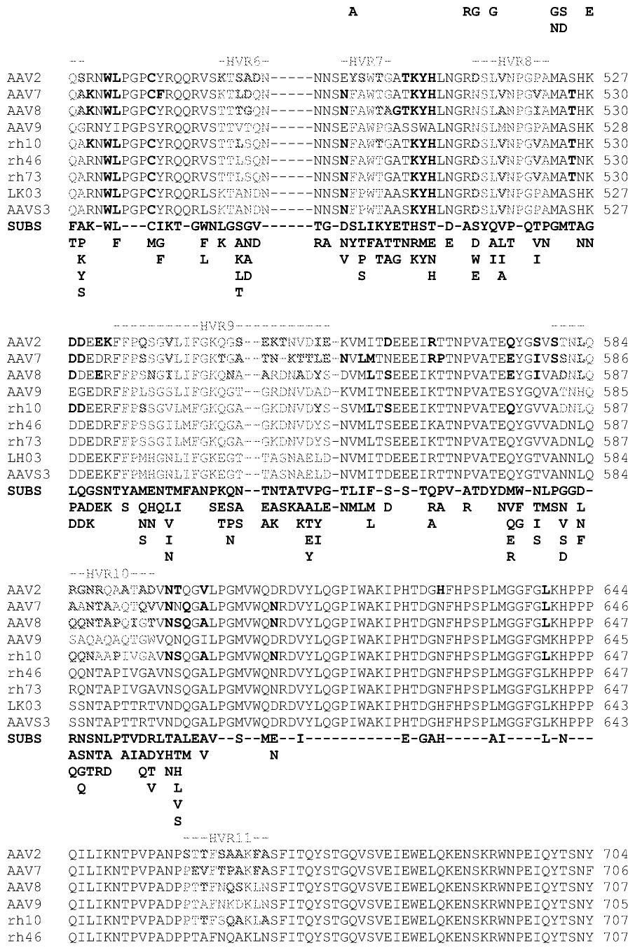

In certain embodiments, the viral vectors provided herein are AAV based viral vectors. In certain embodiments, the AAV-based vectors provided herein do not encode the AAV rep gene (required for replication) and/or the AAV cap gene (required for synthesis of the capsid proteins) (the rep and cap proteins may be provided by the packaging cells in trans). Multiple AAV serotypes have been identified. In certain embodiments, AAV-based vectors provided herein comprise components from one or more serotypes of AAV. In preferred embodiments, AAV-based vectors provided herein comprise components from one or more serotypes of AAV with tropism to liver and/or muscle. In certain embodiments, AAV based vectors provided herein comprise capsid components from one or more of AAV2 (SEQ ID NO:334), AAV7 (SEQ ID NO:1), AAV8 (SEQ ID NO:2), AAV9 (SEQ ID NO: 3), AAVS3 (SEQ ID NO:7), AAVrh46 (SEQ ID NO:5), AAVrh73 (SEQ ID NO:6), AAV-LK03 (SEQ ID NO:8), or AAVrh10 (SEQ ID NO:4). In certain embodiments, AAV based vectors provided herein are or comprise components from one or more of AAV8, AAVS3, AAV-LK03, AAVrh46, AAVrh73, or AAVrh10 serotypes. Provided are viral vectors in which the capsid protein is a variant of the AAV8 capsid protein (SEQ ID NO:2), AAVS3 capsid protein (SEQ ID NO:8), or AAV-LK03 capsid protein (SEQ ID NO:7), and the capsid protein is e.g., at least 95%, 96%, 97%, 98%, 99% or 99.9% identical to the amino acid sequence of the AAV8 capsid protein (SEQ ID NO:2), AAVS3 capsid protein (SEQ ID NO:8), or AAV-LK03 capsid protein (SEQ ID NO:7), while retaining the biological function of the native capsid. In certain embodiments, the encoded AAV capsid has the sequence of SEQ ID NO: 104 with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions and retaining the biological function of the AAV8, AAVS3, or AAV-LK03 capsid. FIG. 4 provides a comparative alignment of the amino acid sequences of the capsid proteins of different AAV serotypes with potential amino acids that may be substituted at certain positions in the aligned sequences based upon the comparison in the row labeled SUBS. Accordingly, in specific embodiments, the AAV vector comprises an AAV8, AAVS3, or AAV-LK03, capsid variant that has 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions that are not present at that position in the native AAV capsid sequence as identified in the SUBS row of FIG. 4. Amino acid sequence for AAV8, AAVS3, or AAV-LK03 capsids are provided in FIG. 4. In specific embodiments, the capsid is a modified capsid as disclosed in PCT application PCT/US2020/026485, which is hereby incorporated by reference in its entirety.

The amino acid sequence of hu37 capsid can be found in international application PCT WO 2005/033321 (SEQ ID NO: 88 thereof) and the amino acid sequence for the rh8 capsid can be found in international application PCT WO 03/042397 (SEQ ID NO:97). The amino acid sequence for the rh64R1 sequence is found in WO2006/110689 (a R697W substitution of the Rh.64 sequence, which is SEQ ID NO: 43 of WO 2006/110689). The rh64R1 sequence is:

(SEQ ID NO: 24)
MAADGYLPDWLEDNLSEGIREWWDLKPGAPKPKANQQKQDDGRGLVLP
GYKYLGPFNGLDKGEPVNAADAAALEHDKAYDQQLKAGDNPYLRYNHA
DAEFQERLQEDTSFGGNLGRAVFQAKKRVLEPLGLVEEGAKTAPGKKR
PVEPSPQRSPDSSTGIGKKGQQPARKRLNFGQTGDSESVPDPQPIGEP
PAAPSSVGSGTMAAGGGAPMADNNEGADGVGSSSGNWHCDSTWLGDRV
ITTSTRTWALPTYNNHLYKQISNGTSGGSTNDNTYFGYSTPWGYFDFN
RFHCHFSPRDWQRLINNNWGFRPKRLSFKLFNIQVKEVTQNEGTKTIA
NNLTSTIQVFTDSEYQLPYVLGSAHQGCLPPFPADVFMIPQYGYLTLN
NGSQAVGRSSFYCLEYFPSQMLRTGNNFSFSYTFEDVPFHSSYAHSQS
LDRLMNPLIDQYLYYLSRTQSTGGTAGTQQLLFSQAGPSNMSAQARNW
LPGPCYRQQRVSTTLSQNNNSNFAWTGATKYHLNGRDSLVNPGVAMAT
NKDDEDRFFPSSGILMFGKQGAGKDNVDYSNVMLTSEEEIKTTNPVAT
EQYGVVADNLQQQNTAPIVGAVNSQGALPGMVWQNRDVYLQGPIWAKI
PHTDGNFHPSPLMGGFGLKHPPPQILIKNTPVPADPPTAFNQAKLNSF
ITQYSTGQVSVEIVWELQKENSKRWNPEIQYTSNYYKSTNVDFAVNTE
GVYSEPRPIGTRYLTRNL.

In some embodiments, AAV-based vectors comprise components from one or more serotypes of AAV. In some embodiments, AAV based vectors provided herein comprise capsid components from one or more of AAV1, AAV2, AAV3, AAV4, AAV5, AAV6, AAV7, AAV8, AAV9, AAV10, AAV11, AAV12, AAV13, AAV14, AAV15, AAV16, AAVS3, AAV.rh8, AAV.rh10, AAV.rh20, AAV.rh39, AAV.rh46, AAV.rh73, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other rAAV particles, or combinations of two or more thereof. In some embodiments, AAV based vectors provided herein comprise components from one or more of AAV1, AAV2, AAV3, AAV4, AAV5, AAV6, AAV7, AAV8, AAV9, AAV10, AAV11, AAV12, AAV13, AAV14, AAV15, AAV16, AAVS3, AAV.rh8, AAV.rh10, AAV.rh20, AAV.rh39, AAV.rh46, AAV.rh73, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other rAAV particles, or combinations of two or more thereof serotypes. In some embodiments, rAAV particles comprise a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to e.g., VP1, VP2 and/or VP3 sequence of an AAV capsid serotype selected from AAV1, AAV2, AAV3, AAV4, AAV5, AAV6, AAV7, AAV8, AAV9, AAV10, AAV11, AAV12, AAV13, AAV14, AAV15, AAV16, AAVS3, AAV.rh8, AAV.rh10, AAV.rh20, AAV.rh39, AAV.rh46, AAV.rh73, AAV.Rh74, AAV.RHM4-1, AAV.hu37, AAV.Anc80, rAAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16, or a derivative, modification, or pseudotype thereof.

In particular embodiments, the recombinant AAV for us in compositions and methods herein is AAVS3 (including variants thereof) (see e.g., U.S. patent application No. 20200079821, which is incorporated herein by reference in its entirety). In particular embodiments, rAAV particles comprise the capsids of AAV-LK03 or AAV3B, as described in Puzzo et al., 2017, Sci. Transl. Med. 29(9): 418, which is incorporated by reference in its entirety. In particular embodiments, the AAV for use in compositions and methods herein is any AAV disclosed in U.S. Pat. No. 10,301,648, such as AAV.rh46 or AAV.rh73. In some embodiments, the recombinant AAV for use in compositions and methods herein is Anc80 or Anc80L65 (see, e.g., Zinn et al., 2015, Cell Rep. 12(6): 1056-1068, which is incorporated by reference in its entirety). In particular embodiments, the AAV for use in compositions and methods herein is any AAV disclosed in U.S. Pat. No. 9,585,971, such as AAV-PHP.B. In particular embodiments, the AAV for use in compositions and methods herein is an AAV2/Rec2 or AAV2/Rec3 vector, which has hybrid capsid sequences derived from AAV8 and serotypes cy5, rh20 or rh39 (see, e.g., Issa et al., 2013, PLOS One 8(4): e60361, which is incorporated by reference herein for these vectors). In particular embodiments, the AAV for use in compositions and methods herein is an AAV disclosed in any of the following, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; 9,587,282; US 2015/0374803; US 2015/0126588; US 2017/0067908; US 2013/0224836; US 2016/0215024; US 2017/0051257; PCT/US2015/034799; and PCT/EP2015/053335. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

In some embodiments, rAAV particles comprise any AAV capsid disclosed in U.S. Pat. No. 9,840,719 and WO 2015/013313, such as AAV.Rh74 and RHM4-1, each of which is incorporated herein by reference in its entirety. In some embodiments, rAAV particles comprise any AAV capsid disclosed in WO 2014/172669, such as AAV rh.74, which is incorporated herein by reference in its entirety. In some embodiments, rAAV particles comprise the capsid of AAV2/5, as described in Georgiadis et al., 2016, Gene Therapy 23:857-862 and Georgiadis et al., 2018, Gene Therapy 25:450, each of which is incorporated by reference in its entirety. In some embodiments, rAAV particles comprise any AAV capsid disclosed in WO 2017/070491, such as AAV2tYF, which is incorporated herein by reference in its entirety. In some embodiments, rAAV particles comprise any AAV capsid disclosed in U.S. Pat. Nos. 8,628,966; 8,927,514; 9,923,120 and WO 2016/049230, such as HSC1, HSC2, HSC3, HSC4, HSC5, HSC6, HSC7, HSC8, HSC9, HSC10, HSC11, HSC12, HSC13, HSC14, HSC15, or HSC16, each of which is incorporated by reference in its entirety.

In some embodiments, rAAV particles have a capsid protein disclosed in Intl. Appl. Publ. No. WO 2003/052051 (see, e.g., SEQ ID NO: 2 of '051 publication), WO 2005/033321 (see, e.g., SEQ ID NOs: 123 and 88 of '321 publication), WO 03/042397 (see, e.g., SEQ ID NOs: 2, 81, 85, and 97 of '397 publication), WO 2006/068888 (see, e.g., SEQ ID NOs: 1 and 3-6 of '888 publication), WO 2006/110689, (see, e.g., SEQ ID NOs: 5-38 of '689 publication) WO2009/104964 (see, e.g., SEQ ID NOs: 1-5, 7, 9, 20, 22, 24 and 31 of '964 publication), WO 2010/127097 (see, e.g., SEQ ID NOs: 5-38 of '097 publication), and WO 2015/191508 (see, e.g., SEQ ID NOs: 80-294 of '508 publication), and U.S. Appl. Publ. No. 20150023924 (see, e.g., SEQ ID NOs: 1, 5-10 of '924 publication), the contents of each of which is herein incorporated by reference in its entirety. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in Intl. Appl. Publ. No. WO 2003/052051 (see, e.g., SEQ ID NO: 2 of '051 publication), WO 2005/033321 (see, e.g., SEQ ID NOs: 123 and 88 of '321 publication), WO 03/042397 (see, e.g., SEQ ID NOs: 2, 81, 85, and 97 of '397 publication), WO 2006/068888 (see, e.g., SEQ ID NOs: 1 and 3-6 of '888 publication), WO 2006/110689 (see, e.g., SEQ ID NOs: 5-38 of '689 publication) WO2009/104964 (see, e.g., SEQ ID NOs: 1-5, 7, 9, 20, 22, 24 and 31 of 964 publication), WO 2010/127097 (see, e.g., SEQ ID NOs: 5-38 of '097 publication), and WO 2015/191508 (see, e.g., SEQ ID NOs: 80-294 of '508 publication), and U.S. Appl. Publ. No. 20150023924 (see, e.g., SEQ ID NOs: 1, 5-10 of '924 publication).

In additional embodiments, rAAV particles comprise a pseudotyped AAV capsid. In some embodiments, the pseudotyped AAV capsids are rAAV2/8 or rAAV2/9 pseudotyped AAV capsids. Methods for producing and using pseudotyped rAAV particles are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

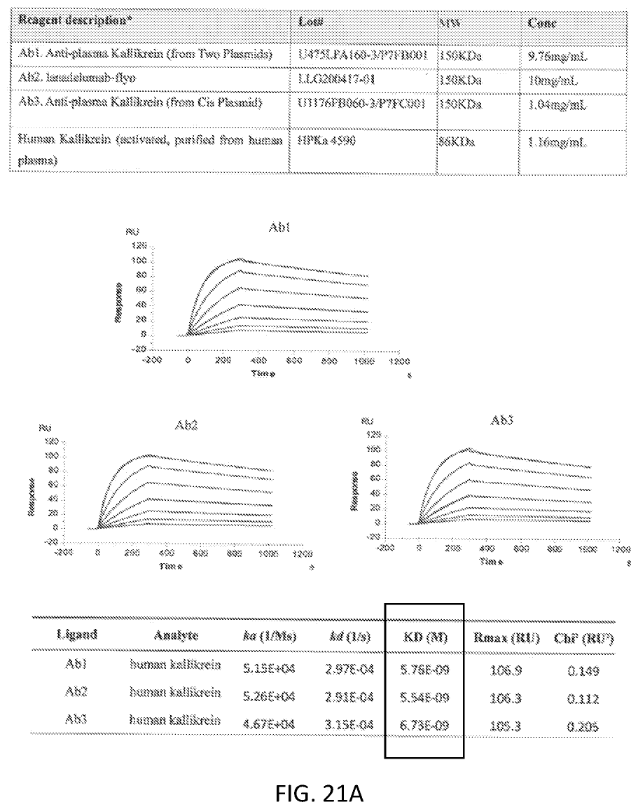

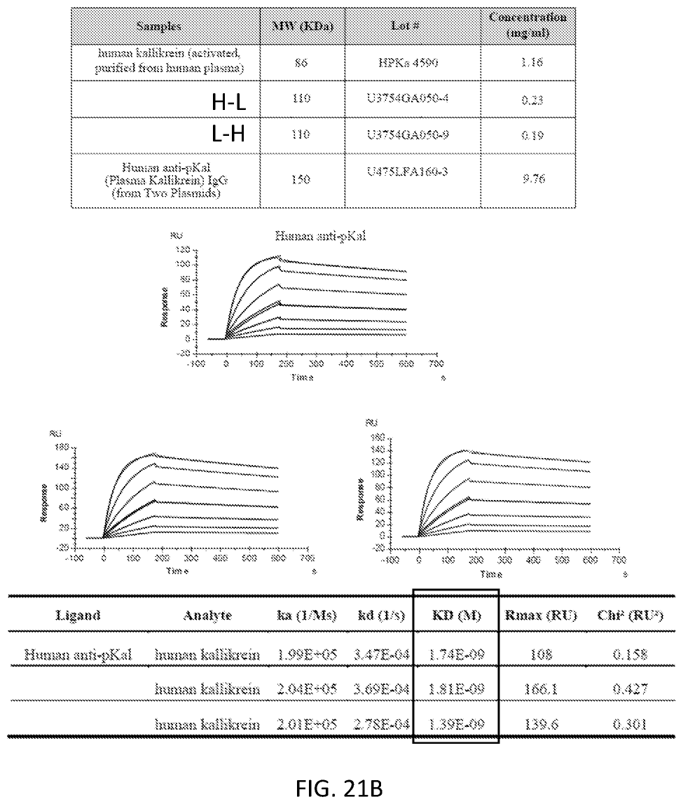

AAV8-based, AAV9-based, and AAVrh10-based viral vectors are used in certain of the methods described herein. Nucleotide sequences of AAV based viral vectors and methods of making recombinant AAV and AAV capsids are taught, for example, in U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety. In one aspect, provided herein are AAV (e.g., AAV8, AAV9 or AAVrh10)-based viral vectors encoding a transgene (e.g., an HuPTM Fab). The amino acid sequences of AAV capsids, including AAV8, AAV9 and AAVrh10 are provided in FIG. 21.

In certain embodiments, a single-stranded AAV (ssAAV) may be used supra. In certain embodiments, a self-complementary vector, e.g., scAAV, may be used (see, e.g., Wu, 2007, Human Gene Therapy, 18(2):171-82, McCarty et al, 2001, Gene Therapy, Vol 8, Number 16, Pages 1248-1254; and U.S. Pat. Nos. 6,596,535; 7,125,717; and 7,456,683, each of which is incorporated herein by reference in its entirety).

In certain embodiments, the viral vectors used in the methods described herein are adenovirus based viral vectors. A recombinant adenovirus vector may be used to transfer in the transgene encoding the HuPTMmAb or HuGlyFab or antigen-binding fragment. The recombinant adenovirus can be a first-generation vector, with an E1 deletion, with or without an E3 deletion, and with the expression cassette inserted into either deleted region. The recombinant adenovirus can be a second-generation vector, which contains full or partial deletions of the E2 and E4 regions. A helper-dependent adenovirus retains only the adenovirus inverted terminal repeats and the packaging signal (phi). The transgene is inserted between the packaging signal and the 3′ITR, with or without stuffer sequences to keep the genome close to wild-type size of approximately 36 kb. An exemplary protocol for production of adenoviral vectors may be found in Alba et al., 2005, “Gutless adenovirus: last generation adenovirus for gene therapy,” Gene Therapy 12:S18-S27, which is incorporated by reference herein in its entirety.

In certain embodiments, the viral vectors used in the methods described herein are lentivirus based viral vectors. A recombinant lentivirus vector may be used to transfer in the transgene encoding the HuPTM mAb antigen binding fragment. Four plasmids are used to make the construct: Gag/pol sequence containing plasmid, Rev sequence containing plasmids, Envelope protein containing plasmid (e.g., VSV-G), and Cis plasmid with the packaging elements and the anti-VEGF antigen-binding fragment gene.

For lentiviral vector production, the four plasmids are co-transfected into cells (e.g., HEK293 based cells), whereby polyethylenimine or calcium phosphate can be used as transfection agents, among others. The lentivirus is then harvested in the supernatant (lentiviruses need to bud from the cells to be active, so no cell harvest needs/should be done). The supernatant is filtered (0.45 μm) and then magnesium chloride and benzonase added. Further downstream processes can vary widely, with using TFF and column chromatography being the most GMP compatible ones. Others use ultracentrifugation with/without column chromatography. Exemplary protocols for production of lentiviral vectors may be found in Lesch et al., 2011, “Production and purification of lentiviral vector generated in 293T suspension cells with baculoviral vectors,” Gene Therapy 18:531-538, and Ausubel et al., 2012, “Production of CGMP-Grade Lentiviral Vectors,” Bioprocess Int. 10(2):32-43, both of which are incorporated by reference herein in their entireties.

In a specific embodiment, a vector for use in the methods described herein is one that encodes an HuPTM mAb, such that, upon introduction of the vector into a relevant cell, a glycosylated and/or tyrosine sulfated variant of the HuPTM mAb is expressed by the cell.

### 5.1.3 Promoters and Modifiers of Gene Expression

In certain embodiments, the vectors provided herein comprise components that modulate gene delivery or gene expression (e.g., “expression control elements”). In certain embodiments, the vectors provided herein comprise components that modulate gene expression. In certain embodiments, the vectors provided herein comprise components that influence binding or targeting to cells. In certain embodiments, the vectors provided herein comprise components that influence the localization of the polynucleotide (e.g., the transgene) within the cell after uptake. In certain embodiments, the vectors provided herein comprise components that can be used as detectable or selectable markers, e.g., to detect or select for cells that have taken up the polynucleotide.

In certain embodiments, the viral vectors provided herein comprise one or more promoters that control expression of the transgene. These promoters (and other regulatory elements that control transcription, such as enhancers) may be constitutive (promote ubiquitous expression) or may specifically or selectively express in the liver (including promoting expression in the liver only or expressing in the liver at least at 1 to 100 fold greater levels than in a non-liver tissue), or may specifically or selectively express in the muscle (including promoting expression in the muscle only or expressing in the muscle at least at 1 to 100 fold greater levels than in a non-muscle tissue) or may specifically or selectively express in the liver and the muscle (including promoting expression in the liver and muscle only or expressing in the liver and muscle at least at 1 to 100 fold greater levels than in a non-liver/muscle tissue). In certain embodiments, the promoter is a constitutive promoter.

In certain embodiments, the promoter is a CB7 (also referred to as a CAG promoter) (see Dinculescu et al., 2005, Hum Gene Ther 16:649-663, incorporated by reference herein in its entirety). In some embodiments, the CAG or CB7 promoter (SEQ ID NO: 128) includes other expression control elements that enhance expression of the transgene driven by the vector. In certain embodiments, the other expression control elements include chicken β-actin intron and/or rabbit β-globin polyA signal. In certain embodiments, the promoter comprises a TATA box. In certain embodiments, the promoter comprises one or more elements. In certain embodiments, the one or more promoter elements may be inverted or moved relative to one another. In certain embodiments, the elements of the promoter are positioned to function cooperatively. In certain embodiments, the elements of the promoter are positioned to function independently. In certain embodiments, the viral vectors provided herein comprise one or more promoters selected from the group consisting of the human CMV immediate early gene promoter, the SV40 early promoter, the Rous sarcoma virus (RS) long terminal repeat, and rat insulin promoter. In certain embodiments, the vectors provided herein comprise one or more long terminal repeat (LTR) promoters selected from the group consisting of AAV, MLV, MMTV, SV40, RSV, HIV-1, and HIV-2 LTRs.

In certain embodiments, the vectors provided herein comprise one or more tissue specific promoters (e.g., a liver-specific promoter or a dual liver-muscle specific promoter). In particular embodiments, the viral vectors provided herein comprises a liver cell specific promoter, such as, a TBG (Thyroxine-binding Globulin) promoter (SEQ ID NO:40), an APOA2 promoter, one of the promoters of SEQ ID NO: 163-293, a SERPINA1 (hAAT) promoter, or an ApoE.hAAT promoter (SEQ ID NO:21). In certain embodiments, the viral vector provided herein comprises a muscle specific promoter, such as a human desmin promoter (Jonuschies et al., 2014, Curr. Gene Ther. 14:276-288), a CK8 promoter (SEQ ID NO:37; Himeda et al., 2011 Muscle Gene Therapy: Methods and Protocols, Methods in Molecular Biology, Dongsheng Duan (ed.), 709:3-19), or a Pitx3 promoter (Coulon et al., 2007, JBC 282:33192). In other embodiments, the viral vector comprises a VMD2 promoter.

Provided are nucleic acid regulatory elements that are chimeric with respect to arrangements of elements in tandem in the expression cassette. Regulatory elements, in general, have multiple functions as recognition sites for transcription initiation or regulation, coordination with cell-specific machinery to drive expression upon signaling, and to enhance expression of the downstream gene.

Also provided are arrangements of combinations of nucleic acid regulatory elements that promote transgene expression in liver tissue, or liver and muscle (skeletal and/or cardiac) tissue. In particular, certain elements are arranged with two or more copies of the individual enhancer and promoter elements arranged in tandem and operably linked to a transgene to promote expression, particularly tissue specific expression. Exemplary nucleotide sequences of the individual promoter and enhancer elements are provided in Table 1. Also provided in Table 1 are exemplary composite nucleic acid regulatory elements comprising the individual tandem promoter and enhancer elements. In certain embodiments the downstream promoter is an hAAT promoter (in certain embodiments the hAAT promoter is an hAAT(ΔATG) promoter) and the other promoter is another hAAT promoter or is a TBG promoter).

These combinations of promoter and enhancer sequences provided herein improve transgene expression while maintaining tissue specificity. Transgene expression from tandem promoters (i.e. two promoter sequences driving expression of the same transgene) is improved by depleting the 3′ promoter sequence of potential ‘ATG’ initiation sites. This approach was employed to improve transgene expression from tandem tissue-specific promoter cassettes (such as those targeting the liver) as well as promoter cassettes to achieve dual expression in two separate tissue populations (such as liver and skeletal muscle, and in certain embodiments cardiac muscle, and liver and bone). Ultimately, these designs aim to improve the therapeutic efficacy of gene transfer by providing more robust levels of transgene expression, improved stability/persistence, and induction of immune tolerance to the transgene product. In certain aspects the hAAT promoter with the start codon deleted (ΔATG) is used in an expression cassette provided herein.

Accordingly, with respect to liver and muscle specific expression, provided are nucleic acid regulatory elements that comprise or consist of promoters and/or other nucleic acid elements, such as enhancers, that promote liver expression, such as liver-specific CIS-regulatory enhancers of SEQ ID Nos: 163-293, ApoE enhancers, Mic/BiKE elements or hAAT promoters. These may be present as single copies or with two or more copies in tandem. The nucleic acid regulatory element may also comprise, in addition to the one or more elements that promote liver specific expression, one or more elements that promote muscle specific expression (including skeletal and/or cardiac muscle), for example, one or more copies, for example two copies, of the MckE element, which may be arranged as two or more copies in tandem or an MckE and MhcE elements arranged in tandem. In certain embodiments, a promoter element is deleted for the initiation codon to prevent translation initiation at that site, and preferably, the element with the modified start codon is the promoter that is the element at the 3′ end or the downstream end of the nucleic acid regulatory element, for example, closest within the nucleic acid sequence of the expression cassette to the transgene. In certain embodiments, the composite nucleic acid regulatory element comprises a hAAT promoter, in embodiments an hAAT which is start-codon modified (ΔATG) as the downstream promoter, and a second promoter in tandem with the hAAT promoter, which is an hAAT promoter, a CK8 promoter, an Spc5.12 promoter or an minSpc5.12 promoter. Nucleotide sequences are provided in Table 1.

In certain embodiments, the nucleotide sequence encoding the anti-pKal antibody heavy and light chains is operably linked to a composite nucleic acid regulatory element comprising a) two copies of Mic/BiKE arranged in tandem or two copies of ApoE arranged in tandem or two copies of Mic/BiKE arranged in tandem with one copy of ApoE, b) one promoter or, in tandem promoter embodiments, two promoters arranged in tandem comprising at least one copy of hAAT which is start-codon modified (ΔATG) (where in certain embodiments the hAAT promoter is the downstream or 3′ promoter). In some embodiments, the composite nucleic acid regulatory element comprises LSPX1, LSPX2, LTP1, LTP2, or LTP3 of Table 1.

Also provided are recombinant expression cassettes in which the nucleotide sequence encoding the heavy and light chains of the anti-pKal antibody is operably linked to a nucleic acid regulatory element comprising a) one copy of ApoE, two or three copies of MckE arranged in tandem, one copy of each MckE, MhcE, and ApoE arrange in tandem, or two or three copies of MckE arranged in tandem with one copy of ApoE, b) two copies of a promoter arranged in tandem comprising at least one copy of hAAT which is start-codon modified (ΔATG). In certain embodiments, the second and upstream promoter is a CK8 promoter, an Spc5.12 promoter or a minSpc5.12 promoter. In some embodiments, the composite nucleic acid regulatory element comprises LMTP6, LMTP13, LMTP14, LMTP15, LMTP18, LMTP19, or LMTP20 of Table 1.

In certain embodiments, the nucleotide sequence encoding the anti-pKal antibody heavy and light chains is operably linked to a composite nucleic acid regulatory element comprising a) two copies of a liver-specific CRE selected SEQ ID Nos 163-293 arranged in tandem or two copies of Mic/BiKE arranged in tandem with one copy of a liver-specific CRE selected from SEQ ID Nos: 163-293, b) one promoter or, in tandem promoter embodiments, two promoters arranged in tandem comprising at least one copy of hAAT which is start-codon modified (ΔATG) (where in certain embodiments the hAAT promoter is the downstream or 3′ promoter). In some embodiments, the composite nucleic acid regulatory element comprises LSPX1, LSPX2, LTP1, LTP2, or LTP3 of Table 1.

Also provided are recombinant expression cassettes in which the nucleotide sequence encoding the heavy and light chains of the anti-pKal antibody or an scFv-Fc is operably linked to a nucleic acid regulatory element comprising a) one copy of a liver-specific CRE selected from SEQ ID Nos 163-293, one copy of each MckE, MhcE, and a liver-specific CRE selected from SEQ ID Nos 163-293 arranged in tandem, or two or three copies of MckE arranged in tandem with one copy of a liver-specific CRE selected from SEQ ID Nos: 163-293, b) two copies of a promoter arranged in tandem comprising at least one copy of hAAT which is start-codon modified (ΔATG). In certain embodiments, the second and upstream promoter is a CK8 promoter, an Spc5.12 promoter or a minSpc5.12 promoter. In some embodiments, the composite nucleic acid regulatory element comprises LMTP6, LMTP13, LMTP14, LMTP15, LMTP18, LMTP19, or LMTP20 of Table 1.

In certain embodiments, the anti-pKal therapeutic antibody coding sequence is operably linked to composite nucleic acid regulatory elements for enhancing gene expression in the liver LSPX1 (SEQ ID NO:9), LSPX2 (SEQ ID NO:10), LTP1 (SEQ ID NO:11), LTP2 (SEQ ID NO: 12), or LTP3 (SEQ ID NO:13), liver and muscle expression, LMTP6 (SEQ ID NO:14), LMTP13 (SEQ ID NO:15), LMTP14 (SEQ ID NO:16), LMTP15 (SEQ ID NO:17), LMTP18 (SEQ ID NO:18), LMTP19 (SEQ ID NO:19), or LMTP20 (SEQ ID NO:20), the sequences of which are provided in Table 1 below. Also included are composite regulatory elements that enhance gene expression in the liver, and in certain embodiments, also muscle or bone, which have 99%, 95%, 90%, 85% or 80% sequence identity with one of nucleic acid sequences LSPX1 (SEQ ID NO:9), LSPX2 (SEQ ID NO: 10), LTP1 (SEQ ID NO:11), LTP2 (SEQ ID NO:12), or LTP3 (SEQ ID NO:13), LMTP6 (SEQ ID NO: 14), LMTP13 (SEQ ID NO:15), LMTP14 (SEQ ID NO:16), LMTP15 (SEQ ID NO:17), LMTP18 (SEQ ID NO:18), LMTP19 (SEQ ID NO:19), or LMTP20 (SEQ ID NO:20).

The tandem and composite promoters described herein result in preferred transcription start sites within the promoter region. Thus, in certain embodiments, the constructs described herein have a tandem or composite nucleic acid regulatory sequence that comprises an hAAT promoter (particularly a modified start codon hAAT promoter) and has a transcription start site of TCTCC (SEQ ID NO: 335) (corresponding to nt 1541-1545 of LMTP6 (SEQ ID NO:14), which overlaps with the active TTS found in hAAT (nt 355-359 of SEQ ID NO:30) or GGTACAATGACTCCTTTCG (SEQ ID NO: 337), which corresponds to nucleotides 139-157 of SEQ ID NO:30, or GGTACAGTGACTCCTTTCG (SEQ ID NO:336), which corresponds to nucleotides 139-157 of SEQ ID NO: 31. In other embodiments, the constructs described herein have a tandem or composite regulatory sequence that comprises a CK8 promoter and has a transcription start site at TCATTCTACC (SEQ ID NO:338), which corresponds to nucleotides 377-386 of SEQ ID NO:37, particularly starting at the nucleotide corresponding to nucleotide 377 of SEQ ID NO:14 or corresponding to nucleotide 1133 of SEQ ID NO:14.

In certain embodiments, the promoter is an inducible promoter. In certain embodiments the promoter is a hypoxia-inducible promoter. In certain embodiments, the promoter comprises a hypoxia-inducible factor (HIF) binding site. In certain embodiments, the promoter comprises a HIF-la binding site. In certain embodiments, the promoter comprises a HIF-2α binding site. In certain embodiments, the HIF binding site comprises an RCGTG motif. For details regarding the location and sequence of HIF binding sites, see, e.g., Schödel, et al., Blood, 2011, 117(23):e207-e217, which is incorporated by reference herein in its entirety. In certain embodiments, the promoter comprises a binding site for a hypoxia induced transcription factor other than a HIF transcription factor. In certain embodiments, the viral vectors provided herein comprise one or more IRES sites that is preferentially translated in hypoxia. For teachings regarding hypoxia-inducible gene expression and the factors involved therein, see, e.g., Kenneth and Rocha, Biochem J., 2008, 414:19-29, which is incorporated by reference herein in its entirety. In specific embodiments, the hypoxia-inducible promoter is the human N-WASP promoter, see, e.g., Salvi, 2017, Biochemistry and Biophysics Reports 9:13-21 (incorporated by reference for the teaching of the N-WASP promoter) or is the hypoxia-induced promoter of human Epo, see, e.g., Tsuchiya et al., 1993, J. Biochem. 113:395-400 (incorporated by reference for the disclosure of the Epo hypoxia-inducible promoter). In other embodiments, the promoter is a drug inducible promoter, for example, a promoter that is induced by administration of rapamycin or analogs thereof. See, e.g., the disclosure of rapamycin inducible promoters in PCT publications WO94/18317, WO 96/20951, WO 96/41865, WO 99/10508, WO 99/10510, WO 99/36553, and WO 99/41258, and U.S. Pat. No. 7,067,526, which are hereby incorporated by reference in their entireties for the disclosure of drug inducible promoters.

Provided herein are constructs containing certain ubiquitous and tissue-specific promoters. Such promoters include synthetic and tandem promoters. Examples and nucleotide sequences of promoters are provided in Table 1 below. Table 1 also includes the nucleotide sequences of other regulatory elements useful for the expression cassettes provided herein

TABLE 1
Promoter and Other Regulatory Element Sequences

| Name/ | |
|---|---|
| SEQ ID NO. | Sequence |
| LSPX1 | aggttaatttttaaaaagcagtcaaaagtccaagtggcccttggcagcatttactctctctgt |
| SEQ ID NO: 9 | ttgctctggttaataatctcaggagcacaaacattccagatccaggttaatttttaaaaagca |
| | gtcaaaagtccaagtggcccttggcagcatttactctctctgtttgctctggttaataatctc |
| | aggagcacaaacattccagatccggcgcgccagggctggaagctacctttgtctagaaggctc |
| | agaggcacacaggagtttctgggctcaccctgcccccttccaacccctcagttcccatcctcc |
| | agcagctgtttgtgtgctgcctctgaagtccacactgaacaaacttcagcctactcatgtccc |
| | taaaatgggcaaacattgcaagcagcaaacagcaaacacacagccctccctgcctgctgacct |
| | tggagctggggcagaggtcagagacctctctgggcccatgccacctccaacatccactcgacc |
| | ccttggaatttcggtggagaggagcagaggttgtcctggcgtggtttaggtagtgtgagaggg |
| | gtacccggggatcttgctaccagtggaacagccactaaggattctgcagtgagagcagagggc |
| | cagctaagtggtactctcccagagactgtctgactcacgccaccccctccaccttggacacag |
| | gacgctgtggtttctgagccaggtacaatgactcctttcggtaagtgcagtggaagctgtaca |
| | ctgcccaggcaaagcgtccgggcagcgtaggcgggcgactcagatcccagccagtggacttag |
| | cccctgtttgctcctccgataactggggtgaccttggttaatattcaccagcagcctcccccg |
| | ttgcccctctggatccactgcttaaatacggacgaggacagggccctgtctcctcagcttcag |
| | gcaccaccactgacctgggacagt |
| LSXP2 | aggctcagaggcacacaggagtttctgggctcaccctgcccccttccaacccctcagttccca |
| SEQ ID NO: 10 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agagggtctagaaggctcagaggcacacaggagtttctgggctcaccctgcccccttccaacc |
| | cctcagttcccatcctccagcagctgtttgtgtgctgcctctgaagtccacactgaacaaact |
| | tcagcctactcatgtccctaaaatgggcaaacattgcaagcagcaaacagcaaacacacagcc |
| | ctccctgcctgctgaccttggagctggggcagaggtcagagacctctctgggcccatgccacc |
| | tccaacatccactcgaccccttggaatttcggtggagaggagcagaggttgtcctggcgtggt |
| | ttaggtagtgtgagaggggtacccggggatcttgctaccagtggaacagccactaaggattct |
| | gcagtgagagcagagggccagctaagtggtactctcccagagactgtctgactcacgccaccc |
| | cctccaccttggacacaggacgctgtggtttctgagccaggtacaatgactcctttcggtaag |
| | tgcagtggaagctgtacactgcccaggcaaagcgtccgggcagcgtaggcgggcgactcagat |
| | cccagccagtggacttagcccctgtttgctcctccgataactggggtgaccttggttaatatt |
| | caccagcagcctcccccgttgcccctctggatccactgcttaaatacggacgaggacagggcc |
| | ctgtctcctcagcttcaggcaccaccactgacctgggacagt |
| LTP1 | aggttaatttttaaaaagcagtcaaaagtccaagtggcccttggcagcatttactctctctgt |
| SEQ ID NO: 11 | ttgctctggttaataatctcaggagcacaaacattccagatccaggttaatttttaaaaagca |
| | gtcaaaagtccaagtggcccttggcagcatttactctctctgtttgctctggttaataatctc |
| | aggagcacaaacattccagatccggcgcgccagggctggaagctacctttgacatcatttcct |
| | ctgcgaatgcatgtataatttctacagaacctattagaaaggatcacccagcctctgcttttg |
| | tacaactttcccttaaaaaactgccaattccactgctgtttggcccaatagtgagaacttttt |
| | cctgctgcctcttggtgcttttgcctatggcccctattctgcctgctgaagacactcttgcca |
| | gcatggacttaaacccctccagctctgacaatcctctttctcttttgttttacatgaagggtc |
| | tggcagccaaagcaatcactcaaagttcaaaccttatcattttttgctttgttcctcttggcc |
| | ttggttttgtacatcagctttgaaaataccatcccagggttaatgctggggttaatttataac |
| | taagagtgctctagttttgcaatacaggacatgctataaaaatggaaagatgttgctttctga |
| | gaggatcttgctaccagtggaacagccactaaggattctgcagtgagagcagagggccagcta |
| | agtggtactctcccagagactgtctgactcacgccaccccctccaccttggacacaggacgct |
| | gtggtttctgagccaggtacagtgactcctttcggtaagtgcagtggaagctgtacactgccc |
| | aggcaaagcgtccgggcagcgtaggcgggcgactcagatcccagccagtggacttagcccctg |
| | tttgctcctccgataactggggtgaccttggttaatattcaccagcagcctcccccgttgccc |
| | ctctggatccactgcttaaatacggacgaggacagggccctgtctcctcagcttcaggcacca |
| | ccactgacctgggacagt |
| LTP2 | aggctcagaggcacacaggagtttctgggctcaccctgcccccttccaacccctcagttccca |
| SEQ ID NO: 12 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agagggtctagagcccttaagctagcaggttaatttttaaaaagcagtcaaaagtccaagtgg |
| | cccttggcagcatttactctctctgtttgctctggttaataatctcaggagcacaaacattcc |
| | agatccaggttaatttttaaaaagcagtcaaaagtccaagtggcccttggcagcatttactct |
| | ctctgtttgctctggttaataatctcaggagcacaaacattccagatccggcgcgccagggct |
| | ggaagctacctttgacatcatttcctctgcgaatgcatgtataatttctacagaacctattag |
| | aaaggatcacccagcctctgcttttgtacaactttcccttaaaaaactgccaattccactgct |
| | gtttggcccaatagtgagaactttttcctgctgcctcttggtgcttttgcctatggcccctat |
| | tctgcctgctgaagacactcttgccagcatggacttaaacccctccagctctgacaatcctct |
| | ttctcttttgttttacatgaagggtctggcagccaaagcaatcactcaaagttcaaaccttat |
| | cattttttgctttgttcctcttggccttggttttgtacatcagctttgaaaataccatcccag |
| | ggttaatgctggggttaatttataactaagagtgctctagttttgcaatacaggacatgctat |
| | aaaaatggaaagatgttgctttctgagaggatcttgctaccagtggaacagccactaaggatt |
| | ctgcagtgagagcagagggccagctaagtggtactctcccagagactgtctgactcacgccac |
| | cccctccaccttggacacaggacgctgtggtttctgagccaggtacagtgactcctttcggta |
| | agtgcagtggaagctgtacactgcccaggcaaagcgtccgggcagcgtaggcgggcgactcag |
| | atcccagccagtggacttagcccctgtttgctcctccgataactggggtgaccttggttaata |
| | ttcaccagcagcctcccccgttgcccctctggatccactgcttaaatacggacgaggacaggg |
| | ccctgtctcctcagcttcaggcaccaccactgacctgggacagt |
| LTP3 | aggttaatttttaaaaagcagtcaaaagtccaagtggcccttggcagcatttactctctctgt |
| SEQ ID NO: 13 | ttgctctggttaataatctcaggagcacaaacattccagatccaggttaatttttaaaaagca |
| | gtcaaaagtccaagtggcccttggcagcatttactctctctgtttgctctggttaataatctc |
| | aggagcacaaacattccagatccggcgcgccagggctggaagctacctttgacatcatttcct |
| | ctgcgaatgcatgtataatttctacagaacctattagaaaggatcacccagcctctgcttttg |
| | tacaactttcccttaaaaaactgccaattccactgctgtttggcccaatagtgagaacttttt |
| | cctgctgcctcttggtgcttttgcctatggcccctattctgcctgctgaagacactcttgcca |
| | gcatggacttaaacccctccagctctgacaatcctctttctcttttgttttacatgaagggtc |
| | tggcagccaaagcaatcactcaaagttcaaaccttatcattttttgctttgttcctcttggcc |
| | ttggttttgtacatcagctttgaaaataccatcccagggttaatgctggggttaatttataac |
| | taagagtgctctagttttgcaatacaggacatgctataaaaatggaaagatgttgctttctga |
| | gaggatcttgctaccagtggaacagccactaaggattctgcagtgagagcagagggccagcta |
| | agtggtactctcccagagactgtctgactcacgccaccccctccaccttggacacaggacgct |
| | gtggtttctgagccaggtacagtgactcctttcggtaagtgcagtggaagctgtacactgccc |
| | aggcaaagcgtccgggcagcgtaggcgggcgactcagatcccagccagtggacttagcccctg |
| | tttgctcctccgataactggggtgaccttggttaatattcaccagcagcctcccccgttgccc |
| | ctctggatccactgcttaaatacggacgaggacagggccctgtctcctcagcttcaggcacca |
| | ccactgacctgggacagtaaaacaggtaagtccgctgtttgtgtgctgcctctgaagtccaca |
| | ctgaacaaacttcagcctactcatgtccctaaaatgggcaaacattgcaagcagcaaacagca |
| | aacacacagccctccctgcctgctgaccttggagctggggcagaggtcagagacctctctggc |
| | ctctactaaccatgttcatgttttctttttttttctacaggtcctgggtgacgaacag |
| LMTP6 | aggctcagaggcacacaggagtttctgggctcaccctgcccccttccaacccctcagttccca |
| SEQ ID NO: 14 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agagggccactacgggtttaggctgcccatgtaaggaggcaaggcctggggacacccgagatg |
| | cctggttataattaacccagacatgtggctgccccccccccccccaacacctgctgcctctaa |
| | aaataaccctgtccctggtggatcccactacgggtttaggctgcccatgtaaggaggcaaggc |
| | ctggggacacccgagatgcctggttataattaacccagacatgtggctgcccccccccccccc |
| | aacacctgctgcctctaaaaataaccctgtccctggtggatcccactacgggtttaggctgcc |
| | catgtaaggaggcaaggcctggggacacccgagatgcctggttataattaacccagacatgtg |
| | gctgccccccccccccccaacacctgctgcctctaaaaataaccctgtccctggtggatcccc |
| | tgcatgcgaagatcttcgaacaaggctgtgggggactgagggcaggctgtaacaggcttgggg |
| | gccagggcttatacgtgcctgggactcccaaagtattactgttccatgttcccggcgaagggc |
| | cagctgtcccccgccagctagactcagcacttagtttaggaaccagtgagcaagtcagccctt |
| | ggggcagcccatacaaggccatggggctgggcaagctgcacgcctgggtccggggtgggcacg |
| | gtgcccgggcaacgagctgaaagctcatctgctctcaggggcccctccctggggacagcccct |
| | cctggctagtcacaccctgtaggctcctctatataacccaggggcacaggggctgccctcatt |
| | ctaccaccacctccacagcacagacagacactcaggagccagccagcgtcgagatcttgctac |
| | cagtggaacagccactaaggattctgcagtgagagcagagggccagctaagtggtactctccc |
| | agagactgtctgactcacgccaccccctccaccttggacacaggacgctgtggtttctgagcc |
| | aggtacagtgactcctttcggtaagtgcagtggaagctgtacactgcccaggcaaagcgtccg |
| | ggcagcgtaggcgggcgactcagatcccagccagtggacttagcccctgtttgctcctccgat |
| | aactggggtgaccttggttaatattcaccagcagcctcccccgttgcccctctggatccactg |
| | cttaaatacggacgaggacagggccctgtctcctcagcttcaggcaccaccactgacctggga |
| | cagt |
| LMTP13 | aggctcagaggcacacaggagtttctgggctcaccctgcccccttccaacccctcagttccca |
| SEQ ID NO: 15 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agaggggtacccggggatcttgctaccagtctagaggccgtccgccctcggcaccatcctcac |
| | gacacccaaatatggcgacgggtgaggaatggtggggagttatttttagagcggtgaggaagg |
| | tgggcaggcagcaggtgttggcgctctaaaaataactcccgggagttatttttagagcggagg |
| | aatggtggacacccaaatatggcgacggttcctcacccgtcgccatatttgggtgtccgccct |
| | cggccggggccgcattcctgggggccgggcggtgctcccgcccgcctcgataaaaggctccgg |
| | ggccggcggcggcccacgagctacccggaggagcgggaggcgccaagcgtgagtatcgatctt |
| | gctaccagtggaacagccactaaggattctgcagtgagagcagagggccagctaagtggtact |
| | ctcccagagactgtctgactcacgccaccccctccaccttggacacaggacgctgtggtttct |
| | gagccaggtacagtgactcctttcggtaagtgcagtggaagctgtacactgcccaggcaaagc |
| | gtccgggcagcgtaggcgggcgactcagatcccagccagtggacttagcccctgtttgctcct |
| | ccgataactggggtgaccttggttaatattcaccagcagcctcccccgttgcccctctggatc |
| | cactgcttaaatacggacgaggacagggccctgtctcctcagcttcaggcaccaccactgacc |
| | tgggacagt |
| LMTP14 | gaatggtggacacccaaatatggcgacggttcctcacccgtcgccatatttgggtgtccgccc |
| SEQ ID NO: 16 | tcggccggggccgcattcctgggggccgggcggtgctcccgcccgcctcgataaaaggctccg |
| | gggccggcggcggcccacgagctacccggaggagcgggaggcgccaagcgatcttgctaccag |
| | tggaacagccactaaggattctgcagtgagagcagagggccagctaagtggtactctcccaga |
| | gactgtctgactcacgccaccccctccaccttggacacaggacgctgtggtttctgagccagg |
| | tacagtgactcctttcggtaagtgcagtggaagctgtacactgcccaggcaaagcgtccgggc |
| | agcgtaggcgggcgactcagatcccagccagtggacttagcccctgtttgctcctccgataac |
| | tggggtgaccttggttaatattcaccagcagcctcccccgttgcccctctggatccactgctt |
| | aaatacggacgaggacagggccctgtctcctcagcttcaggcaccaccactgacctgggacag |
| | t |
| LMTP15 | aggctcagaggcacacaggagtttctgggctcaccctgcccccttccaacccctcagttccca |
| SEQ ID NO: 17 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agagggtctagagaatggtggacacccaaatatggcgacggttcctcacccgtcgccatattt |
| | gggtgtccgccctcggccggggccgcattcctgggggccgggcggtgctcccgcccgcctcga |
| | taaaaggctccggggccggcggcggcccacgagctacccggaggagcgggaggcgccaagcga |
| | tcttgctaccagtggaacagccactaaggattctgcagtgagagcagagggccagctaagtgg |
| | tactctcccagagactgtctgactcacgccaccccctccaccttggacacaggacgctgtggt |
| | ttctgagccaggtacagtgactcctttcggtaagtgcagtggaagctgtacactgcccaggca |
| | aagcgtccgggcagcgtaggcgggcgactcagatcccagccagtggacttagcccctgtttgc |
| | tcctccgataactggggtgaccttggttaatattcaccagcagcctcccccgttgcccctctg |
| | gatccactgcttaaatacggacgaggacagggccctgtctcctcagcttcaggcaccaccact |
| | gacctgggacagt |
| LMTP18 | aggctcagaggcacacaggagtttctgggctcaccctgcccccttccaacccctcagttccca |
| SEQ ID NO: 18 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agagggccactacgggtttaggctgcccatgtaaggaggcaaggcctggggacacccgagatg |
| | cctggttataattaacccagacatgtggctgccccccccccccccaacacctgctgcctctaa |
| | aaataaccctgtccctggtggatcccctgcatgcgaagatcttcgaacaaggctgtgggggac |
| | tgagggcaggctgtaacaggcttgggggccagggcttatacgtgcctgggactcccaaagtat |
| | tactgttccatgttcccggcgaagggccagctgtcccccgccagctagactcagcacttagtt |
| | taggaaccagtgagcaagtcagcccttggggcagcccatacaaggccatggggctgggcaagc |
| | tgcacgcctgggtccggggtgggcacggtgcccgggcaacgagctgaaagctcatctgctctc |
| | aggggcccctccctggggacagcccctcctggctagtcacaccctgtaggctcctctatataa |
| | cccaggggcacaggggctgccctcattctaccaccacctccacagcacagacagacactcagg |
| | agccagccagcgtcgagatcttgctaccagtggaacagccactaaggattctgcagtgagagc |
| | agagggccagctaagtggtactctcccagagactgtctgactcacgccaccccctccaccttg |
| | gacacaggacgctgtggtttctgagccaggtacagtgactcctttcggtaagtgcagtggaag |
| | ctgtacactgcccaggcaaagcgtccgggcagcgtaggcgggcgactcagatcccagccagtg |
| | gacttagcccctgtttgctcctccgataactggggtgaccttggttaatattcaccagcagcc |
| | tcccccgttgcccctctggatccactgcttaaatacggacgaggacagggccctgtctcctca |
| | gcttcaggcaccaccactgacctgggacagt |
| LMTP19 | aggctcagaggcacacaggagtttctgggctcaccctgcccccttccaacccctcagttccca |
| SEQ ID NO: 19 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agagggccctgcatgcgaagatcttcgaacaaggctgtgggggactgagggcaggctgtaaca |
| | ggcttgggggccagggcttatacgtgcctgggactcccaaagtattactgttccatgttcccg |
| | gcgaagggccagctgtcccccgccagctagactcagcacttagtttaggaaccagtgagcaag |
| | tcagcccttggggcagcccatacaaggccatggggctgggcaagctgcacgcctgggtccggg |
| | gtgggcacggtgcccgggcaacgagctgaaagctcatctgctctcaggggcccctccctgggg |
| | acagcccctcctggctagtcacaccctgtaggctcctctatataacccaggggcacaggggct |
| | gccctcattctaccaccacctccacagcacagacagacactcaggagccagccagcgtcgaga |
| | tcttgctaccagtggaacagccactaaggattctgcagtgagagcagagggccagctaagtgg |
| | tactctcccagagactgtctgactcacgccaccccctccaccttggacacaggacgctgtggt |
| | ttctgagccaggtacagtgactcctttcggtaagtgcagtggaagctgtacactgcccaggca |
| | aagcgtccgggcagcgtaggcgggcgactcagatcccagccagtggacttagcccctgtttgc |
| | tcctccgataactggggtgaccttggttaatattcaccagcagcctcccccgttgcccctctg |
| | gatccactgcttaaatacggacgaggacagggccctgtctcctcagcttcaggcaccaccact |
| | gacctgggacagt |
| LMTP20 | aggctcagaggcacacaggagtttctgggctcaccctgcccccttccaacccctcagttccca |
| SEQ ID NO: 20 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agagggcccttcagattaaaaataactgaggtaagggcctgggtaggggaggtggtgtgagac |
| | gctcctgtctctcctctatctgcccatcggccctttggggaggaggaatgtgcccaaggacta |
| | aaaaaaggccatggagccagaggggcgagggcaacagacctttcatgggcaaaccttggggcc |
| | ctgctgaagctttggcccactacgggtttaggctgcccatgtaaggaggcaaggcctggggac |
| | acccgagatgcctggttataattaacccagacatgtggctgccccccccccccccaacacctg |
| | ctgcctctaaaaataaccctgtccctggtggatcccctgcatgcgaagatcttcgaacaaggc |
| | tgtgggggactgagggcaggctgtaacaggcttgggggccagggcttatacgtgcctgggact |
| | cccaaagtattactgttccatgttcccggcgaagggccagctgtcccccgccagctagactca |
| | gcacttagtttaggaaccagtgagcaagtcagcccttggggcagcccatacaaggccatgggg |
| | ctgggcaagctgcacgcctgggtccggggtgggcacggtgcccgggcaacgagctgaaagctc |
| | atctgctctcaggggcccctccctggggacagcccctcctggctagtcacaccctgtaggctc |
| | ctctatataacccaggggcacaggggctgccctcattctaccaccacctccacagcacagaca |
| | gacactcaggagccagccagcgtcgagatcttgctaccagtggaacagccactaaggattctg |
| | cagtgagagcagagggccagctaagtggtactctcccagagactgtctgactcacgccacccc |
| | ctccaccttggacacaggacgctgtggtttctgagccaggtacagtgactcctttcggtaagt |
| | gcagtggaagctgtacactgcccaggcaaagcgtccgggcagcgtaggcgggcgactcagatc |
| | ccagccagtggacttagcccctgtttgctcctccgataactggggtgaccttggttaatattc |
| | accagcagcctcccccgttgcccctctggatccactgcttaaatacggacgaggacagggccc |
| | tgtctcctcagcttcaggcaccaccactgacctgggacagt |
| ApoE.hAAT | aggctcagaggcacacaggagtttctgggctcaccctgcccccttccaacccctcagttccca |
| SEQ ID NO: 21 | tcctccagcagctgtttgtgtgctgcctctgaagtccacactgaacaaacttcagcctactca |
| | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agaggggtacccggggatcttgctaccagtggaacagccactaaggattctgcagtgagagca |
| | gagggccagctaagtggtactctcccagagactgtctgactcacgccaccccctccaccttgg |
| | acacaggacgctgtggtttctgagccaggtacaatgactcctttcggtaagtgcagtggaagc |
| | tgtacactgcccaggcaaagcgtccgggcagcgtaggcgggcgactcagatcccagccagtgg |
| | acttagcccctgtttgctcctccgataactggggtgaccttggttaatattcaccagcagcct |
| | cccccgttgcccctctggatccactgcttaaatacggacgaggacagggccctgtctcctcag |
| | cttcaggcaccaccactgacctgggacagt |
| Alpha-Mic/Bik | aggttaatttttaaaaagcagtcaaaagtccaagtggcccttggcagcatttactctctctgt |
| Enhancer | ttgctctggttaataatctcaggagcacaaacattcc |
| (Mic/BikE) | |
| SEQ ID NO: 26 | |
| Tandem (2) | aggttaatttttaaaaagcagtcaaaagtccaagtggcccttggcagcatttactctctctgt |
| alpha-Mic/Bik | ttgctctggttaataatctcaggagcacaaacattccaggttaatttttaaaaagcagtcaaa |
| Enhancers | agtccaagtggcccttggcagcatttactctctctgtttgctctggttaataatctcaggagc |
| (2 Mic/BikE) | acaaacattcc |
| SEQ ID NO: 27 | |
| ApoE Hepatic | aggctcagaggcacacaggagtttctgggctcaccctgcccccttccaacccctcagttccca |
| Control Region | tcctccagcagctgtttgtgtgctgcctctgaagtccacactgaacaaacttcagcctactca |
| containing ApoE | tgtccctaaaatgggcaaacattgcaagcagcaaacagcaaacacacagccctccctgcctgc |
| Enhancer | tgaccttggagctggggcagaggtcagagacctctctgggcccatgccacctccaacatccac |
| SEQ ID NO: 28 | tcgaccccttggaatttcggtggagaggagcagaggttgtcctggcgtggtttaggtagtgtg |
| | agaggg |
| Tandem (2) | aggctcagaggcacacaggagtttctgggctcaccctgcccccttccaacccctcagttccca |
| ApoE Enhancers | tcctccagcagctgtttgtgtgctgcctctgaagtccacactgaacaaacttcagcctactca |
| SEQ ID NO: 29 | tgtccctaaaatgggcaaacattgcaagcagcaaacagcaaacacacagccctccctgcctgc |
| | tgaccttggagctggggcagaggtcagagacctctctgggcccatgccacctccaacatccac |
| | tcgaccccttggaatttcggtggagaggagcagaggttgtcctggcgtggtttaggtagtgtg |
| | agagggtctagaaggctcagaggcacacaggagtttctgggctcaccctgcccccttccaacc |
| | cctcagttcccatcctccagcagctgtttgtgtgctgcctctgaagtccacactgaacaaact |
| | tcagcctactcatgtccctaaaatgggcaaacattgcaagcagcaaacagcaaacacacagcc |
| | ctccctgcctgctgaccttggagctggggcagaggtcagagacctctctg |
| hAAT Promoter | gatcttgctaccagtggaacagccactaaggattctgcagtgagagcagagggccagctaagt |
| SEQ ID NO: 30 | ggtactctcccagagactgtctgactcacgccaccccctccaccttggacacaggacgctgtg |
| | gtttctgagccaggtacaatgactcctttcggtaagtgcagtggaagctgtacactgcccag |
| | gcaaagcgtccgggcagcgtaggcgggcgactcagatcccagccagtggacttagcccctgtt |
| | tgctcctccgataactggggtgaccttggttaatattcaccagcagcctcccccgttgcccct |
| | ctggatccactgcttaaatacggacgaggacagggccctgtctcctcagcttcaggcaccacc |
| | actgacctgggacagt |
| hAAT(AATG) | gatcttgctaccagtggaacagccactaaggattctgcagtgagagcagagggccagctaagt |
| Promoter | ggtactctcccagagactgtctgactcacgccaccccctccaccttggacacaggacgctgtg |
| SEQ ID NO: 31 | gtttctgagccaggtacagtgactcctttcggtaagtgcagtggaagctgtacactgcccag |
| | gcaaagcgtccgggcagcgtaggcgggcgactcagatcccagccagtggacttagcccctgtt |
| | tgctcctccgataactggggtgaccttggttaatattcaccagcagcctcccccgttgcccct |
| | ctggatccactgcttaaatacggacgaggacagggccctgtctcctcagcttcaggcaccacc |
| | actgacctgggacagt |
| Mck Enhancer | ccactacgggtttaggctgcccatgtaaggaggcaaggcctggggacacccgagatgcctggt |
| (MckE) | tataattaacccagacatgtggctgccccccccccccccaacacctgctgcctctaaaaataa |
| SEQ ID NO: 32 | ccctgtccctggtggatc |
| Tandem (2) Mck | ccactacgggtttaggctgcccatgtaaggaggcaaggcctggggacacccgagatgcctggt |
| Enhancers | tataattaacccagacatgtggctgccccccccccccccaacacctgctgcctctaaaaataa |
| (2 MckE) | ccctgtccctggtggatcccactacgggtttaggctgcccatgtaaggaggcaaggcctgggg |
| SEQ ID NO: 33 | acacccgagatgcctggttataattaacccagacatgtggctgccccccccccccccaacacc |
| | tgctgcctctaaaaataaccctgtccctggtggatc |
| Tandem Mck (3) | ccactacgggtttaggctgcccatgtaaggaggcaaggcctggggacacccgagatgcctggt |
| Enhancers | tataattaacccagacatgtggctgccccccccccccccaacacctgctgcctctaaaaataa |
| (3 MckE) | ccctgtccctggtggatcccactacgggtttaggctgcccatgtaaggaggcaaggcctgggg |
| SEQ ID NO: 34 | acacccgagatgcctggttataattaacccagacatgtggctgccccccccccccccaacacc |
| | tgctgcctctaaaaataaccctgtccctggtggatcccactacgggtttaggctgcccatgta |
| | aggaggcaaggcctggggacacccgagatgcctggttataattaacccagacatgtggctgcc |
| | ccccccccccccaacacctgctgcctctaaaaataaccctgtccctggtggatc |
| Myosin heavy | cccttcagattaaaaataactgaggtaagggcctgggtaggggaggtggtgtgagacgctcct |
| chain enhancer | gtctctcctctatctgcccatcggccctttggggaggaggaatgtgcccaaggactaaaaaaa |
| (MhcE) | ggccatggagccagaggggcgagggcaacagacctttcatgggcaaaccttggggccctgctg |
| SEQ ID NO: 35 | aagctttggc |
| CAG/CB7 | gacattgattattgactagttattaatagtaatcaattacggggtcattagttcatagcccat |
| SEQ ID NO: 36 | atatggagttccgcgttacataacttacggtaaatggcccgcctggctgaccgcccaacgacc |
| | cccgcccattgacgtcaataatgacgtatgttcccatagtaacgccaatagggactttccatt |
| | gacgtcaatgggtggagtatttacggtaaactgcccacttggcagtacatcaagtgtatcata |
| | tgccaagtacgccccctattgacgtcaatgacggtaaatggcccgcctggcattatgcccagt |
| | acatgaccttatgggactttcctacttggcagtacatctacgtattagtcatcgctattacca |
| | tggtcgaggtgagccccacgttctgcttcactctccccatctcccccccctccccacccccaa |
| | ttttgtatttatttattttttaattattttgtgcagcgatgggggcggggggggggggggggc |
| | gcgcgccaggcggggcggggcggggcgaggggcggggcggggcgaggcggagaggtgcggcgg |
| | cagccaatcagagcggcgcgctccgaaagtttccttttatggcgaggcggcggcggcggcggc |
| | cctataaaaagcgaagcgcgcggcgggcgggagtcgctgcgcgctgccttcgccccgtgcccc |
| | gctccgccgccgcctcgcgccgcccgccccggctctgactgaccgcgttactcccacaggtga |
| | gcgggcgggacggcccttctcctccgggctgtaattagcgcttggtttaatgacggcttgttt |
| | cttttctgtggctgcgtgaaagccttgaggggctccgggagggccctttgtgcggggggagcg |
| | gctcggggggtgcgtgcgtgtgtgtgtgcgtggggagcgccgcgtgcggctccgcgctgcccg |
| | gcggctgtgagcgctgcgggcgcggcgcggggctttgtgcgctccgcagtgtgcgcgagggga |
| | gcgcggccgggggcggtgccccgcggtgcggggggggctgcgaggggaacaaaggctgcgtgc |
| | ggggtgtgtgcgtgggggggtgagcagggggtgtgggcgcgtcggtcgggctgcaaccccccc |
| | tgcacccccctccccgagttgctgagcacggcccggcttcgggtgcggggctccgtacggggc |
| | gtggcgcggggctcgccgtgccgggcggggggtggcggcaggtgggggtgccgggcggggcgg |
| | ggccgcctcgggccggggagggctcgggggaggggcgcggcggcccccggagcgccggcggct |
| | gtcgaggcgcggcgagccgcagccattgccttttatggtaatcgtgcgagagggcgcagggac |
| | ttcctttgtcccaaatctgtgcggagccgaaatctgggaggcgccgccgcaccccctctagcg |
| | ggcgcggggcgaagcggtgcggcgccggcaggaaggaaatgggcggggagggccttcgtgcgt |
| | cgccgcgccgccgtccccttctccctctccagcctcggggctgtccgcggggggacggctgcc |
| | ttcgggggggacggggcagggcggggttcggcttctggcgtgtgaccggcggctctagagcct |
| | ctgctaaccatgttcatgccttcttctttttcctacagctcctgggcaacgtgctggttattg |
| | tgctgtctcatcattttggcaaag |
| CK8 | ccactacgggtttaggctgcccatgtaaggaggcaaggcctggggacacccgagatgcctggt |
| SEQ ID NO: 37 | tataattaacccagacatgtggctgccccccccccccccaacacctgctgcctctaaaaataa |
| | ccctgtccctggtggatcccactacgggtttaggctgcccatgtaaggaggcaaggcctgggg |
| | acacccgagatgcctggttataattaacccagacatgtggctgccccccccccccccaacacc |
| | tgctgcctctaaaaataaccctgtccctggtggatcccactacgggtttaggctgcccatgta |
| | aggaggcaaggcctggggacacccgagatgcctggttataattaacccagacatgtggctgcc |
| | ccccccccccccaacacctgctgcctctaaaaataaccctgtccctggtggatcccctgcatg |
| | cgaagatcttcgaacaaggctgtgggggactgagggcaggctgtaacaggcttgggggccagg |
| | gcttatacgtgcctgggactcccaaagtattactgttccatgttcccggcgaagggccagctg |
| | tcccccgccagctagactcagcacttagtttaggaaccagtgagcaagtcagcccttggggca |
| | gcccatacaaggccatggggctgggcaagctgcacgcctgggtccggggtgggcacggtgccc |
| | gggcaacgagctgaaagctcatctgctctcaggggcccctccctggggacagcccctcctggc |
| | tagtcacaccctgtaggctcctctatataacccaggggcacaggggctgccctcattctacca |
| | ccacctccacagcacagacagacactcaggagccagccagcgtcga |
| mU1a | atggaggcggtactatgtagatgagaattcaggagcaaactgggaaaagcaactgcttccaaa |
| SEQ ID NO: 38 | tatttgtgatttttacagtgtagttttggaaaaactcttagcctaccaattcttctaagtgtt |
| | ttaaaatgtgggagccagtacacatgaagttatagagtgttttaatgaggcttaaatatttac |
| | cgtaactatgaaatgctacgcatatcatgctgttcaggctccgtggccacgcaactcatact |
| EF-1α | gggcagagcgcacatcgcccacagtccccgagaagttggggggaggggtcggcaattgaacgg |
| SEQ ID NO: 39 | gtgcctagagaaggtggcgcggggtaaactgggaaagtgatgtcgtgtactggctccgccttt |
| | ttcccgagggtgggggagaaccgtatataagtgcagtagtcgccgtgaacgttctttttcgca |
| | acgggtttgccgccagaacacag |
| TBG | gggctggaagctacctttgacatcatttcctctgcgaatgcatgtataatttctacagaacct |
| SEQ ID NO: 40 | attagaaaggatcacccagcctctgcttttgtacaactttcccttaaaaaactgccaattcca |
| | ctgctgtttggcccaatagtgagaactttttcctgctgcctcttggtgcttttgcctatggcc |
| | cctattctgcctgctgaagacactcttgccagcatggacttaaacccctccagctctgacaat |
| | cctctttctcttttgttttacatgaagggtctggcagccaaagcaatcactcaaagttcaaac |
| | cttatcattttttgctttgttcctcttggccttggttttgtacatcagctttgaaaataccat |
| | cccagggttaatgctggggttaatttataactaagagtgctctagttttgcaatacaggacat |
| | gctataaaaat |
| | ggaaagat |
| Chimeric Intron | gtaagtatcaaggttacaagacaggtttaaggagaccaatagaaactgggcttgtcgagacag |
| SEQ ID NO: 41 | agaagactcttgcgtttctgataggcacctattggtcttactgacatccactttgcctttctc |
| | tccacag |
| VH4 Intron | gtgagtatctcagggatccagacatggggatatgggaggtgcctctgatcccagggctcactg |
| SEQ ID NO: 42 | tgggtctctctgttcacag |
| SV40 Intron | gtaagtttagtctttttgtcttttatttcaggtcccggatccggtggtggtgcaaatcaaaga |
| SEQ ID NO: 43 | actgctcctcagtggatgttgcctttacttctag |
| β-globin PolyA | ataaaggaaatttattttcattgcaatagtgtgttggaattttttgtgtctctca |
| signal | |
| SEQ ID NO: 44 | |
| Rabbit β-globin | gatctttttccctctgccaaaaattatggggacatcatgaagccccttgagcatctgacttct |
| polyA | ggctaataaaggaaatttattttcattgcaatagtgtgttggaattttttgtgtctctcactc |
| SEQ ID NO: 45 | g |
| Poly A sequence | aataaaggaaatttattttcattgcaatagtgtgttggaattttttgtgtctctca |
| SEQ ID NO 305 | |
| 5′ITR | ctgcgcgctcgctcgctcactgaggccgcccgggcaaagcccgggcgtcgggcgacctttggt |
| SEQ ID NO: 46 | cgcccggcctcagtgagcgagcgagcgcgcagagagggagtggccaactccatcactaggggt |
| | tcct |
| 5′-ITR | ctgcgcgctcgctcgctcactgaggccgcccgggcaaagcccgggcgtcgggcgacctttggt |
| (Deleted D- | cgcccggcctcagtgagcgagcgagcgcgcagagagggagtgg |
| sequence for self- | |
| complimentary | |
| AAV) | |
| SEQ ID NO: 47 | |
| 3′-ITR AAV | gaacccctagtgatggagttggccactccctctctgcgcgctcgctcgctcactgaggccgcc |
| SEQ ID NO: 48 | cgggcaaagcccgggcgtcgggcgacctttggtcgcccggcctcagtgagcgagcgagcgcgc |
| | agagagggagtggccaa |
| 3′ ITR | aggaacccctagtgatggagttggccactccctctctgcgcgctcgctcgctcactg |
| SEQ ID NO: 307 | aggccgggcgaccaaaggtcgcccgacgcccgggctttgcccgggcggcctcagtga |
| | gcgagcgagcgcgcag |
| 3′-ITR | ttggccactccctctctgcgcgctcgctcgctcactgaggccgggcgaccaaaggtcgcccga |
| (Deleted D- | cgcccgggctttgcccgggcggcctcagtgagcgagcgagcgcgcag |
| sequence for self- | |
| complimentary | |
| AAV) | |
| SEQ ID NO: 49 | |

In certain embodiments, the viral vectors provided herein comprise one or more regulatory elements other than a promoter. In certain embodiments, the viral vectors provided herein comprise an enhancer. In certain embodiments, the viral vectors provided herein comprise a repressor. In certain embodiments, the viral vectors provided herein comprise an intron (e.g. VH4 intron (SEQ ID NO: 42) SV40 Intron (SEQ ID NO:43) or a chimeric intron (β-globin/Ig Intron) (SEQ ID NO: 41).

In certain embodiments, the viral vectors provided herein comprise a polyadenylation sequence downstream of the coding region of the transgene. Any polyA site that signals termination of transcription and directs the synthesis of a poly A tail is suitable for use in AAV vectors of the present disclosure. Exemplary poly A signals are derived from, but not limited to, the following: the SV40 late gene, the rabbit β-globin gene (SEQ ID NO:45), the bovine growth hormone (BPH) gene, the human growth hormone (hGH) gene, the synthetic polyA (SPA) site (e.g., SEQ ID NO: 305), and the bovine growth hormone (bGH) gene. See, e.g., Powell and Rivera-Soto, 2015, Discov. Med., 19(102):49-57.

### 5.1.4 Signal Peptides

In certain embodiments, the vectors provided herein comprise components that modulate protein delivery. In certain embodiments, the viral vectors provided herein comprise one or more signal peptides. Signal peptides (also referred to as “signal sequences”) may also be referred to herein as “leader sequences” or “leader peptides”. In certain embodiments, the signal peptides allow for the transgene product to achieve the proper packaging (e.g., glycosylation) in the cell. In certain embodiments, the signal peptides allow for the transgene product to achieve the proper localization in the cell. In certain embodiments, the signal peptides allow for the transgene product to achieve secretion from the cell.

There are two general approaches to select a signal sequence for protein production in a gene therapy context or in cell culture. One approach is to use a signal peptide from proteins homologous to the protein being expressed. For example, a human antibody signal peptide may be used to express IgGs in CHO or other cells. Another approach is to identify signal peptides optimized for the particular host cells used for expression. Signal peptides may be interchanged between different proteins or even between proteins of different organisms, but usually the signal sequences of the most abundant secreted proteins of that cell type are used for protein expression. For example, the signal peptide of human albumin, the most abundant protein in plasma, was found to substantially increase protein production yield in CHO cells. However, certain signal peptides may retain function and exert activity after being cleaved from the expressed protein as “post-targeting functions”. Thus, in specific embodiments, the signal peptide is selected from signal peptides of the most abundant proteins secreted by the cells used for expression to avoid the post-targeting functions. In a certain embodiment, the signal sequence is fused to both the heavy and light chain sequences. An exemplary sequence is MYRMQLLLLIALSLALVINS (SEQ ID NO:50) which can be encoded by a nucleotide sequence of SEQ ID NO: 146 (see Table 2, FIG. 1). Alternatively, signal sequences that are appropriate for expression, and may cause selective expression or directed expression of the HuPTM mAb or Fab or scFv in muscle, or liver are provided in Tables 2 and 3, respectively, below.

TABLE 2
Signal peptides for expression in liver cells.

| | SEQ ID | |
|---|---|---|
| Signal Peptide Origin | NO: | Sequence |
| Mutant interleukin 2 | 50 | MYRMQLLLLIALSLALVINS |
| signal peptide | | |
| Mutant interleukin 2 | 51 | atgtataggatgcaactgctcctcctgattgctctgagcctggctcttgtgacc |
| signal peptide coding | | aactct |
| sequence | | |
| Human Serum albumin | 52 | MKWVTFISLLFLFSSAYS |
| Human α-1 Antitrypsin | 53 | MPSSVSWGILLLAGLCCLVPVSLA |
| (SERPINA1) | | |
| Human Apolipoprotein | 54 | MKAAVLTLAVLFLTGSQA |
| A-1 | | |
| Human Apolipoprotein | 55 | MKLLAATVLLLTICSLEG |
| A-2 | | |
| Human Apolipoprotein | 56 | MDPPRPALLALLALPALLLLLLAGARA |
| B-100 | | |
| Human Coagulation | 57 | MQRVNMIMAESPGLITICLLGYLLSAEC |
| Factor IX | | |
| Human Complement | 58 | MGPLMVLFCLLFLYPGLADS |
| C2 | | |
| Human Complement | 59 | MWLLVSVILISRISSVGG |
| Factor H-related | | |
| Protein 2 (CFHR2) | | |
| Human Complement | 60 | MLLLFSVILISWVSTVGG |
| Factor H-related | | |
| Protein 5 (CFHR5) | | |
| Human Fibrinogen α- | 61 | MFSMRIVCLVLSVVGTAWT |
| chain (FGA) | | |
| Human Fibrinogen β- | 62 | MKRMVSWSFHKLKTMKHLLLLLLCVFLVKS |
| chain (FGB) | | |
| Human Fibrinogen γ- | 63 | MSWSLHPRNLILYFYALLFLSSTCVA |
| chain (FGG) | | |
| Human α-2-HS- | 64 | MKSLVLLLCLAQLWGCHS |
| Glycoprotein (AHSG) | | |
| Human Hemopexin | 65 | MARVLGAPVALGLWSLCWSLAIA |
| (HPX) | | |
| Human Kininogen-1 | 66 | MKLITILFLCSRLLLSLT |
| Human Mannose- | 67 | MSLFPSLPLLLLSMVAASYS |
| binding protein C | | |
| (MBL2) | | |
| Human Plasminogen | 68 | MEHKEVVLLLLLFLKSGQG |
| (PLMN) | | |
| Human Prothrombin | 69 | MAHVRGLQLPGCLALAALCSLVHS |
| (Coagulation Factor II) | | |
| Human Secreted | 70 | MISRMEKMTMMMKILIMFALGMNYWSCSG |
| Phosphoprotein 24 | | |
| Human Anti-thrombin- | 71 | MYSNVIGTVTSGKRKVYLLSLLLIGFWDCVTC |
| III (SERPINC1) | | |
| Human Serotransferrin | 72 | MRLAVGALLVCAVLGLCLA |
| (TF) | | |

TABLE 3
Signal peptides for expression in muscle cells.

| | SEQ ID | |
|---|---|---|
| Signal Peptide Origin | NO: | Sequence |
| Human SPARC | 73 | MRAWIFFLLCLAGRALA |
| Human Collagen alpha-1(I) | 74 | MFSFVDLRLLLLLAATALLTHG |
| chain | | |
| Human Lactotransferrin | 75 | MKLVFLVLLFLGALGLCLA |
| Human Complement C3 | 76 | MGPTSGPSLLLLLLTHLPLALG |
| Human Lumican | 77 | MSLSAFTLFLALIGGTSG |
| Human Gelsolin isoform 1 | 78 | MAPHRPAPALLCALSLALCALSLPVRA |
| Human Pro-cathepsin H | 79 | MWATLPLLCAGAWLLGVPVCGA |
| Human SERPINF1 | 80 | MQALVLLLCIGALLGHSSC |
| Human SERPINE1 | 81 | MQMSPALTCLVLGLALVFGEGSA |
| Human Cathepsin D | 82 | MQPSSLLPLALCLLAAPASA |
| Human TIMP1 | 83 | MAPFEPLASGILLLLWLIAPSRA |
| Human Fibronectin | 84 | MLRGPGPGLLLLAVQCLGTAVPSTGASKSKR |
| Human Complement Cls | 85 | MWCIVLFSLLAWVYA |
| subcomponent | | |
| Human Cathepsin L1 | 86 | MNPTLILAAFCLGIASA |
| Human Cathepsin B | 87 | MWQLWASLCCLLVLANA |
| Human Salivary acidic | 88 | MLLILLSVALLAFSSA |
| proline-rich | | |
| phosphoprotein ½ | | |
| Human Follistatin-related | 89 | MWKRWLALALALVAVAWVRA |
| protein 1 | | |

5.1.5 Polycistronic Messages—IRES and 2A Linkers and scFv Constructs

Internal ribosome entry sites. A single construct can be engineered to encode both the heavy and light chains separated by a cleavable linker or IRES so that separate heavy and light chain polypeptides are expressed by the transduced cells. In certain embodiments, the viral vectors provided herein provide polycistronic (e.g., bicistronic) messages. For example, the viral construct can encode the heavy and light chains separated by an internal ribosome entry site (IRES) elements (for examples of the use of IRES elements to create bicistronic vectors see, e.g., Gurtu et al., 1996, Biochem. Biophys. Res. Comm. 229(1):295-8, which is herein incorporated by reference in its entirety). IRES elements bypass the ribosome scanning model and begin translation at internal sites. The use of IRES in AAV is described, for example, in Furling et al., 2001, Gene Ther 8(11):854-73, which is herein incorporated by reference in its entirety. In certain embodiments, the bicistronic message is contained within a viral vector with a restraint on the size of the polynucleotide(s) therein. In certain embodiments, the bicistronic message is contained within an AAV virus-based vector (e.g., an AAV8-based, AAV9-based or AAVrh10-based vector).

Furin-2A linkers. In other embodiments, the viral vectors provided herein encode the heavy and light chains separated by a cleavable linker such as the self-cleaving 2A and 2A-like peptides, with or without upstream furin cleavage sites, e.g. Furin/2A linkers, such as furin/F2A (F/F2A) or furin/T2A (F/T2A) linkers (Fang et al., 2005, Nature Biotechnology 23:584-590, Fang, 2007, Mol Ther 15:1153-9, and Chang, J. et al, MAbs 2015, 7(2):403-412, each of which is incorporated by reference herein in its entirety). For example, a furin/2A linker may be incorporated into an expression cassette to separate the heavy and light chain coding sequences, resulting in a construct with the structure:
Signal sequence—Heavy chain—Furin site—2A site—Signal Sequence—Light chain—PolyA.
   A 2A site or 2A-like site, such as an F2A site comprising the amino acid sequence RKRR(GSG)APVKQTLNFDLLKLAGDVESNPGP (SEQ ID NOS: 105 or 106) or a T2A site comprising the amino acid sequence RKRR(GSG)EGRGSLLTCGDVEENPGP (SEQ ID NOS: 103 or 104), is self-processing, resulting in “cleavage” between the final G and P amino acid residues. Several linkers, with or without an upstream flexible Gly-Ser-Gly (GSG) linker sequence (SEQ ID NO:90), that could be used include but are not limited to:

T2A:
(SEQ ID NOS: 95 or 96)
(GSG)EGRGSLLTCGDVEENPGP;
P2A:
(SEQ ID NOS: 97 or 98)
(GSG)ATNFSLLKQAGDVEENPGP;
E2A:
(SEQ ID NOS: 99 or 100)
(GSG)QCTNYALLKLAGDVESNPGP;
F2A:
(SEQ ID NOS: 101 or 102)
(GSG)APVKQTLNFDLLKLAGDVESNPGP

(see also, e.g., Szymczak, et al., 2004, Nature Biotechnol 22(5):589-594, and Donnelly, et al., 2001, J Gen Virol, 82:1013-1025, each of which is incorporated herein by reference). Exemplary amino acid and nucleotide sequences encoding different parts of the flexible linker are described in Table 4. Linker sequences may also be used to link the VH, VL and Fc domains of scFvs or scFv-Fc constructs.

TABLE 4
Linker Sequences

| | SEQ ID | |
|---|---|---|
| ID | NO: | Sequence |
| GSG linker | 90 | GSG |
| Furin linker | 91 | RKRR |
| Furin linker | 92 | RRRR |
| Furin linker | 93 | RRKR |
| Furin linker | 94 | RKKR |
| T2A | 95 | EGRGSLLTCGDVEENPGP |
| T2A | 96 | GSGEGRGSLLTCGDVEENPGP |
| P2A | 97 | ATNFSLLKQAGDVEENPGP |
| P2A | 98 | GSGATNFSLLKQAGDVEENPGP |
| E2A | 99 | QCTNYALLKLAGDVESNPGP |
| E2A | 100 | GSGQCTNYALLKLAGDVESNPGP |
| F2A | 101 | APVKQTLNFDLLKLAGDVESNPGP |
| F2A | 102 | GSGAPVKQTLNFDLLKLAGDVESNPGP |
| Furin-T2A | 103 | RKRREGRGSLLTCGDVEENPGP |
| Furin-GSG-T2A | 104 | RKRRGSGEGRGSLLTCGDVEENPGP |
| Furin-F2A | 105 | RKRRAPVKQTLNFDLLKLAGDVESNPGP |
| Furin-GSG-F2A | 106 | RKRRGSGAPVKQTLNFDLLKLAGDVESNPGP |
| Furin-GSG-T2A | 107 | agaaagagaagaggctctggagaaggcagaggctccctgct |
| | | gacatgtggggatgttgaagagaatcctgggcct |
| Furin | 108 | agaaagagaaga |
| Furin-GSG linker | 109 | agaaagagaagaggctctgga |
| GSG linker | 110 | ggctctgga |
| T2A | 111 | gaaggcagaggctccctgctgacatgtggggatgttgaaga |
| | | gaatcctgggcct |
| GGS(3X) Linker | 315 | ggtggaggcggttcaggcggaggtggcagcggcggtggagg |
| (nucleotide) | | gtcg |
| GGS(3X) Linker | 316 | GGGGSGGGGSGGGGS |
| 9G Linker | 319 | ggtggaggcggttcaggcggaggtggcagcggcggtggagg |
| | | gtcg |
| 9G linker | 320 | GGGGGGGGG |
| (nucleotide) | | |

In certain embodiments an additional proteolytic cleavage site, e.g. a furin cleavage site, is incorporated into the expression construct adjacent to the self-processing cleavage site (e.g. 2A or 2A like sequence), thereby providing a means to remove additional amino acids that remain following cleavage by the self processing cleavage sequence. Without being bound to any one theory, a peptide bond is skipped when the ribosome encounters the 2A sequence in the open reading frame, resulting in the termination of translation, or continued translation of the downstream sequence (the light chain). This self-processing sequence results in a string of additional amino acids at the end of the C-terminus of the heavy chain. However, such additional amino acids can then be cleaved by host cell Furin at the furin cleavage site(s), e.g. located immediately prior to the 2A site and after the heavy chain sequence, and further cleaved by carboxypeptidases. The resultant heavy chain may have one, two, three, or more additional amino acids included at the C-terminus, or it may not have such additional amino acids, depending on the sequence of the Furin linker used and the carboxypeptidase that cleaves the linker in vivo (See, e.g., Fang et al., 17 Apr. 2005, Nature Biotechnol. Advance Online Publication; Fang et al., 2007, Molecular Therapy 15(6):1153-1159; Luke, 2012, Innovations in Biotechnology, Ch. 8, 161-186). Furin linkers that may be used comprise a series of four basic amino acids, for example, RKRR (SEQ ID NO:91), RRRR (SEQ ID NO:92), RRKR (SEQ ID NO:93), or RKKR (SEQ ID NO:94). Once this linker is cleaved by a carboxypeptidase, additional amino acids may remain, such that an additional zero, one, two, three or four amino acids may remain on the C-terminus of the heavy chain, for example, R, RR, RK, RKR, RRR, RRK, RKK, RKRR (SEQ ID NO: 91), RRRR (SEQ ID NO:92), RRKR (SEQ ID NO:93), or RKKR (SEQ ID NO:94). In certain embodiments, once the linker is cleaved by a carboxypeptidase, no additional amino acids remain. In certain embodiments, 0.5% to 1%, 1% to 2%, 5%, 10%, 15%, or 20% of the antibody, e.g., antigen-binding fragment, population produced by the constructs for use in the methods described herein has one, two, three, or four amino acids remaining on the C-terminus of the heavy chain after cleavage. In certain embodiments, the furin linker has the sequence R-X-K/R-R, such that the additional amino acids on the C-terminus of the heavy chain are R, RX, RXK, RXR, RXKR (SEQ ID NO:22), or RXRR (SEQ ID NO:23), where X is any amino acid, for example, alanine (A). In certain embodiments, no additional amino acids may remain on the C-terminus of the heavy chain.

Flexible peptide linker. In some embodiments, a single construct can be engineered to encode both the heavy and light chains (e.g. the heavy and light chain variable domains) separated by a flexible peptide linker such as those encoding a scFv and the scFv and Fc domain of an scFv-Fc domain. A flexible peptide linker can be composed of flexible residues like glycine and serine so that the adjacent heavy chain and light chain domains are free to move relative to one another. The construct may be arranged such that the heavy chain variable domain is at the N-terminus of the scFv, followed by the linker and then the light chain variable domain. Alternatively, the construct may be arranged such that the light chain variable domain is at the N-terminus of the scFv, followed by the linker and then the heavy chain variable domain. That is, the components may be arranged as NH₂-V_{L}-linker-V_{H}—COOH or NH₂-V_{H}-linker-V_{L}-COOH. Alternatively, the construct may be arranged to include an Fc domain linked to the scFv as NH₂-V_{L}-linker-V_{H}-linker-Fc-COOH or NH₂-V_{H}-linker-V_{L}-linker-Fc-COOH. In certain embodiments the linker has a GGGS repeat (for example, 1, 2, 3, 4 or 5 repeats) and exemplary linker is the GGGS(3×) linker having an amino acid sequence of SEQ ID NO: 316 and encoded by nucleotide sequence SEQ ID NO: 315. The linker may alternatively comprise only glycines, for example, 5, 6, 7, 8, 9, 10, 11, 12 or more glycines. An exemplary linker is the 9G linker having an amino acid sequence of SEQ ID NO: 320 with a nucleotide sequence of SEQ ID NO: 319.

In certain embodiments, an expression cassette described herein is contained within a viral vector with a restraint on the size of the polynucleotide(s) therein. In certain embodiments, the expression cassette is contained within an AAV virus-based vector. Due to the size restraints of certain vectors, the vector may or may not accommodate the coding sequences for the full heavy and light chains of the therapeutic antibody but may accommodate the coding sequences of the heavy and light chains of antigen binding fragments, such as the heavy and light chains of a Fab or F(ab′)₂ fragment or an scFv or scFv-Fc. In particular, the AAV vectors described herein may accommodate a transgene of approximately 4.7 kilobases. Substitution of smaller expression elements would permit the expression of larger protein products, such as full-length therapeutic antibodies.

### 5.1.6 Untranslated Regions

In certain embodiments, the viral vectors provided herein comprise one or more untranslated regions (UTRs), e.g., 3′ and/or 5′ UTRs. In certain embodiments, the UTRs are optimized for the desired level of protein expression. In certain embodiments, the UTRs are optimized for the mRNA half-life of the transgene. In certain embodiments, the UTRs are optimized for the stability of the mRNA of the transgene. In certain embodiments, the UTRs are optimized for the secondary structure of the mRNA of the transgene.

### 5.1.7 Inverted Terminal Repeats

In certain embodiments, the viral vectors provided herein comprise one or more inverted terminal repeat (ITR) sequences. ITR sequences may be used for packaging the recombinant gene expression cassette into the virion of the viral vector. In certain embodiments, the ITR is from an AAV, e.g., AAV8 or AAV2 (see, e.g., Yan et al., 2005, J. Virol., 79(1):364-379; U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety). In preferred embodiments, nucleotide sequences encoding the ITRs may, for example, comprise the nucleotide sequences of SEQ ID NOS: 138 (5′-ITR) or 140 (3′-ITR). In certain embodiments, the modified ITRs used to produce self-complementary vector, e.g., scAAV, may be used (see, e.g., Wu, 2007, Human Gene Therapy, 18(2):171-82, McCarty et al, 2001, Gene Therapy, Vol 8, Number 16, Pages 1248-1254; and U.S. Pat. Nos. 6,596,535; 7,125,717; and 7,456,683, each of which is incorporated herein by reference in its entirety). In preferred embodiments, nucleotide sequences encoding the modified ITRs may, for example, comprise the nucleotide sequences of SEQ ID NOS: 46 (5′-ITR) or 48 or 307 (3′-ITR). Alternatively, one of the ITRs may be altered to result in a self-complementary or double stranded AAV genome and may have a nucleotide sequence of SEQ ID NO: 47 (5′) or SEQ ID NO: 49 (3′).

### 5.1.8 Transgenes

The transgenes encode a HuPTM mAb, either as a full-length antibody or an antigen binding fragment thereof, e.g. a Fab fragment (an HuGlyFab) or a F(ab′)₂, nanobody, or an scFv or scFv-Fc based upon a therapeutic antibody disclosed herein. In specific embodiments, the HuPTM mAb or antigen binding fragment, particularly the HuGlyFab, or HuPTMscFv-Fc are engineered to contain additional glycosylation sites on the Fab domain (e.g., see Courtois et al., 2016, mAbs 8:99-112 which is incorporated by reference herein in its entirety for it description of sites of hyperglycosylation on a Fab domain). In addition, for the HuPTM mAb or scFv-Fc comprising an Fc domain, the Fc domain may be engineered to alter the glycosylation site at N297 to prevent glycosylation at that site (for example, a substitution at N297 for another amino acid and/or a substitution at T297 for a residue that is not a T or S to knock out the glycosylation site). Such Fc domains are “aglycosylated”.

5.1.8.1 Constructs for Expression of Full length HuPTM mAb In certain embodiments, the transgenes encode a full length heavy chain (including the heavy chain variable domain, the heavy chain constant domain 1 (C_{H}1), the hinge and Fc domain) and a full length light chain (light chain variable domain and light chain constant domain) that upon expression associate to form antigen-binding antibodies with Fc domains. The recombinant AAV constructs express the intact (i.e., full length) or substantially intact HuPTM mAb in a cell, cell culture, or in a subject. (“Substantially intact” refers to mAb having a sequence that is at least 95% identical to the full-length mAb sequence.) The nucleotide sequences encoding the heavy and light chains may be codon optimized for expression in human cells and have reduced incidence of CpG dimers in the sequence to promote expression in human cells. The transgenes may encode any full-length antibody. Certain of these nucleotide sequences are codon optimized for expression in human cells. See for example, the codon optimized sequences of L01, L02, and L03 (SEQ ID NOs: 148, 149 and 150) of Table 7. In preferred embodiments, the transgenes encode a full-length form of any of the therapeutic antibodies disclosed herein, for example, the Fab fragment of which depicted in FIG. 3 herein and including, in certain embodiments, the associated Fc domain provided in Table 6.

The full length mAb encoded by the transgene described herein preferably have the Fc domain of the full-length therapeutic antibody or is an Fc domain of the same type of immunoglobulin as the therapeutic antibody to be expressed. In other embodiments the scFv-Fc construct has an Fc region disclosed herein. In certain embodiments, the Fc region is an IgG Fc region, but in other embodiments, the Fc region may be an IgA, IgD, IgE, or IgM. The Fc domain is preferably of the same isotype as the therapeutic antibody to be expressed, for example, if the therapeutic antibody is an IgG1 isotype, then the antibody expressed by the transgene comprises an IgG1 Fc domain. The antibody expressed from the transgene may have an IgG1, IgG2, IgG3 or IgG4 Fc domain. The Fc domain may be the lanadelumab Fc domain with an amino acid sequence of SEQ ID NO: 25.

The Fc region of the intact mAb or the scFv-Fc has one or more effector functions that vary with the antibody isotype. The effector functions can be the same as that of the wild-type or the therapeutic antibody or can be modified therefrom to add, enhance, modify, or inhibit one or more effector functions using the Fc modifications disclosed in Section 5.1.9, infra. In certain embodiments, the HuPTM mAb or scFv-Fc transgene encodes a mAb or scFv-Fc comprising an Fc polypeptide comprising an amino acid sequence that is at least 85%, 86%, 87%, 88%, 89%, 90%, 91%, 92%, 93%, 94%, 95%, 96%, 97%, 98% or 99% identical to the sequence set forth in the Fc domain polypeptides of the therapeutic antibodies described herein as set forth in Table 6 for lanadelumab or an exemplary Fc domain of an IgG1, IgG2 or IgG4 isotype as set forth in Table 6. In some embodiments, the HuPTM mAb or scFv-Fc comprises a Fc polypeptide of a sequence that is a variant of the Fc polypeptide sequence in Table 6 in that the sequence has been modified with one or more of the techniques described in Section 5.1.9, infra, to alter the Fc polypeptide's effector function.

In specific embodiments, provided are recombinant AAV constructs such as the constructs shown in FIGS. 1 and 2, for gene therapy administration to a human subject in order to express an intact or substantially intact HuPTM mAb in the subject. Gene therapy constructs are designed such that both the heavy and light chains are expressed in tandem from the vector including the Fc domain polypeptide of the heavy chain. In certain embodiments, the transgene encodes a transgene with heavy and light chain Fab fragment polypeptides as shown in Table 7, yet have a heavy chain that further comprises an Fc domain polypeptide C terminal to the hinge region of the heavy chain (including an IgG1, IgG2 or IgG4 Fc domain or the lanadelumab Fc as in Table 6). In specific embodiments, the transgene is a nucleotide sequence that encodes the following: Signal sequence-heavy chain Fab portion (including hinge region)-heavy chain Fc polypeptide-Furin-2A linker-signal sequence-light chain Fab portion.

In specific embodiments for expressing an intact or substantially intact mAb in muscle or liver cell types, the constructs described herein comprise the following components: (1) AAV2 inverted terminal repeats that flank the expression cassette; (2) Control elements, which include a) an inducible promoter, preferably a hypoxia-inducible promoter, b) a chicken β-actin intron and c) a rabbit β-globin poly A signal; and (3) nucleic acid sequences coding for the heavy chain Fab of an anti-pKal mAb (e.g., lanadelumab); an Fc polypeptide associated with the therapeutic antibody (Table 6) or of the same isotype as the native form of the therapeutic antibody, such as an IgG isotype amino acid sequence from Table 6; and the light chain of an anti-pKal mAb (e.g. lanadelumab), wherein the heavy chain (Fab and Fc region) and the light chain are separated by a self-cleaving furin (F)/F2A or T2A or flexible linker, ensuring expression of equal amounts of the heavy and the light chain polypeptides. Exemplary constructs are provided in FIGS. 1 and 2.

In specific embodiments, provided are AAV vectors comprising a viral capsid that is at least 95% identical to the amino acid sequence of an AAV8 capsid (SEQ ID NO:1); and an artificial genome comprising an expression cassette flanked by AAV inverted terminal repeats (ITRs), wherein the expression cassette comprises a transgene encoding an intact or substantially intact anti-pKal mAb; operably linked to one or more regulatory sequences that control expression of the transgene in human liver or muscle cells.

The rAAV vectors that encode and express the full-length therapeutic antibodies may be administered to treat or prevent or ameliorate symptoms of a disease or condition amenable to treatment, prevention or amelioration of symptoms with the therapeutic antibodies. Also provided are methods of expressing HuPTM mAbs in human cells using the rAAV vectors and constructs encoding them.

### 5.1.8.2 Constructs for Expression of Antigen Binding Fragments

In some embodiments, the transgenes express antigen binding fragments, e.g. a Fab fragment (an HuGlyFab) or a F(ab′)₂, nanobody, or an scFv based upon a therapeutic antibody disclosed herein. FIG. 3 and section 5.4. provide the amino acid sequence of the heavy and light chains of the Fab fragments of the therapeutic antibodies (see also Table 7, which provides the amino acid sequences of the Fab heavy and light chains of the therapeutic antibodies).

Certain of these nucleotide sequences are codon optimized for expression in human cells. See for example, the codon optimized sequences of L01, L02, and L03 (SEQ ID NOs: 148, 149 and 150) for the full length antibodies (from which codon optimized sequences of the Fab fragments can be derived) of Table 7. The transgene may encode a Fab fragment using nucleotide sequences encoding the amino acid sequences provided in Table 7, but not including the portion of the hinge region on the heavy chain that forms interchain di-sulfide bonds (e.g., the portion containing the sequence CPPCPA (SEQ ID NO:113)). Heavy chain Fab domain sequences that do not contain a CPPCP (SEQ ID NO:112) sequence of the hinge region at the C-terminus will not form intrachain disulfide bonds and, thus, will form Fab fragments with the corresponding light chain Fab domain sequences, whereas those heavy chain Fab domain sequences with a portion of the hinge region at the C-terminus containing the sequence CPPCP (SEQ ID NO:112) will form intrachain disulfide bonds and, thus, will form Fab₂ fragments.

For example, in some embodiments, the transgene may encode a scFv comprising a light chain variable domain and a heavy chain variable domain connected by a flexible linker in between (where the heavy chain variable domain may be either at the N-terminal end or the C-terminal end of the scFv), and optionally, may further comprise a Fc polypeptide (e.g., IgG1, IgG2, IgG3, or IgG4) on the C-terminal end of the heavy chain. scFvs may be generated using the VH and VL amino acid sequences for lanadelumab (for example, a VH having an amino acid sequence of SEQ ID NO: 314, which may be encoded by a codon-optimized and CpG deleted nucleotide sequence of SEQ ID NO: 313 and a VL having an amino acid sequence of SEQ ID NO 318, which may be encoded by a codon-optimized, CpG deleted nucleotide sequence of SEQ ID NO 317) linked by a flexible non-cleavable linker, such as a linker in Table 4, for example a GGGGS linker (such as the GGGGS(3×) having an amino acid sequence os SEQ ID NO: 316) to form either N-VH-linker-VL-C or N-VL-linker-VH-C (which may have a signal sequence at the amino terminus). The scFv may be linked at the C terminus to an Fc domain though a flexible, non-cleavable linker (such as linkers in Table 4) including a glycine linker, 9G linker (amino acid sequence SEQ ID NO: 320). Alternatively, if the hinge region is linked to the Fc domain, a flexible linker may not be necessary to link the Fc domain to the scFv. The Fc domain may be the Fc of lanadelumab (for example, having the amino acid sequence of SEQ ID NO: 25) and may also include a hinge sequence (see Table 5) (or may not if a flexible linker is used) and may, in particular, have an amino acid sequence of SEQ ID NO: 322 (which may be encoded by a codon-optimized and CpG deleted sequence of SEQ ID NO: 321). The encoded scFv may be a VH-VL-Fc having an amino acid sequence of SEQ ID NO 324 (which may be encoded by a codon-optimized and CpG deleted sequence of SEQ ID NO: 323) or a VL-VH-Fc having an amino acid sequence of SEQ ID NO: 393 (which may be encoded by a codon-optimized and CpG deleted sequence of SEQ ID NO: 392). The Fc domain may also be modified, for example, as described in Section 5.1.9, herein. The scFv or scFv-Fc may have a signal sequence at the N-terminus, for example, a sequence provided in Table 1.

Alternatively, in other embodiments, the transgene may encode F(ab′)₂ fragments comprising a nucleotide sequence that encodes the light chain and the heavy chain sequence that includes at least the sequence CPPCA (SEQ ID NO: 114) of the hinge region, as depicted in FIGS. 2A and 2B which depict various regions of the hinge region that may be included at the C-terminus of the heavy chain sequence. Pre-existing anti-hinge antibodies (AHA) may cause immunogenicity and reduce efficacy. Thus, in certain embodiments, for the IgG1 isotype, C-terminal ends with D221 or ends with a mutation T225L or with L242 can reduce binding to AHA. (See, e.g., Brezski, 2008, J Immunol 181:3183-92 and Kim, 2016, 8:1536-1547). For IgG2, the risk of AHA is lower since the hinge region of IgG2 is not as susceptible to enzymatic cleavage required to generate endogenous AHA. (See, e.g., Brezski, 2011, MAbs 3:558-567).

TABLE 5
Hinge Regions

| SEQ ID NO: | Sequence |
|---|---|
| 112 | CPPCP |
| 113 | CPPCPA |
| 114 | CPPCA |
| 115 | EPKSCDKTHTCPPCPAPELLGG |
| 116 | EPKSCDKTHLCPPCPAPELLGG |
| 117 | EPKSCDKTHL |
| 118 | EPKSCDKTHT |
| 119 | EPKSCDKTHTCPPCPA |
| 120 | EPKSCDKTHLCPPCPA |
| 121 | EPKSCDKTHTCPPCPAPELLGGPSVFL |
| 122 | EPKSCDKTHLCPPCPAPELLGGPSVFL |
| 123 | EPKSCDKTHTCPPCPAPEAAGG |
| 124 | EPKSCDKTHTCPPCPAPEAAGGPSVFL |
| 125 | EPKSCDKTHLCPPCPAPEAAGGPSVFL |
| 126 | ERKSCVECPPCPAPPVAG |
| 127 | ERKSCVECPPCPA |
| 128 | ESKYGPPCPPCPAPEFLGG |
| 129 | ESKYGPPCPPCPA |
| 130 | ESKYGPPCPSCPA |
| 131 | ESKYGPPCPSCPAPEFLGGPSVFL |
| 132 | ESKYGPPCPPCPAPEFLGGPSVFL |
| 133 | ERKCCVECPPCPAPPVAG |
| 134 | ERKCCVECPPCPA |
| 135 | EPKSCDKTHTCPPCPAPELAGA |
| 136 | EPKSCDKTHTCPPCPAPELAGAPSVFL |
| 137 | EPKSCDKTHLCPPCPAPELAGAPSVFL |
| 138 | EPKSCDKTHTCPPCPAPEFEGG |
| 139 | EPKSCDKTHTCPPCPAPEFEGGPSVFL |
| 140 | EPKSCDKTHLCPPCPAPEFEGGPSVFL |

In certain embodiments, the viral vectors provided herein comprise the following elements in the following order: a) a constitutive or inducible (e.g., hypoxia-inducible or rifamycin-inducible) promoter sequence or a tissue specific promoter/regulatory region, for example, one of the regulatory regions provided in Table 1, and b) a sequence encoding the transgene (e.g., a HuGlyFab). In certain embodiments, the sequence encoding the transgene comprises multiple ORFs separated by IRES elements. In certain embodiments, the ORFs encode the heavy and light chain domains of the HuGlyFab. In certain embodiments, the sequence encoding the transgene comprises multiple subunits in one ORF separated by F/F2A sequences or F/T2A sequences. In certain embodiments, the sequence comprising the transgene encodes the heavy and light chain domains of the HuGlyFab separated by an F/F2A sequence or a F/T2A sequence. In certain embodiments, the sequence comprising the transgene encodes the heavy and light chain variable domains of the HuGlyFab separated by a flexible peptide linker (as an scFv). In certain embodiments, the viral vectors provided herein comprise the following elements in the following order: a) a constitutive or an inducible promoter sequence or a tissue specific promoter, such as one of the promoters or regulatory regions in Table 1, and b) a sequence encoding the transgene (e.g., a HuGlyFab), wherein the transgene comprises a nucleotide sequence encoding a signal peptide, a light chain and a heavy chain Fab portion separated by an IRES element. In certain embodiments, the viral vectors provided herein comprise the following elements in the following order: a) a constitutive or a hypoxia-inducible promoter sequence or regulatory element listed in Table 1, and b) a sequence encoding the transgene comprising a signal peptide, a light chain and a heavy chain sequence separated by a cleavable F/F2A sequence (SEQ ID NOS: 105 or 106) or a F/T2A sequence (SEQ ID NOS: 103 or 104) or a flexible peptide linker.

In certain embodiments, the viral vectors provided herein comprise the following elements in the following order: a) a first ITR sequence, b) a first linker sequence, c) a constitutive or an inducible promoter sequence or a tissue specific promoter or regulatory region, d) a second linker sequence, e) an intron sequence, f) a third linker sequence, g) a first UTR sequence, h) a sequence encoding the transgene (e.g., a HuGlyFab), i) a second UTR sequence, j) a fourth linker sequence, k) a poly A sequence, 1) a fifth linker sequence, and m) a second ITR sequence.

In certain embodiments, the viral vectors provided herein comprise the following elements in the following order: a) a first ITR sequence, b) a first linker sequence, c) a constitutive or an inducible promoter sequence or a tissue specific regulatory region, d) a second linker sequence, e) an intron sequence, f) a third linker sequence, g) a first UTR sequence, h) a sequence encoding the transgene (e.g., HuGlyFab), i) a second UTR sequence, j) a fourth linker sequence, k) a poly A sequence, 1) a fifth linker sequence, and m) a second ITR sequence, wherein the transgene comprises a signal, and wherein the transgene encodes a light chain and a heavy chain sequence separated by a cleavable F/2A sequence.

Provided are expression constructs that express full length lanadelumab (see Table 7, SEQ ID NOS: 239-247) or scFv-Fc constructs (see Table 14, SEQ ID NOS: 308, 325, 332, and 333). The sequences encoding the full length lanadelumab are operably linked to regulatory sequences which include promoters (see Table 1), polyadenylation sequences, optionally intron sequences, flanked by 5′ITR and 3′ITR sequences.

### 5.1.9. Fc Region Modifications

In certain embodiments, the transgenes encode full length or substantially full length heavy and light chains that associate to form a full length or intact antibody. (“Substantially intact” or “substantially full length” refers to a mAb having a heavy chain sequence that is at least 95% identical to the full-length heavy chain mAb amino acid sequence and a light chain sequence that is at least 95% identical to the full-length light chain mAb amino acid sequence). Accordingly, the transgenes comprise nucleotide sequences that encode, for example, the light and heavy chains of the Fab fragments including the hinge region of the heavy chain and C-terminal of the heavy chain of the Fab fragment, an Fc domain peptide. Table 6 provides the amino acid sequence of the Fc polypeptides for lanadelumab. Alternatively, an IgG1, IgG2, or IgG4 Fc domain, the sequences of which are provided in Table 6 may be utilized.

The term “Fc region” refers to a dimer of two “Fc polypeptides” (or “Fc domains”), each “Fc polypeptide” comprising the heavy chain constant region of an antibody excluding the first constant region immunoglobulin domain. In some embodiments, an “Fc region” includes two Fc polypeptides linked by one or more disulfide bonds, chemical linkers, or peptide linkers. “Fc polypeptide” refers to at least the last two constant region immunoglobulin domains of IgA, IgD, and IgG, or the last three constant region immunoglobulin domains of IgE and IgM and may also include part or all of the flexible hinge N-terminal to these domains. For IgG, e.g., “Fc polypeptide” comprises immunoglobulin domains Cgamma2 (Cγ2, often referred to as CH2 domain) and Cgamma3 (Cγ3, also referred to as CH3 domain) and may include the lower part of the hinge domain between Cgamma1 (Cγ1, also referred to as CH1 domain) and CH2 domain. Although the boundaries of the Fc polypeptide may vary, the human IgG heavy chain Fc polypeptide is usually defined to comprise residues starting at T223 or C226 or P230, to its carboxyl-terminus, wherein the numbering is according to the EU index as in Kabat et al. (1991, NIH Publication 91-3242, National Technical Information Services, Springfield, Va.). For IgA, e.g., Fc polypeptide comprises immunoglobulin domains Calpha2 (Cα2) and Calpha3 (Cα3) and may include the lower part of the hinge between Calpha1 (Cα1) and Cα2.

In certain embodiments, the Fc polypeptide is that of the therapeutic antibody or is the Fc polypeptide corresponding to the isotype of the therapeutic antibody). In still other embodiments, the Fc polypeptide is an IgG Fc polypeptide. The Fc polypeptide may be from the IgG1, IgG2, or IgG4 isotype (see Table 6) or may be an IgG3 Fc domain, depending, for example, upon the desired effector activity of the therapeutic antibody. In some embodiments, the engineered heavy chain constant region (CH), which includes the Fc domain, is chimeric. As such, a chimeric CH region combines CH domains derived from more than one immunoglobulin isotype and/or subtype. For example, the chimeric (or hybrid) CH region comprises part or all of an Fc region from IgG, IgA and/or IgM. In other examples, the chimeric CH region comprises part or all a CH2 domain derived from a human IgG1, human IgG2, or human IgG4 molecule, combined with part or all of a CH3 domain derived from a human IgG1, human IgG2, or human IgG4 molecule. In other embodiments, the chimeric CH region contains a chimeric hinge region.

TABLE 6
Table of Fc Domain Amino Acid Sequences

| | Chain/ | |
|---|---|---|
| | SEQ ID | |
| mAb | NO. | Sequence |
| IgG1 | SEQ ID | EPKSCDKTHTCPPCPAPELLGGPSVFLFPPKPKDTLMISRTPEVTCVVVDVSHEDPEV |
| | NO: 141 | KFNWYVDGVEVHNAKTKPREEQYNSTYRVVSVLTVLHQDWLNGKEYKCKVSNKALPAP |
| | | IEKTISKAKGQPREPQVYTLPPSRDELTKNQVSLTCLVKGFYPSDIAVEWESNGQPEN |
| | | NYKTTPPVLDSDGSFFLYSKLTVDKSRWQQGNVFSCSVMHEALHNHYTQKSLSLSPGK |
| IgG2 | SEQ ID | ERKCCVECPPCPAPPVAGPSVFLFPPKPKDTLMISRTPEVTCVVVDVSHEDPEVQFNW |
| | NO: 142 | YVDGVEVHNAKTKPREEQFNSTFRVVSVLTVVHQDWLNGKEYKCKVSNKGLPAPIEKT |
| | | ISKTKGQPREPQVYTLPPSREEMTKNQVSLTCLVKGFYPSDISVEWESNGQPENNYKT |
| | | TPPMLDSDGSFFLYSKLTVDKSRWQQGNVFSCSVMHEALHNHYTQKSLSLSPGK |
| IgG4 | SEQ ID | ESKYGPPCPSCPAPEFLGGPSVFLFPPKPKDTLMISRTPEVTCVVVDVSQEDPEVQFN |
| | NO: 143 | WYVDGVEVHNAKTKPREEQFNSTYRVVSVLTVLHQDWLNGKEYKCKVSNKGLPSSIEK |
| | | TISKAKGQPREPQVYTLPPSQEEMTKNQVSLTCLVKGFYPSDIAVEWESNGQPENNYK |
| | | TTPPVLDSDGSFFLYSRLTVDKSRWQEGNVFSCSVMHEALHNHYTQKSLSLSLGK |
| Lanadelumab | SEQ ID | FPPKPKDTLM ISRTPEVTCV VVDVSHEDPE VKFNWYVDGV |
| Fc domain | NO: 25 | EVHNAKTKPR EEQYNSTYRV VSVLTVLHQD WLNGKEYKCK |
| | | VSNKALPAPI EKTISKAKGQ PREPQVYTLP PSREEMTKNQ |
| | | VSLTCLVKGF YPSDIAVEWE SNGQPENNYK TTPPVLDSDG |
| | | SFFLYSKLTV DKSRWQQGNV FSCSVMHEAL HNHYTQKSLS LSPG |

In some embodiments, the recombinant vectors encode therapeutic antibodies comprising an engineered (mutant) Fc regions, e.g. engineered Fc regions of an IgG constant region. Modifications to an antibody constant region, Fc region or Fc fragment of an IgG antibody may alter one or more effector functions such as Fc receptor binding or neonatal Fc receptor (FcRn) binding and thus half-life, CDC activity, ADCC activity, and/or ADPC activity, compared to a corresponding antibody having a wild-type IgG constant region, or an IgG heavy chain constant region without the recited modification(s). Accordingly, in some embodiments, the antibody may be engineered to provide an antibody constant region, Fc region or Fc fragment of an IgG antibody that exhibits altered binding (as compared to a reference or wild-type constant region without the recited modification(s)) to one or more Fc receptors (e.g., FcγRI, FcγRIIA, FcγRIIB, FcγRIIIA, FcγRIIIB, FcγRIV, or FcRn receptor). In some embodiments, the antibody an antibody constant region, Fc region or Fc fragment of an IgG antibody that exhibits a one or more altered effector functions such as CDC, ADCC, or ADCP activity, compared to a corresponding antibody having a wild-type IgG constant region, or an IgG constant without the recited modification(s).

“Effector function” refers to a biochemical event that results from the interaction of an antibody Fc region with an Fc receptor or ligand. Effector functions include FcγR-mediated effector functions such as ADCC and ADCP and complement-mediated effector functions such as CDC.

In other embodiments, the immunoglobulin constant regions are engineered to provide “effectorless” function. In some embodiments, the disclosed antibodies or constructs having an Fc can have an IgG4 or IgG2 isotype constant region, such that antibodies or constructs having an Fc domain of the IgG4 or IgG2 isotype exhibit reduced effector function as compared to antibodies having an Fc domain of the IgG1 isotype. In some embodiments, the effectorless Fc domain is an aglycosylated IgG1, IgG2, or IgG4 Fc domain that has a substitution at residue 297 or 299 to alter the glycosylation site at 297 such that the Fc domain exhibits reduced ADCC or other effector activity. Amino acid numbering of immunoglobulin constant regions described throughout the present disclosure is according to the EU index as in Kabat et al. (1991, NIH Publication 91-3242, National Technical Information Services, Springfield, Va., which is hereby incorporated by reference). In some embodiments, amino acids at positions 234, 235, 329 of the IgG1 constant region are modified (or mutated) in order to reduce effector function, also known as Fc function. As such, the L234A, L235A, P329G (LALA-PG) variant eliminates complement binding and fixation as well as Fc-γ dependent antibody-dependent cell-mediated cytotoxity (ADCC) in both murine IgG2a and human IgG1. Other non-limiting Fc modifications are described herein.

An “effector cell” refers to a cell of the immune system that expresses one or more Fc receptors and mediates one or more effector functions. Effector cells include but are not limited to monocytes, macrophages, neutrophils, dendritic cells, eosinophils, mast cells, platelets, B cells, large granular lymphocytes, Langerhans' cells, natural killer (NK) cells, and T cells, and may be from any organism including but not limited to humans, mice, rats, rabbits, and monkeys.

“ADCC” or “antibody dependent cell-mediated cytotoxicity” refers to the cell-mediated reaction wherein nonspecific cytotoxic effector (immune) cells that express FcγRs recognize bound antibody on a target cell and subsequently cause lysis of the target cell.

“ADCP” or “antibody dependent cell-mediated phagocytosis” refers to the cell-mediated reaction wherein nonspecific cytotoxic effector (immune) cells that express FcγRs recognize bound antibody on a target cell and subsequently cause phagocytosis of the target cell.

“CDC” or “complement-dependent cytotoxicity” refers to the reaction wherein one or more complement protein components recognize bound antibody on a target cell and subsequently cause lysis of the target cell.

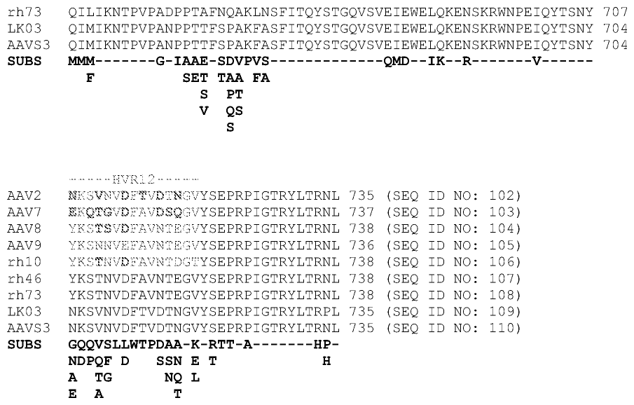

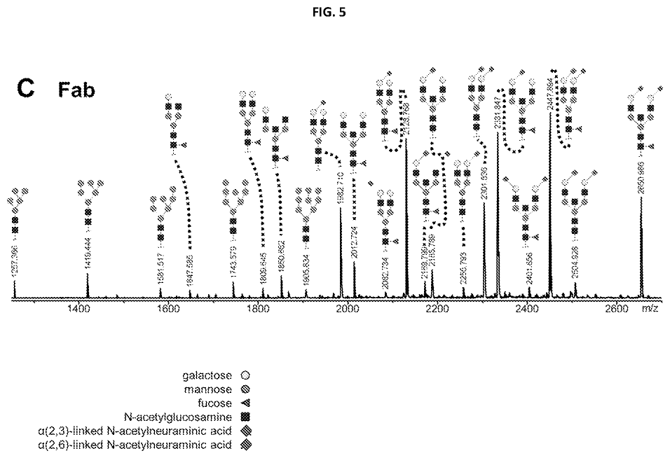

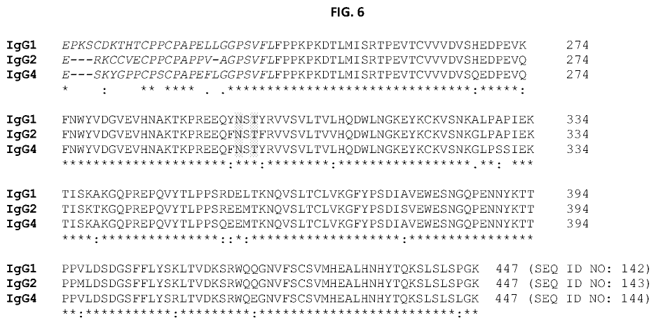

In some embodiments, the modifications of the Fc domain include, but are not limited to, the following modifications and combinations thereof, with reference to EU numbering of an IgG constant region (see FIG. 6): 233, 234, 235, 236, 237, 238, 239, 248, 249, 250, 252, 254, 255, 256, 258, 265, 267, 268, 269, 270, 272, 276, 278, 280, 283, 285, 286, 289, 290, 292, 293, 294, 295, 296, 297, 298, 301, 303, 305, 307, 308, 309, 311, 312, 315, 318, 320, 322, 324, 326, 327, 328, 329, 330, 331, 332, 333, 334, 335, 337, 338, 339, 340, 342, 344, 356, 358, 359, 360, 361, 362, 373, 375, 376, 378, 380, 382, 383, 384, 386, 388, 389, 398, 414, 416, 419, 428, 430, 433, 434, 435, 437, 438, and 439.

In certain embodiments, the Fc region comprises an amino acid addition, deletion, or substitution of one or more of amino acid residues 251-256, 285-290, 308-314, 385-389, and 428-436 of the IgG. In some embodiments, 251-256, 285-290, 308-314, 385-389, and 428-436 (EU numbering of Kabat; see FIG. 6) is substituted with histidine, arginine, lysine, aspartic acid, glutamic acid, serine, threonine, asparagine, or glutamine. In some embodiments, a non-histidine residue is substituted with a histidine residue. In some embodiments, a histidine residue is substituted with a non-histidine residue.

Enhancement of FcRn binding by an antibody having an engineered Fc leads to preferential binding of the affinity-enhanced antibody to FcRn as compared to antibody having wild-type Fc, and thus leads to a net enhanced recycling of the FcRn-affinity-enhanced antibody, which results in further increased antibody half-life. An enhanced recycling approach allows highly effective targeting and clearance of antigens, including e.g. “high titer” circulating antigens, such as C5, cytokines, or bacterial or viral antigens.

Provided in certain embodiments are modified constant region, Fc region or Fc fragment of an IgG antibody with enhanced binding to FcRn in serum as compared to a wild-type Fc region (without engineered modifications). In some instances, antibodies, e.g. IgG antibodies, are engineered to bind to FcRn at a neutral pH, e.g., at or above pH 7.4, to enhance pH-dependence of binding to FcRn as compared to a wild-type Fc region (without engineered modifications). In some instances, antibodies, e.g. IgG antibodies, are engineered to exhibit enhanced binding (e.g. increased affinity or K_{D}) to FcRn in endosomes (e.g., at an acidic pH, e.g., at or below pH 6.0) relative to a wild-type IgG and/or reference antibody binding to FcRn at an acidic pH, as well as in comparison to binding to FcRn in serum (e.g., at a neutral pH, e.g., at or above pH 7.4). Provided are antibodies with an engineered antibody constant region, Fc region or Fc fragment of an IgG antibody that exhibits an improved serum or resident tissue half-life, compared to a corresponding antibody having a wild-type IgG constant region, or an IgG constant without the recited modification(s);

Non-limiting examples of such Fc modifications include, e.g., a modification at position 250 (e.g., E or Q); 250 and 428 (e.g., L or F); 252 (e.g., LN/Y/W or T), 254 (e.g., S or T), and 256 (e.g., S/R/Q/E/D or T); or a modification at position 428 and/or 433 (e.g., H/L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V2591), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P) (EU numbering; see FIG. 6).

In some embodiments, the Fc region can be a mutant form such as hIgG1 Fc including M252 mutations, e.g. M252Y and S254T and T256E (“YTE mutation”) exhibit enhanced affinity for human FcRn (Dall'Acqua, et al., 2002, J Immunol 169:5171-5180) and subsequent crystal structure of this mutant antibody bound to hFcRn resulting in the creation of two salt bridges (Oganesyan, et al. 2014, JBC 289(11): 7812-7824). Antibodies having the YTE mutation have been administered to monkeys and humans, and have significantly improved pharmacokinetic properties (Haraya, et al., 2019, Drug Metabolism and Pharmacokinetics, 34(1):25-41).

In some embodiments, modifications to one or more amino acid residues in the Fc region may reduce half-life in systemic circulation (serum), however result in improved retainment in tissues (e.g. in the eye) by disabling FcRn binding (e.g. H435A, EU numbering of Kabat) (Ding et al., 2017, MAbs 9:269-284; and Kim, 1999, Eur J Immunol 29:2819).

In some embodiments, the Fc domain may be engineered to activate all, some, or none of the normal Fc effector functions, without affecting the Fc polypeptide's (e.g. antibody's) desired pharmacokinetic properties. Fc polypeptides having altered effector function may be desirable as they may reduce unwanted side effects, such as activation of effector cells, by the therapeutic protein.

Methods to alter or even ablate effector function may include mutation(s) or modification(s) to the hinge region amino acid residues of an antibody. For example, IgG Fc domain mutants comprising 234A, 237A, and 238S substitutions, according to the EU numbering system, exhibit decreased complement dependent lysis and/or cell mediated destruction. Deletions and/or substitutions in the lower hinge, e.g. where positions 233-236 within a hinge domain (EU numbering) are deleted or modified to glycine, have been shown in the art to significantly reduce ADCC and CDC activity.

In specific embodiments, the Fc domain is an aglycosylated Fc domain that has a substitution at residue 297 or 299 to alter the glycosylation site at 297 such that the Fc domain is not glycosylated. Such aglycosylated Fc domains may have reduced ADCC or other effector activity.

Non-limiting examples of proteins comprising mutant and/or chimeric CH regions having altered effector functions, and methods of engineering and testing mutant antibodies, are described in the art, e.g. K. L. Amour, et al., Eur. J. Immunol. 1999, 29:2613-2624; Lazar et al., Proc. Natl. Acad. Sci. USA 2006, 103:4005; US Patent Application Publication No. 20070135620A1 published Jun. 14, 2007; US Patent Application Publication No. 20080154025 A1, published Jun. 26, 2008; US Patent Application Publication No. 20100234572 A1, published Sep. 16, 2010; US Patent Application Publication No. 20120225058 A1, published Sep. 6, 2012; US Patent Application Publication No. 20150337053 A1, published Nov. 26, 2015; International Publication No. WO20/16161010A2 published Oct. 6, 2016; U.S. Pat. No. 9,359,437, issued Jun. 7,2016; and U.S. Pat. No. 10,053,517, issued Aug. 21, 2018, all of which are herein incorporated by reference.

The C-terminal lysines (-K) conserved in the heavy chain genes of all human IgG subclasses are generally absent from antibodies circulating in serum—the C-terminal lysines are cleaved off in circulation, resulting in a heterogeneous population of circulating IgGs. (van den Bremer et al., 2015, mAbs 7:672-680). In the vectored constructs for full length mAbs, the DNA encoding the C-terminal lysine (-K) or glycine-lysine (-GK) of the Fc terminus can be deleted to produce a more homogeneous antibody product in situ. (See, Hu et al., 2017 Biotechnol. Prog. 33:786-794 which is incorporated by reference herein in its entirety).

### 5.1.11 Manufacture and Testing of Vectors

The viral vectors provided herein may be manufactured using host cells. The viral vectors provided herein may be manufactured using mammalian host cells, for example, A549, WEHI, 10T1/2, BHK, MDCK, COS1, COS7, BSC 1, BSC 40, BMT 10, VERO, W138, HeLa, 293, Saos, C2C12, L, HT1080, HepG2, primary fibroblast, hepatocyte, and myoblast cells. The viral vectors provided herein may be manufactured using host cells from human, monkey, mouse, rat, rabbit, or hamster.

The host cells are stably transformed with the sequences encoding the transgene and associated elements (e.g., the vector genome), and the means of producing viruses in the host cells, for example, the replication and capsid genes (e.g., the rep and cap genes of AAV). For a method of producing recombinant AAV vectors with AAV8 capsids, see Section IV of the Detailed Description of U.S. Pat. No. 7,282,199 B2, which is incorporated herein by reference in its entirety. Genome copy titers of said vectors may be determined, for example, by TAQMAN® analysis. Virions may be recovered, for example, by CsCl₂ sedimentation.

Alternatively, baculovirus expression systems in insect cells may be used to produce AAV vectors. For a review, see Aponte-Ubillus et al., 2018, Appl. Microbiol. Biotechnol. 102:1045-1054 which is incorporated by reference herein in its entirety for manufacturing techniques.

In vitro assays, e.g., cell culture assays, can be used to measure transgene expression from a vector described herein, thus indicating, e.g., potency of the vector. In addition, in vitro neutralization assays can be used to measure the activity of the transgene expressed from a vector described herein. For example, Vero-E6 cells, a cell line derived from the kidney of an African green monkey, or HeLa cells engineered to stably express the ACE2 receptor (HeLa-ACE2), can be used to assess neutralization activity of transgenes expressed from a vector described herein. In addition, other characteristics of the expressed product can be determined, for example determination of the glycosylation and tyrosine sulfation patterns associated with the HuGlyFab. Glycosylation patterns and methods of determining the same are discussed in Section 5.3, while tyrosine sulfation patterns and methods of determining the same are discussed in Section 5.3. In addition, benefits resulting from glycosylation/sulfation of the cell-expressed HuGlyFab can be determined using assays known in the art, e.g., the methods described in Section 5.3.

Vector genome concentration (GC) or vector genome copies can be evaluated using digital PCR (dPCR) or ddPCR™ (BioRad Technologies, Hercules, CA, USA). In one example, liver biopsies are obtained at several timepoints. In another example, several mice are sacrificed at various timepoints post injection. Liver tissue samples are subjected to total DNA extraction and dPCR assay for vector copy numbers. Copies of vector genome (transgene) per gram of tissue may be measured in a single biopsy sample, or measured in various tissue sections at sequential timepoints will reveal spread of AAV throughout the liver. Total DNA from collected liver tissue is extracted with the DNeasy Blood & Tissue Kit and the DNA concentration measured using a Nanodrop spectrophotometer. To determine the vector copy numbers in each tissue sample, digital PCR was performed with Naica Crystal Digital PCR system (Stilla technologies). Two color multiplexing system were applied here to simultaneously measure the transgene AAV and an endogenous control gene. In brief, the transgene probe can be labelled with FAM (6-carboxyfluorescein) dye while the endogenous control probe can be labelled with VIC fluorescent dye. The copy number of delivered vector in a specific tissue section per diploid cell is calculated as: (vector copy number)/(endogenous control)×2. Vector copy in specific cell types, such as liver cells, over time may indicate sustained expression of the transgene by the tissue. Sampling of muscle may be accomplished similarly.

### 5.1.12 Compositions

Pharmaceutical compositions suitable for administration to human subjects comprise a suspension of the recombinant vector in a formulation buffer comprising a physiologically compatible aqueous buffer, a surfactant and optional excipients. Such formulation buffer can comprise one or more of a polysaccharide, a surfactant, polymer, or oil. In some embodiments, the pharmaceutical composition comprises rAAV combined with a pharmaceutically acceptable carrier for administration to a subject. In one embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's complete and incomplete adjuvant), excipient, or vehicle with which the agent is administered. Such pharmaceutical carriers can be sterile liquids, such as water and oils, including those of petroleum, animal, vegetable, or synthetic origin, including, e.g., peanut oil, soybean oil, mineral oil, sesame oil and the like. Water is a common carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Additional examples of pharmaceutically acceptable carriers, excipients, and stabilizers include, but are not limited to, buffers such as phosphate, citrate, and other organic acids; antioxidants including ascorbic acid; low molecular weight polypeptides; proteins, such as serum albumin and gelatin; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™ as known in the art. The pharmaceutical composition of the present invention can also include a lubricant, a wetting agent, a sweetener, a flavoring agent, an emulsifier, a suspending agent, and a preservative, in addition to the above ingredients. These compositions can take the form of solutions, suspensions, emulsion, tablets, pills, capsules, powders, sustained-release formulations and the like.

### 5.2 Methods of Treatment

In another aspect, methods for treating hereditary angioedema or other indication that can be treated with an anti-pKal antibody in a subject in need thereof comprising the administration of recombinant AAV particles comprising an expression cassette encoding anti-pKal antibodies and antibody-binding fragments and variants thereof, such as scFv or scFv-Fcs are provided. A subject in need thereof includes a subject suffering from hereditary angioedama, or a subject pre-disposed thereto, e.g., a subject at risk of developing or having a recurrence of the hereditary angioedema, or other indication that may be treated with an anti-pKal antibody, such as ocular indications such as diabetic retinopathy and diabetic macular edema. Subjects to whom such gene therapy is administered can be those responsive to lanadelumab therapy. In particular embodiments, the methods encompass treating patients who have been diagnosed with hereditary angioedema, and, in certain embodiments, identified as responsive to treatment with an anti-pKal antibody or considered a good candidate for therapy with an anti-pKal antibody. In specific embodiments, the patients have previously been treated with an anti-pKal antibody. To determine responsiveness, the anti-pKal antibody or antigen-binding fragment transgene product (e.g., produced in human cell culture, bioreactors, etc.) may be administered directly to the subject.

In specific embodiments, provided are methods of treating hereditary angioedema or other indication amenable to treatment with an anti-pKal antibody in a human subject in need thereof comprising: administering to the liver or muscle of said subject a therapeutically effective amount of a recombinant nucleotide expression vector comprising a transgene encoding a substantially full-length or full-length anti-pKal mAb having an Fc region, or an antigen-binding fragment thereof, or a peptide, operably linked to one or more regulatory sequences that control expression of the transgene in human liver and/or muscle cells, so that a depot is formed that releases a HuPTM form of mAb or antigen-binding fragment thereof. Recombinant vectors and pharmaceutical compositions for treating diseases or disorders in a subject in need thereof are described in Section 5.1. Such vectors should have a tropism for human liver and/or muscle cells and can include non-replicating rAAV, particularly those bearing an AAV3B, AAVrh8, AAVru37, AAV64R, AAV8, AAAV9, AAVS3, AAV-LK03, AAVrh46, or AAVrh73 capsid. The recombinant vectors can be administered in any manner such that the recombinant vector enters liver and or muscle cells, e.g., by introducing the recombinant vector into circulation. Such vectors should further comprise one or more regulatory sequences that control expression of the transgene in human liver cells and/or human liver and muscle cells include, but are not limited to, liver-specific CREs of SEQ ID NO: 163-293, an ApoE.hAAT (SEQ ID NO:21) regulatory sequence, a LSPX1 promoter (SEQ ID NO:9), a LSPX2 promoter (SEQ ID NO:10), a LTP1 promoter (SEQ ID NO:11), a LTP2 (SEQ ID NO:12) promoter, or a LTP3 (SEQ ID NO:13) promoter (see also Table 1).

### 5.3. N-Glycosylation, Tyrosine Sulfation, and O-Glycosylation

The amino acid sequence (primary sequence) of HuGlyFabs or HuPTM Fabs, HuPTMmAbs, and HuPTM scFvs disclosed herein each comprises at least one site at which N-glycosylation or tyrosine sulfation takes place (see exemplary FIG. 3) for glycosylation and/or sulfation positions within the amino acid sequences of the Fab fragments of the therapeutic antibodies). Post-translational modification also occurs in the Fc domain of full length antibodies, particularly at residue N297 (by EU numbering, see Table 6).

Alternatively, mutations may be introduced into the Fc domain to alter the glycosylation site at residue N297 (EU numbering, see Table 6), in particular substituting another amino acid for the asparagine at 297 or the threonine at 299 to remove the glycosylation site resulting in an aglycosylated Fc domain.

### 5.3.1. N-Glycosylation

### Reverse Glycosylation Sites

The canonical N-glycosylation sequence is known in the art to be Asn-X-Ser (or Thr), wherein X can be any amino acid except Pro. However, it recently has been demonstrated that asparagine (Asn) residues of human antibodies can be glycosylated in the context of a reverse consensus motif, Ser (or Thr)-X-Asn, wherein X can be any amino acid except Pro. See Valliere-Douglass et al., 2009, J. Biol. Chem. 284:32493-32506; and Valliere-Douglass et al., 2010, J. Biol. Chem. 285:16012-16022. As disclosed herein, certain HuGlyFabs and HuPTM scFvs disclosed herein comprise such reverse consensus sequences.

### Non-Consensus Glycosylation Sites

In addition to reverse N-glycosylation sites, it recently has been demonstrated that glutamine (Gln) residues of human antibodies can be glycosylated in the context of a non-consensus motif, Gln-Gly-Thr. See Valliere-Douglass et al., 2010, J. Biol. Chem. 285:16012-16022. Surprisingly, certain of the HuGlyFab fragments disclosed herein comprise such non-consensus sequences. In addition, O-glycosylation comprises the addition of N-acetyl-galactosamine to serine or threonine residues by the enzyme. It has been demonstrated that amino acid residues present in the hinge region of antibodies can be O-glycosylated. The possibility of O-glycosylation confers another advantage to the therapeutic antibodies provided herein, as compared to, e.g., antigen-binding fragments produced in E. coli, again because the E. coli naturally does not contain machinery equivalent to that used in human O-glycosylation. (Instead, O-glycosylation in E. coli has been demonstrated only when the bacteria is modified to contain specific O-glycosylation machinery. See, e.g., Farid-Moayer et al., 2007, J. Bacteriol. 189:8088-8098)

### Engineered N-Glycosylation Sites

In certain embodiments, a nucleic acid encoding a HuPTM mAb, HuGlyFab or HuPTM scFv is modified to include 1, 2, 3, 4, 5, 6, 7, 8, 9, 10, or more N-glycosylation sites (including the canonical N-glycosylation consensus sequence, reverse N-glycosylation site, and non-consensus N-glycosylation sites) than would normally be associated with the HuPTM mAb, HuGlyFab or HuPTM scFv (e.g., relative to the number of N-glycosylation sites associated with the HuPTM mAb, HuGlyFab or HuPTM scFv in its unmodified state). In specific embodiments, introduction of glycosylation sites is accomplished by insertion of N-glycosylation sites (including the canonical N-glycosylation consensus sequence, reverse N-glycosylation site, and non-consensus N-glycosylation sites) anywhere in the primary structure of the antigen-binding fragment, so long as said introduction does not impact binding of the antibody or antigen-binding fragment to its antigen. Introduction of glycosylation sites can be accomplished by, e.g., adding new amino acids to the primary structure of the antigen-binding fragment, or the antibody from which the antigen-binding fragment is derived (e.g., the glycosylation sites are added, in full or in part), or by mutating existing amino acids in the antigen-binding fragment, or the antibody from which the antigen-binding fragment is derived, in order to generate the N-glycosylation sites (e.g., amino acids are not added to the antigen-binding fragment/antibody, but selected amino acids of the antigen-binding fragment/antibody are mutated so as to form N-glycosylation sites). Those of skill in the art will recognize that the amino acid sequence of a protein can be readily modified using approaches known in the art, e.g., recombinant approaches that include modification of the nucleic acid sequence encoding the protein.

In a specific embodiment, a HuGlyMab or antigen-binding fragment is modified such that, when expressed in mammalian cells, such as retina, CNS, liver or muscle cells, it can be hyperglycosylated. See Courtois et al., 2016, mAbs 8:99-112 which is incorporated by reference herein in its entirety.

N-Glycosylation of HuPTM mAbs and HuPTM Antigen-Binding Fragments

Unlike small molecule drugs, biologics usually comprise a mixture of many variants with different modifications or forms that could have a different potency, pharmacokinetics, and/or safety profile. It is not essential that every molecule produced either in the gene therapy or protein therapy approach be fully glycosylated and sulfated. Rather, the population of glycoproteins produced should have sufficient glycosylation (including 2,6-sialylation) and sulfation to demonstrate efficacy. The goal of gene therapy treatment provided herein can be, for example, to slow or arrest the progression of a disease or abnormal condition or to reduce the severity of one or more symptoms associated with the disease or abnormal condition.

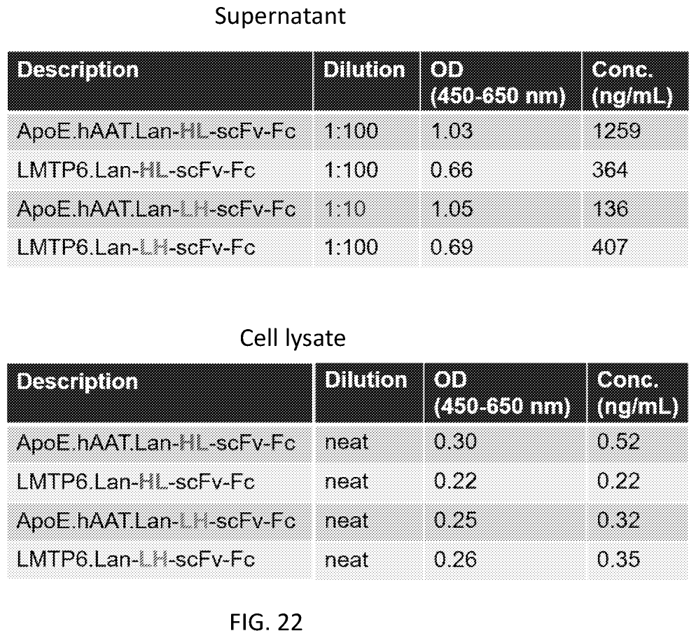

When a HuPTM mAb, HuGlyFab or HuPTM scFv is expressed in a human cell, the N-glycosylation sites of the antigen-binding fragment can be glycosylated with various different glycans. N-glycans of antigen-binding fragments and the Fc domain have been characterized in the art. For example, Bondt et al., 2014, Mol. & Cell. Proteomics 13.11:3029-3039 (incorporated by reference herein in its entirety for its disclosure of Fab-associated N-glycans; see also, FIG. 22) characterizes glycans associated with Fabs, and demonstrates that Fab and Fc portions of antibodies comprise distinct glycosylation patterns, with Fab glycans being high in galactosylation, sialylation, and bisection (e.g., with bisecting GlcNAc) but low in fucosylation with respect to Fc glycans. Like Bondt, Huang et al., 2006, Anal. Biochem. 349:197-207 (incorporated by reference herein in its entirety for it disclosure of Fab-associated N-glycans) found that most glycans of Fabs are sialylated. However, in the Fab of the antibody examined by Huang (which was produced in a murine cell background), the identified sialic residues were N-Glycolylneuraminic acid (“Neu5Gc” or “NeuGc”) (which is not natural to humans) instead of N-acetylneuraminic acid (“Neu5Ac,” the predominant human sialic acid). In addition, Song et al., 2014, Anal. Chem. 86:5661-5666 (incorporated by reference herein in its entirety for its disclosure of Fab-associated N-glycans) describes a library of N-glycans associated with commercially available antibodies.

Glycosylation of the Fc domain has been characterized and is a single N-linked glycan at asparagine 297 (EU numbering; see Table 6). The glycan plays an integral structural and functional role, impacting antibody effector function, such as binding to Fc receptor (see, for example, Jennewein and Alter, 2017, Trends In Immunology 38:358 for a discussion of the role of Fc glycosylation in antibody function). Removal of the Fc region glycan almost completely ablates effector function (Jennewien and Alter at 362). The composition of the Fc glycan has been shown to impact effector function, for example hypergalactosylation and reduction in fucosylation have been shown to increase ADCC activity while sialylation correlates with anti-inflammatory effects (Id. at 364). Disease states, genetics and even diet can impact the composition of the Fc glycan in vivo. For recombinantly expressed antibodies, the glycan composition can differ significantly by the type of host cell used for recombinant expression and strategies are available to control and modify the composition of the glycan in therapeutic antibodies recombinantly expressed in cell culture, such as CHO to alter effector function (see, for example, US 2014/0193404 by Hansen et al.). Accordingly, the HuPTM mAbs provided herein may advantageously have a glycan at N297 that is more like the native, human glycan composition than antibodies expressed in non-human host cells.

Importantly, when the HuPTM mAb, HuGlyFab or HuPTM scFv are expressed in human cells, the need for in vitro production in prokaryotic host cells (e.g., E. coli) or eukaryotic host cells (e.g., CHO cells or NS0 cells) is circumvented. Instead, as a result of the methods described herein, N-glycosylation sites of the HuPTM mAb, HuGlyFab or HuPTM scFv are advantageously decorated with glycans relevant to and beneficial to treatment of humans. Such an advantage is unattainable when CHO cells, NS0 cells, or E. coli are utilized in antibody/antigen-binding fragment production, because e.g., CHO cells (1) do not express 2,6 sialyltransferase and thus cannot add 2,6 sialic acid during N-glycosylation; (2) can add Neu5Gc as sialic acid instead of Neu5Ac; and (3) can also produce an immunogenic glycan, the α-Gal antigen, which reacts with anti-α-Gal antibodies present in most individuals, which at high concentrations can trigger anaphylaxis; and because (4) E. coli does not naturally contain components needed for N-glycosylation.

Assays for determining the glycosylation pattern of antibodies, including antigen-binding fragments are known in the art. For example, hydrazinolysis can be used to analyze glycans. First, polysaccharides are released from their associated protein by incubation with hydrazine (the Ludger Liberate Hydrazinolysis Glycan Release Kit, Oxfordshire, UK can be used). The nucleophile hydrazine attacks the glycosidic bond between the polysaccharide and the carrier protein and allows release of the attached glycans. N-acetyl groups are lost during this treatment and have to be reconstituted by re-N-acetylation. Glycans may also be released using enzymes such as glycosidases or endoglycosidases, such as PNGase F and Endo H, which cleave cleanly and with fewer side reactions than hydrazines. The free glycans can be purified on carbon columns and subsequently labeled at the reducing end with the fluorophor 2-amino benzamide. The labeled polysaccharides can be separated on a GlycoSep-N column (GL Sciences) according to the HPLC protocol of Royle et al, Anal Biochem 2002, 304(1):70-90. The resulting fluorescence chromatogram indicates the polysaccharide length and number of repeating units. Structural information can be gathered by collecting individual peaks and subsequently performing MS/MS analysis. Thereby the monosaccharide composition and sequence of the repeating unit can be confirmed and additionally in homogeneity of the polysaccharide composition can be identified. Specific peaks of low or high molecular weight can be analyzed by MALDI-MS/MS and the result used to confirm the glycan sequence. Each peak in the chromatogram corresponds to a polymer, e.g., glycan, consisting of a certain number of repeat units and fragments, e.g., sugar residues, thereof. The chromatogram thus allows measurement of the polymer, e.g., glycan, length distribution. The elution time is an indication for polymer length, while fluorescence intensity correlates with molar abundance for the respective polymer, e.g., glycan. Other methods for assessing glycans associated with antigen-binding fragments include those described by Bondt et al., 2014, Mol. & Cell. Proteomics 13.11:3029-3039, Huang et al., 2006, Anal. Biochem. 349:197-207, and/or Song et al., 2014, Anal. Chem. 86:5661-5666.

Homogeneity or heterogeneity of the glycan patterns associated with antibodies (including antigen-binding fragments), as it relates to both glycan length or size and numbers glycans present across glycosylation sites, can be assessed using methods known in the art, e.g., methods that measure glycan length or size and hydrodynamic radius. HPLC, such as size exclusion, normal phase, reversed phase, and anion exchange HPLC, as well as capillary electrophoresis, allows the measurement of the hydrodynamic radius. Higher numbers of glycosylation sites in a protein lead to higher variation in hydrodynamic radius compared to a carrier with less glycosylation sites. However, when single glycan chains are analyzed, they may be more homogenous due to the more controlled length. Glycan length can be measured by hydrazinolysis, SDS PAGE, and capillary gel electrophoresis. In addition, homogeneity can also mean that certain glycosylation site usage patterns change to a broader/narrower range. These factors can be measured by Glycopeptide LC-MS/MS.

In certain embodiments, the HuPTM mAbs, or antigen binding fragments thereof, also do not contain detectable NeuGc and/or α-Gal. By “detectable NeuGc” or “detectable α-Gal” or “does not contain or does not have NeuGc or α-Gal” means herein that the HuPTM mAb or antigen-binding fragment, does not contain NeuGc or α-Gal moieties detectable by standard assay methods known in the art. For example, NeuGc may be detected by HPLC according to Hara et al., 1989, “Highly Sensitive Determination of N-Acetyl- and N-Glycolylneuraminic Acids in Human Serum and Urine and Rat Serum by Reversed-Phase Liquid Chromatography with Fluorescence Detection.” J. Chromatogr., B: Biomed. 377, 111-119, which is hereby incorporated by reference for the method of detecting NeuGc. Alternatively, NeuGc may be detected by mass spectrometry. The α-Gal may be detected using an ELISA, see, for example, Galili et al., 1998, “A sensitive assay for measuring α-Gal epitope expression on cells by a monoclonal anti-Gal antibody.” Transplantation. 65(8):1129-32, or by mass spectrometry, see, for example, Ayoub et al., 2013, “Correct primary structure assessment and extensive glyco-profiling of cetuximab by a combination of intact, middle-up, middle-down and bottom-up ESI and MALDI mass spectrometry techniques.” Landes Bioscience. 5(5):699-710. See also the references cited in Platts-Mills et al., 2015, “Anaphylaxis to the Carbohydrate Side-Chain Alpha-gal” Immunol Allergy Clin North Am. 35(2): 247-260.

### Benefits of N-Glycosylation

N-glycosylation confers numerous benefits on the HuPTM mAb, HuGlyFab or HuPTM scFv described herein. Such benefits are unattainable by production of antigen-binding fragments in E. coli, because E. coli does not naturally possess components needed for N-glycosylation. Further, some benefits are unattainable through antibody production in, e.g., CHO cells (or murine cells such as NS0 cells), because CHO cells lack components needed for addition of certain glycans (e.g., 2,6 sialic acid and bisecting GlcNAc) and because either CHO or murine cell lines add N-N-Glycolylneuraminic acid (“Neu5Gc” or “NeuGc”) which is not natural to humans (and potentially immunogenic), instead of N-Acetylneuraminic acid (“Neu5Ac”) the predominant human sialic acid. See, e.g., Dumont et al., 2015, Crit. Rev. Biotechnol. 36(6):1110-1122; Huang et al., 2006, Anal. Biochem. 349:197-207 (NeuGc is the predominant sialic acid in murine cell lines such as SP2/0 and NS0); and Song et al., 2014, Anal. Chem. 86:5661-5666, each of which is incorporated by reference herein in its entirety). Moreover, CHO cells can also produce an immunogenic glycan, the α-Gal antigen, which reacts with anti-α-Gal antibodies present in most individuals, which at high concentrations can trigger anaphylaxis. See, e.g., Bosques, 2010, Nat. Biotech. 28:1153-1156. The human glycosylation pattern of the HuGlyFab of HuPTM scFv described herein should reduce immunogenicity of the transgene product and improve efficacy.

While non-canonical glycosylation sites usually result in low level glycosylation (e.g., 1-5%) of the antibody population, the functional benefits may be significant (See, e.g., van de Bovenkamp et al., 2016, J. Immunol. 196:1435-1441). For example, Fab glycosylation may affect the stability, half-life, and binding characteristics of an antibody. To determine the effects of Fab glycosylation on the affinity of the antibody for its target, any technique known to one of skill in the art may be used, for example, enzyme linked immunosorbent assay (ELISA), or surface plasmon resonance (SPR). To determine the effects of Fab glycosylation on the half-life of the antibody, any technique known to one of skill in the art may be used, for example, by measurement of the levels of radioactivity in the blood or organs in a subject to whom a radiolabelled antibody has been administered. To determine the effects of Fab glycosylation on the stability, for example, levels of aggregation or protein unfolding, of the antibody, any technique known to one of skill in the art may be used, for example, differential scanning calorimetry (DSC), high performance liquid chromatography (HPLC), e.g., size exclusion high performance liquid chromatography (SEC-HPLC), capillary electrophoresis, mass spectrometry, or turbidity measurement.

The presence of sialic acid on HuPTM mAb, HuGlyFab or HuPTM scFv used in the methods described herein can impact clearance rate of the HuPTM mAb, HuGlyFab or HuPTM scFv. Accordingly, sialic acid patterns of a HuPTM mAb, HuGlyFab or HuPTM scFv can be used to generate a therapeutic having an optimized clearance rate. Methods of assessing antigen-binding fragment clearance rate are known in the art. See, e.g., Huang et al., 2006, Anal. Biochem. 349:197-207.

In another specific embodiment, a benefit conferred by N-glycosylation is reduced aggregation. Occupied N-glycosylation sites can mask aggregation prone amino acid residues, resulting in decreased aggregation. Such N-glycosylation sites can be native to an antigen-binding fragment used herein or engineered into an antigen-binding fragment used herein, resulting in HuGlyFab or HuPTM scFv that is less prone to aggregation when expressed, e.g., expressed in human cells. Methods of assessing aggregation of antibodies are known in the art. See, e.g., Courtois et al., 2016, mAbs 8:99-112 which is incorporated by reference herein in its entirety.

In another specific embodiment, a benefit conferred by N-glycosylation is reduced immunogenicity. Such N-glycosylation sites can be native to an antigen-binding fragment used herein or engineered into an antigen-binding fragment used herein, resulting in HuPTM mAb, HuGlyFab or HuPTM scFv that is less prone to immunogenicity when expressed, e.g., expressed in human retinal cells, human CNS cells, human liver cells or human muscle cells.

In another specific embodiment, a benefit conferred by N-glycosylation is protein stability. N-glycosylation of proteins is well-known to confer stability on them, and methods of assessing protein stability resulting from N-glycosylation are known in the art. See, e.g., Sola and Griebenow, 2009, J Pharm Sci., 98(4): 1223-1245.

In another specific embodiment, a benefit conferred by N-glycosylation is altered binding affinity. It is known in the art that the presence of N-glycosylation sites in the variable domains of an antibody can increase the affinity of the antibody for its antigen. See, e.g., Bovenkamp et al., 2016, J. Immunol. 196:1435-1441. Assays for measuring antibody binding affinity are known in the art. See, e.g., Wright et al., 1991, EMBO J. 10:2717-2723; and Leibiger et al., 1999, Biochem. J. 338:529-538.

### 5.3.2 Tyrosine Sulfation

Tyrosine sulfation occurs at tyrosine (Y) residues with glutamate (E) or aspartate (D) within +5 to −5 position of Y, and where position −1 of Y is a neutral or acidic charged amino acid, but not a basic amino acid, e.g., arginine (R), lysine (K), or histidine (H) that abolishes sulfation. The HuGlyFabs and HuPTM scFvs described herein comprise tyrosine sulfation sites (see exemplary FIGS. 2A and 2B).

Importantly, tyrosine-sulfated antigen-binding fragments cannot be produced in E. coli, which naturally does not possess the enzymes required for tyrosine-sulfation. Further, CHO cells are deficient for tyrosine sulfation-they are not secretory cells and have a limited capacity for post-translational tyrosine-sulfation. See, e.g., Mikkelsen & Ezban, 1991, Biochemistry 30:1533-1537. Advantageously, the methods provided herein call for expression of HuPTM Fab in human cells that are secretory and have capacity for tyrosine sulfation.

Tyrosine sulfation is advantageous for several reasons. For example, tyrosine-sulfation of the antigen-binding fragment of therapeutic antibodies against targets has been shown to dramatically increase avidity for antigen and activity. See, e.g., Loos et al., 2015, PNAS 112:12675-12680, and Choe et al., 2003, Cell 114:161-170. Assays for detection tyrosine sulfation are known in the art. See, e.g., Yang et al., 2015, Molecules 20:2138-2164.

### 5.3.3 O-Glycosylation

O-glycosylation comprises the addition of N-acetyl-galactosamine to serine or threonine residues by the enzyme. It has been demonstrated that amino acid residues present in the hinge region of antibodies can be O-glycosylated. In certain embodiments, the HuGlyFab comprise all or a portion of their hinge region, and thus are capable of being O-glycosylated when expressed in human cells. The possibility of O-glycosylation confers another advantage to the HuGlyFab provided herein, as compared to, e.g., antigen-binding fragments produced in E. coli, again because the E. coli naturally does not contain machinery equivalent to that used in human O-glycosylation. (Instead, O-glycosylation in E. coli has been demonstrated only when the bacteria is modified to contain specific O-glycosylation machinery. See, e.g., Farid-Moayer et al., 2007, J. Bacteriol. 189:8088-8098) O-glycosylated HuGlyFab, by virtue of possessing glycans, shares advantageous characteristics with N-glycosylated HuGlyFab (as discussed above).

### 5.4 Anti-pKal HuPTM Constructs and Formulations for Angioedema and Diabetic

### Retinopathy and Methods of Treatment

Compositions and methods are described for the delivery of HuPTM mAbs and antigen-binding fragments thereof, such as HuPTM Fabs, that bind to kallikrein (pKal), derived from an anti-pKal antibody and indicated for treating angioedema, such as hereditary angioedema. In other embodiments, compositions and methods are provided for treating diabetic retinopathy and diabetic macular edema. In certain embodiments, the HuPTM mAb has the amino acid sequence of lanadelumab or an antigen binding fragment thereof. The amino acid sequence of Fab fragment of this antibody is provided in FIG. 3. Alternatively, the antigen binding fragment is an scFv or an scFv-Fc. Delivery may be accomplished via gene therapy—e.g., by administering a viral vector or other DNA expression construct encoding an pKal-binding HuPTM mAb (or an antigen binding fragment and/or a hyperglycosylated derivative or other derivative, thereof) to patients (human subjects) diagnosed with angioedema or diabetic retinopathy and diabetic macular edema to create a permanent depot that continuously supplies the human PTM, e.g., human-glycosylated, transgene product.

### Transgenes

Provided are recombinant vectors containing a transgene encoding a HuPTM mAb or HuPTM Fab (or other antigen binding fragment of the HuPTM mAb, such as an scFv or scFv-Fc) that binds to pKal that can be administered to deliver the HuPTM mAb or antigen binding fragment in a patient. The transgene is a nucleic acid comprising the nucleotide sequences encoding an antigen binding fragment of an antibody that binds to pKal, such as lanadelumab or variants thereof as detailed herein. The transgene may also encode an anti-pKal antigen binding fragment that contains additional glycosylation sites (e.g., see Courtois et al.).

In certain embodiments, the anti-pKal antigen-binding fragment transgene comprises the nucleotide sequences encoding the heavy and light chains of the Fab portion of lanadelumab (having amino acid sequences of SEQ ID NOs: 144 and 145, respectively, see Table 7 and FIG. 3). The nucleotide sequences may be codon optimized for expression in human cells. Nucleotide sequences may, for example, comprise the nucleotide sequences of SEQ ID NO:146 (encoding the lanadelumab heavy chain Fab portion) and SEQ ID NO:147 (encoding the lanadelumab light chain Fab portion) as set forth in Table 7. The heavy and light chain sequences both have a signal or leader sequence at the N-terminus appropriate for expression and secretion in human cells, in particular, human liver cells (e.g., hepatocytes) or muscle cells. The signal sequence may have the amino acid sequence of MYRMQLLLLIALSLALVTNS (SEQ ID NO:50). Alternatively, the signal sequence may have an amino acid sequence selected from any one of the signal sequences set forth in Table 2 or 3 that correspond to the proteins secreted by myocytes or hepatocytes, respectively.

In addition to the heavy and light chain variable domain and C_{H}1 and C_{L} domain sequences, the transgenes may comprise, at the C-terminus of the heavy chain C_{H}1 domain sequence, all or a portion of the hinge region. In specific embodiments, the anti-pKal-antigen binding domain has a heavy chain Fab domain of SEQ ID NO: 248 with additional hinge region sequence starting after the C-terminal valine (V), contains all or a portion of the amino acid sequence EPKSCDKTHTCPPCPAPELLGG (SEQ ID NO:115), and specifically, EPKSCDKTHL (SEQ ID NO: 117), EPKSCDKTHT (SEQ ID NO:118), EPKSCDKTHTCPPCPA (SEQ ID NO:119), EPKSCDKTHLCPPCPA (SEQ ID NO:120), EPKSCDKTHTCPPCPAPELLGGPSVFL (SEQ ID NO: 121) or EPKSCDKTHLCPPCPAPELLGGPSVFL (SEQ ID NO:122) as set forth in Table 5 and FIG. 3. These hinge regions may be encoded by nucleotide sequences at the 3′ end of SEQ ID NO:146 by the hinge region encoding sequences set forth in Table 7. In another embodiment, the transgenes comprise the amino acid sequences encoding the full length (or substantially full length) heavy and light chains of the antibody, comprising the Fc domain at the C terminus of the heavy chain, e.g. having an amino acid sequence of SEQ ID NO:215 (Table 6) or an IgG1 Fc domain, such as SEQ ID NO: 141 or as depicted in Table 6, or a mutant or variant thereof. The Fc domain may be engineered for altered binding to one or more Fc receptors and/or effector function as disclosed in Section 5.1.9, infra.

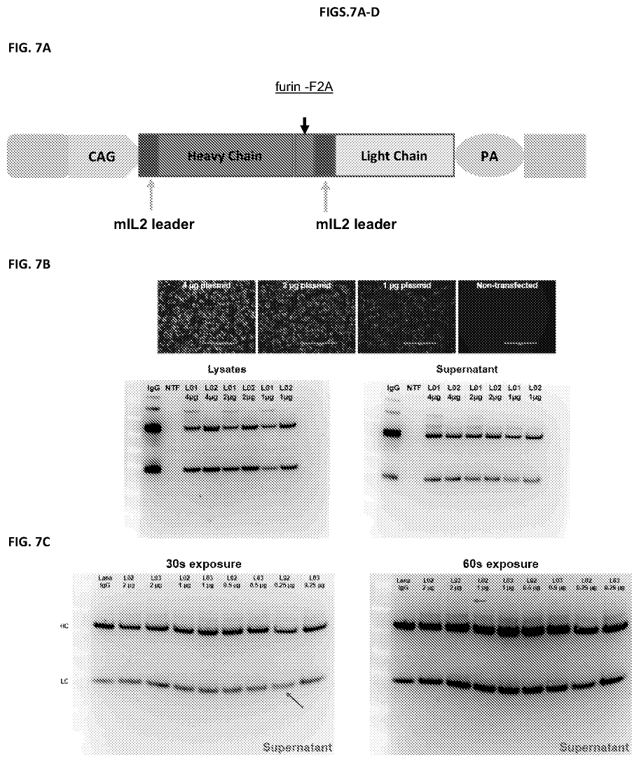

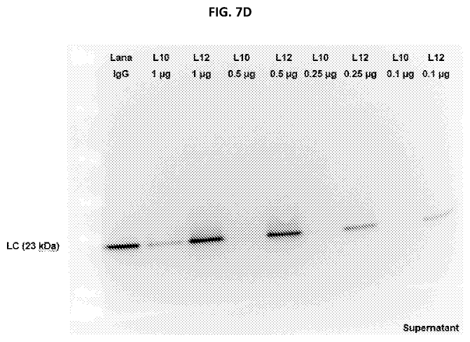

In specific embodiments, provided are constructs encoding a full length lanadelumab, including the Fc domain, particularly nucleotide sequence L01, L02 or L03 (SEQ ID NOs: 148, 149 or 150, respectively) as set forth in Table 7, herein, which are codon optimized and, in the case of L02 and L03 depleted for CpG dimers. The transgene may also comprises a nucleotide sequence that encodes a signal peptide MYRMQLLLLIALSLALVINS (SEQ ID NO:50); for example at the N-terminal of the heavy and/or the light chain) which may be encoded by the nucleotide sequence of SEQ ID NO:50. The nucleotide sequences encoding the light chain and heavy chain may be separated by a Furin-2A linker (SEQ ID NOs: 105 or 106) to create a bicistronic vector. Alternatively, the nucleotide sequences of the light chain and heavy chain are separated by a Furin-T2A linker, such as SEQ ID NO:103 or 104. Expression of the lanadelumab may be directed by a constitutive or a tissue specific promoter. In certain embodiments, the transgene contains a CAG promoter (SEQ ID NO:36) or a TBG (SEQ ID NO:40) promoter. Alternatively, the promoter may be a tissue specific promoter (or regulatory sequence including promoter and enhancer elements) such as liver-specific CREs of SEQ ID Nos 163-293, the APOE.hAAT regulatory sequence (SEQ ID NO:21), LSPX1 (SEQ ID NO: 9), LSPX2 (SEQ ID NO10), LTP1 (SEQ ID NO:11) or LMTP6 (SEQ ID NO:14) promoter, or CK8 (SEQ ID NO:37) promoter. See FIG. 7 for a schematic showing the genomic configuration. The transgenes may contain elements provided in Table 1. Exemplary transgenes encoding full length lanadelumab are provided in Table 7 and include CAG.LAN.F2A (SEQ ID NO:239 or 240); CAG.LAN.T2A (SEQ ID NO:241); TBG.LAN.T2A (SEQ ID NO:242); APOE.hAAT.LAN.T2A (SEQ ID NO: 243); LSPX1.LAN.T2A (SEQ ID NO:244); LSPX2.LAN.T2A (SEQ ID NO:245); LTP1.LAN.T2A (SEQ ID NO:246); and LMTP6.LAN.T2A (SEQ ID NO:247). ITR sequences are added to the 5′ and 3; ends of the constructs to generate the genomes. The transgenes may be packaged into AAV, particularly AAV8.

In certain embodiments, the anti-pKal antigen-binding fragment transgene encodes an pKal antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:145. In certain embodiments, the anti-pKal antigen-binding fragment transgene encodes an pKal antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:144. In certain embodiments, the anti-pKal antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:145 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO:144. In specific embodiments, the pKal antigen binding fragment comprises a heavy chain comprising an amino acid sequence of SEQ ID NO: 145 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 3). In specific embodiments, the pKal antigen binding fragment comprises a light chain comprising an amino acid sequence of SEQ ID NO: 145 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 3).

In certain embodiments, the anti-pKal antigen-binding fragment transgene encodes a hyperglycosylated lanadelumab Fab, comprising a heavy chain and a light chain of SEQ ID NOs: 144 and 145, respectively, with one or more of the following mutations: M117N (heavy chain) and/or Q159N, Q159S, and/or E194N (light chain).

In certain embodiments, the anti-pKal antigen-binding fragment transgene encodes an antigen-binding fragment and comprises the nucleotide sequences encoding the six lanadelumab CDRs which are underlined in the heavy and light chain variable domain sequences of FIG. 3 which are spaced between framework regions, generally human framework regions, and associated with constant domains depending upon the form of the antigen-binding molecule, as is known in the art to form the heavy and/or light chain variable domain of an anti-pKal antibody or antigen-binding fragment thereof.

In certain embodiments, the anti-pKal antigen-binding fragment transgene comprises a nucleotide sequence encoding an scFv or scFv-Fc comprising the heavy and light chain variable domains of lanadelumab (SEQ ID Nos: 314 and 318, respectfully, see Table 14) and, optionally the lanadelumab Fc domain, for example SEQ ID NO: 322. The nucleotide sequences may be codon optimized for expression in human cells. Nucleotide sequences may, for example, comprise the nucleotide sequences of SEQ ID NO:313 (encoding the lanadelumab heavy chain variable domain) and SEQ ID NO:317 (encoding the lanadelumab light chain variable domain) and SEQ ID NO: 321 (encoding the lanadelumab Fc domain) as set forth in Table 14. A leader sequence may be at the N terminus of the scFv or scFv-Fc, appropriate for expression and secretion in human cells, in particular, human liver cells (e.g., hepatocytes) or muscle cells. The signal sequence may have the amino acid sequence of MYRMQLLLLIALSLALVINS (SEQ ID NO:50). Alternatively, the signal sequence may have an amino acid sequence selected from any one of the signal sequences set forth in Table 2 or 3 that correspond to the proteins secreted by myocytes or hepatocytes, respectively.

The heavy and light chain variable domains are linked by flexible, non-cleavable linkers, for example, GGGGSGGGGSGGGGS (SEQ ID NO 316; encoded by SEQ ID NO: 315) or other linker in Table 4 or otherwise known in the art, and may be arranged as either N-terminus-VH-linker-VL-C-terminus or N-terminus-VL-linker-VL-C terminus. In embodiments, provided is an scFv-Fc in which an Fc domain is fused to the scFv by a flexible, non-cleavable linker (for example GGGGGGGGG (SEQ ID NO: 320)), which may be encoded by SEQ ID NO: 319) to the scFv. The Fc domain may have all or a portion of the hinge region (for example, one of the hinge sequences in Table 5): EPKSCDKTHTCPPCPAPELLGG (SEQ ID NO:115), EPKSCDKTHL (SEQ ID NO:117), EPKSCDKTHT (SEQ ID NO:118), EPKSCDKTHTCPPCPA (SEQ ID NO:119), EPKSCDKTHLCPPCPA (SEQ ID NO:120), EPKSCDKTHTCPPCPAPELLGGPSVFL (SEQ ID NO: 121) or EPKSCDKTHLCPPCPAPELLGGPSVFL (SEQ ID NO:122). The Fc domain may be a lanadelumab Fc domain having an amino acid sequence of SEQ ID NO: 25 or SEQ ID NO: 322. The Fc domain may be encoded by the nucleotide sequence of SEQ ID NO: 321. Alternatively the Fc domain may be an IgG1 Fc domain, such as SEQ ID NO:141 or as depicted in Table 6, or a mutant or variant thereof. The Fc domain may be engineered for altered binding to one or more Fc receptors and/or effector function as disclosed in Section 5.1.9, infra. In embodiments, the lanadelumab scFv-Fc is a VH-VL-Fc having an amino acid sequence of SEQ ID NO: 324, which may be encoded by the nucleotide sequence of SEQ ID NO: 323 or is a VL-VH-Fc having an amino acid sequence of SEQ ID NO: 393, which may be encoded by the nucleotide sequence of SEQ ID NO: 392.

The transgene may also comprises a nucleotide sequence that encodes a signal peptide MYRMQLLLLIALSLALVINS (SEQ ID NO:50); for example at the N-terminal of the heavy and/or the light chain) which may be encoded by the nucleotide sequence of SEQ ID NO:50. Expression of the lanadelumab scFv may be directed by a constitutive or a tissue specific promoter. In certain embodiments, the transgene contains a CAG promoter (SEQ ID NO:36) or a TBG (SEQ ID NO:40) promoter. Alternatively, the promoter may be a tissue specific promoter (or regulatory sequence including promoter and enhancer elements) such as liver-specific CREs of SEQ ID NO: 163-293, the APOE.hAAT regulatory sequence (SEQ ID NO:21), LSPX1 (SEQ ID NO:9), LSPX2 (SEQ ID NO10), LTP1 (SEQ ID NO:11) or LMTP6 (SEQ ID NO:14) promoter, or CK8 (SEQ ID NO:37) promoter. See FIG. 20 for a schematic showing the genomic configuration. The transgenes may contain elements provided in Table 1, such as polyadenylation signals, introns, and ITR sequences. Exemplary transgenes encoding lanadelumab scFv-Fcs are provided in Table 14 (see also FIG. 20) and include ApoE.hAAT.Lan-HL-scFv-Fc (SEQ ID NO: 308), or LMTP6.Lan.HL-scFv-Fc (SEQ ID NO: 325), ApoE.hAAT.Lan-LH-scFv-Fc (SEQ ID NO: 332), and LMTP6.Lan-LH-scFv-Fc (SEQ ID NO: 333). ITR sequences are added to the 5′ and 3; ends of the constructs to generate the genomes. The transgenes may be packaged into AAV, particularly AAV8.

In certain embodiments, the anti-pKal scFv-Fc transgene encodes an pKal antigen-binding fragment that is at least 85%, 86%, 87%, 88%, 89%, 90%, 91%, 92%, 93%, 94%, 95%, 96%, 97%, 98% or 99% identical to the sequence set forth in SEQ ID NO:324 or 393. In specific embodiments, the pKal antigen binding fragment scFv-Fc comprises an amino acid sequence of SEQ ID NO: 324 or 393 with 1, 2, 3, 4, 5, 6, 7, 8, 9, 10, 11, 12, 13, 14, 15 or more amino acid substitutions, insertions or deletions, and, in certain embodiments, the substitutions, insertions or deletions are made, e.g., in the framework regions (e.g., those regions outside of the CDRs, which CDRs are underlined in FIG. 3).

### Gene Therapy Methods

Provided are methods of treating human subjects for angioedema by administration of a viral vector containing a transgene encoding an anti-pKal antibody, or antigen binding fragment thereof. The antibody may be lanadelumab and is, e.g., a full length or substantially full length antibody or Fab fragment thereof, or other antigen-binding fragment thereof, such as an scFv or scFv-Fc. In embodiments, the patient has been diagnosed with and/or has symptoms associated with angioedema. Recombinant vectors used for delivering the transgene are described in above and in Section 5.1 and exemplary transgenes are provided above. Such vectors should have a tropism for human liver or muscle cells and can include non-replicating rAAV, particularly those bearing an AAV8 capsid. The recombinant vectors, such as shown in FIG. 3 or FIG. 20, can be administered in any manner such that the recombinant vector enters the liver tissue and/or the muscle tissue, e.g., by introducing the recombinant vector into the bloodstream, for example by intravenous or intramuscular administration. See below for details regarding the methods of treatment.

Provided are methods of treating human subjects for diabetic retinopathy or diabetic macular edema by administration of a viral vector containing a transgene encoding an anti-pKal antibody, or antigen binding fragment thereof. The antibody may be lanadelumab and is, e.g., a full length or substantially full length antibody or Fab fragment thereof, or other antigen-binding fragment thereof. In embodiments, the patient has been diagnosed with and/or has symptoms associated with diabetic retinopathy or diabetic macular edema. Recombinant vectors used for delivering the transgene are described in Section 5.1 and exemplary transgenes are provided above. Such vectors should have a tropism for human retinal cells and can include non-replicating rAAV, particularly those bearing an AAV8 or AAV9 capsid. The recombinant vectors, such as shown in FIG. 3, can be administered in any manner such that the recombinant vector enters the retinal tissue. In particular embodiments, the transgene is CAG.LAN.F2A (SEQ ID NO:239 or 1240); CAG.LAN.T2A (SEQ ID NO: 241); TBG.LAN.T2A (SEQ ID NO:242); APOE.hAAT.LAN.T2A (SEQ ID NO:243); LSPX1.LAN.T2A (SEQ ID NO:244); LSPX2.LAN.T2A (SEQ ID NO:245); LTP1.LAN.T2A (SEQ ID NO: 246); and LMTP6.LAN.T2A (SEQ ID NO:247) or ApoE.hAAT.Lan-HL-scFv-Fc (SEQ ID NO: 308), or LMTP6.Lan.HL-scFv-Fc (SEQ ID NO: 325), ApoE.hAAT.Lan-LH-scFv-Fc (SEQ ID NO: 332), and LMTP6.Lan-LH-scFv-Fc (SEQ ID NO: 333) in an AAV8 vector.

The example provide results of serum levels of lanadelumab in mice, rats and non-human primates administered AAV vectors encoding full length lanadelumab to assess different promoters and other regulatory elements, linkers, AAV types, modes of administration, etc. Such results inform dosage of a recombinant AAV vector encoding lanadelumab to achieve serum levels, particularly, steady state serum levels, sufficient for therapeutic efficacy. Steady state serum levels of sufficient therapeutic efficacy may be determined through clinical studies, for example, as provided in the prescribing information for lanadelumab (see TAKHZYRO® Prescribing Information). In particular embodiments, the AAV8 lanadelumab vector is administered to a patient in need thereof, for example, a patient diagnosed with or suffering from HAE, at a dosage (vector genomes) sufficient for to expression of therapeutically effective levels of lanadelumab in the patient serum while minimizing side effects such as transaminitis or the development of anti-drug antibodies. In particular embodiments, the dosages 1E11 vg/kg to 1E14 vg/kg, including 1E11 vg/kg, 1E12 vg/kg, 1E13 vg/kg, or 1E14 vg/kg. In specific embodiments, the administration results in Cₘₐₓ of 9 μg/ml to 35 μg/ml, including between 12 μg/ml to 25 μg/ml, or between 20 μg/ml and 35 μg/ml; and a Cₘᵢₙ of 1 μg/ml, 2 μg/ml or 4 μg/ml to 25 μg/ml or a Cₘᵢₙ greater than 1 μg/ml, 2 μg/ml or 4 μg/ml, 10 μg/ml or 20 μg/ml, but in certain embodiments less than 200 μg/ml or 500 μg/ml. The serum or plasma concentration is preferably achieved as a steady state concentration, for example, maintaining serum or plasma levels within the Cₘₐₓ and Cₘᵢₙ for at least 1 month, 2 months, 3 months, or greater than 3 months, or 1 year. In specific embodiments, administration of the AAV vector results in steady state lanadelumab plasma concentration of 1.0 μg/ml, 2.0 μg/ml, 5 μg/ml to 30 μg/ml or 10 μg/ml to 20 μg/ml; or 15 μg/ml to 30 μg/ml or greater than 20 μg/ml, but in certain embodiments less than 200 μg/ml or 500 μg/ml. In particular embodiments, the lanadelumab antibody secreted into the plasma exhibits a greater than at least 40%, 45%, 50%, 55%, 60%, 65% or 70 reduction in pKal activity as measured by a kinetic enzymatic functional assay, for example, the assay described in Example 9. In certain embodiments, the activity of the lanadelumab antibody is measured at 2 weeks, 3 weeks, 4 weeks, 5 weeks, 6 weeks, 7 weeks, 8 weeks, 9 weeks, 10 weeks, 11 weeks or 12 weeks after administration of the AAV vector. In certain embodiments, the lanadelumab plasma concentration of 1.0 μg/ml, 2.0 μg/ml, 5 μg/ml to 30 μg/ml, or 10 μg/ml to 20 μg/ml, or 15 μg/ml to 30 μg/ml is sufficient to relieve or ameliorate the symptoms of hereditary angioedema in a human subject. The methods of treatment provided herein reduce the incidence or severity of angioedema occurrences or attacks. In particular embodiments, the angioedema occurs in the skin, the gastrointestinal tract or the upper airway.

Subjects to whom such gene therapy is administered can be those responsive to anti-pKal therapy. In certain embodiments, the methods encompass treating patients who have been diagnosed with angioedema or diabetic retinopathy, or have one or more symptoms associated therewith, and identified as responsive to treatment with an anti-pKal antibody or considered a good candidate for therapy with an anti-pKal antibody. In specific embodiments, the patients have previously been treated with lanadelumab, and have been found to be responsive to lanadelumab. To determine responsiveness, the anti-pKal antibody or antigen-binding fragment transgene product (e.g., produced in cell culture, bioreactors, etc.) may be administered directly to the subject.

### Human Post Translationally Modified Antibodies

The production of the anti-pKal HuPTM mAb or HuPTM Fab, should result in a “biobetter” molecule for the treatment of angioedema accomplished via gene therapy—e.g., by administering a viral vector or other DNA expression construct encoding the anti-pKal HuPTM Fab, intravenously to human subjects (patients) diagnosed with or having one or more symptoms of angioedema, to create a permanent depot in the liver or muscle tissue that continuously supplies the fully-human post-translationally modified, e.g., human-glycosylated, sulfated transgene product produced by transduced liver or muscle cells.

In specific embodiments, the anti-pKal HuPTM mAb or antigen-binding fragment thereof has heavy and light chains with the amino acid sequences of the heavy and light chain Fab portions of lanadelumab as set forth in FIG. 3 (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N77, Q114 and/or N164 of the heavy chain (SEQ ID NO:144) or Q99, N157, and/or N209 of the light chain (SEQ ID NO:145). Alternatively or in addition to, the HuPTM mAb or antigen binding-fragment thereof with the heavy and light chain variable domain sequences of lanadelumab has a sulfation group at Y94 and/or Y95 of the heavy chain (SEQ ID NO:144) and/or Y86 and/or Y87 of the light chain (SEQ ID NO:145). In other embodiments, the anti-pKal HuPTM mAb or antigen-binding fragment thereof does not contain detectable NeuGc moieties and/or does not contain detectable alpha-Gal moieties. In certain embodiments, the HuPTM mAb is a full length or substantially full length mAb with an Fc region.

In certain embodiments, the HuPTM mAb or Fab (or a hyperglycosylated derivative of either) is therapeutically effective and is at least 0.5%, 1% or 2% glycosylated and/or sulfated and may be at least 5%, 10% or even 50% or 100% glycosylated and/or sulfated. The goal of gene therapy treatment provided herein is to slow or arrest the progression of angioedema, reduce the levels of pain or discomfort for the patient, or reduce levels of autoreactive B cells and immunoglobulin producing plasma cells. Efficacy may be monitored by scoring the function, symptoms, or degree of inflammation in the affected tissue or area of the body, e.g., such as the skin, joints, kidneys, lungs, blood cells, heart, and brain. For example, efficacy can be monitored by assessing changes in attack severity or frequency.

Combinations of delivery of the anti-pKal HuPTM mAb or antigen-binding fragment thereof, to the liver or muscle accompanied by delivery of other available treatments are encompassed by the methods provided herein. The additional treatments may be administered before, concurrently, or subsequent to the gene therapy treatment. Available treatments for angioedema that could be combined with the gene therapy provided herein include but are not limited to danazol, bradykinin receptor antagonist (e.g., icatibant), plasma kallikrein inhibitor (e.g., ecallantide), C1 esterase inhibitor, conestat alfa, anti-fibrinolytic agents (e.g., tranexamic acid), omalizumab, and fresh frozen plasma transfusions, antihistamines, and corticosteroids and administration with anti-pKal agents, including but not limited to lanadelumab.

5.4.2. Dose Administration of pKal Antibodies

Section 5.2. and 5.4.1 describe recombinant vectors that contain a transgene encoding a HuPTM mAb or HuPTM Fab (or other antigen binding fragment of the HuPTM mAb) that binds to pKal. Therapeutically effective doses of any such recombinant vector should be administered in any manner such that the recombinant vector enters the liver or muscle (e.g., skeletal muscle), e.g. by introducing the recombinant vector into the bloodstream by intravenous or intramuscular administration. Alternatively, the vector may be administered directly to the liver through hepatic blood flow, e.g., via the suprahepatic veins or via the hepatic artery. In specific, embodiments, the vector is administered subcutaneously, intramuscularly or intravenously. Intramuscular, subcutaneous, intravenous or hepatic administration should result in expression of the soluble transgene product in cells of the liver or muscle. Alternatively, the vector may be administered directly to the liver through hepatic blood flow, e.g., via the suprahepatic veins or via the hepatic artery. The expression of the transgene encoding an anti-pKal antibody creates a permanent depot in liver and/or muscle of the patient that continuously supplies the anti-pKal HuPTM mAb, or antigen binding fragment of the anti-pKal mAb to the circulation of the subject.

In certain embodiments, the range of a therapeutically or prophylactically effective amount of an AAV gene therapy vector or pharmaceutical composition is 1E11 to 1E14 genome copies (gc)/kg, preferably between 1E11 to 1E13, and even more preferably 1E12. The dose ranges described herein are exemplary only and do not limit the dose ranges that can be selected.

In certain embodiments, intravenous administration of an AAV gene therapy vector encoding an anti-pKal antibody (lanadelumab) results in at least 1.5 μg/mL, 2 μg/mL, 5 μg/mL, 10 μg/mL, or at least 15 μg/mL transgene product expression in human serum at least 20, 30, 40, 50 or 60 days after administration. In certain embodiments, the target human serum concentration (Cₘᵢₙ) of the transgene product is about 1.5 μg/mL to about 15 μg/mL mAb.

In certain embodiments, doses that maintain a serum concentration of the anti-pKal antibody transgene product at a Cₘᵢₙ of at least 1.5 μg/mL or at least 15 μg/mL (e.g., Cₘᵢₙ of 1.5 to 5 μg/ml, 5 to 10 μg/ml or 10 to 15 μg/mL) at least 30, 40, 50 or 69 days after administration are provided. In preferred embodiment, a dose of 1E11 maintains a serum concentration of the anti-pKal antibody transgene product of at least 1.5 μg/mL. In another embodiment, a dose of 1E12 maintains a serum concentration of the anti-pKal antibody transgene product of at least 1.5 μg/mL.

However, in all cases because the transgene product is continuously produced, maintenance of lower concentrations can be effective. Notwithstanding, because the transgene product is continuously produced, maintenance of lower concentrations can be effective. The concentration of the transgene product can be measured in patient blood serum samples.

Pharmaceutical compositions suitable for intravenous, intramuscular, subcutaneous or hepatic administration comprise a suspension of the recombinant vector comprising the transgene encoding the anti-pKal antibody, or antigen-binding fragment thereof, in a formulation buffer comprising a physiologically compatible aqueous buffer. The formulation buffer can comprise one or more of a polysaccharide, a surfactant, polymer, or oil.

### 6. EXAMPLES

### Example 1: Lanadelumab Fab cDNA-Based Vector

A lanadelumab Fab cDNA-based vector is constructed comprising a transgene comprising nucleotide sequences encoding the Fab portion of the heavy and light chain sequences of lanadelumab (amino acid sequences being SEQ ID NOs: 144 and 145, respectively). The nucleotide sequence coding for the Fab portion of the heavy and light chain may be the nucleotide sequence of SEQ ID NOs: 146 and 147, respectively. The transgene also comprises nucleotide sequences that encodes a signal peptide, e.g., MYRMQLLLLIALSLALVTNS (SEQ ID NO:50). The nucleotide sequences encoding the light chain and heavy chain are separated by IRES elements or 2A cleavage sites (See Table 4, particularly, SEQ ID NO:105 or 106) to create a bicistronic vector. The vector additionally includes a constitutive promoter, such as CB7, a tissue-specific promoter, such as a liver specific promoter, particularly liver-specific CREs of SEQ ID Nos: 163-293, ApoE.hAAT promoter (SEQ ID NO:21), an inducible promoter, such as a hypoxia-inducible promoter.

### Example 2: Protein Expression Analysis of Lanadelumab in Cell Lysates and Supernatant

Cell culture studies were performed to assess the expression of full length mAb sequences (containing Fc region) from AAV constructs in human cells.

### Methods

A lanadelumab cDNA-based vector was constructed comprising a transgene comprising a nucleotide sequence encoding the heavy and light chain sequences of lanadelumab (amino acid sequences being SEQ ID NOs: 144 and 145, respectively). The nucleotide sequence coding for the heavy and light chain of lanadelumab was codon optimized to generate the three nucleotide sequences provided in Table 7 below, L01 (SEQ ID NO:148), L02 (SEQ ID NO:149), and L03 (SEQ ID NO:150). L02 and L03 also have reduced incidence of CpG dimers in the sequence. The transgene also comprised a nucleotide sequence that encodes the signal peptide MYRMQLLLLIALSLALVINS (SEQ ID NO:50). The nucleotide sequences encoding the light chain and heavy chain were separated by a Furin-F2A linker (SEQ ID NOS: 105 or 106) or a Furin T2A linker (SEQ ID NOS: 103 or 104) to create a bicistronic vector. The vector additionally included a constitutive CAG promoter (SEQ ID NO:36). See FIG. 7A for a schematic showing the genomic configuration and sequences of the constructs are provided in Table 7 (SEQ ID NOS: 151-159).

Table 1 (and also SEQ ID Nos; 163-293) provides the sequences of composite nucleic acid regulatory sequences that may be incorporated into expression cassettes and be operably linked to the transgene to promote liver-specific expression (LSPX1, LSPX2, LTP1, LTP2, or LTP3, SEQ ID NOS: 9-13, respectively, and all sequences of SEQ ID Nos: 163-293) and liver and muscle expression (LMTP6, LMTP13, LMTP15, LMTP18, LMTP19 or LMTP20, SEQ ID NOS: 14-20 respectively). Other promoter sequences provided, include the ApoE.hAAT (SEQ ID NO:21, Table 1 above) promoter, wherein four copies of the liver-specific apolipoprotein E (ApoE) enhancer were placed upstream of the human alpha 1-antitrypsin (hAAT) promoter). Alternatively, a promoter sequence can include a CRE sequence selected from Table 14 upstream of a hAAT promoter, such as four copies of a liver-specific CRE selected from Table 14 placed upstream of the human alpha 1-antitrypsin (hAAT) promoter).

TABLE 7

| Name/ | |
|---|---|
| SEQ ID NO. | Sequence |
| Lanadelumab | EVQLLESGGG LVQPGGSLRL SCAASGFTFS HYIMMWVRQA PGKGLEWVSG |
| Heavy/SEQ ID | IYSSGGITVY ADSVKGRFTI SRDNSKNTLY LQMNSLRAED TAVYYCAYRR |
| NO: 144 | IGVPRRDEFD IWGQGTMVTV SSASTKGPSV FPLAPSSKST SGGTAALGCL |
| | VKDYFPEPVT VSWNSGALTS GVHTFPAVLQ SSGLYSLSSV VTVPSSSLGT |
| | QTYICNVNHK PSNTKVDKRV EPKSCD +/− KTHT (or KTHL) +/− CPPCPA |
| | +/− PELLGGPSVFL |
| Lanadelumab | DIQMTQSPST LSASVGDRVT ITCRASQSIS SWLAWYQQKP GKAPKLLIYK |
| Light/SEQ ID | ASTLESGVPS RFSGSGSGTE FTLTISSLQP DDFATYYCQQ YNTYWTFGQG |
| NO: 145 | TKVEIKRTVA APSVFIFPPS DEQLKSGTAS VVCLLNNFYP REAKVQWKVD |
| | NALQSGNSQE SVTEQDSKDS TYSLSSTLTL SKADYEKHKV YACEVTHQGL |
| | SSPVTKSENR GEC |
| Lanadelumab | GAGGTGCAGCTGCTGGAGAGCGGCGGCGGCCTGGTGCAGCCCGGCGGCAGCCTGAGGCTG |
| Heavy/SEQ ID | AGCTGCGCCGCCAGCGGCTTCACCTTCAGCCACTACATCATGATGTGGGTGAGGCAGGCC |
| NO: 146 | CCCGGCAAGGGCCTGGAGTGGGTGAGCGGCATCTACAGCAGCGGCGGCATCACCGTGTAC |
| | GCCGACAGCGTGAAGGGCAGGTTCACCATCAGCAGGGACAACAGCAAGAACACCCTGTAC |
| | CTGCAGATGAACAGCCTGAGGGCCGAGGACACCGCCGTGTACTACTGCGCCTACAGGAGG |
| | ATCGGCGTGCCCAGGAGGGACGAGTTCGACATCTGGGGCCAGGGCACCATGGTGACCGTG |
| | AGCAGCGCCAGCACCAAGGGCCCCAGCGTGTTCCCCCTGGCCCCCAGCAGCAAGAGCACC |
| | AGCGGCGGCACCGCCGCCCTGGGCTGCCTGGTGAAGGACTACTTCCCCGAGCCCGTGACC |
| | GTGAGCTGGAACAGCGGCGCCCTGACCAGCGGCGTGCACACCTTCCCCGCCGTGCTGCAG |
| | AGCAGCGGCCTGTACAGCCTGAGCAGCGTGGTGACCGTGCCCAGCAGCAGCCTGGGCACC |
| | CAGACCTACATCTGCAACGTGAACCACAAGCCCAGCAACACCAAGGTGGACAAGAGGGTG |
| | GAGCCCAAGAGCTGCGAC+/− AAGACCCACACC (or AAGACCCACCTG) +/− |
| | TGCCCCCCCTGCCCCGCC+/− CCCGAGCTGCTGGGCGGCCCCAGCGTGT TCCTG |
| Lanadelumab | GACATCCAGATGACCCAGAGCCCCAGCACCCTGAGCGCCAGCGTGGGCGACAGGGTGACC |
| Light/SEQ ID | ATCACCTGCAGGGCCAGCCAGAGCATCAGCAGCTGGCTGGCCTGGTACCAGCAGAAGCCC |
| NO: 147 | GGCAAGGCCCCCAAGCTGCTGATCTACAAGGCCAGCACCCTGGAGAGCGGCGTGCCCAGC |
| | AGGTTCAGCGGCAGCGGCAGCGGCACCGAGTTCACCCTGACCATCAGCAGCCTGCAGCCC |
| | GACGACTTCGCCACCTACTACTGCCAGCAGTACAACACCTACTGGACCTTCGGCCAGGGC |
| | ACCAAGGTGGAGATCAAGAGGACCGTGGCCGCCCCCAGCGTGTTCATCTTCCCCCCCAGC |
| | GACGAGCAGCTGAAGAGCGGCACCGCCAGCGTGGTGTGCCTGCTGAACAACTTCTACCCC |
| | AGGGAGGCCAAGGTGCAGTGGAAGGTGGACAACGCCCTGCAGAGCGGCAACAGCCAGGAG |
| | AGCGTGACCGAGCAGGACAGCAAGGACAGCACCTACAGCCTGAGCAGCACCCTGACCCTG |
| | AGCAAGGCCGACTACGAGAAGCACAAGGTGTACGCCTGCGAGGTGACCCACCAGGGCCTG |
| | AGCAGCCCCGTGACCAAGAGCTTCAACAGGGGCGAGTGC |
| L01 | ATGTACCGGATGCAGCTGCTGCTGCTCATTGCCCTGTCTCTGGCCCTGGTCACCAATAGC |
| SEQ ID NO: 148 | GAAGTCCAGCTGCTGGAATCTGGCGGCGGACTTGTTCAACCTGGCGGCTCTCTGAGACTG |
| | AGCTGTGCCGCTTCCGGCTTCACCTTCAGCCACTATATCATGATGTGGGTCCGACAGGCC |
| | CCTGGCAAAGGCCTTGAATGGGTGTCCGGCATCTATAGCAGCGGCGGCATCACAGTGTAC |
| | GCCGACTCTGTGAAGGGCAGATTCACCATCAGCCGGGACAACAGCAAGAACACCCTGTAC |
| | CTGCAGATGAACAGCCTGAGAGCCGAGGACACCGCCGTGTACTACTGCGCCTATAGAAGA |
| | ATCGGCGTGCCCAGACGGGACGAGTTCGATATTTGGGGCCAGGGCACCATGGTCACCGTG |
| | TCTAGCGCCTCTACAAAGGGCCCTAGCGTGTTCCCTCTGGCTCCTAGCAGCAAGAGCACA |
| | AGCGGAGGAACAGCCGCTCTGGGCTGTCTGGTCAAGGACTACTTTCCCGAGCCTGTGACC |
| | GTGTCCTGGAATAGCGGAGCACTGACAAGCGGCGTGCACACCTTTCCAGCTGTGCTGCAA |
| | AGCAGCGGCCTGTACTCTCTGAGCAGCGTGGTCACAGTGCCAAGCTCTAGCCTGGGCACC |
| | CAGACCTACATCTGCAATGTGAACCACAAGCCTAGCAACACCAAGGTGGACAAGAGAGTG |
| | GAACCCAAGAGCTGCGACAAGACCCACACCTGTCCTCCATGTCCTGCTCCAGAACTGCTC |
| | GGCGGACCTTCCGTGTTCCTGTTTCCTCCAAAGCCTAAGGACACCCTGATGATCAGCAGA |
| | ACCCCTGAAGTGACCTGCGTGGTGGTGGATGTGTCCCACGAGGATCCCGAAGTGAAGTTC |
| | AATTGGTACGTGGACGGCGTGGAAGTGCACAACGCCAAGACCAAGCCTAGAGAGGAACAG |
| | TACAACAGCACCTACAGAGTGGTGTCCGTGCTGACCGTGCTGCACCAGGATTGGCTGAAC |
| | GGCAAAGAGTACAAGTGCAAGGTGTCCAACAAGGCCCTGCCTGCTCCTATCGAGAAAACC |
| | ATCAGCAAGGCCAAGGGCCAGCCTAGGGAACCCCAGGTTTACACACTGCCTCCAAGCCGG |
| | GAAGAGATGACCAAGAACCAGGTGTCCCTGACCTGCCTCGTGAAGGGCTTCTACCCTTCC |
| | GATATCGCCGTGGAATGGGAGAGCAATGGCCAGCCAGAGAACAACTACAAGACAACCCCT |
| | CCTGTGCTGGACAGCGACGGCTCATTCTTCCTGTACAGCAAGCTGACAGTGGACAAGTCC |
| | AGATGGCAGCAGGGCAACGTGTTCAGCTGCAGCGTGATGCACGAGGCCCTGCACAACCAC |
| | TACACCCAGAAGAGTCTGAGCCTGTCTCCAGGCCTGCGGAAGAGAAGGGCTCCTGTGAAG |
| | CAGACCCTGAACTTCGACCTGCTGAAACTGGCCGGCGACGTGGAAAGCAACCCCGGACCT |
| | ATGTATAGAATGCAGCTCCTCCTGCTGATCGCACTGAGCCTGGCTCTCGTGACCAACAGC |
| | GACATCCAGATGACACAGAGCCCCAGCACACTGTCTGCCAGCGTGGGAGACAGAGTGACC |
| | ATCACCTGTAGAGCCAGCCAGTCCATCTCCTCTTGGCTGGCCTGGTATCAGCAAAAGCCT |
| | GGCAAGGCCCCTAAGCTGCTGATCTACAAGGCCTCCACACTGGAAAGCGGGGTGCCCTCC |
| | AGATTTTCTGGCAGCGGATCTGGCACCGAGTTCACCCTGACCATCAGTAGCCTGCAGCCT |
| | GACGACTTCGCCACCTACTACTGCCAGCAGTACAATACCTACTGGACCTTCGGCCAGGGA |
| | ACAAAGGTGGAAATCAAGCGGACTGTGGCCGCTCCAAGCGTGTTCATCTTTCCACCTAGC |
| | GACGAGCAGCTGAAGTCCGGCACAGCCTCTGTTGTGTGCCTGCTGAACAACTTCTACCCC |
| | AGAGAAGCCAAGGTGCAGTGGAAAGTGGACAATGCCCTGCAGAGCGGCAACTCCCAAGAG |
| | AGCGTGACAGAGCAGGACTCCAAGGATTCCACCTACAGCCTGTCTAGCACCCTGACACTG |
| | AGCAAGGCCGACTACGAGAAGCACAAAGTGTATGCCTGCGAAGTGACCCACCAGGGCCTT |
| | AGCTCTCCAGTGACCAAGAGCTTCAACCGGGGCGAGTGTTGATAA |
| L02 | ATGTACAGAATGCAGCTGCTGCTGCTCATTGCCCTGTCTCTGGCCCTGGTCACCAATTCT |
| SEQ ID NO: 149 | GAGGTCCAGCTGCTTGAGAGTGGTGGTGGACTGGTTCAGCCTGGTGGCAGCCTGAGACTG |
| | TCTTGTGCTGCCTCTGGCTTCACCTTCAGCCACTATATCATGATGTGGGTCAGACAGGCC |
| | CCTGGCAAAGGCCTGGAATGGGTGTCAGGCATCTACAGCAGTGGTGGCATCACAGTGTAT |
| | GCTGACTCTGTGAAGGGCAGATTCACCATCAGCAGAGACAACAGCAAGAACACCCTGTAC |
| | CTGCAGATGAACTCCCTGAGAGCTGAGGACACAGCAGTGTACTACTGTGCCTATAGAAGA |
| | ATTGGGGTGCCCAGAAGGGATGAGTTTGACATCTGGGGCCAGGGCACCATGGTTACAGTG |
| | TCCTCTGCCAGCACAAAGGGCCCCTCTGTTTTTCCACTGGCTCCCAGCAGCAAGAGCACC |
| | AGTGGTGGAACAGCTGCCCTG |
| | GGCTGTCTGGTCAAGGATTACTTCCCTGAGCCTGTGACTGTGTCCTGGAACTCTGGGGCT |
| | CTGACTTCTGGGGTGCACACCTTTCCAGCTGTGCTGCAGTCCTCTGGCCTGTACTCTCTG |
| | TCCTCTGTGGTCACAGTGCCTAGCTCTAGCCTGGGCACACAGACCTACATCTGCAATGTG |
| | AACCACAAGCCTAGCAACACCAAGGTGGACAAGAGAGTGGAACCCAAGAGCTGTGACAAG |
| | ACCCACACCTGTCCTCCATGTCCTGCTCCAGAACTGCTTGGAGGCCCTTCTGTGTTCCTG |
| | TTTCCTCCAAAGCCTAAGGACACCCTGATGATCAGCAGAACCCCTGAAGTGACCTGTGTG |
| | GTGGTTGATGTGTCCCATGAGGACCCAGAAGTGAAGTTCAATTGGTATGTGGATGGGGTT |
| | GAAGTGCACAATGCCAAGACCAAGCCTAGAGAGGAACAGTACAACAGCACCTACAGAGTG |
| | GTTTCTGTGCTGACAGTGCTGCACCAGGACTGGCTGAATGGCAAAGAGTACAAGTGCAAG |
| | GTGTCCAACAAGGCCCTGCCTGCTCCTATTGAGAAAACCATCTCCAAGGCCAAGGGCCAG |
| | CCAAGAGAACCCCAGGTTTACACCCTGCCACCTAGCAGAGAAGAGATGACCAAGAACCAG |
| | GTGTCCCTGACCTGCCTGGTTAAGGGCTTCTACCCCTCTGACATTGCTGTGGAATGGGAG |
| | AGCAATGGCCAGCCTGAAAACAACTACAAGACAACCCCTCCTGTGCTGGACTCTGATGGC |
| | TCATTCTTCCTGTACAGCAAGCTGACTGTGGACAAGTCCAGATGGCAGCAGGGAAATGTG |
| | TTCAGCTGCTCTGTGATGCATGAGGCCCTGCACAACCACTACACCCAGAAAAGTCTGTCT |
| | CTGTCCCCTGGCAGAAAGAGAAGAGGCTCTGGAGAAGGCAGAGGCTCCCTGCTGACATGT |
| | GGGGATGTTGAAGAGAATCCTGGGCCTATGTATAGGATGCAACTGCTCCTCCTGATTGCT |
| | CTGAGCCTGGCTCTTGTGACCAACTCTGACATCCAGATGACACAGAGCCCCTCCACACTG |
| | TCTGCATCTGTGGGAGACAGAGTGACCATCACCTGTAGAGCCAGCCAGTCTATCTCTAGC |
| | TGGCTGGCCTGGTATCAGCAAAAGCCTGGCAAGGCCCCTAAGCTGCTGATCTACAAGGCC |
| | AGCACACTTGAGTCAGGGGTGCCCTCCAGATTTTCTGGCTCTGGATCTGGCACAGAGTTC |
| | ACCCTGACCATCAGCTCCCTGCAGCCAGATGACTTTGCCACCTACTACTGCCAGCAGTAC |
| | AATACCTACTGGACCTTTGGCCAGGGAACAAAGGTGGAAATCAAGAGAACAGTGGCTGCC |
| | CCATCTGTGTTCATCTTCCCACCATCTGATGAACAGCTGAAGTCTGGCACTGCCTCTGTT |
| | GTGTGCCTGCTGAACAACTTTTACCCCAGAGAAGCCAAGGTGCAGTGGAAAGTGGATAAT |
| | GCCCTGCAGTCTGGCAATAGCCAAGAATCTGTGACAGAGCAGGACTCCAAGGATTCCACC |
| | TACAGCCTGAGCAGCACCCTGACACTGAGCAAGGCTGACTATGAGAAGCACAAAGTGTAT |
| | GCCTGTGAAGTGACACACCAGGGACTGAGCAGCCCAGTGACCAAGAGCTTCAACAGGGGA |
| | GAGTGCTGATAA |
| L03 | ATGTACAGAATGCAGCTGCTGCTGCTCATTGCCCTGTCTCTGGCCCTGGTCACCAATTCT |
| SEQ ID NO: 150 | GAGGTCCAGCTGCTTGAGAGTGGTGGTGGACTGGTTCAGCCTGGTGGCAGCCTGAGACTG |
| | TCTTGTGCTGCCTCTGGCTTCACCTTCAGCCACTATATCATGATGTGGGTCAGACAGGCC |
| | CCTGGCAAAGGCCTGGAATGGGTGTCAGGCATCTACAGCAGTGGTGGCATCACAGTGTAT |
| | GCTGACTCTGTGAAGGGCAGATTCACCATCAGCAGAGACAACAGCAAGAACACCCTGTAC |
| | CTGCAGATGAACTCCCTGAGAGCTGAGGACACAGCAGTGTACTACTGTGCCTATAGAAGA |
| | ATTGGGGTGCCCAGAAGGGATGAGTTTGACATCTGGGGCCAGGGCACCATGGTTACAGTG |
| | TCCTCTGCCAGCACAAAGGGCCCCTCTGTTTTTCCACTGGCTCCCAGCAGCAAGAGCACC |
| | AGTGGTGGAACAGCTGCCCTGGGCTGTCTGGTCAAGGATTACTTCCCTGAGCCTGTGACT |
| | GTGTCCTGGAACTCTGGGGCTCTGACCTCTGGGGTGCACACCTTTCCAGCTGTGCTGCAG |
| | TCCTCTGGCCTGTACTCTCTGTCCTCTGTGGTCACAGTGCCTAGCTCTAGCCTGGGCACA |
| | CAGACCTACATCTGCAATGTGAACCACAAGCCTAGCAACACCAAGGTGGACAAGAGAGTG |
| | GAACCCAAGAGCTGTGACAAGACCCACACCTGTCCTCCATGTCCTGCTCCAGAACTGCTT |
| | GGAGGCCCTTCTGTGTTCCTGTTTCCTCCAAAGCCTAAGGACACCCTGATGATCAGCAGA |
| | ACCCCTGAAGTGACCTGTGTGGTGGTTGATGTGTCCCATGAGGACCCAGAAGTGAAGTTC |
| | AATTGGTATGTGGATGGGGTTGAAGTGCACAATGCCAAGACCAAGCCTAGAGAGGAACAG |
| | TACAACAGCACCTACAGAGTGGTTTCTGTGCTGACAGTGCTGCACCAGGACTGGCTGAAT |
| | GGCAAAGAGTACAAGTGCAAGGTGTCCAACAAGGCCCTGCCTGCTCCTATTGAGAAAACC |
| | ATCTCCAAGGCCAAGGGCCAGCCAAGAGAACCCCAGGTTTACACCCTGCCACCTAGCAGA |
| | GAAGAGATGACCAAGAACCAGGTGTCCCTGACCTGCCTGGTTAAGGGCTTCTACCCCTCT |
| | GACATTGCTGTGGAATGGGAGAGCAATGGCCAGCCTGAAAACAACTACAAGACAACCCCT |
| | CCTGTGCTGGACTCTGATGGCTCATTCTTCCTGTACAGCAAGCTGACTGTGGACAAGTCC |
| | AGATGGCAGCAGGGAAATGTGTTCAGCTGCTCTGTGATGCATGAGGCCCTGCACAACCAC |
| | TACACCCAGAAAAGTCTGTCTCTGTCCCCTGGCAGAAAGAGAAGAGGATCAGGGGCCCCA |
| | GTGAAGCAGACCCTGAACTTTGATCTGCTGAAGCTGGCTGGGGATGTTGAGAGCAACCCT |
| | GGACCTATGTATAGGATGCAACTGCTCCTCCTGATTGCTCTGAGCCTGGCTCTTGTGACC |
| | AACTCTGACATCCAGATGACACAGAGCCCCTCCACACTGTCTGCATCTGTGGGAGACAGA |
| | GTGACCATCACCTGTAGAGCCAGCCAGTCTATCTCTTCCTGGCTGGCCTGGTATCAGCAA |
| | AAGCCTGGCAAGGCCCCTAAGCTGCTGATCTACAAGGCCAGCACACTTGAGTCAGGGGTG |
| | CCCTCCAGATTTTCTGGCTCTGGATCTGGCACAGAGTTCACCCTGACCATCAGCTCCCTG |
| | CAGCCAGATGACTTTGCCACCTACTACTGCCAGCAGTACAATACCTACTGGACCTTTGGC |
| | CAGGGAACAAAGGTGGAAATCAAGAGAACAGTGGCTGCCCCATCTGTGTTCATCTTCCCA |
| | CCATCTGATGAACAGCTGAAGTCTGGCACTGCCTCTGTTGTGTGCCTGCTGAACAACTTT |
| | TACCCTAGAGAAGCCAAGGTGCAGTGGAAAGTGGATAATGCCCTGCAGTCTGGCAATAGC |
| | CAAGAATCTGTGACAGAGCAGGACTCCAAGGATTCCACCTACAGCCTGAGCAGCACCCTG |
| | ACACTGAGCAAGGCTGACTATGAGAAGCACAAAGTGTATGCCTGTGAAGTGACACACCAG |
| | GGACTGAGCAGCCCAGTGACCAAGAGCTTCAACAGGGGAGAGTGCTGATAA |
| CAG.LAN.F2A | ctagtcgacattgattattgactagttattaatagtaatcaattacggggtcattagttc |
| (CAG.L01) | atagcccatatatggagttccgcgttacataacttacggtaaatggcccgcctggctgac |
| SEQ ID NO: 151 | cgcccaacgacccccgcccattgacgtcaataatgacgtatgttcccatagtaacgccaa |
| | tagggactttccattgacgtcaatgggtggagtatttacggtaaactgcccacttggcag |
| | tacatcaagtgtatcatatgccaagtacgccccctattgacgtcaatgacggtaaatggc |
| | ccgcctggcattatgcccagtacatgaccttatgggactttcctacttggcagtacatct |
| | acgtattagtcatcgctattaccatggtcgaggtgagccccacgttctgcttcactctcc |
| | ccatctcccccccctccccacccccaattttgtatttatttattttttaattattttgtg |
| | cagcgatgggggcggggggggggggggggcgcgcgccaggcggggcggggcggggcgagg |
| | ggcggggcggggcgaggcggagaggtgcggcggcagccaatcagagcggcgcgctccgaa |
| | agtttccttttatggcgaggcggcggcggcggcggccctataaaaagcgaagcgcgcggc |
| | gggcgggagtcgctgcgcgctgccttcgccccgtgccccgctccgccgccgcctcgcgcc |
| | gcccgccccggctctgactgaccgcgttactcccacaggtgagcgggcgggacggccctt |
| | ctcctccgggctgtaattagcgcttggtttaatgacggcttgtttcttttctgtggctgc |
| | gtgaaagccttgaggggctccgggagggccctttgtgcggggggagcggctcggggggtg |
| | cgtgcgtgtgtgtgtgcgtggggagcgccgcgtgcggctccgcgctgcccggcggctgtg |
| | agcgctgcgggcgcggcgcggggctttgtgcgctccgcagtgtgcgcgaggggagcgcgg |
| | ccgggggcggtgccccgcggtgcggggggggctgcgaggggaacaaaggctgcgtgcggg |
| | gtgtgtgcgtgggggggtgagcagggggtgtgggcgcgtcggtcgggctgcaaccccccc |
| | tgcacccccctccccgagttgctgagcacggcccggcttcgggtgcggggctccgtacgg |
| | ggcgtggcgcggggctcgccgtgccgggcggggggtggcggcaggtgggggtgccgggcg |
| | gggCggggccgcctcgggccggggagggctcgggggaggggcgcggcggcccccggagcg |
| | ccggcggctgtcgaggcgcggcgagccgcagccattgccttttatggtaatcgtgcgaga |
| | gggcgcagggacttcctttgtcccaaatctgtgcggagccgaaatctgggaggcgccgcc |
| | gcaccccctctagcgggcgcggggcgaagcggtgcggcgccggcaggaaggaaatgggcg |
| | gggagggccttcgtgcgtcgccgcgccgccgtccccttctccctctccagcctcggggct |
| | gtccgcggggggacggctgccttcgggggggacggggcagggcggggttcggcttctggc |
| | gtgtgaccggcggctctagagcctctgctaaccatgttcatgccttcttctttttcctac |
| | agctcctgggcaacgtgctggttattgtgctgtctcatcattttggcaaagaattcgcta |
| | gcgggcactttgcactggaacttacaacacccgagcaaggacgcgactctccaccatgta |
| | ccggatgcagctgctgctgctcattgccctgtctctggccctggtcaccaatagcgaagt |
| | ccagctgctggaatctggcggcggacttgttcaacctggcggctctctgagactgagctg |
| | tgccgcttccggcttcaccttcagccactatatcatgatgtgggtccgacaggcccctgg |
| | caaaggccttgaatgggtgtccggcatctatagcagcggcggcatcacagtgtacgccga |
| | ctctgtgaagggcagattcaccatcagccgggacaacagcaagaacaccctgtacctgca |
| | gatgaacagcctgagagccgaggacaccgccgtgtactactgcgcctatagaagaatcgg |
| | cgtgcccagacgggacgagttcgatatttggggccagggcaccatggtcaccgtgtctag |
| | cgcctctacaaagggccctagcgtgttccctctggctcctagcagcaagagcacaagcgg |
| | aggaacagccgctctgggctgtctggtcaaggactactttcccgagcctgtgaccgtgtc |
| | ctggaatagcggagcactgacaagcggcgtgcacacctttccagctgtgctgcaaagcag |
| | cggcctgtactctctgagcagcgtggtcacagtgccaagctctagcctgggcacccagac |
| | ctacatctgcaatgtgaaccacaagcctagcaacaccaaggtggacaagagagtggaacc |
| | caagagctgcgacaagacccacacctgtcctccatgtcctgctccagaactgctcggcgg |
| | accttccgtgttcctgtttcctccaaagcctaaggacaccctgatgatcagcagaacccc |
| | tgaagtgacctgcgtggtggtggatgtgtcccacgaggatcccgaagtgaagttcaattg |
| | gtacgtggacggcgtggaagtgcacaacgccaagaccaagcctagagaggaacagtacaa |
| | cagcacctacagagtggtgtccgtgctgaccgtgctgcaccaggattggctgaacggcaa |
| | agagtacaagtgcaaggtgtccaacaaggccctgcctgctcctatcgagaaaaccatcag |
| | caaggccaagggccagcctagggaaccccaggtttacacactgcctccaagccgggaaga |
| | gatgaccaagaaccaggtgtccctgacctgcctcgtgaagggcttctacccttccgatat |
| | cgccgtggaatgggagagcaatggccagccagagaacaactacaagacaacccctcctgt |
| | gctggacagcgacggctcattcttcctgtacagcaagctgacagtggacaagtccagatg |
| | gcagcagggcaacgtgttcagctgcagcgtgatgcacgaggccctgcacaaccactacac |
| | ccagaagagtctgagcctgtctccaggcctgcggaagagaagggctcctgtgaagcagac |
| | cctgaacttcgacctgctgaaactggccggcgacgtggaaagcaaccccggacctatgta |
| | tagaatgcagctcctcctgctgatcgcactgagcctggctctcgtgaccaacagcgacat |
| | ccagatgacacagagccccagcacactgtctgccagcgtgggagacagagtgaccatcac |
| | ctgtagagccagccagtccatctcctcttggctggcctggtatcagcaaaagcctggcaa |
| | ggcccctaagctgctgatctacaaggcctccacactggaaagcggggtgccctccagatt |
| | ttctggcagcggatctggcaccgagttcaccctgaccatcagtagcctgcagcctgacga |
| | cttcgccacctactactgccagcagtacaatacctactggaccttcggccagggaacaaa |
| | ggtggaaatcaagcggactgtggccgctccaagcgtgttcatctttccacctagcgacga |
| | gcagctgaagtccggcacagcctctgttgtgtgcctgctgaacaacttctaccccagaga |
| | agccaaggtgcagtggaaagtggacaatgccctgcagagcggcaactcccaagagagcgt |
| | gacagagcaggactccaaggattccacctacagcctgtctagcaccctgacactgagcaa |
| | ggccgactacgagaagcacaaagtgtatgcctgcgaagtgacccaccagggccttagctc |
| | tccagtgaccaagagcttcaaccggggcgagtgttgataaagcggccgcggtacctctag |
| | agtcgacccgggcggcctcgaggacggggtgaactacgcctgaggatccgatctttttcc |
| | ctctgccaaaaattatggggacatcatgaagccccttgagcatctgacttctggctaata |
| | aaggaaatttattttcattgcaatagtgtgttggaattttttgtgtctctcactcg |
| CAG.LANv2.F2A | gacattgattattgactagttattaatagtaatcaattacggggtcattagttcatagcc |
| (CAG.L03) | catatatggagttccgcgttacataacttacggtaaatggcccgcctggctgaccgccca |
| SEQ ID NO:152 | acgacccccgcccattgacgtcaataatgacgtatgttcccatagtaacgccaataggga |
| | ctttccattgacgtcaatgggtggagtatttacggtaaactgcccacttggcagtacatc |
| | aagtgtatcatatgccaagtacgccccctattgacgtcaatgacggtaaatggcccgcct |
| | ggcattatgcccagtacatgaccttatgggactttcctacttggcagtacatctacgtat |
| | tagtcatcgctattaccatggtcgaggtgagccccacgttctgcttcactctccccatct |
| | cccccccctccccacccccaattttgtatttatttattttttaattattttgtgcagcga |
| | tgggggcggggggggggggggggcgcgcgccaggcggggcggggcggggcgaggggcggg |
| | gcggggcgaggcggagaggtgcggcggcagccaatcagagcggcgcgctccgaaagtttc |
| | cttttatggcgaggcggcggcggcggcggccctataaaaagcgaagcgcgcggcgggcgg |
| | gagtcgctgcgcgctgccttcgccccgtgccccgctccgccgccgcctcgcgccgcccgc |
| | cccggctctgactgaccgcgttactcccacaggtgagcgggcgggacggcccttctcctc |
| | cgggctgtaattagcgcttggtttaatgacggcttgtttcttttctgtggctgcgtgaaa |
| | gccttgaggggctccgggagggccctttgtgcggggggagcggctcggggggtgcgtgcg |
| | tgtgtgtgtgcgtggggagcgccgcgtgcggctccgcgctgcccggcggctgtgagcgct |
| | gcgggcgcggcgcggggctttgtgcgctccgcagtgtgcgcgaggggagcgcggccgggg |
| | gcggtgccccgcggtgcggggggggctgcgaggggaacaaaggctgcgtgcggggtgtgt |
| | gcgtgggggggtgagcagggggtgtgggcgcgtcggtcgggctgcaaccccccctgcacc |
| | cccctccccgagttgctgagcacggcccggcttcgggtgcggggctccgtacggggcgtg |
| | gcgcggggctcgccgtgccgggcggggggtggcggcaggtgggggtgccgggcggggcgg |
| | ggccgcctcgggccggggagggctcgggggaggggcgcggcggcccccggagcgccggcg |
| | gctgtcgaggcgcggcgagccgcagccattgccttttatggtaatcgtgcgagagggcgc |
| | agggacttcctttgtcccaaatctgtgcggagccgaaatctgggaggcgccgccgcaccc |
| | cctctagcgggcgcggggcgaagcggtgcggcgccggcaggaaggaaatgggcggggagg |
| | gccttcgtgcgtcgccgcgccgccgtccccttctccctctccagcctcggggctgtccgc |
| | ggggggacggctgccttcgggggggacggggcagggcggggttcggcttctggcgtgtga |
| | ccggcggctctagagcctctgctaaccatgttcatgccttcttctttttcctacagctcc |
| | tgggcaacgtgctggttattgtgctgtctcatcattttggcaaagaattcgccgccacca |
| | tgtacagaatgcagctgctgctgctcattgccctgtctctggccctggtcaccaattctg |
| | aggtccagctgcttgagagtggtggtggactggttcagcctggtggcagcctgagactgt |
| | cttgtgctgcctctggcttcaccttcagccactatatcatgatgtgggtcagacaggccc |
| | ctggcaaaggcctggaatgggtgtcaggcatctacagcagtggtggcatcacagtgtatg |
| | ctgactctgtgaagggcagattcaccatcagcagagacaacagcaagaacaccctgtacc |
| | tgcagatgaactccctgagagctgaggacacagcagtgtactactgtgcctatagaagaa |
| | ttggggtgcccagaagggatgagtttgacatctggggccagggcaccatggttacagtgt |
| | cctctgccagcacaaagggcccctctgtttttccactggctcccagcagcaagagcacca |
| | gtggtggaacagctgccctgggctgtctggtcaaggattacttccctgagcctgtgactg |
| | tgtcctggaactctggggctctgacctctggggtgcacacctttccagctgtgctgcagt |
| | cctctggcctgtactctctgtcctctgtggtcacagtgcctagctctagcctgggcacac |
| | agacctacatctgcaatgtgaaccacaagcctagcaacaccaaggtggacaagagagtgg |
| | aacccaagagctgtgacaagacccacacctgtcctccatgtcctgctccagaactgcttg |
| | gaggcccttctgtgttcctgtttcctccaaagcctaaggacaccctgatgatcagcagaa |
| | cccctgaagtgacctgtgtggtggttgatgtgtcccatgaggacccagaagtgaagttca |
| | attggtatgtggatggggttgaagtgcacaatgccaagaccaagcctagagaggaacagt |
| | acaacagcacctacagagtggtttctgtgctgacagtgctgcaccaggactggctgaatg |
| | gcaaagagtacaagtgcaaggtgtccaacaaggccctgcctgctcctattgagaaaacca |
| | tctccaaggccaagggccagccaagagaaccccaggtttacaccctgccacctagcagag |
| | aagagatgaccaagaaccaggtgtccctgacctgcctggttaagggcttctacccctctg |
| | acattgctgtggaatgggagagcaatggccagcctgaaaacaactacaagacaacccctc |
| | ctgtgctggactctgatggctcattcttcctgtacagcaagctgactgtggacaagtcca |
| | gatggcagcagggaaatgtgttcagctgctctgtgatgcatgaggccctgcacaaccact |
| | acacccagaaaagtctgtctctgtcccctggcagaaagagaagaggatcaggggccccag |
| | tgaagcagaccctgaactttgatctgctgaagctggctggggatgttgagagcaaccctg |
| | gacctatgtataggatgcaactgctcctcctgattgctctgagcctggctcttgtgacca |
| | actctgacatccagatgacacagagcccctccacactgtctgcatctgtgggagacagag |
| | tgaccatcacctgtagagccagccagtctatctcttcctggctggcctggtatcagcaaa |
| | agcctggcaaggcccctaagctgctgatctacaaggccagcacacttgagtcaggggtgc |
| | cctccagattttctggctctggatctggcacagagttcaccctgaccatcagctccctgc |
| | agccagatgactttgccacctactactgccagcagtacaatacctactggacctttggcc |
| | agggaacaaaggtggaaatcaagagaacagtggctgccccatctgtgttcatcttcccac |
| | catctgatgaacagctgaagtctggcactgcctctgttgtgtgcctgctgaacaactttt |
| | accctagagaagccaaggtgcagtggaaagtggataatgccctgcagtctggcaatagcc |
| | aagaatctgtgacagagcaggactccaaggattccacctacagcctgagcagcaccctga |
| | cactgagcaaggctgactatgagaagcacaaagtgtatgcctgtgaagtgacacaccagg |
| | gactgagcagcccagtgaccaagagcttcaacaggggagagtgctgataactcgaggacg |
| | gggtgaactacgcctgaggatccgatctttttccctctgccaaaaattatggggacatca |
| | tgaagccccttgagcatctgacttctggctaataaaggaaatttattttcattgcaatag |
| | tgtgttggaattttttgtgtctctca |
| CAG.LANv2.T2A | gacattgattattgactagttattaatagtaatcaattacggggtcattagttcatagcc |
| (CAG.L02) | catatatggagttccgcgttacataacttacggtaaatggcccgcctggctgaccgccca |
| SEQ ID NO: 153 | acgacccccgcccattgacgtcaataatgacgtatgttcccatagtaacgccaataggga |
| | ctttccattgacgtcaatgggtggagtatttacggtaaactgcccacttggcagtacatc |
| | aagtgtatcatatgccaagtacgccccctattgacgtcaatgacggtaaatggcccgcct |
| | ggcattatgcccagtacatgaccttatgggactttcctacttggcagtacatctacgtat |
| | tagtcatcgctattaccatggtcgaggtgagccccacgttctgcttcactctccccatct |
| | cccccccctccccacccccaattttgtatttatttattttttaattattttgtgcagcga |
| | tgggggcggggggggggggggggcgcgcgccaggcggggcggggcggggcgaggggcggg |
| | gcggggcgaggcggagaggtgcggcggcagccaatcagagcggcgcgctccgaaagtttc |
| | cttttatggcgaggcggcggcggcggcggccctataaaaagcgaagcgcgcggcgggcgg |
| | gagtcgctgcgcgctgccttcgccccgtgccccgctccgccgccgcctcgcgccgcccgc |
| | cccggctctgactgaccgcgttactcccacaggtgagcgggcgggacggcccttctcctc |
| | cgggctgtaattagcgcttggtttaatgacggcttgtttcttttctgtggctgcgtgaaa |
| | gccttgaggggctccgggagggccctttgtgcggggggagcggctcggggggtgcgtgcg |
| | tgtgtgtgtgcgtggggagcgccgcgtgcggctccgcgctgcccggcggctgtgagcgct |
| | gcgggcgcggcgcggggctttgtgcgctccgcagtgtgcgcgaggggagcgcggccgggg |
| | gcggtgccccgcggtgcggggggggctgcgaggggaacaaaggctgcgtgcggggtgtgt |
| | gcgtgggggggtgagcagggggtgtgggcgcgtcggtcgggctgcaaccccccctgcacc |
| | cccctccccgagttgctgagcacggcccggcttcgggtgcggggctccgtacggggcgtg |
| | gcgcggggctcgccgtgccgggcggggggtggcggcaggtgggggtgccgggcggggcgg |
| | ggccgcctcgggccggggagggctcgggggaggggcgcggcggcccccggagcgccggcg |
| | gctgtcgaggcgcggcgagccgcagccattgccttttatggtaatcgtgcgagagggcgc |
| | agggacttcctttgtcccaaatctgtgcggagccgaaatctgggaggcgccgccgcaccc |
| | cctctagcgggcgcggggcgaagcggtgcggcgccggcaggaaggaaatgggcggggagg |
| | gccttcgtgcgtcgccgcgccgccgtccccttctccctctccagcctcggggctgtccgc |
| | ggggggacggctgccttcgggggggacggggcagggcggggttcggcttctggcgtgtga |
| | ccggcggctctagagcctctgctaaccatgttcatgccttcttctttttcctacagctcc |
| | tgggcaacgtgctggttattgtgctgtctcatcattttggcaaagaattcgccgccacca |
| | tgtacagaatgcagctgctgctgctcattgccctgtctctggccctggtcaccaattctg |
| | aggtccagctgcttgagagtggtggtggactggttcagcctggtggcagcctgagactgt |
| | cttgtgctgcctctggcttcaccttcagccactatatcatgatgtgggtcagacaggccc |
| | ctggcaaaggcctggaatgggtgtcaggcatctacagcagtggtggcatcacagtgtatg |
| | ctgactctgtgaagggcagattcaccatcagcagagacaacagcaagaacaccctgtacc |
| | tgcagatgaactccctgagagctgaggacacagcagtgtactactgtgcctatagaagaa |
| | ttggggtgcccagaagggatgagtttgacatctggggccagggcaccatggttacagtgt |
| | cctctgccagcacaaagggcccctctgtttttccactggctcccagcagcaagagcacca |
| | gtggtggaacagctgccctgggctgtctggtcaaggattacttccctgagcctgtgactg |
| | tgtcctggaactctggggctctgacttctggggtgcacacctttccagctgtgctgcagt |
| | cctctggcctgtactctctgtcctctgtggtcacagtgcctagctctagcctgggcacac |
| | agacctacatctgcaatgtgaaccacaagcctagcaacaccaaggtggacaagagagtgg |
| | aacccaagagctgtgacaagacccacacctgtcctccatgtcctgctccagaactgcttg |
| | gaggcccttctgtgttcctgtttcctccaaagcctaaggacaccctgatgatcagcagaa |
| | cccctgaagtgacctgtgtggtggttgatgtgtcccatgaggacccagaagtgaagttca |
| | attggtatgtggatggggttgaagtgcacaatgccaagaccaagcctagagaggaacagt |
| | acaacagcacctacagagtggtttctgtgctgacagtgctgcaccaggactggctgaatg |
| | gcaaagagtacaagtgcaaggtgtccaacaaggccctgcctgctcctattgagaaaacca |
| | tctccaaggccaagggccagccaagagaaccccaggtttacaccctgccacctagcagag |
| | aagagatgaccaagaaccaggtgtccctgacctgcctggttaagggcttctacccctctg |
| | acattgctgtggaatgggagagcaatggccagcctgaaaacaactacaagacaacccctc |
| | ctgtgctggactctgatggctcattcttcctgtacagcaagctgactgtggacaagtcca |
| | gatggcagcagggaaatgtgttcagctgctctgtgatgcatgaggccctgcacaaccact |
| | acacccagaaaagtctgtctctgtcccctggcagaaagagaagaggctctggagaaggca |
| | gaggctccctgctgacatgtggggatgttgaagagaatcctgggcctatgtataggatgc |
| | aactgctcctcctgattgctctgagcctggctcttgtgaccaactctgacatccagatga |
| | cacagagcccctccacactgtctgcatctgtgggagacagagtgaccatcacctgtagag |
| | ccagccagtctatctctagctggctggcctggtatcagcaaaagcctggcaaggccccta |
| | agctgctgatctacaaggccagcacacttgagtcaggggtgccctccagattttctggct |
| | ctggatctggcacagagttcaccctgaccatcagctccctgcagccagatgactttgcca |
| | cctactactgccagcagtacaatacctactggacctttggccagggaacaaaggtggaaa |
| | tcaagagaacagtggctgccccatctgtgttcatcttcccaccatctgatgaacagctga |
| | agtctggcactgcctctgttgtgtgcctgctgaacaacttttaccccagagaagccaagg |
| | tgcagtggaaagtggataatgccctgcagtctggcaatagccaagaatctgtgacagagc |
| | aggactccaaggattccacctacagcctgagcagcaccctgacactgagcaaggctgact |
| | atgagaagcacaaagtgtatgcctgtgaagtgacacaccagggactgagcagcccagtga |
| | ccaagagcttcaacaggggagagtgctgataactcgaggacggggtgaactacgcctgag |
| | gatccgatctttttccctctgccaaaaattatggggacatcatgaagccccttgagcatc |
| | tgacttctggctaataaaggaaatttattttcattgcaatagtgtgttggaattttttgt |
| | gtctctcactcg |
| TBG.LANv2.T2A | aggttaatttttaaaaagcagtcaaaagtccaagtggcccttggcagcatttactctctc |
| (TBG.L02) | tgtttgctctggttaataatctcaggagcacaaacattccagatccaggttaatttttaa |
| SEQ ID NO: 154 | aaagcagtcaaaagtccaagtggcccttggcagcatttactctctctgtttgctctggtt |
| | aataatctcaggagcacaaacattccagatccggcgcgccagggctggaagctacctttg |
| | acatcatttcctctgcgaatgcatgtataatttctacagaacctattagaaaggatcacc |
| | cagcctctgcttttgtacaactttcccttaaaaaactgccaattccactgctgtttggcc |
| | caatagtgagaactttttcctgctgcctcttggtgcttttgcctatggcccctattctgc |
| | ctgctgaagacactcttgccagcatggacttaaacccctccagctctgacaatcctcttt |
| | ctcttttgttttacatgaagggtctggcagccaaagcaatcactcaaagttcaaacctta |
| | tcattttttgctttgttcctcttggccttggttttgtacatcagctttgaaaataccatc |
| | ccagggttaatgctggggttaatttataactaagagtgctctagttttgcaatacaggac |
| | atgctataaaaatggaaagatgttgctttctgagagacagctttattgcggtagtttatc |
| | acagttaaattgctaacgcagtcagtgcttctgacacaacagtctcgaacttaagctgca |
| | gaagttggtcgtgaggcactgggcaggtaagtatcaaggttacaagacaggtttaaggag |
| | accaatagaaactgggcttgtcgagacagagaagactcttgcgtttctgataggcaccta |
| | ttggtcttactgacatccactttgcctttctctccacaggtgtccactcccagttcaatt |
| | acagctcttaaggctagagtacttaatacgactcactataggctagcctcgagaattcac |
| | gcgtgccgccaccatgtacagaatgcagctgctgctgctcattgccctgtctctggccct |
| | ggtcaccaattctgaggtccagctgcttgagagtggtggtggactggttcagcctggtgg |
| | cagcctgagactgtcttgtgctgcctctggcttcaccttcagccactatatcatgatgtg |
| | ggtcagacaggcccctggcaaaggcctggaatgggtgtcaggcatctacagcagtggtgg |
| | catcacagtgtatgctgactctgtgaagggcagattcaccatcagcagagacaacagcaa |
| | gaacaccctgtacctgcagatgaactccctgagagctgaggacacagcagtgtactactg |
| | tgcctatagaagaattggggtgcccagaagggatgagtttgacatctggggccagggcac |
| | catggttacagtgtcctctgccagcacaaagggcccctctgtttttccactggctcccag |
| | cagcaagagcaccagtggtggaacagctgccctgggctgtctggtcaaggattacttccc |
| | tgagcctgtgactgtgtcctggaactctggggctctgacttctggggtgcacacctttcc |
| | agctgtgctgcagtcctctggcctgtactctctgtcctctgtggtcacagtgcctagctc |
| | tagcctgggcacacagacctacatctgcaatgtgaaccacaagcctagcaacaccaaggt |
| | ggacaagagagtggaacccaagagctgtgacaagacccacacctgtcctccatgtcctgc |
| | tccagaactgcttggaggcccttctgtgttcctgtttcctccaaagcctaaggacaccct |
| | gatgatcagcagaacccctgaagtgacctgtgtggtggttgatgtgtcccatgaggaccc |
| | agaagtgaagttcaattggtatgtggatggggttgaagtgcacaatgccaagaccaagcc |
| | tagagaggaacagtacaacagcacctacagagtggtttctgtgctgacagtgctgcacca |
| | ggactggctgaatggcaaagagtacaagtgcaaggtgtccaacaaggccctgcctgctcc |
| | tattgagaaaaccatctccaaggccaagggccagccaagagaaccccaggtttacaccct |
| | gccacctagcagagaagagatgaccaagaaccaggtgtccctgacctgcctggttaaggg |
| | cttctacccctctgacattgctgtggaatgggagagcaatggccagcctgaaaacaacta |
| | caagacaacccctcctgtgctggactctgatggctcattcttcctgtacagcaagctgac |
| | tgtggacaagtccagatggcagcagggaaatgtgttcagctgctctgtgatgcatgaggc |
| | cctgcacaaccactacacccagaaaagtctgtctctgtcccctggcagaaagagaagagg |
| | ctctggagaaggcagaggctccctgctgacatgtggggatgttgaagagaatcctgggcc |
| | tatgtataggatgcaactgctcctcctgattgctctgagcctggctcttgtgaccaactc |
| | tgacatccagatgacacagagcccctccacactgtctgcatctgtgggagacagagtgac |
| | catcacctgtagagccagccagtctatctctagctggctggcctggtatcagcaaaagcc |
| | tggcaaggcccctaagctgctgatctacaaggccagcacacttgagtcaggggtgccctc |
| | cagattttctggctctggatctggcacagagttcaccctgaccatcagctccctgcagcc |
| | agatgactttgccacctactactgccagcagtacaatacctactggacctttggccaggg |
| | aacaaaggtggaaatcaagagaacagtggctgccccatctgtgttcatcttcccaccatc |
| | tgatgaacagctgaagtctggcactgcctctgttgtgtgcctgctgaacaacttttaccc |
| | cagagaagccaaggtgcagtggaaagtggataatgccctgcagtctggcaatagccaaga |
| | atctgtgacagagcaggactccaaggattccacctacagcctgagcagcaccctgacact |
| | gagcaaggctgactatgagaagcacaaagtgtatgcctgtgaagtgacacaccagggact |
| | gagcagcccagtgaccaagagcttcaacaggggagagtgctgataagtcgacccgggcgg |
| | cctcgaggacggggtgaactacgcctgaggatccgatctttttccctctgccaaaaatta |
| | tggggacatcatgaagccccttgagcatctgacttctggctaataaaggaaatttatttt |
| | cattgcaatagtgtgttggaattttttgtgtctctca |
| ApoE.hAAT. | ggctcagaggcacacaggagtttctgggctcaccctgcccccttccaacccctcagttcc |
| LANv2.T2A | catcctccagcagctgtttgtgtgctgcctctgaagtccacactgaacaaacttcagcct |
| (ApoE.hAAT.L02) | actcatgtccctaaaatgggcaaacattgcaagcagcaaacagcaaacacacagccctcc |
| SEQ ID NO: 155 | ctgcctgctgaccttggagctggggcagaggtcagagacctctctgggcccatgccacct |
| | ccaacatccactcgaccccttggaatttcggtggagaggagcagaggttgtcctggcgtg |
| | gtttaggtagtgtgagaggggtacccggggatcttgctaccagtggaacagccactaagg |
| | attctgcagtgagagcagagggccagctaagtggtactctcccagagactgtctgactca |
| | cgccaccccctccaccttggacacaggacgctgtggtttctgagccaggtacaatgactc |
| | ctttcggtaagtgcagtggaagctgtacactgcccaggcaaagcgtccgggcagcgtagg |
| | cgggcgactcagatcccagccagtggacttagcccctgtttgctcctccgataactgggg |
| | tgaccttggttaatattcaccagcagcctcccccgttgcccctctggatccactgcttaa |
| | atacggacgaggacagggccctgtctcctcagcttcaggcaccaccactgacctgggaca |
| | gtcaggtaagtatcaaggttacaagacaggtttaaggagaccaatagaaactgggcttgt |
| | cgagacagagaagactcttgcgtttctgataggcacctattggtcttactgacatccact |
| | ttgcctttctctccacaggttcgaattcgccgccaccatgtacagaatgcagctgctgct |
| | gctcattgccctgtctctggccctggtcaccaattctgaggtccagctgcttgagagtgg |
| | tggtggactggttcagcctggtggcagcctgagactgtcttgtgctgcctctggcttcac |
| | cttcagccactatatcatgatgtgggtcagacaggcccctggcaaaggcctggaatgggt |
| | gtcaggcatctacagcagtggtggcatcacagtgtatgctgactctgtgaagggcagatt |
| | caccatcagcagagacaacagcaagaacaccctgtacctgcagatgaactccctgagagc |
| | tgaggacacagcagtgtactactgtgcctatagaagaattggggtgcccagaagggatga |
| | gtttgacatctggggccagggcaccatggttacagtgtcctctgccagcacaaagggccc |
| | ctctgtttttccactggctcccagcagcaagagcaccagtggtggaacagctgccctggg |
| | ctgtctggtcaaggattacttccctgagcctgtgactgtgtcctggaactctggggctct |
| | gacttctggggtgcacacctttccagctgtgctgcagtcctctggcctgtactctctgtc |
| | ctctgtggtcacagtgcctagctctagcctgggcacacagacctacatctgcaatgtgaa |
| | ccacaagcctagcaacaccaaggtggacaagagagtggaacccaagagctgtgacaagac |
| | ccacacctgtcctccatgtcctgctccagaactgcttggaggcccttctgtgttcctgtt |
| | tcctccaaagcctaaggacaccctgatgatcagcagaacccctgaagtgacctgtgtggt |
| | ggttgatgtgtcccatgaggacccagaagtgaagttcaattggtatgtggatggggttga |
| | agtgcacaatgccaagaccaagcctagagaggaacagtacaacagcacctacagagtggt |
| | ttctgtgctgacagtgctgcaccaggactggctgaatggcaaagagtacaagtgcaaggt |
| | gtccaacaaggccctgcctgctcctattgagaaaaccatctccaaggccaagggccagcc |
| | aagagaaccccaggtttacaccctgccacctagcagagaagagatgaccaagaaccaggt |
| | gtccctgacctgcctggttaagggcttctacccctctgacattgctgtggaatgggagag |
| | caatggccagcctgaaaacaactacaagacaacccctcctgtgctggactctgatggctc |
| | attcttcctgtacagcaagctgactgtggacaagtccagatggcagcagggaaatgtgtt |
| | cagctgctctgtgatgcatgaggccctgcacaaccactacacccagaaaagtctgtctct |
| | gtcccctggcagaaagagaagaggctctggagaaggcagaggctccctgctgacatgtgg |
| | ggatgttgaagagaatcctgggcctatgtataggatgcaactgctcctcctgattgctct |
| | gagcctggctcttgtgaccaactctgacatccagatgacacagagcccctccacactgtc |
| | tgcatctgtgggagacagagtgaccatcacctgtagagccagccagtctatctctagctg |
| | gctggcctggtatcagcaaaagcctggcaaggcccctaagctgctgatctacaaggccag |
| | cacacttgagtcaggggtgccctccagattttctggctctggatctggcacagagttcac |
| | cctgaccatcagctccctgcagccagatgactttgccacctactactgccagcagtacaa |
| | tacctactggacctttggccagggaacaaaggtggaaatcaagagaacagtggctgcccc |
| | atctgtgttcatcttcccaccatctgatgaacagctgaagtctggcactgcctctgttgt |
| | gtgcctgctgaacaacttttaccccagagaagccaaggtgcagtggaaagtggataatgc |
| | cctgcagtctggcaatagccaagaatctgtgacagagcaggactccaaggattccaccta |
| | cagcctgagcagcaccctgacactgagcaaggctgactatgagaagcacaaagtgtatgc |
| | ctgtgaagtgacacaccagggactgagcagcccagtgaccaagagcttcaacaggggaga |
| | gtgctgataactcgaggacggggtgaactacgcctgaggatccgatctttttccctctgc |
| | caaaaattatggggacatcatgaagccccttgagcatctgacttctggctaataaaggaa |
| | atttattttcattgcaatagtgtgttggaattttttgtgtctctca |
| LSPX1.LANv2.T2A | aggttaatttttaaaaagcagtcaaaagtccaagtggcccttggcagcatttactctctc |
| (LSPX.L02) | tgtttgctctggttaataatctcaggagcacaaacattccagatccaggttaatttttaa |
| SEQ ID NO: 156 | aaagcagtcaaaagtccaagtggcccttggcagcatttactctctctgtttgctctggtt |
| | aataatctcaggagcacaaacattccagatccggcgcgccagggctggaagctacctttg |
| | tctagaaggctcagaggcacacaggagtttctgggctcaccctgcccccttccaacccct |
| | cagttcccatcctccagcagctgtttgtgtgctgcctctgaagtccacactgaacaaact |
| | tcagcctactcatgtccctaaaatgggcaaacattgcaagcagcaaacagcaaacacaca |
| | gccctccctgcctgctgaccttggagctggggcagaggtcagagacctctctgggcccat |
| | gccacctccaacatccactcgaccccttggaatttcggtggagaggagcagaggttgtcc |
| | tggcgtggtttaggtagtgtgagaggggtacccggggatcttgctaccagtggaacagcc |
| | actaaggattctgcagtgagagcagagggccagctaagtggtactctcccagagactgtc |
| | tgactcacgccaccccctccaccttggacacaggacgctgtggtttctgagccaggtaca |
| | atgactcctttcggtaagtgcagtggaagctgtacactgcccaggcaaagcgtccgggca |
| | gcgtaggcgggcgactcagatcccagccagtggacttagcccctgtttgctcctccgata |
| | actggggtgaccttggttaatattcaccagcagcctcccccgttgcccctctggatccac |
| | tgcttaaatacggacgaggacagggccctgtctcctcagcttcaggcaccaccactgacc |
| | tgggacagtcaggtaagtatcaaggttacaagacaggtttaaggagaccaatagaaactg |
| | ggcttgtcgagacagagaagactcttgcgtttctgataggcacctattggtcttactgac |
| | atccactttgcctttctctccacaggttgaattcgccgccaccatgtacagaatgcagct |
| | gctgctgctcattgccctgtctctggccctggtcaccaattctgaggtccagctgcttga |
| | gagtggtggtggactggttcagcctggtggcagcctgagactgtcttgtgctgcctctgg |
| | cttcaccttcagccactatatcatgatgtgggtcagacaggcccctggcaaaggcctgga |
| | atgggtgtcaggcatctacagcagtggtggcatcacagtgtatgctgactctgtgaaggg |
| | cagattcaccatcagcagagacaacagcaagaacaccctgtacctgcagatgaactccct |
| | gagagctgaggacacagcagtgtactactgtgcctatagaagaattggggtgcccagaag |
| | ggatgagtttgacatctggggccagggcaccatggttacagtgtcctctgccagcacaaa |
| | gggcccctctgtttttccactggctcccagcagcaagagcaccagtggtggaacagctgc |
| | cctgggctgtctggtcaaggattacttccctgagcctgtgactgtgtcctggaactctgg |
| | ggctctgacttctggggtgcacacctttccagctgtgctgcagtcctctggcctgtactc |
| | tctgtcctctgtggtcacagtgcctagctctagcctgggcacacagacctacatctgcaa |
| | tgtgaaccacaagcctagcaacaccaaggtggacaagagagtggaacccaagagctgtga |
| | caagacccacacctgtcctccatgtcctgctccagaactgcttggaggcccttctgtgtt |
| | cctgtttcctccaaagcctaaggacaccctgatgatcagcagaacccctgaagtgacctg |
| | tgtggtggttgatgtgtcccatgaggacccagaagtgaagttcaattggtatgtggatgg |
| | ggttgaagtgcacaatgccaagaccaagcctagagaggaacagtacaacagcacctacag |
| | agtggtttctgtgctgacagtgctgcaccaggactggctgaatggcaaagagtacaagtg |
| | caaggtgtccaacaaggccctgcctgctcctattgagaaaaccatctccaaggccaaggg |
| | ccagccaagagaaccccaggtttacaccctgccacctagcagagaagagatgaccaagaa |
| | ccaggtgtccctgacctgcctggttaagggcttctacccctctgacattgctgtggaatg |
| | ggagagcaatggccagcctgaaaacaactacaagacaacccctcctgtgctggactctga |
| | tggctcattcttcctgtacagcaagctgactgtggacaagtccagatggcagcagggaaa |
| | tgtgttcagctgctctgtgatgcatgaggccctgcacaaccactacacccagaaaagtct |
| | gtctctgtcccctggcagaaagagaagaggctctggagaaggcagaggctccctgctgac |
| | atgtggggatgttgaagagaatcctgggcctatgtataggatgcaactgctcctcctgat |
| | tgctctgagcctggctcttgtgaccaactctgacatccagatgacacagagcccctccac |
| | actgtctgcatctgtgggagacagagtgaccatcacctgtagagccagccagtctatctc |
| | tagctggctggcctggtatcagcaaaagcctggcaaggcccctaagctgctgatctacaa |
| | ggccagcacacttgagtcaggggtgccctccagattttctggctctggatctggcacaga |
| | gttcaccctgaccatcagctccctgcagccagatgactttgccacctactactgccagca |
| | gtacaatacctactggacctttggccagggaacaaaggtggaaatcaagagaacagtggc |
| | tgccccatctgtgttcatcttcccaccatctgatgaacagctgaagtctggcactgcctc |
| | tgttgtgtgcctgctgaacaacttttaccccagagaagccaaggtgcagtggaaagtgga |
| | taatgccctgcagtctggcaatagccaagaatctgtgacagagcaggactccaaggattc |
| | cacctacagcctgagcagcaccctgacactgagcaaggctgactatgagaagcacaaagt |
| | gtatgcctgtgaagtgacacaccagggactgagcagcccagtgaccaagagcttcaacag |
| | gggagagtgctgataactcgaggacggggtgaactacgcctgaggatccgatctttttcc |
| | ctctgccaaaaattatggggacatcatgaagccccttgagcatctgacttctggctaata |
| | aaggaaatttattttcattgcaatagtgtgttggaattttttgtgtctctca |
| LSPX2.LANv2.T2A | aggctcagaggcacacaggagtttctgggctcaccctgcccccttccaacccctcagttc |
| (LSPX2.L02) | ccatcctccagcagctgtttgtgtgctgcctctgaagtccacactgaacaaacttcagcc |
| SEQ ID NO: 157 | tactcatgtccctaaaatgggcaaacattgcaagcagcaaacagcaaacacacagccctc |
| | cctgcctgctgaccttggagctggggcagaggtcagagacctctctgggcccatgccacc |
| | tccaacatccactcgaccccttggaatttcggtggagaggagcagaggttgtcctggcgt |
| | ggtttaggtagtgtgagagggtctagaaggctcagaggcacacaggagtttctgggctca |
| | ccctgcccccttccaacccctcagttcccatcctccagcagctgtttgtgtgctgcctct |
| | gaagtccacactgaacaaacttcagcctactcatgtccctaaaatgggcaaacattgcaa |
| | gcagcaaacagcaaacacacagccctccctgcctgctgaccttggagctggggcagaggt |
| | cagagacctctctgggcccatgccacctccaacatccactcgaccccttggaatttcggt |
| | ggagaggagcagaggttgtcctggcgtggtttaggtagtgtgagaggggtacccggggat |
| | cttgctaccagtggaacagccactaaggattctgcagtgagagcagagggccagctaagt |
| | ggtactctcccagagactgtctgactcacgccaccccctccaccttggacacaggacgct |
| | gtggtttctgagccaggtacaatgactcctttcggtaagtgcagtggaagctgtacactg |
| | cccaggcaaagcgtccgggcagcgtaggcgggcgactcagatcccagccagtggacttag |
| | cccctgtttgctcctccgataactggggtgaccttggttaatattcaccagcagcctccc |
| | ccgttgcccctctggatccactgcttaaatacggacgaggacagggccctgtctcctcag |
| | cttcaggcaccaccactgacctgggacagtcaggtaagtatcaaggttacaagacaggtt |
| | taaggagaccaatagaaactgggcttgtcgagacagagaagactcttgcgtttctgatag |
| | gcacctattggtcttactgacatccactttgcctttctctccacaggttgaattcgccgc |
| | caccatgtacagaatgcagctgctgctgctcattgccctgtctctggccctggtcaccaa |
| | ttctgaggtccagctgcttgagagtggtggtggactggttcagcctggtggcagcctgag |
| | actgtcttgtgctgcctctggcttcaccttcagccactatatcatgatgtgggtcagaca |
| | ggcccctggcaaaggcctggaatgggtgtcaggcatctacagcagtggtggcatcacagt |
| | gtatgctgactctgtgaagggcagattcaccatcagcagagacaacagcaagaacaccct |
| | gtacctgcagatgaactccctgagagctgaggacacagcagtgtactactgtgcctatag |
| | aagaattggggtgcccagaagggatgagtttgacatctggggccagggcaccatggttac |
| | agtgtcctctgccagcacaaagggcccctctgtttttccactggctcccagcagcaagag |
| | caccagtggtggaacagctgccctgggctgtctggtcaaggattacttccctgagcctgt |
| | gactgtgtcctggaactctggggctctgacttctggggtgcacacctttccagctgtgct |
| | gcagtcctctggcctgtactctctgtcctctgtggtcacagtgcctagctctagcctggg |
| | cacacagacctacatctgcaatgtgaaccacaagcctagcaacaccaaggtggacaagag |
| | agtggaacccaagagctgtgacaagacccacacctgtcctccatgtcctgctccagaact |
| | gcttggaggcccttctgtgttcctgtttcctccaaagcctaaggacaccctgatgatcag |
| | cagaacccctgaagtgacctgtgtggtggttgatgtgtcccatgaggacccagaagtgaa |
| | gttcaattggtatgtggatggggttgaagtgcacaatgccaagaccaagcctagagagga |
| | acagtacaacagcacctacagagtggtttctgtgctgacagtgctgcaccaggactggct |
| | gaatggcaaagagtacaagtgcaaggtgtccaacaaggccctgcctgctcctattgagaa |
| | aaccatctccaaggccaagggccagccaagagaaccccaggtttacaccctgccacctag |
| | cagagaagagatgaccaagaaccaggtgtccctgacctgcctggttaagggcttctaccc |
| | ctctgacattgctgtggaatgggagagcaatggccagcctgaaaacaactacaagacaac |
| | ccctcctgtgctggactctgatggctcattcttcctgtacagcaagctgactgtggacaa |
| | gtccagatggcagcagggaaatgtgttcagctgctctgtgatgcatgaggccctgcacaa |
| | ccactacacccagaaaagtctgtctctgtcccctggcagaaagagaagaggctctggaga |
| | aggcagaggctccctgctgacatgtggggatgttgaagagaatcctgggcctatgtatag |
| | gatgcaactgctcctcctgattgctctgagcctggctcttgtgaccaactctgacatcca |
| | gatgacacagagcccctccacactgtctgcatctgtgggagacagagtgaccatcacctg |
| | tagagccagccagtctatctctagctggctggcctggtatcagcaaaagcctggcaaggc |
| | ccctaagctgctgatctacaaggccagcacacttgagtcaggggtgccctccagattttc |
| | tggctctggatctggcacagagttcaccctgaccatcagctccctgcagccagatgactt |
| | tgccacctactactgccagcagtacaatacctactggacctttggccagggaacaaaggt |
| | ggaaatcaagagaacagtggctgccccatctgtgttcatcttcccaccatctgatgaaca |
| | gctgaagtctggcactgcctctgttgtgtgcctgctgaacaacttttaccccagagaagc |
| | caaggtgcagtggaaagtggataatgccctgcagtctggcaatagccaagaatctgtgac |
| | agagcaggactccaaggattccacctacagcctgagcagcaccctgacactgagcaaggc |
| | tgactatgagaagcacaaagtgtatgcctgtgaagtgacacaccagggactgagcagccc |
| | agtgaccaagagcttcaacaggggagagtgctgataactcgaggacggggtgaactacgc |
| | ctgaggatccgatctttttccctctgccaaaaattatggggacatcatgaagccccttga |
| | gcatctgacttctggctaataaaggaaatttattttcattgcaatagtgtgttggaattt |
| | tttgtgtctctca |
| LTP1.LANv2.T2A | aggttaatttttaaaaagcagtcaaaagtccaagtggcccttggcagcatttactctctc |
| (LTP1.L02) | tgtttgctctggttaataatctcaggagcacaaacattccagatccaggttaatttttaa |
| SEQ ID NO: 158 | aaagcagtcaaaagtccaagtggcccttggcagcatttactctctctgtttgctctggtt |
| | aataatctcaggagcacaaacattccagatccggcgcgccagggctggaagctacctttg |
| | acatcatttcctctgcgaatgcatgtataatttctacagaacctattagaaaggatcacc |
| | cagcctctgcttttgtacaactttcccttaaaaaactgccaattccactgctgtttggcc |
| | caatagtgagaactttttcctgctgcctcttggtgcttttgcctatggcccctattctgc |
| | ctgctgaagacactcttgccagcatggacttaaacccctccagctctgacaatcctcttt |
| | ctcttttgttttacatgaagggtctggcagccaaagcaatcactcaaagttcaaacctta |
| | tcattttttgctttgttcctcttggccttggttttgtacatcagctttgaaaataccatc |
| | ccagggttaatgctggggttaatttataactaagagtgctctagttttgcaatacaggac |
| | atgctataaaaatggaaagatgttgctttctgagaggatcttgctaccagtggaacagcc |
| | actaaggattctgcagtgagagcagagggccagctaagtggtactctcccagagactgtc |
| | tgactcacgccaccccctccaccttggacacaggacgctgtggtttctgagccaggtaca |
| | gtgactcctttcggtaagtgcagtggaagctgtacactgcccaggcaaagcgtccgggca |
| | gcgtaggcgggcgactcagatcccagccagtggacttagcccctgtttgctcctccgata |
| | actggggtgaccttggttaatattcaccagcagcctcccccgttgcccctctggatccac |
| | tgcttaaatacggacgaggacagggccctgtctcctcagcttcaggcaccaccactgacc |
| | tgggacagtcaggtaagtatcaaggttacaagacaggtttaaggagaccaatagaaactg |
| | ggcttgtcgagacagagaagactcttgcgtttctgataggcacctattggtcttactgac |
| | atccactttgcctttctctccacaggttgaattcgccgccaccatgtacagaatgcagct |
| | gctgctgctcattgccctgtctctggccctggtcaccaattctgaggtccagctgcttga |
| | gagtggtggtggactggttcagcctggtggcagcctgagactgtcttgtgctgcctctgg |
| | cttcaccttcagccactatatcatgatgtgggtcagacaggcccctggcaaaggcctgga |
| | atgggtgtcaggcatctacagcagtggtggcatcacagtgtatgctgactctgtgaaggg |
| | cagattcaccatcagcagagacaacagcaagaacaccctgtacctgcagatgaactccct |
| | gagagctgaggacacagcagtgtactactgtgcctatagaagaattggggtgcccagaag |
| | ggatgagtttgacatctggggccagggcaccatggttacagtgtcctctgccagcacaaa |
| | gggcccctctgtttttccactggctcccagcagcaagagcaccagtggtggaacagctgc |
| | cctgggctgtctggtcaaggattacttccctgagcctgtgactgtgtcctggaactctgg |
| | ggctctgacttctggggtgcacacctttccagctgtgctgcagtcctctggcctgtactc |
| | tctgtcctctgtggtcacagtgcctagctctagcctgggcacacagacctacatctgcaa |
| | tgtgaaccacaagcctagcaacaccaaggtggacaagagagtggaacccaagagctgtga |
| | caagacccacacctgtcctccatgtcctgctccagaactgcttggaggcccttctgtgtt |
| | cctgtttcctccaaagcctaaggacaccctgatgatcagcagaacccctgaagtgacctg |
| | tgtggtggttgatgtgtcccatgaggacccagaagtgaagttcaattggtatgtggatgg |
| | ggttgaagtgcacaatgccaagaccaagcctagagaggaacagtacaacagcacctacag |
| | agtggtttctgtgctgacagtgctgcaccaggactggctgaatggcaaagagtacaagtg |
| | caaggtgtccaacaaggccctgcctgctcctattgagaaaaccatctccaaggccaaggg |
| | ccagccaagagaaccccaggtttacaccctgccacctagcagagaagagatgaccaagaa |
| | ccaggtgtccctgacctgcctggttaagggcttctacccctctgacattgctgtggaatg |
| | ggagagcaatggccagcctgaaaacaactacaagacaacccctcctgtgctggactctga |
| | tggctcattcttcctgtacagcaagctgactgtggacaagtccagatggcagcagggaaa |
| | tgtgttcagctgctctgtgatgcatgaggccctgcacaaccactacacccagaaaagtct |
| | gtctctgtcccctggcagaaagagaagaggctctggagaaggcagaggctccctgctgac |
| | atgtggggatgttgaagagaatcctgggcctatgtataggatgcaactgctcctcctgat |
| | tgctctgagcctggctcttgtgaccaactctgacatccagatgacacagagcccctccac |
| | actgtctgcatctgtgggagacagagtgaccatcacctgtagagccagccagtctatctc |
| | tagctggctggcctggtatcagcaaaagcctggcaaggcccctaagctgctgatctacaa |
| | ggccagcacacttgagtcaggggtgccctccagattttctggctctggatctggcacaga |
| | gttcaccctgaccatcagctccctgcagccagatgactttgccacctactactgccagca |
| | gtacaatacctactggacctttggccagggaacaaaggtggaaatcaagagaacagtggc |
| | tgccccatctgtgttcatcttcccaccatctgatgaacagctgaagtctggcactgcctc |
| | tgttgtgtgcctgctgaacaacttttaccccagagaagccaaggtgcagtggaaagtgga |
| | taatgccctgcagtctggcaatagccaagaatctgtgacagagcaggactccaaggattc |
| | cacctacagcctgagcagcaccctgacactgagcaaggctgactatgagaagcacaaagt |
| | gtatgcctgtgaagtgacacaccagggactgagcagcccagtgaccaagagcttcaacag |
| | gggagagtgctgataactcgaggacggggtgaactacgcctgaggatccgatctttttcc |
| | ctctgccaaaaattatggggacatcatgaagccccttgagcatctgacttctggctaata |
| | aaggaaatttattttcattgcaatagtgtgttggaattttttgtgtctctca |
| LMTP6.LANv2.T2A | aggctcagaggcacacaggagtttctgggctcaccctgcccccttccaacccctcagttc |
| (LMTP6.L02) | ccatcctccagcagctgtttgtgtgctgcctctgaagtccacactgaacaaacttcagcc |
| SEQ ID NO: 159 | tactcatgtccctaaaatgggcaaacattgcaagcagcaaacagcaaacacacagccctc |
| | cctgcctgctgaccttggagctggggcagaggtcagagacctctctgggcccatgccacc |
| | tccaacatccactcgaccccttggaatttcggtggagaggagcagaggttgtcctggcgt |
| | ggtttaggtagtgtgagagggccactacgggtttaggctgcccatgtaaggaggcaaggc |
| | ctggggacacccgagatgcctggttataattaacccagacatgtggctgccccccccccc |
| | cccaacacctgctgcctctaaaaataaccctgtccctggtggatcccactacgggtttag |
| | gctgcccatgtaaggaggcaaggcctggggacacccgagatgcctggttataattaaccc |
| | agacatgtggctgccccccccccccccaacacctgctgcctctaaaaataaccctgtccc |
| | tggtggatcccactacgggtttaggctgcccatgtaaggaggcaaggcctggggacaccc |
| | gagatgcctggttataattaacccagacatgtggctgccccccccccccccaacacctgc |
| | tgcctctaaaaataaccctgtccctggtggatcccctgcatgcgaagatcttcgaacaag |
| | gctgtgggggactgagggcaggctgtaacaggcttgggggccagggcttatacgtgcctg |
| | ggactcccaaagtattactgttccatgttcccggcgaagggccagctgtcccccgccagc |
| | tagactcagcacttagtttaggaaccagtgagcaagtcagcccttggggcagcccataca |
| | aggccatggggctgggcaagctgcacgcctgggtccggggtgggcacggtgcccgggcaa |
| | cgagctgaaagctcatctgctctcaggggcccctccctggggacagcccctcctggctag |
| | tcacaccctgtaggctcctctatataacccaggggcacaggggctgccctcattctacca |
| | ccacctccacagcacagacagacactcaggagccagccagcgtcgagatcttgctaccag |
| | tggaacagccactaaggattctgcagtgagagcagagggccagctaagtggtactctccc |
| | agagactgtctgactcacgccaccccctccaccttggacacaggacgctgtggtttctga |
| | gccaggtacagtgactcctttcggtaagtgcagtggaagctgtacactgcccaggcaaag |
| | cgtccgggcagcgtaggcgggcgactcagatcccagccagtggacttagcccctgtttgc |
| | tcctccgataactggggtgaccttggttaatattcaccagcagcctcccccgttgcccct |
| | ctggatccactgcttaaatacggacgaggacagggccctgtctcctcagcttcaggcacc |
| | accactgacctgggacagtcaggtaagtatcaaggttacaagacaggtttaaggagacca |
| | atagaaactgggcttgtcgagacagagaagactcttgcgtttctgataggcacctattgg |
| | tcttactgacatccactttgcctttctctccacaggttcgaattcgccgccaccatgtac |
| | agaatgcagctgctgctgctcattgccctgtctctggccctggtcaccaattctgaggtc |
| | cagctgcttgagagtggtggtggactggttcagcctggtggcagcctgagactgtcttgt |
| | gctgcctctggcttcaccttcagccactatatcatgatgtgggtcagacaggcccctggc |
| | aaaggcctggaatgggtgtcaggcatctacagcagtggtggcatcacagtgtatgctgac |
| | tctgtgaagggcagattcaccatcagcagagacaacagcaagaacaccctgtacctgcag |
| | atgaactccctgagagctgaggacacagcagtgtactactgtgcctatagaagaattggg |
| | gtgcccagaagggatgagtttgacatctggggccagggcaccatggttacagtgtcctct |
| | gccagcacaaagggcccctctgtttttccactggctcccagcagcaagagcaccagtggt |
| | ggaacagctgccctgggctgtctggtcaaggattacttccctgagcctgtgactgtgtcc |
| | tggaactctggggctctgacttctggggtgcacacctttccagctgtgctgcagtcctct |
| | ggcctgtactctctgtcctctgtggtcacagtgcctagctctagcctgggcacacagacc |
| | tacatctgcaatgtgaaccacaagcctagcaacaccaaggtggacaagagagtggaaccc |
| | aagagctgtgacaagacccacacctgtcctccatgtcctgctccagaactgcttggaggc |
| | ccttctgtgttcctgtttcctccaaagcctaaggacaccctgatgatcagcagaacccct |
| | gaagtgacctgtgtggtggttgatgtgtcccatgaggacccagaagtgaagttcaattgg |
| | tatgtggatggggttgaagtgcacaatgccaagaccaagcctagagaggaacagtacaac |
| | agcacctacagagtggtttctgtgctgacagtgctgcaccaggactggctgaatggcaaa |
| | gagtacaagtgcaaggtgtccaacaaggccctgcctgctcctattgagaaaaccatctcc |
| | aaggccaagggccagccaagagaaccccaggtttacaccctgccacctagcagagaagag |
| | atgaccaagaaccaggtgtccctgacctgcctggttaagggcttctacccctctgacatt |
| | gctgtggaatgggagagcaatggccagcctgaaaacaactacaagacaacccctcctgtg |
| | ctggactctgatggctcattcttcctgtacagcaagctgactgtggacaagtccagatgg |
| | cagcagggaaatgtgttcagctgctctgtgatgcatgaggccctgcacaaccactacacc |
| | cagaaaagtctgtctctgtcccctggcagaaagagaagaggctctggagaaggcagaggc |
| | tccctgctgacatgtggggatgttgaagagaatcctgggcctatgtataggatgcaactg |
| | ctcctcctgattgctctgagcctggctcttgtgaccaactctgacatccagatgacacag |
| | agcccctccacactgtctgcatctgtgggagacagagtgaccatcacctgtagagccagc |
| | cagtctatctctagctggctggcctggtatcagcaaaagcctggcaaggcccctaagctg |
| | ctgatctacaaggccagcacacttgagtcaggggtgccctccagattttctggctctgga |
| | tctggcacagagttcaccctgaccatcagctccctgcagccagatgactttgccacctac |
| | tactgccagcagtacaatacctactggacctttggccagggaacaaaggtggaaatcaag |
| | agaacagtggctgccccatctgtgttcatcttcccaccatctgatgaacagctgaagtct |
| | ggcactgcctctgttgtgtgcctgctgaacaacttttaccccagagaagccaaggtgcag |
| | tggaaagtggataatgccctgcagtctggcaatagccaagaatctgtgacagagcaggac |
| | tccaaggattccacctacagcctgagcagcaccctgacactgagcaaggctgactatgag |
| | aagcacaaagtgtatgcctgtgaagtgacacaccagggactgagcagcccagtgaccaag |
| | agcttcaacaggggagagtgctgataactcgaggacggggtgaactacgcctgaggatcc |
| | gatctttttccctctgccaaaaattatggggacatcatgaagccccttgagcatctgact |
| | tctggctaataaaggaaatttattttcattgcaatagtgtgttggaattttttgtgtctc |
| | tca |

HEK293 cells were plated at a density of 7.5×10⁵ cells/well in each well of a standard 6-well dish containing Dulbecco's modified eagle medium (DMEM) supplied with 10% fetal bovine serum (FBS). The next day, cells were transfected with CAG.L01 (SEQ ID NO:148), CAG.L02 (SEQ ID NO: 149), and CAG.L03 (SEQ ID NO:150) AAV constructs using Lifpofectamine 2000 (Invitrogen) according the manufacturer's protocol). Non-transfected cells were used as negative control. Cell culture medium was changed 24 hours post-transfection to opti-mem I reduced serum media (2 mL/well). Cell culture supernatant was harvested at 48 hours post-transfection, and cell lysates were harvested with RIPA buffer (Pierce) supplemented with EDTA-free protease inhibitor tablets (Pierce). Supernatant and lysates samples were stored at −80 C.

Proteins from supernatant or cell lysate samples were separated via the NuPAGE electrophoresis system (Thermo Fisher Scientific). For samples derived from cell lysates, 40 μg of protein was loaded unless indicated otherwise. Purified human IgG or Lanadelumab IgG (produced by Genscript) were used as loading controls (50-100 ng). Samples were heated with LDS sample buffer and NuPAGE reducing agent at 70C for 10 minutes and then loaded into NuPAGE 4-12% Bis-Tris protein gels. Separated proteins were transferred to PVDF membranes using the iBlot2 dry blotting system according to manufacturer's instructions (P3 default setting was used for the protein transfer). Membranes were immediately washed in phosphate buffer saline with 0.1% v/v Tween-20 (PBST). Membranes were then incubated in blocking solution containing PBST and 1% Clear Milk Blocking Buffer (Thermo Scientific) for 1 hour at room temperature. Membranes were then incubated in fresh blocking solution supplemented with goat anti-human kappa light chain-HRP antibody (Bethyl Laboratories; 1:2000 dilution) and goat anti-human IgG Fc-HRP antibody (1:2000 dilution). Following antibody incubation, membranes were washed three times in PBST for 5 minutes per wash. Finally, membranes were incubated in SuperSignal West Pico PLUS chemiluminescent substrate for 5 minutes and imaged on the BioRad Universal Hood II gel doc system for detection of horseradish peroxidase (HRP) signal.

### Results

Expression analysis of reporter transgene (eGFP) following transfection of different plasmid quantities (4 μg-nontransfected) showed a dose dependent increase in eGFP levels (FIG. 7B). Protein expression analysis of lanadelumab in the cell lysate (FIG. 7C) and in the cell supernatant (FIG. 7D) showed dose-dependent levels of lanadelumab in cell lysates and supernatant. Transfection with the construct containing the L02 transgene (SEQ ID NO:149, CAG.L02 (SEQ ID NO:153)), a codon-optimized and depleted of CpG dinucleotide sequences construction, resulted in higher expression levels compared to L01 transgene (SEQ ID NO:148, CAG.L01 SEQ ID NO:149). Transfection of CAG.L02 (SEQ ID NO:153) and CAG.L03 (SEQ ID NO:152) resulted in similar expression levels.

### Example 3: Serum Expression of Lanadelumab in Mice

### Methods

A. Mouse experiments were performed with either AAV8 or AAV9 containing an AAV construct (as depicted in FIG. 7A) comprising the L01 sequence (SEQ ID NO:148), which contains the Furin and F2A sequence (SEQ ID NO:106). AAV8 and AAV9 vectors (n=5 mice per group; 2e11 genome copies (gc)) were administered to immunocompromised NSG mice via either intravenous (IV) or intramuscular (IM) routes. IV administrations were into the tail vein and IM administrations were bilateral into the gastrocnemius muscles. Mice injected with vehicle were included as controls. Seven weeks post administration mice were sacrificed and serum human antibody levels were determined by enzyme-linked immunosorbent assay (ELISA).

Lanadelumab levels in NSG mouse serum was assessed by ELISA. Briefly, mouse serum was obtained before treatment and at 1, 3, 5 and 7 weeks post in vivo gene transfection and stored at −80° C. 96-well plate was coated with 1 μg/ml human IgG-Fc fragment antibody (Bethyl, Montgomery, TX) in carbonate bicarbonate buffer (0.05M, pH 9.6, Sigma-Aldrich, St. Louis, MO) and incubated overnight at 4° C. After washing with Tween 20 washing buffer (PBST, 0.05%, Alfa Aesar, Haverhill, MA), plate was incubated with blocking buffer (3% BSA in PBS, ThermoFisher Scientific, Waltham, MA) for 1 h at 37° C. followed by washing. Mouse serum samples diluted in sample dilution buffer (0.1% Tween 20 and 3% BSA in PBS) was added to the plate (50 μl/well) and incubated for 2 h at 37° C. A standard curve of known lanadelumab concentrations ranging from 360 to 0.001 ng/mL was included in each plate. Plate was washed with PBST for five times after incubation. The levels of lanadelumab was detected by incubation with horseradish peroxidase-conjugated goat anti-human IgG (H+L) (200 ng/mL; Bethyl, Montgomery, TX) for 1 h at 37° C. The optical density was assessed using KPL TMB Microwell Peroxidase Substrate System (Seracare, Milford, MA) following the manufacturer's specifications. Data analysis was performed with SoftMax Pro version 7.0.2 software (Molecular Devices, Sunnyvale, CA).

### Results

A. Results from a representative experiment are shown in FIG. 8. Serum analysis of AAV8-, AAV9-injected and control (vehicle) NSG mice at 7 weeks post gene transfer showed expression and serum accumulation of Lanadelumab following AAV9 delivery (2E¹¹ gc). Serum Lanadelumab concentration was 100-fold higher in AAV9-injected mice compared to AAV8-injected mice and slightly higher in IV-AAV9-injected compared to IM-AAV9-injected mice. Serum human antibody levels in control mice were undetectable at 7 week time point.

B. In an analogous experiment, a time course of lanadelumab serum levels in NSG mice post-AAV9 administration (n=5 per group) was performed. AAV9 vectors (2E¹¹ gc) were injected either IV or IM (as above, in experiment A), and serum antibody levels were determined by ELISA at day 7 (D7), day 21 (D21), day 35 (D35), and day 49 (D49).

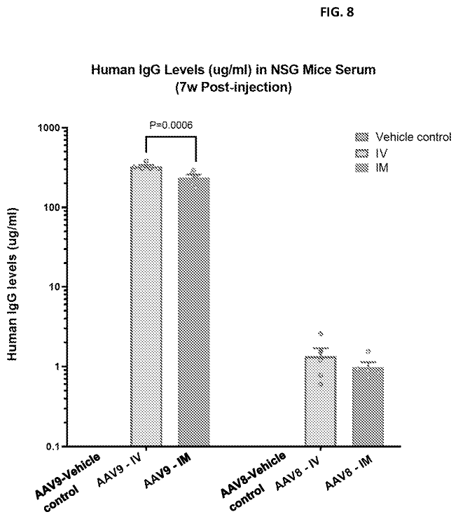

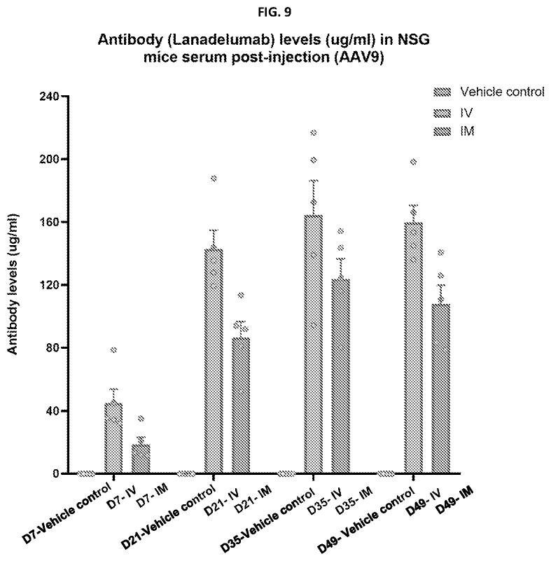

Serum Lanadelumab expression is detectable as early as 1 week (D7) after AAV9 administration in NSG mice. The expression levels increased at 3 weeks (D2), peaked at 5 weeks (D35) and then sustained up to 7 week post-injection (D49). It was observed that serum lanadelumab concentration is higher in IV vs. IM injected mice over the entire time course. See FIG. 9.

C. In an analogous experiment, a time course of lanadelumab serum levels in C/57BL6 mice post AAV8 administration was performed. The optimized expression cassette containing a liver-specific promoter and a codon optimized and CpG depleted transgene with a modified furin-2A processing signal resulted in robust serum antibody concentration when delivered intravenously using an AAV8 vector. Very high (>1 mg/ml) and sustained levels of functional anti-kallikrein antibody were achieved in the serum of C57BL/6 mice following IV vector administration at a dose of 1E¹³ gc/kg.

### Example 4: Analysis of In Vitro Transduction and Expression of Tandem Liver- and Tandem Liver/Muscle-Specific Promoters Driving Expression of Lanadelumab

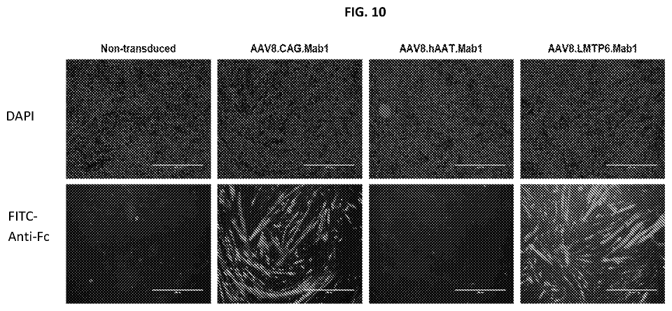

Cis plasmids expressing vectorized lanadelumab were packaged in AAV, then rAAV particles evaluated for potency of the transduction by AAV. Each cis plasmid contained lanadelumab (Mab1) antibody light chain and heavy chain which are multicistrons driven by the CAG, ApoE.hAAT (SEQ ID NO:21) or LMTP6 (SEQ ID NO:14) promoter. Full-length lanadelumab antibody light chain and antibody heavy chain genes were separated by a furin 2A linker to ensure separate expression of each antibody chain. The entire cassette is flanked by AAV2 ITRs, and the genome is encapsidated in an AAV8 capsid for delivery to C2C12 cells (1E¹⁰ vg per well). For detection of antibody protein, following transduction, the cells are treated with FITC conjugated anti-Fc (IgG) antibody. The AAV8.CAG.Mab1 and AAV8.LMTP6.Mab1 infected cells show high expression in muscle cells, whereas the AAV8.hAAT.Mab1 infection does not result in expression of the antibody in muscle cells (FIG. 10). Cells appeared to be equally confluent and viable in all test wells, as seen by DAPI (DNA) staining (FIG. 10).

### Example 5: Antibody Expression and Vector Biodistribution in Mouse Treated with AAV8 Lanadelumab Vectors Driven by Various Promoters

Thyroxine binding globulin (TBG) and alpha-1 antitrypsin (hAAT) promoters have been widely used as liver-specific promoters in previous pre-clinical and clinical gene therapy studies. A panel of designed promoter cassettes derived from multiple promoters and enhancers were generated and tested them in vitro by transfecting Huh7 cells, a human liver cell line. Promoter candidates were selected, which include ApoE.hAAT (SEQ ID NO:21), LSPX1 (SEQ ID NO:9), LSPX2 (SEQ ID NO: 10), LTP1 (SEQ ID NO:11) and LMTP6 (SEQ ID NO:14). AAV8 vectors encoding vectorized lanadelumab regulated by these promoter candidates were then generated. CAG (SEQ ID NO:36) and TBG (SEQ ID NO:40) promoters served as controls for ubiquitous and liver-specific promoters, respectfully. Strength of these promoters and vector biodistribution were tested in vivo by measuring lanadelumab protein expression compared to vector genome copy in each wild type mouse.

Vectors were administered intravenously to C57Bl/6 mice at equivalent doses (2.5×10¹² vg/kg). Mouse serum was collected biweekly, and lanadelumab protein expression levels were determined by ELISA. Liver samples were harvested at 49 days post vector administration. The presence of viral genomes in each sample was quantified using Lanadelumab probe and primer by Droplet Digital PCR (ddPCR) (the NAICA™ system from Stilla). The genome copy number of glucagon was also measured simultaneously in each sample, the viral genomes were then normalized and demonstrated as vector genome copy number per cell (assuming 2 glucagon/cell). Statistical analysis was performed using one-way ANOVA in GraphPad Prism 8.

Among the AAV8 vectors with liver-specific promoters, the vectors driven by the ApoE.hAAT (SEQ ID NO:21) and LMTP6 (SEQ ID NO:14) promoters provided the highest amount of protein expression at all time points (FIG. 11A). While for the biodistribution data, there was no significant difference of vector genome copy number per cell in liver samples in animals treated with vectors driven by different promoters (FIG. 11B).

All liver-specific promoters outperform the TBG promoter (SEQ ID NO:40), and the dual-specific LMTP6 promoter (SEQ ID NO:14) consistently shows the highest expression in the serum (μg/ml) (FIG. 11).

### Example 6: Lanadelumab Expression in Rat Serum Following Administration of Vectorized Antibody

| Rat | Male 8-10 week old Wistar Rats (180-200 g) | | | Bleeds - Day sampled (X) | | |
|---|---|---|---|---|---|---|
| group | Treatment | N | ROA | Day −7 | Day 0 | Day 7 |
| 1 | AAV8.CAG.LANv2.T2A | 3 | IM | X | Administration | X |
| | Dose: 1e13 vg/kg | | | | | |
| 2 | AAV8.CAG.LANv2.T2A | 3 | IV | X | Administration | X |
| | Dose: 1e13 vg/kg | | | | | |
| 3 | AAV8.CAG.LANv2.T2A | 3 | IV | X | Administration | X |
| | Dose: 1e14 vg/kg | | | | | |

| Rat | Bleeds - Day sampled (X) | | | | | | | |
|---|---|---|---|---|---|---|---|---|
| group | Day 10 | Day 14 | Day 17 | Day 21 | Day 28 | Day 35 | Day 42 | Day 49 |
| 1 | X | X | X | X | X | X | X | sacrifice |
| 2 | X | X | X | X | X | X | X | sacrifice |
| 3 | X | X | X | X | X | X | X | sacrifice |

A high level of Lanadelumab expression was detected in the serum of mice treated with AAV-Lanadelumab via IV administration. In parts of the study, the lanadelumab expression levels in different rat strains treated with different doses of AAV-Lanadelumab vectors and controls were examined.

### Experiment 1 (Wistar Rats)

To evaluate the route and the dose of vector administration in rats, a control vector AAV.CAG-LANv2.T2A (CAG.L02; SEQ ID NO: 241) was tested in Wistar rat. Eight to ten weeks old male Wistar rats were assigned into three groups (n=3 per group) to receive vector administration via IM or IV injection at a dose of 1×10¹³ vg/kg or 1×10¹⁴ vg/kg. Blood was collected at 7 days before treatment and 7, 10, 14, 17, 21, 28, 35, 42 and 49 days post vector administration and processed into serum.

Table 8. Study details for Lanadelumab expression in rat serum, Experiment 1.

Levels of human IgG antibody in collected rat serum were detected by ELISA.

| Rat | Male 8-10 week old Wistar Rats (180-200 g) | | | Bleeds - Day sampled (X) | | |
|---|---|---|---|---|---|---|
| group | Treatment | N | ROA | Day −7 | Day 0 | Day 7 |
| 1 | AAV8.CAG.LANv2.T2A | 3 | IM | X | Administration | X |
| | Dose: 1e13 vg/kg | | | | | |
| 2 | AAV8.CAG.LANv2.T2A | 3 | IV | X | Administration | X |
| | Dose: 1e13 vg/kg | | | | | |
| 3 | AAV8.CAG.LANv2.T2A | 3 | IV | X | Administration | X |
| | Dose: 1e14 vg/kg | | | | | |

| Rat | Bleeds - Day sampled (X) | | | | | | | |
|---|---|---|---|---|---|---|---|---|
| group | Day 10 | Day 14 | Day 17 | Day 21 | Day 28 | Day 35 | Day 42 | Day 49 |
| 1 | X | X | X | X | X | X | X | sacrifice |
| 2 | X | X | X | X | X | X | X | sacrifice |
| 3 | X | X | X | X | X | X | X | sacrifice |

Statistical analysis was done by one-way ANOVA with multiple comparisons at each time point using Prism

TABLE 9
Results of Lanadelumab expression in Wistar rats, Experiment 1

| | AAV8.CAG.Lanv2.T2A | | | AAV8.CAG.Lanv2.T2A | | | AAV8.CAG.Lanv2.T2A | | |
|---|---|---|---|---|---|---|---|---|---|
| Day | 1e13 vg/kg- IM | | | 1e13 vg/kg- IV | | | 1e14 vg/kg- IM | | |
| sampled | Mean | SEM | N | Mean | SEM | N | Mean | SEM | N |
| D 7 | 4.1 | 0.96 | 3 | 8.43 | 0.64 | 3 | 33.6 | 13.86 | 3 |
| D 10 | 7.67 | 1.91 | 3 | 12.3 | 0.81 | 3 | 64.5 | 30.2 | 3 |
| D 14 | 9.37 | 0.73 | 3 | 23.13 | 0.18 | 3 | 123.23 | 69.12 | 3 |
| D 17 | 4.97 | 2.23 | 3 | 92 | 53.52 | 3 | 245.17 | 151.09 | 3 |
| D 21 | 2.04 | 0.9 | 3 | 33.06 | 5.84 | 3 | 252.63 | 149.41 | 3 |
| D 28 | 92.08 | 87.07 | 3 | 65.23 | 41.69 | 3 | 117.97 | 112.47 | 3 |
| D 35 | 14.46 | 12.31 | 3 | 88.1 | 56.68 | 3 | 122.97 | 74.94 | 3 |
| D 42 | 82.29 | 80.46 | 3 | 40.43 | 26.77 | 3 | 108.02 | 99.26 | 3 |
| D 49 | 1.66 | 0.95 | 3 | 81.4 | 39.96 | 3 | 216.30 | 118.18 | 3 |

The levels of antibody in rat serum were detectable at 7 days post treatment. It increased over time and reached the peak level at 17 (lower dose) and 21 (higher dose) days post treatment in IV groups and 28 days in IM group. The antibody levels gradually decreased and sustains up to 48 days post treatment in all groups. For animals treated with lower dose (1×10¹³ vg/kg) vector, the antibody expression levels in IV groups are significantly higher than that in IM group at 7, 14 and 21 days post vector administration. For animals received IV administration, the antibody expression levels were dose-dependent at all time points. The highest level of lanadelumab expression was 252.6±149.4 μg/ml, which was detected in animals treated with higher dose (1×10¹⁴ vg/kg) at 21 days post IV administration. See FIG. 12A.

### Experiment 2 (Wistar and Sprague-Dawley Rats)

The aim of this experiment was to investigate the rat strain and the vector dose that will be used for a rat efficacy study. Eight to ten weeks old male Wistar and Sprague-Dawley (SD) rats were assigned into four groups (n=3 per group) to receive treatment of AAV8 vector carrying genome encoding lanadelumab driven by a universal promoter, CAG.L02 (SEQ ID NO:153), or a liver-specific promoter, ApoE.hAAT.L02 (SEQ ID NO:155). Vectors were administered via IV injection at a dose of 5×10¹³ vg/kg. Blood was collected at 7 days before treatment and 7, 10, 14, 17, 21, 28, 35, 42 and 49 days post vector administration and processed into the serum (Table 10). Levels of human IgG antibody in collected rat serum were detected by ELISA. Statistical analysis was done by one-way ANOVA with multiple comparisons at each time point using Prism.

TABLE 10
Study details for lanadelumab expression in rat serum, Experiment 2.

| | Male•8-10•week•old•¶ | | | | | |
|---|---|---|---|---|---|---|
| | Wistar• or• Sprague• Dawley• (SD)• Rats (180-200 g)¤ | | | | | |
| Rat• | Treatment¶ | | | → •Bleeds•-•Day•sampled•(X)¤ | | |
| group¤ | Dose: •5e13•vg/kg¤ | N¤ | ROA¤ | Day• −1¤ | Day•0¶ | Day•7¶•¤ |
| 1¤ | CAG.L02¶ | 3¤ | IV¤ | X¤ | Administration¤ | X¤ |
| | Wistar¤ | | | | | |
| 2¤ | ApoE.hAAT.L02¶ | 3¤ | IV¤ | X¤ | Administration¤ | X¤ |
| | Wistar¤ | | | | | |
| 3¤ | CAG.L02¶ | 3¤ | IV¤ | X¤ | Administration¤ | X¤ |
| | Sprague•Dawley•(SD)¤ | | | | | |
| 4¤ | ApoE.hAAT.L02¶ | 3¤ | IV¤ | X¤ | Administration¤ | X¤ |
| | Sprague•Dawley•(SD)¤ | | | | | |

| Rat• | → •Bleeds•-•Day•sampled•(X)¤ | | | | | | |
|---|---|---|---|---|---|---|---|
| group¤ | Day•14¤ | Day•17¤ | Day•21•¤ | Day•28¤ | Day•35•¤ | Day•42¤ | Day•49¤ |
| 1¤ | X¤ | X¤ | X¤ | X¤ | X¤ | X¤ | sacrifice•¤ |
| 2¤ | X¤ | X¤ | X¤ | X¤ | X¤ | X¤ | sacrifice¤ |
| 3¤ | X¤ | X¤ | X¤ | X¤ | X¤ | X¤ | sacrifice¤ |
| 4¤ | X¤ | X¤ | X¤ | X¤ | X¤ | X¤ | sacrifice¤ |

In this experiment, a control vector (CAG.L02, SEQ ID NO: 241) and vector ApoE.hAAT.L02 (SEQ ID NO: 243) were tested in Wistar and SD rats, respectively. Lanadelumab expression levels were higher in Wistar rat than SD rat in both vector groups at all time points. At the early time points, animals treated with control vector showed significant higher serum antibody levels than those treated with the liver-specific promoter containing vector. This was observed in Wistar rat at 7 days post treatment, and in SD rat at 7, 14 and 17 days post treatment. In Wistar rats, the concentrations of antibody gradually increased over time in both vectors group. The highest antibody levels were 173.1±78.8 μg/ml and 109.57±18.9 μg/ml at 35 and 49 days respectively in control CAG-Lanadelumab and hAAT-Lanadelumab vector-treated animals. In SD rats, however, the levels of antibody reached peaks at 14 and 21 days in control and lead vector-treated animals, respectively, and decreased gradually afterward in both groups. The highest antibody concentrations were 48.23±3.1 μg/ml and 22.33±8.98 μg/ml in CAG.L02 (SEQ ID NO: 241) and ApoE.hAAT.L02 (SEQ ID NO: 243) vector groups, respectively. See Table 11 and FIG. 12B.

TABLE 11
Results of Lanadelumab expression in Wistar rats, Experiment 2:

| | | |
|---|---|---|
| | CAG-Wistar | ApoE.hAAT- Wistar |
| Day | 5e13 vg/kg- IV | 5e13 vg/kg- IV |

| sampled | Mean | SEM | N | Mean | SEM | N |
|---|---|---|---|---|---|---|
| D 7 | 25.04 | 5.26 | 3 | 5.80 | 0.61 | 3 |
| D 14 | 91.50 | 39.90 | 3 | 29.73 | 6.36 | 3 |
| D 17 | 111.30 | 51.29 | 3 | 55.10 | 18.60 | 3 |
| D 21 | 132.03 | 58.76 | 3 | 75.80 | 17.94 | 3 |
| D 28 | 159.90 | 69.08 | 3 | 74.27 | 21.84 | 3 |
| D 35 | 173.10 | 78.76 | 3 | 97.67 | 33.89 | 3 |
| D 42 | 173.00 | 57.73 | 3 | 83.10 | 27.67 | 3 |
| D 49 | 163.57 | 39.45 | 3 | 109.57 | 18.87 | 3 |

### Example 7: Characterization of Vectorized Lanadelumab Regulated by Tissue-Specific Promoters Following Intramuscular Administration

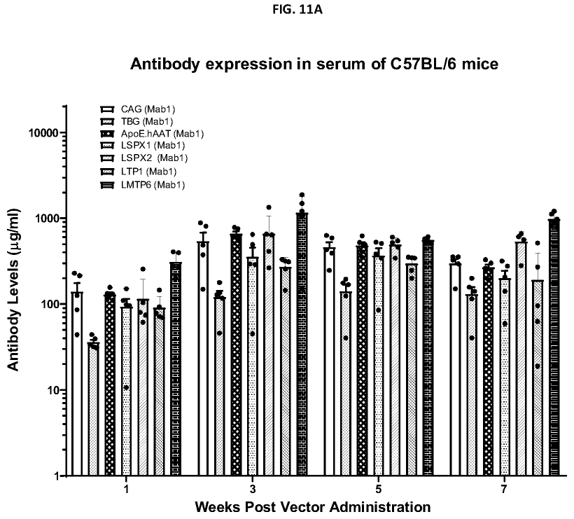

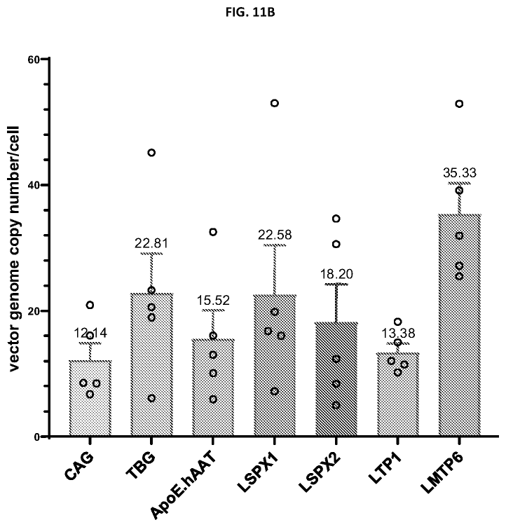

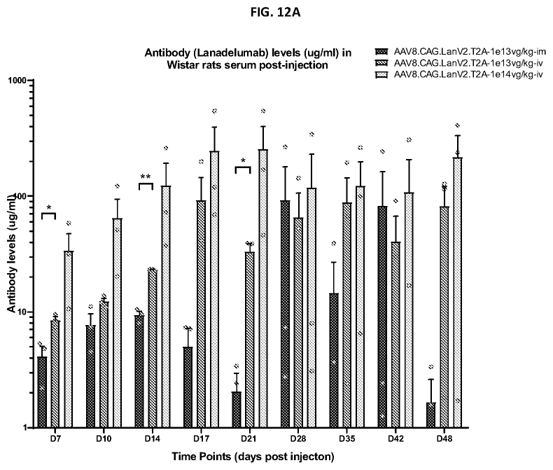

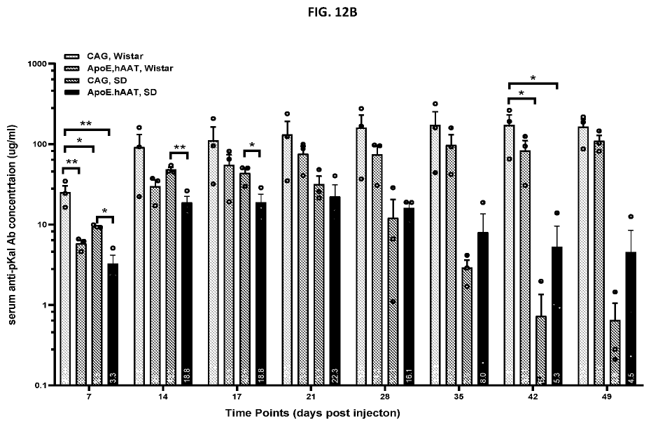

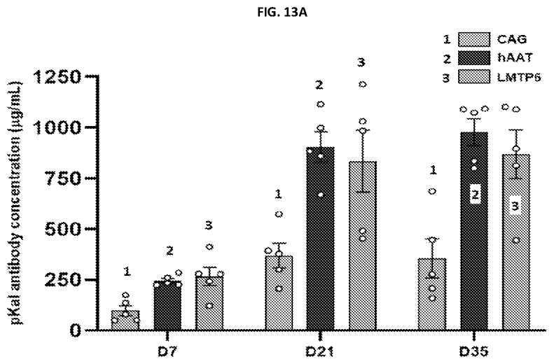

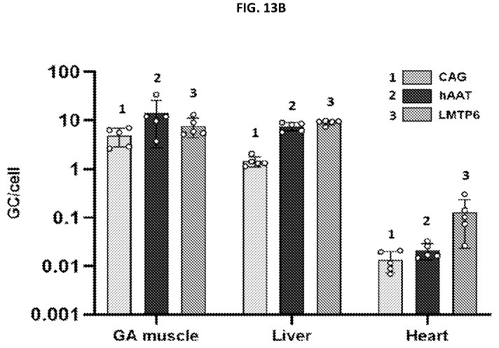

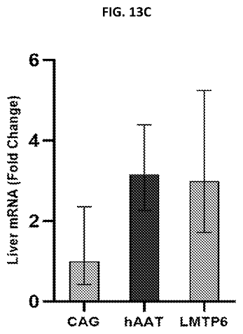

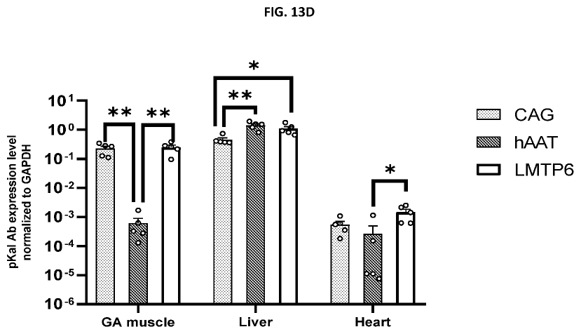

In a previous study, high liver-driven expression of vectorized lanadelumab with AAV8 regulated by the ApoE.hAAT or LMTP6 promoters was identified. The goal of this study was to characterize muscle-driven expression of the LMTP6 promoter following direct injection of lanadelumab vectors into the gastrocnemius (GA) muscle. Animals received bilateral injections of 5×10¹⁰ vg into the GA muscle. Serum was collected biweekly to measure systemic lanadelumab concentration (FIG. 13A). Animals were harvested at 49 days post-injection, and relevant tissues (liver, GA muscle, heart) were analyzed for vector biodistribution and transgene expression.

Vectors regulated by the hAAT and LMTP6 promoters demonstrated significantly increased antibody concentrations in serum compared to CAG at all time points (FIG. 13A). The hAAT and LMTP6 were not significantly different from each other in this experiment. Vector genome copies per cell of vectorized lanadelumab was detected and quantified in GA, liver and heart (FIG. 13B) with a notable difference of higher quantity of genome detected in heart for the dual muscle/liver promoter, LMTP6 vector. Increased liver RNA expression was also detected for all test vectors directly injected into GA muscle at 49 days (relative fold gene expression compared to a reference gene) (FIG. 13C). Gene expression (mRNA μg/mL) data from each of liver, GA muscle, and heart (FIG. 13D) indicates the dual specificity of LMPT6 in liver and muscle tissues following IM administration, whereas the hAAT-driven samples were reduced in muscle compared to LMTP6 and CAG. Significant differences were also seen between the hAAT and LMTP6 groups.

### Example 8: Comparison of Lanadelumab Protein Levels in Mouse Serum Derived from Mice Treated with AAV-Lanadelumab Vectors Produced with Different Production Systems

Different AAV production protocols were developed to identify methods that can increase AAV titer and scalability, as well as assess the quality of vector product. Cis and trans plasmids to generate AAV8.Lanadelumab rAAV vectors (all having the same transgene driven by a CAG promoter) were constructed by well-known methods suitable for HEK293-transfected cell and also baculovirus (BV)/Sf9 insect cell production methods. Three different BV/Sf9 vector systems, BV1, BV2 and BV3, were provided as well as rAAV vector produced by an HEK293 method as a control. Purified rAAV product was injected into wild-type mice for this protein expression study (Table 12).

Young adult C57BL/6 mice (aged 8-10 weeks) were administered with above-mentioned vectors at 2.5E¹² vg/kg via tail vein injection (n=5 per group). Serum was collected from each animal at 7, 21, 35, and 49 days post vector administration. Serum collected two days before injection (Day 0) served as baseline control. Levels of antibody (lanadelumab) expression were detected via ELISA. Data analysis was done by one-way ANOVA with multiple comparisons at each time point using Prism.

TABLE 12
Production system expression study design

| | AAV8.Lanadelumab | | | | | | | | | |
|---|---|---|---|---|---|---|---|---|---|---|
| | 5e10 vg (2.5e12 vg/kg) | | | | | | | | | |
| | rAAV | | | Bleeds sampled (X) | | | | | | |
| Mouse | Production | | | | Day 0 | Day 7 | Day 21 | Day 35 | Day 49 | |
| Group | method | N | ROA | Day −2 | (Week 0) | (Week 1) | (Week 3) | (Week 5) | (Week 7) | Day 50 |
| 1 | HEK cells | 5 | IV | X | Injection | X | X | X | X | Serum |
| 2 | BV1 | 5 | IV | X | Injection | X | X | X | X | Serum |
| Werum | BV2 | 5 | IV | X | Injection | X | X | X | X | Serum |
| 3 | | | | | | | | | | |
| 4 | BV3 | 5 | IV | X | Injection | X | X | X | X | Serum |

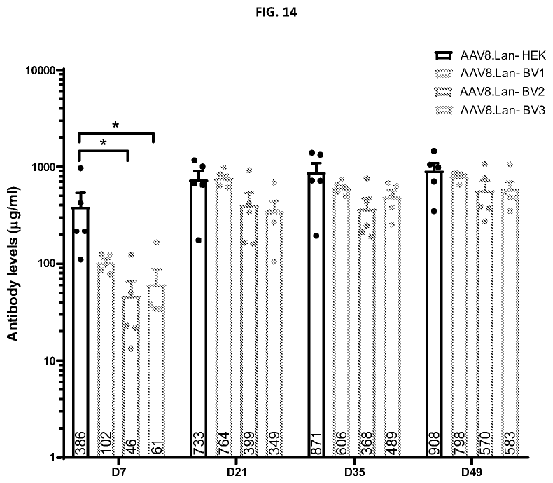

All production methods tested are viable based on this study, with greater yields from the HEK cell production method at the time points tested (see FIG. 14). Antibody expression in serum is detectable as early as 7 days post administration in all groups. The average of antibody concentration at Day 7 in the HEK production group is 386 μg/ml, which is significantly higher than other groups (61-102 μg/ml). The levels of antibody expression increase at day 21 by 1-, 6-, 7-, and 4-fold in BV1, BV2 and BV3 groups, respectively. Antibody expression levels sustained at 35 and 49 days post administration. There is no significant difference in between HEK produced vector and BV3 produced vectors at day 21, 35 and 49 time points.

### Example 9: Vectorized Human Anti-pKal Antibody, Lanadelumab, Derived from Mouse Serum Suppressed Human pKal Function

In order to measure pKal function of lanadelumab derived from mouse serum following AAV-lanadelumab administration, a fluorescence-based kinetic enzymatic functional assay was. First, activated human plasma kallikrein (Enzyme Research Laboratories) was diluted in sample dilution buffer (SDB; 1×PBS, 3% BSA, 0.1% Tween-20) to top concentration of 100 nM. This pKal was two-fold serially diluted for a total of 12 concentrations in the dilution series (100 nM-0.05 nM). From each dilution, and in duplicate, 25 μL was placed in one well of a 96-well, opaque flat-bottomed plate along with 25 μL of SDB. Then, 50 μL of the fluorogenic substrate Pro-Phe-Arg-7-Amino-4-Methylcoumarin (PFR-AMC) (Bachem) prepared at 100 μM in assay buffer (50 mM Tris, 250 mM NaCl, pH 7.5) was added to each well. The samples were immediately run in kinetic mode for AMC fluorescence at excitation/emission wavelengths of 380/460 nm, respectively, for 3 hours using a SpectraMax 3 fluorescent plate reader.

The signal-to-noise ratio for each pKal concentration RFU (last RFU fluorescent value chosen) was calculated by dividing its RFU by background PFR-AMC substrate fluorescence. The two lowest pKal concentrations with a signal-to-noise ratio ≥2 (6.25 nM and 12.5 nM) were then chosen to evaluate the suppressive effect and range of lanadelumab antibody of pKal function in a lanadelumab dose response. Lanadelumab (GenScript) or human IgG control antibody was diluted in SDB to top concentration of 200 nM and two-fold serially diluted to 0.39 nM. Next, 25 μL pKal (each of two chosen concentrations) was incubated with 25 μL lanadelumab or human IgG at 30° C. for 1 hour. Antibody-pKal mixture was then given PFR-AMC and immediately run in kinetic mode for AMC fluorescence at excitation/emission wavelengths of 380/460 nm, respectively, for 3 hours using a SpectraMax fluorescent plate reader.

In vitro pKal functional assay. When used, mouse serum was diluted in sample dilution buffer and incubated 1:1 with 6.25 nM (1.56 nM in-well) pKal for 30° C./1 hour. For total IgG purification from mouse serum, antibody was purified using the Protein A Spin Antibody Purification Kit (BioVision) according to manufacturer's protocol. Total antibody concentration was measured using a Nanodrop spectrophotometer, with OD absorbance=280 nM. AMC standard curve was generated by a two-fold downward dilution series of AMC (500 nM, eleven dilutions and blank subtracted) diluted in assay buffer. AMC was read as end point fluorescence at excitation/emission wavelengths of 380/460 nm, respectively. Specific plasma kallikrein activity was calculated as: (adjusted experimental sample Vmax, RFU/sec)×(Conversion factor, AMC standard curve μM/RFU)/(pKal concentration, nM). Percent reduction in pKal activity was derived from calculating day 49 by day −7 pKal activity.

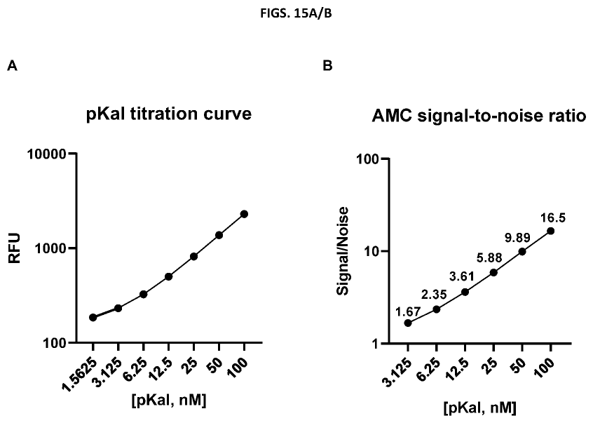

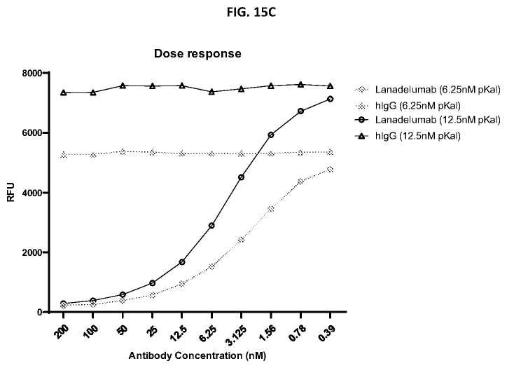

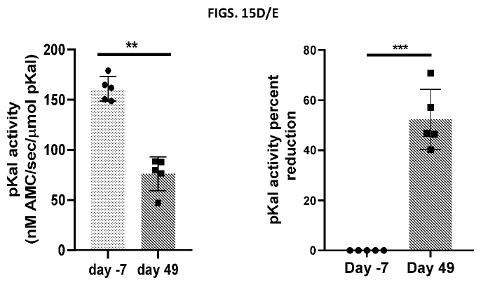

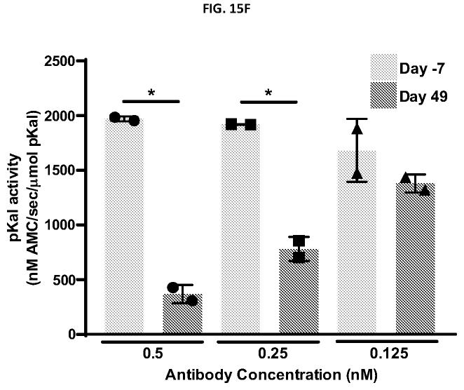

To determine whether AAV-derived lanadelumab can suppress plasma kallikrein function, we developed the in vitro AMC substrate-based functional assay to address this in a proof-of-concept study (FIGS. 15A and 15B). In this assay, antibody-containing medium is incubated with activated human pKal, as described above. Antibody-bound pKal is then given the synthetic peptide substrate Pro-Phe-Arg conjugated to AMC (PFR-AMC) and amount of released AMC is measured over time at excitation/emission wavelengths of 380/460 nm, respectively, for 3 hours. The assay showed noticeable lanadelumab-mediated suppression of pKal activity down to 0.1 nM (in-well concentration) (FIG. 15C) at a defined enzyme concentration. We first sought to determine whether serum from mice administered lanadelumab-encoded AAV could suppress pKal activity. Serum from mice 49 days post-administration was diluted 1:25 (in range predicted to be suppressive), incubated with pKal in vitro, and pKal activity was assayed. Serum from mice post-vector administration, as opposed to 7 days pre-administration, suppressed pKal activity, as reflected in a significant reduction of enzyme activity and a ˜50% percent reduction in pKal activity between the two time points (FIGS. 15D and 15E).

Further experiments show that suppression was due to the lanadelumab within the serum. Reasoning that the human IgG, namely lanadelumab, would only be found in the day 49 post-administration IgG fraction, but not the day −7 pre-administration samples, purified and total IgG antibody was used from the aforementioned day −7 and day 49 mouse serum samples to test pKal suppression. Indeed, only lanadelumab-containing purified IgG from day 49 post-administration serum, but not IgG from the pre-administration time point, suppressed human pKal function (FIG. 15F).

### Example 10: Effects of AAV-Lanadelumab in Carrageenan Animal Models

### Example 14A: Effects of AAV-Lanadelumab in the Carrageenan Paw Edema Model in

Inflammation models induced by carrageenan are frequently used acute inflammation models. Carrageenan (Cg) is a strong chemical agent that functions in stimulating the release of inflammatory and proinflammatory mediators, including bradykinin, histamine, tachykinins, reactive oxygen, and nitrogen species. Typical signs of inflammation include edema, hyperalgesia, and erythema, which develop immediately following the treatment of carrageenan. This example evaluated the effect of AAV-mediated gene delivery of Lanadelumab on carrageenan-induced paw edema in mice.

In total eighty young adult (8-9 weeks old) male C57BL/6 mice were used for this study. Animals were divided into eight groups as listed in Table 13. Paw edema was induced by a single subcutaneous (s.c.) injection of 30 μL of 0.7% or 1% carrageenan solution. Test vectors and positive control Diclofenac were administered at 21 days and 30 minutes prior to carrageenan treatment. Blood was collected before vectors injection and at 7 and 21 days post injection from mice in groups 1, 3, 4, 5, 7 and 8. Paw volume was measured using a digital Plethysmometer prior to carrageenan injection, and at 2, 4, 6, 8, 24 and 48 hours after injection. All animals were sacrificed 48 hours after carrageenan injection. Liver and paw specimens were also collected at the necropsy.

TABLE 13
Carrageenan (Cg) Paw Edema Mouse Study design

| | | Test Items | | Route, Dose | | |
|---|---|---|---|---|---|---|
| *Gr. | Group/treatment | Dose level | | volume | Pre-treatment time | N |
| 1 | Cg (0.7%)/Vehicle of | 0 | | I.P., 10 mL/kg | 30 min before Cg | 10 |
| | diclofenac | | | | | |
| 2 | Cg (0.7%)/Diclofenac | 60 | mg/kg | I.P., 10 mL/kg | 30 min before Cg | 10 |
| 3 | Cg (0.7%)/Vector 1 | 1e13 | vg/kg | I.V., | 3 weeks before Cg | 10 |
| | | | | 3.333 mL/kg | | |
| 4 | Cg (0.7%)/ | 1e13 | vg/kg | I.V., | 3 weeks before Cg | 10 |
| | AAV8.ApoE.hAAT.Lan | | | 3.333 mL/kg | | |
| 5 | Cg (1.0%)/Vehicle of | 0 | | I.P., 10 mL/kg | 30 min before Cg | 10 |
| | diclofenac | | | | | |
| 6 | Cg (1.0%)/Diclofenac | 60 | mg/kg | I.P., 10 mL/kg | 30 min before Cg | 10 |
| 7 | Cg (1.0%)/Vector 1 | 1e13 | vg/kg | I.V., | 3 weeks before Cg | 10 |
| | | | | 3.333 mL/kg | | |
| 8 | Cg (1.0%)/ | 1e13 | vg/kg | I.V., | 3 weeks before Cg | 10 |

| | |
|---|---|
| AAV8.ApoE.hAAT.Lan | 3.333 mL/kg |

Vector 1: AAV8-GFP

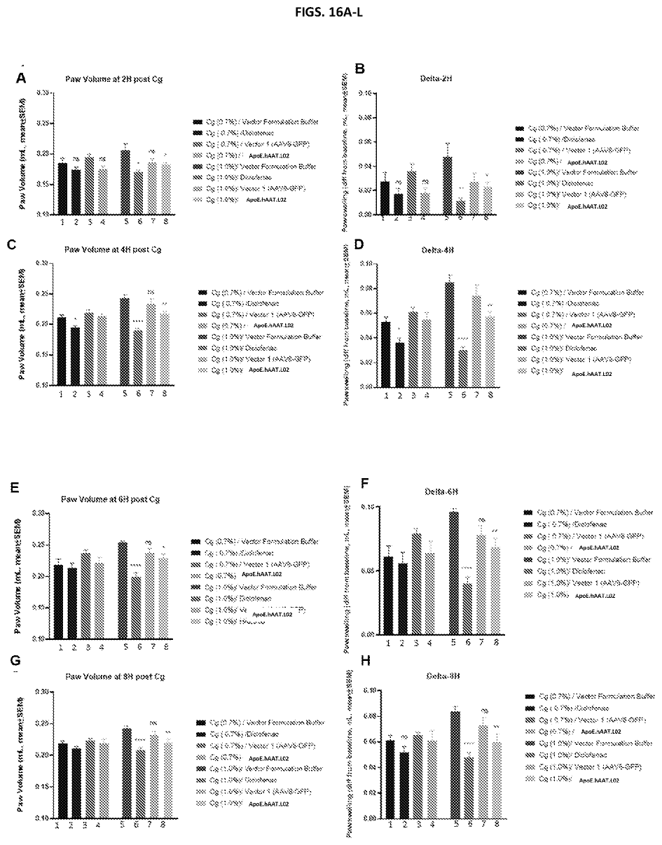

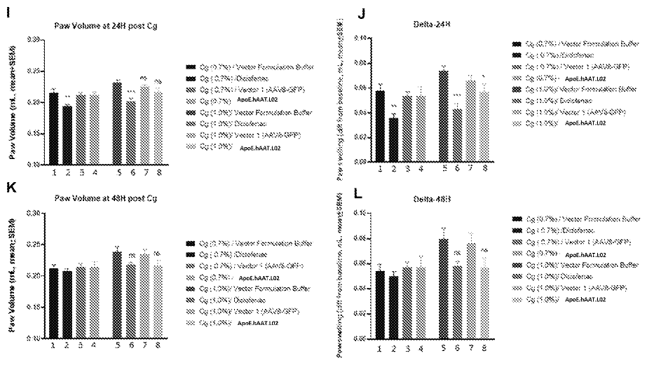

Both 0.7% and 1.0% carrageenan induced swelling in the injected paw; however, swelling was more pronounced with 1.0% carrageenan injection (FIGS. 16, A-L). In the positive control groups (Group 2 and 6), diclofenac treatment significantly decreased the paw volume at 2, 4, 6, 8 and 24 hours post carrageenan injection in 1.0% Cg model (group 2), while a significant decrease on paw volume was observed only at 4 and 24 hours post injection in 0.7% Cg model (group 6).

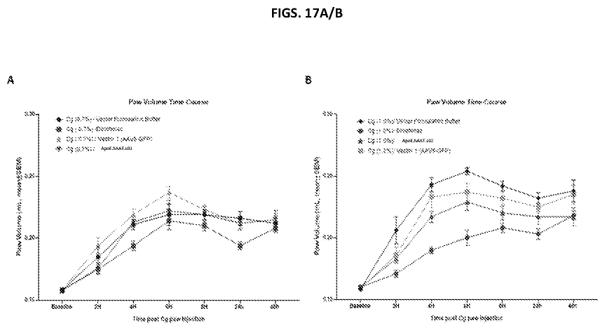

ApoE.hAAT.L02 (SEQ ID NO:155) treatment significantly reduced the paw volume at 2, 4, 6 and 8 hours post carrageenan injection in 1.0% Cg model when compared with the vehicle control group (group 1, vector formulation buffer) (FIGS. 17A and 17B). However, no effect of ApoE.hAAT.L02 treatment was observed in 0.7% Cg model at any time points (FIGS. 17A and 17B). There is no significant difference in between groups treated with vehicle (groups 1 and 4) or control vector (AAV-GFP, groups 3 and 7) in both 1.0% and 0.7% Cg models (FIGS. 16A-L). All data was analyzed with One-way ANOVA with Dunnett's post-hoc test for multiple comparisons.

These data indicate that acute inflammation can be successfully induced in mouse paw with a single subcutaneous injection of 1% carrageenan solution. Lanadelumab, a human IgG antibody produced in mouse serum via AAV-mediated gene delivery significantly reduces the severity of inflammation in mouse 1% carrageenan model.

### Example 11: Characterization of Tissue-Restricted Transgene Immunogenicity

The goal of this study is to understand transgene immunogenicity and/or tolerance induction in the context of ubiquitous, tissue-specific, or tandem promoters. Hypothesis: Vectors driven by liver-specific and liver-muscle tandem promoters will demonstrate reduced immunogenicity compared to vectors driven by a ubiquitous promoter. To test this hypothesis, four AAV vectors that drive expression of a highly immunogenic membrane-bound ovalbumin (mOVA) were constructed. These vectors differ in their promoter sequences which includes: a) a ubiquitous CAG promoter (SEQ ID NO: 36) b) the liver-specific hAAT promoter with upstream ApoE enhancer (SEQ ID NO:21) c), the muscle-specific CK8 promoter cassette composed of the CK core promoter and three copies of a modified MCK enhancer (SEQ ID NO:37), and d) liver-muscle tandem promoter 6 (LMTP6, SEQ ID NO: 14) that contains sequence elements derived from hAAT and CK8. Initial experiments will measure the immune response following intravenous (IV) vector administration within mice. Study endpoints will include characterization of humoral and cell-mediated immune responses against the mOVA transgene product. In addition, tissues will be harvested for vector biodistribution and transgene expression analysis.

### Example 12: Plasma Expression of Vectorized Lanadelumab in Cynomolgus Monkeys Methods

Plasma kinetics of lanadelumab expression in non-human primates administered AAV vectors encoding lanadelumab antibodies were assessed. The goal of this study was to assess and select the dose of AAV8.ApoE.hAAT.Lan vector that results in sustained lanadelumab expression of at least 200 μg/ml lanadelumab by three months or more. The cynomolgus monkey were chosen as the test system because of its established usefulness and acceptance as a model for AAV biodistribution studies in a large animal species and for further translation to human. All animals on this study were naïve with respect to prior treatment.

Nine cynomolgus animals were used. Animals judged suitable for experimentation based on clinical sign data and prescreening antibody titers were placed in three study groups, each receiving a different dosage of AAV vector, by body weight using computer-generated random numbers. Each set of three animals were administered a single i.v. dose of the vector AAV8.ApoE.hAAT.Lan vector (described above) at the dose of 1E12 gc/kg (Group 1), 1E13 gc/kg (Group 2), and 1E14 gc/kg (Group 3).

Clinical signs were recorded at least once daily beginning approximately two weeks prior to initiation of dosing and continuing throughout the study period. The animals were observed for signs of clinical effects, illness, and/or death. Additional observations were recorded based upon the condition of the animal at the discretion of the Study Director and/or technicians.

Blood samples were collected from a peripheral vein for bioanalytical analysis prior to dose administration and then at weekly intervals for 10 weeks. The samples were collected in clot tubes and the times were recorded. The tubes were maintained at room temperature until fully clotted, then centrifuged at approximately 2400 rpm at room temperature for 15 minutes. The serum was harvested, placed in labeled vials, frozen in liquid nitrogen, and stored at −60° C. or below.

All animals were sedated with 8 mg/kg of ketamine HCl IM, maintained on an isoflurane/oxygen mixture and provided with an intravenous bolus of heparin sodium, 200 IU/kg. The animals were perfused via the left cardiac ventricle with 0.001% sodium nitrite in saline.

As primary endpoint analysis, plasma samples were assayed for lanadelumab concentration by ELISA and/or western blot, to be reported at least as ug lanadelumab per ml plasma; and lanadelumab activity, for example, kallikrein inhibition, by fluorogenic assay.

The presence of antibodies against lanadelumab (ADAs) in the serum were evaluated by ELISA and lanadelumab binding assays. Biodistribution of the vector and lanadelumab coding transcripts were assessed in necroscopy samples by quantitative PCR and NGS methods. Tissues to be assayed included liver, muscle, and heart. Toxicity assessment was done by full pathology, including assaying liver enzymes, urinalysis, cardiovascular health, and more.

### Results

The optimized expression cassette containing a liver-specific promoter and a codon optimized and CpG depleted transgene with a modified furin-T2A processing signal resulted in dose-dependent serum antibody concentrations when delivered intravenously using an AAV8 vector. Sustained levels of functional anti-kallikrein antibody were achieved in the serum of 7 out of 9 cynomolgus monkeys following IV vector administration at all three doses (1E12 gc/kg, 1E13 gc/kg, and 1E14 gc/kg) (FIG. 18). Functional anti-kallikrein antibody was detected in the serum of all animals regardless of the administered dose. Serum levels were reached at 29 days after dose administration with mean maximum levels during this time period of 0.144 μg/mL, 0.635 μg/mL, and 35.16 μg/mL being detected in animals 29 days after receiving 1E12 gc/kg, 1E13 gc/kg, and 1E14 gc/kg, respectively.

### Example 13: Liver-Specific Cis-Regulatory Element.hAAT Screening

Cis-regulatory elements (CREs) are non-coding regions of DNA that regulate transcription of proximal or distal gene regions. Based on the CREs' specific function, CREs may be further classified as promoters, enhancers, and silencers. Putative CREs are typically identified based upon structural features such as chromatin accessibility. In addition, these regions may be characterized via density of epigenetic marks commonly associated with high transcriptional activity. In this work, genomic locations for candidate CREs found proximal to genes that are highly expressed and specific to liver were obtained from the ENCODE database (www.encodeproject.org/). Sequences were isolated from the current human genome assembly (GRCh38) using the NCBI Gene portal (www.ncbi.nlm.nih.gov/gene/). These sequences were synthesized and cloned into AAV reporter cassettes upstream of the liver-specific hAAT promoter. In addition, each construct contained a unique 10-bp DNA barcode between the eGFP coding sequence and polyadenylation signal to allow characterization of transgene expression using next generation sequencing. Cis plasmids containing individual CREs were pooled and produced into an AAV8 vector library.

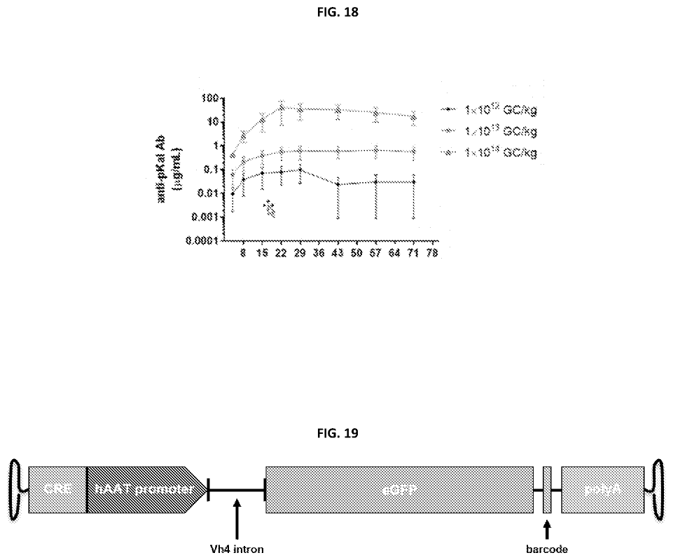

FIG. 19 is a schematic of the cassette construct used in the screening study. Each cassette is flanked by the canonical AAV2 inverted terminal repeats (ITRs). The promoter region is composed of the liver-specific hAAT promoter coupled with a Vh4 intron. Upstream of the hAAT promoter is one of the individual CRE candidate sequence (SEQ ID Nos: 163-293). As mentioned above, a unique 10 basepair DNA barcode is placed between the eGFP coding sequence and rabbit beta globin (RBG) polyadenylation signal inorder to identify which cassette was expressing the eGFP.

A Cis plasmid library containing a mixture of up to 55 cassettes was transfected with rep2/cap8 and helper plasmids, and thus packaged in AAV8, resulting in a pool concentration of 1.69e13 vg/mL. Barcodes were identified for even distribution within the pool.

The pool was then applied to Huh7 cells (expressing AAVR) to allow for transduction. The cells were then harvested.

A sample of the pool was also administered systemically to C57B16 mice (3 dose groups, 5 mice/group). Study animals were euthanized, and tissues were collected.

The AAV8 Liv-CRE vector library will also be produced in a manufacturing process at 2 L scale so that material can be administered systemically to two non-human primates for evaluation of expression of each vector in the pool.

### Example 14: Lanadelumab scFV-Fc Contructs

Several lanadelumab scFv-Fc constructs were made that have expression cassettes encoding scFv-Fc constructs with the heavy and light chain variable domains of lanadelumab linked by a flexible, non-cleavable linker and then linked to the lanadelumab hinge-Fc region by a flexible, non-cleavable linker. Constructs either have the arrangement N-LanVH-linker-Lan VL-linker-LanhingeFc-C or N-Lan VL-linker-LanVH-linker-LanhingeFc-C. The constructs include regulatory elements, including polyA signal sequences (SEQ ID NO: 305) and a chimeric intron (SEQ ID NO: 41) (see also Table 1). Promoter elements including ApoE.hAAT promoter (SEQ ID NO: 21) or LMTP6 promoter (SEQ ID NO: 14) are included (see also Table 1). The expression cassettes may be flanked by ITRs, including 5′ITR sequence of SEQ ID NO: 46 and 3′ITR sequence of SEQ ID NO: 307 (see Table 1). Exemplary constructs of Table 14 are depicted in FIG. 20. The amino acid and nucleotide sequences of the components, expressed scFv-Fcs and transgenes are provided in Table 14 below:

TABLE 14

| Name and SEQ ID | Sequence |
|---|---|
| Lanadelumab VH (codon- | gaggtccagctgcttgagagtggtggtggactggttcagcctggtg |
| optimized and CpG- | gcagcctgagactgtcttgtgctgcctctggcttcaccttcagcca |
| deleted) (SEQ ID NO: | ctatatcatgatgtgggtcagacaggcccctggcaaaggcctggaa |
| 313) | tgggtgtcaggcatctacagcagtggtggcatcacagtgtatgctg |
| | actctgtgaagggcagattcaccatcagcagagacaacagcaagaa |
| | caccctgtacctgcagatgaactccctgagagctgaggacacagca |
| | gtgtactactgtgcctatagaagaattggggtgcccagaagggatg |
| | agtttgacatctggggccagggcaccatggttacagtgtcctct |
| Lanadelumab VH (SEQ | EVQLLESGGGLVQPGGSLRLSCAASGFTFSHYIMMWVRQAPGKGLE |
| ID NO: 314) | WVSGIYSSGGITVYADSVKGRFTISRDNSKNTLYLQMNSLRAEDTA |
| | VYYCAYRRIGVPRRDEFDIWGQGTMVTVSS |
| Lanadelumab VL (codon- | gacatccagatgacacagagcccctccacactgtctgcatctgtgg |
| optimized and CpG- | gagacagagtgaccatcacctgtagagccagccagtctatctctag |
| deleted) (SEQ ID NO: | ctggctggcctggtatcagcaaaagcctggcaaggcccctaagctg |
| 317) | ctgatctacaaggccagcacacttgagtcaggggtgccctccagat |
| | tttctggctctggatctggcacagagttcaccctgaccatcagctc |
| | cctgcagccagatgactttgccacctactactgccagcagtacaat |
| | acctactggacctttggccagggaacaaaggtggaaatcaag |
| Lanadelumab VL (SEQ | DIQMTQSPSTLSASVGDRVTITCRASQSISSWLAWYQQKPGKAPKL |
| ID NO: 318) | LIYKASTLESGVPSRFSGSGSGTEFTLTISSLQPDDFATYYCQQYN |
| | TYWTFGQGTKVEIK |
| Lanadelumab Fc (codon- | Gaacccaagagctgtgacaagacccacacctgtcctccatgtcctg |
| optimized and CpG- | ctccagaactgcttggaggcccttctgtgttcctgtttcctccaaa |
| deleted) (SEQ ID NO: | gcctaaggacaccctgatgatcagcagaacccctgaagtgacctgt |
| 321) (*=stop codon) | gtggtggttgatgtgtcccatgaggacccagaagtgaagttcaatt |
| | ggtatgtggatggggttgaagtgcacaatgccaagaccaagcctag |
| | agaggaacagtacaacagcacctacagagtggtttctgtgctgaca |
| | gtgctgcaccaggactggctgaatggcaaagagtacaagtgcaagg |
| | tgtccaacaaggccctgcctgctcctattgagaaaaccatctccaa |
| | ggccaagggccagccaagagaaccccaggtttacaccctgccacct |
| | agcagagaagagatgaccaagaaccaggtgtccctgacctgcctgg |
| | ttaagggcttctacccctctgacattgctgtggaatgggagagcaa |
| | tggccagcctgaaaacaactacaagacaacccctcctgtgctggac |
| | tctgatggctcattcttcctgtacagcaagctgactgtggacaagt |
| | ccagatggcagcagggaaatgtgttcagctgctctgtgatgcatga |
| | ggccctgcacaaccactacacccagaaaagtctgtctctgtcccct |
| | ggc* |
| Lanadelumab Fc (SEQ ID | EPKSCDKTHTCPPCPAPELLGGPSVFLFPPKPKDTLMISRTPEVTC |
| NO: 322) | VVVDVSHEDPEVKFNWYVDGVEVHNAKTKPREEQYNSTYRVVSVLT |
| | VLHQDWLNGKEYKCKVSNKALPAPIEKTISKAKGQPREPQVYTLPP |
| | SREEMTKNQVSLTCLVKGFYPSDIAVEWESNGQPENNYKTTPPVLD |
| | SDGSFFLYSKLTVDKSRWQQGNVFSCSVMHEALHNHYTQKSLSLSP |
| | G |
| VH-VL-Fc sequence | atgtacagaatgcagctgctgctgctcattgccctgtctctggccc |
| (including leader and | tggtcaccaattctgaggaggtccagctgcttgagagtggtggtgg |
| linkers) (SEQ ID NO: | actggttcagcctggtggcagcctgagactgtcttgtgctgcctct |
| 323) (*Stop Codon) | ggcttcaccttcagccactatatcatgatgtgggtcagacaggccc |
| | ctggcaaaggcctggaatgggtgtcaggcatctacagcagtggtgg |
| | catcacagtgtatgctgactctgtgaagggcagattcaccatcagc |
| | agagacaacagcaagaacaccctgtacctgcagatgaactccctga |
| | gagctgaggacacagcagtgtactactgtgcctatagaagaattgg |
| | ggtgcccagaagggatgagtttgacatctggggccagggcaccatg |
| | gttacagtgtcctctggtggaggcggttcaggcggaggtggcagcg |
| | gcggtggagggtcggacatccagatgacacagagcccctccacact |
| | gtctgcatctgtgggagacagagtgaccatcacctgtagagccagc |
| | cagtctatctctagctggctggcctggtatcagcaaaagcctggca |
| | aggcccctaagctgctgatctacaaggccagcacacttgagtcagg |
| | ggtgccctccagattttctggctctggatctggcacagagttcacc |
| | ctgaccatcagctccctgcagccagatgactttgccacctactact |
| | gccagcagtacaatacctactggacctttggccagggaacaaaggt |
| | ggaaatcaagggcggaggaggcggaggtggaggaggagaacccaag |
| | agctgtgacaagacccacacctgtcctccatgtcctgctccagaac |
| | tgcttggaggcccttctgtgttcctgtttcctccaaagcctaagga |
| | caccctgatgatcagcagaacccctgaagtgacctgtgtggtggtt |
| | gatgtgtcccatgaggacccagaagtgaagttcaattggtatgtgg |
| | atggggttgaagtgcacaatgccaagaccaagcctagagaggaaca |
| | gtacaacagcacctacagagtggtttctgtgctgacagtgctgcac |
| | caggactggctgaatggcaaagagtacaagtgcaaggtgtccaaca |
| | aggccctgcctgctcctattgagaaaaccatctccaaggccaaggg |
| | ccagccaagagaaccccaggtttacaccctgccacctagcagagaa |
| | gagatgaccaagaaccaggtgtccctgacctgcctggttaagggct |
| | tctacccctctgacattgctgtggaatgggagagcaatggccagcc |
| | tgaaaacaactacaagacaacccctcctgtgctggactctgatggc |
| | tcattcttcctgtacagcaagctgactgtggacaagtccagatggc |
| | agcagggaaatgtgttcagctgctctgtgatgcatgaggccctgca |
| | caaccactacacccagaaaagtctgtctctgtcccctggc* |
| VH-VL-Fc (including | MYRMQLLLLIALSLALVINSEEVQLLESGGGLVQPGGSLRLSCAAS |
| leader and linkers) (SEQ | GFTFSHYIMMWVRQAPGKGLEWVSGIYSSGGITVYADSVKGRATIS |
| ID NO: 324) | RDNSKNTLYLQMNSLRAEDTAVYYCAYRRIGVPRRDEFDIWGQGTM |
| | VTVSSGGGGSGGGGSGGGGSDIQMTQSPSTLSASVGDRVTITCRAS |
| | QSISSWLAWYQQKPGKAPKLLIYKASTLESGVPSRFSGSGSGTEFT |
| | LTISSLQPDDFATYYCQQYNTYWTFGQGTKVEIKGGGGGGGGGEPK |
| | SCDKTHTCPPCPAPELLGGPSVFLFPPKPKDTLMISRTPEVTCVVV |
| | DVSHEDPEVKENWYVDGVEVHNAKTKPREEQYNSTYRVVSVLTVLH |
| | QDWLNGKEYKCKVSNKALPAPIEKTISKAKGQPREPQVYTLPPSRE |
| | EMTKNQVSLTCLVKGFYPSDIAVEWESNGQPENNYKTTPPVLDSDG |
| | SFFLYSKLTVDKSRWQQGNVFSCSVMHEALHNHYTQKSLSLSPG |
| VL-VH-Fc sequence | atgtacagaatgcagctgctgctgctcattgccctgtctctggccc |
| (including leader and | tggtcaccaattctgaggacatccagatgacacagagcccctccac |
| linkers) (SEQ ID NO: 392) | actgtctgcatctgtgggagacagagtgaccatcacctgtagagcc |
| | agccagtctatctctagctggctggcctggtatcagcaaaagcctg |
| | gcaaggcccctaagctgctgatctacaaggccagcacacttgagtc |
| | aggggtgccctccagattttctggctctggatctggcacagagttc |
| | accctgaccatcagctccctgcagccagatgactttgccacctact |
| | actgccagcagtacaatacctactggacctttggccagggaacaaa |
| | ggtggaaatcaagggtggaggcggttcaggcggaggtggcagcggc |
| | ggtggagggtcggaggtccagctgcttgagagtggtggtggactgg |
| | ttcagcctggtggcagcctgagactgtcttgtgctgcctctggctt |
| | caccttcagccactatatcatgatgtgggtcagacaggcccctggc |
| | aaaggcctggaatgggtgtcaggcatctacagcagtggtggcatca |
| | cagtgtatgctgactctgtgaagggcagattcaccatcagcagaga |
| | caacagcaagaacaccctgtacctgcagatgaactccctgagagct |
| | gaggacacagcagtgtactactgtgcctatagaagaattggggtgc |
| | ccagaagggatgagtttgacatctggggccagggcaccatggttac |
| | agtgtcctctggcggaggaggcggaggtggaggaggagaacccaag |
| | agctgtgacaagacccacacctgtcctccatgtcctgctccagaac |
| | tgcttggaggcccttctgtgttcctgtttcctccaaagcctaagga |
| | caccctgatgatcagcagaacccctgaagtgacctgtgtggtggtt |
| | gatgtgtcccatgaggacccagaagtgaagttcaattggtatgtgg |
| | atggggttgaagtgcacaatgccaagaccaagcctagagaggaaca |
| | gtacaacagcacctacagagtggtttctgtgctgacagtgctgcac |
| | caggactggctgaatggcaaagagtacaagtgcaaggtgtccaaca |
| | aggccctgcctgctcctattgagaaaaccatctccaaggccaaggg |
| | ccagccaagagaaccccaggtttacaccctgccacctagcagagaa |
| | gagatgaccaagaaccaggtgtccctgacctgcctggttaagggct |
| | tctacccctctgacattgctgtggaatgggagagcaatggccagcc |
| | tgaaaacaactacaagacaacccctcctgtgctggactctgatggc |
| | tcattcttcctgtacagcaagctgactgtggacaagtccagatggc |
| | agcagggaaatgtgttcagctgctctgtgatgcatgaggccctgca |
| | caaccactacacccagaaaagtctgtctctgtcccctggc |
| VL-VH-Fc Sequence | MYRMQLLLLIALSLALVINSEDIQMTQSPSTLSASVGDRVTITCRA |
| (SEQ ID NO: 393) | SQSISSWLAWYQQKPGKAPKLLIYKASTLESGVPSRFSGSGSGTEF |
| | TLTISSLQPDDFATYYCQQYNTYWTFGQGTKVEIKGGGGSGGGGSG |
| | GGGSEVQLLESGGGLVQPGGSLRLSCAASGFTFSHYIMMWVRQAPG |
| | KGLEWVSGIYSSGGITVYADSVKGRFTISRDNSKNTLYLQMNSLRA |
| | EDTAVYYCAYRRIGVPRRDEFDIWGQGTMVTVSSGGGGGGGGGEPK |
| | SCDKTHTCPPCPAPELLGGPSVFLFPPKPKDTLMISRTPEVTCVVV |
| | DVSHEDPEVKFNWYVDGVEVHNAKTKPREEQYNSTYRVVSVLTVLH |
| | QDWLNGKEYKCKVSNKALPAPIEKTISKAKGQPREPQVYTLPPSRE |
| | EMTKNQVSLTCLVKGFYPSDIAVEWESNGQPENNYKTTPPVLDSDG |
| | SFFLYSKLTVDKSRWQQGNVFSCSVMHEALHNHYTQKSLSLSPG |
| ApoE.hAAT.HL-scFv-Fc | ctgcgcgctcgctcgctcactgaggccgcccgggcaaagcccgggc |
| 5′ITR-ApoE.hAAT | gtcgggcgacctttggtcgcccggcctcagtgagcgagcgagcgcg |
| promoter-chimeric | cagagagggagtggccaactccatcactaggggttccttgtagtta |
| intron-leader-VH-4GSx3 | atgattaacccgccatgctacttatctaccagggtaatggggatcc |
| linker-VL-9G linker-Fc- | tctagaaggctcagaggcacacaggagtttctgggctcaccctgcc |
| poly A-3′ITR | cccttccaacccctcagttcccatcctccagcagctgtttgtgtgc |
| 3005 nucleotides | tgcctctgaagtccacactgaacaaacttcagcctactcatgtccc |
| (SEQ ID NO: 308) | taaaatgggcaaacattgcaagcagcaaacagcaaacacacagccc |
| | tccctgcctgctgaccttggagctggggcagaggtcagagacctct |
| | ctgggcccatgccacctccaacatccactcgaccccttggaatttc |
| | ggtggagaggagcagaggttgtcctggcgtggtttaggtagtgtga |
| | gaggggtacccggggatcttgctaccagtggaacagccactaagga |
| | ttctgcagtgagagcagagggccagctaagtggtactctcccagag |
| | actgtctgactcacgccaccccctccaccttggacacaggacgctg |
| | tggtttctgagccaggtacaatgactcctttcggtaagtgcagtgg |
| | aagctgtacactgcccaggcaaagcgtccgggcagcgtaggcgggc |
| | gactcagatcccagccagtggacttagcccctgtttgctcctccga |
| | taactggggtgaccttggttaatattcaccagcagcctcccccgtt |
| | gcccctctggatccactgcttaaatacggacgaggacagggccctg |
| | tctcctcagcttcaggcaccaccactgacctgggacagtcaggtaa |
| | gtatcaaggttacaagacaggtttaaggagaccaatagaaactggg |
| | cttgtcgagacagagaagactcttgcgtttctgataggcacctatt |
| | ggtcttactgacatccactttgcctttctctccacaggttgaattc |
| | gccaccatgtacagaatgcagctgctgctgctcattgccctgtctc |
| | tggccctggtcaccaattctgaggaggtccagctgcttgagagtgg |
| | tggtggactggttcagcctggtggcagcctgagactgtcttgtgct |
| | gcctctggcttcaccttcagccactatatcatgatgtgggtcagac |
| | aggcccctggcaaaggcctggaatgggtgtcaggcatctacagcag |
| | tggtggcatcacagtgtatgctgactctgtgaagggcagattcacc |
| | atcagcagagacaacagcaagaacaccctgtacctgcagatgaact |
| | ccctgagagctgaggacacagcagtgtactactgtgcctatagaag |
| | aattggggtgcccagaagggatgagtttgacatctggggccagggc |
| | accatggttacagtgtcctctggtggaggcggttcaggcggaggtg |
| | gcagcggcggtggagggtcggacatccagatgacacagagcccctc |
| | cacactgtctgcatctgtgggagacagagtgaccatcacctgtaga |
| | gccagccagtctatctctagctggctggcctggtatcagcaaaagc |
| | ctggcaaggcccctaagctgctgatctacaaggccagcacacttga |
| | gtcaggggtgccctccagattttctggctctggatctggcacagag |
| | ttcaccctgaccatcagctccctgcagccagatgactttgccacct |
| | actactgccagcagtacaatacctactggacctttggccagggaac |
| | aaaggtggaaatcaagggcggaggaggcggaggtggaggaggagaa |
| | cccaagagctgtgacaagacccacacctgtcctccatgtcctgctc |
| | cagaactgcttggaggcccttctgtgttcctgtttcctccaaagcc |
| | taaggacaccctgatgatcagcagaacccctgaagtgacctgtgtg |
| | gtggttgatgtgtcccatgaggacccagaagtgaagttcaattggt |
| | atgtggatggggttgaagtgcacaatgccaagaccaagcctagaga |
| | ggaacagtacaacagcacctacagagtggtttctgtgctgacagtg |
| | ctgcaccaggactggctgaatggcaaagagtacaagtgcaaggtgt |
| | ccaacaaggccctgcctgctcctattgagaaaaccatctccaaggc |
| | caagggccagccaagagaaccccaggtttacaccctgccacctagc |
| | agagaagagatgaccaagaaccaggtgtccctgacctgcctggtta |
| | agggcttctacccctctgacattgctgtggaatgggagagcaatgg |
| | ccagcctgaaaacaactacaagacaacccctcctgtgctggactct |
| | gatggctcattcttcctgtacagcaagctgactgtggacaagtcca |
| | gatggcagcagggaaatgtgttcagctgctctgtgatgcatgaggc |
| | cctgcacaaccactacacccagaaaagtctgtctctgtcccctggc |
| | taatgactcgaggacggggtgaactacgcctgaggatccgatcttt |
| | ttccctctgccaaaaattatggggacatcatgaagccccttgagca |
| | tctgacttctggctaataaaggaaatttattttcattgcaatagtg |
| | tgttggaattttttgtgtctctcactcggcggccgcccgagtttaa |
| | ttggtttatagaactcttcaagctagcgaagcaattcgttgatctg |
| | aatttcgaccacccataatacccattaccctggtagataagtagca |
| | tggcgggttaatcattaactacaaggaacccctagtgatggagttg |
| | gccactccctctctgcgcgctcgctcgctcactgaggccgggcgac |
| | caaaggtcgcccgacgcccgggctttgcccgggcggcctcagtgag |
| | cgagcgagcgcgcag |
| LMTP6.HL-scFv-Fc | ctgcgcgctcgctcgctcactgaggccgcccgggcaaagcccgggc |
| 5′ITR-LMTP6 promoter- | gtcgggcgacctttggtcgcccggcctcagtgagcgagcgagcgcg |
| chimeric intron - leader- | cagagagggagtggccaactccatcactaggggttccttgtagtta |
| VH-4GSx3 linker-VL- | atgattaacccgccatgctacttatctaccagggtaatggggatcc |
| 9G linker-Fc-polyA- | tctagaaggctcagaggcacacaggagtttctgggctcaccctgcc |
| 3′ITR (SEQ ID NO: 325) | cccttccaacccctcagttcccatcctccagcagctgtttgtgtgc |
| 3862 nucleotides | tgcctctgaagtccacactgaacaaacttcagcctactcatgtccc |
| | taaaatgggcaaacattgcaagcagcaaacagcaaacacacagccc |
| | tccctgcctgctgaccttggagctggggcagaggtcagagacctct |
| | ctgggcccatgccacctccaacatccactcgaccccttggaatttc |
| | ggtggagaggagcagaggttgtcctggcgtggtttaggtagtgtga |
| | gagggccactacgggtttaggctgcccatgtaaggaggcaaggcct |
| | ggggacacccgagatgcctggttataattaacccagacatgtggct |
| | gccccccccccccccaacacctgctgcctctaaaaataaccctgtc |
| | cctggtggatcccactacgggtttaggctgcccatgtaaggaggca |
| | aggcctggggacacccgagatgcctggttataattaacccagacat |
| | gtggctgccccccccccccccaacacctgctgcctctaaaaataac |
| | cctgtccctggtggatcccactacgggtttaggctgcccatgtaag |
| | gaggcaaggcctggggacacccgagatgcctggttataattaaccc |
| | agacatgtggctgccccccccccccccaacacctgctgcctctaaa |
| | aataaccctgtccctggtggatcccctgcatgcgaagatcttcgaa |
| | caaggctgtgggggactgagggcaggctgtaacaggcttgggggcc |
| | agggcttatacgtgcctgggactcccaaagtattactgttccatgt |
| | tcccggcgaagggccagctgtcccccgccagctagactcagcactt |
| | agtttaggaaccagtgagcaagtcagcccttggggcagcccataca |
| | aggccatggggctgggcaagctgcacgcctgggtccggggtgggca |
| | cggtgcccgggcaacgagctgaaagctcatctgctctcaggggccc |
| | ctccctggggacagcccctcctggctagtcacaccctgtaggctcc |
| | tctatataacccaggggcacaggggctgccctcattctaccaccac |
| | ctccacagcacagacagacactcaggagccagccagcgtcgagatc |
| | ttgctaccagtggaacagccactaaggattctgcagtgagagcaga |
| | gggccagctaagtggtactctcccagagactgtctgactcacgcca |
| | ccccctccaccttggacacaggacgctgtggtttctgagccaggta |
| | cagtgactcctttcggtaagtgcagtggaagctgtacactgcccag |
| | gcaaagcgtccgggcagcgtaggcgggcgactcagatcccagccag |
| | tggacttagcccctgtttgctcctccgataactggggtgaccttgg |
| | ttaatattcaccagcagcctcccccgttgcccctctggatccactg |
| | cttaaatacggacgaggacagggccctgtctcctcagcttcaggca |
| | ccaccactgacctgggacagtcaggtaagtatcaaggttacaagac |
| | aggtttaaggagaccaatagaaactgggcttgtcgagacagagaag |
| | actcttgcgtttctgataggcacctattggtcttactgacatccac |
| | tttgcctttctctccacaggttcgaattcgccaccatgtacagaat |
| | gcagctgctgctgctcattgccctgtctctggccctggtcaccaat |
| | tctgaggaggtccagctgcttgagagtggtggtggactggttcagc |
| | ctggtggcagcctgagactgtcttgtgctgcctctggcttcacctt |
| | cagccactatatcatgatgtgggtcagacaggcccctggcaaaggc |
| | ctggaatgggtgtcaggcatctacagcagtggtggcatcacagtgt |
| | atgctgactctgtgaagggcagattcaccatcagcagagacaacag |
| | caagaacaccctgtacctgcagatgaactccctgagagctgaggac |
| | acagcagtgtactactgtgcctatagaagaattggggtgcccagaa |
| | gggatgagtttgacatctggggccagggcaccatggttacagtgtc |
| | ctctggtggaggcggttcaggcggaggtggcagcggcggtggaggg |
| | tcggacatccagatgacacagagcccctccacactgtctgcatctg |
| | tgggagacagagtgaccatcacctgtagagccagccagtctatctc |
| | tagctggctggcctggtatcagcaaaagcctggcaaggcccctaag |
| | ctgctgatctacaaggccagcacacttgagtcaggggtgccctcca |
| | gattttctggctctggatctggcacagagttcaccctgaccatcag |
| | ctccctgcagccagatgactttgccacctactactgccagcagtac |
| | aatacctactggacctttggccagggaacaaaggtggaaatcaagg |
| | gcggaggaggcggaggtggaggaggagaacccaagagctgtgacaa |
| | gacccacacctgtcctccatgtcctgctccagaactgcttggaggc |
| | ccttctgtgttcctgtttcctccaaagcctaaggacaccctgatga |
| | tcagcagaacccctgaagtgacctgtgtggtggttgatgtgtccca |
| | tgaggacccagaagtgaagttcaattggtatgtggatggggttgaa |
| | gtgcacaatgccaagaccaagcctagagaggaacagtacaacagca |
| | cctacagagtggtttctgtgctgacagtgctgcaccaggactggct |
| | gaatggcaaagagtacaagtgcaaggtgtccaacaaggccctgcct |
| | gctcctattgagaaaaccatctccaaggccaagggccagccaagag |
| | aaccccaggtttacaccctgccacctagcagagaagagatgaccaa |
| | gaaccaggtgtccctgacctgcctggttaagggcttctacccctct |
| | gacattgctgtggaatgggagagcaatggccagcctgaaaacaact |
| | acaagacaacccctcctgtgctggactctgatggctcattcttcct |
| | gtacagcaagctgactgtggacaagtccagatggcagcagggaaat |
| | gtgttcagctgctctgtgatgcatgaggccctgcacaaccactaca |
| | cccagaaaagtctgtctctgtcccctggctaatgactcgaggacgg |
| | ggtgaactacgcctgaggatccgatctttttccctctgccaaaaat |
| | tatggggacatcatgaagccccttgagcatctgacttctggctaat |
| | aaaggaaatttattttcattgcaatagtgtgttggaattttttgtg |
| | tctctcactcggcggccgcccgagtttaattggtttatagaactct |
| | tcaagctagcgaagcaattcgttgatctgaatttcgaccacccata |
| | atacccattaccctggtagataagtagcatggcgggttaatcatta |
| | actacaaggaacccctagtgatggagttggccactccctctctgcg |
| | cgctcgctcgctcactgaggccgggcgaccaaaggtcgcccgacgc |
| | ccgggctttgcccgggcggcctcagtgagcgagcgagcgcgcag |
| ApoE.hAAT.LH-scFv-Fc | ctgcgcgctcgctcgctcactgaggccgcccgggcaaagcccgggc |
| 5′ITR-ApoE.hAAT | gtcgggcgacctttggtcgcccggcctcagtgagcgagcgagcgcg |
| promoter-chimeric | cagagagggagtggccaactccatcactaggggttccttgtagtta |
| intron-leader-VL-4GSx3 | atgattaacccgccatgctacttatctaccagggtaatggggatcc |
| linker-VH-9G linker-Fc- | tctagaaggctcagaggcacacaggagtttctgggctcaccctgcc |
| polyA-3′ITR (SEQ ID | cccttccaacccctcagttcccatcctccagcagctgtttgtgtgc |
| NOL 332) | tgcctctgaagtccacactgaacaaacttcagcctactcatgtccc |
| 3005 nucleotides | taaaatgggcaaacattgcaagcagcaaacagcaaacacacagccc |
| | tccctgcctgctgaccttggagctggggcagaggtcagagacctct |
| | ctgggcccatgccacctccaacatccactcgaccccttggaatttc |
| | ggtggagaggagcagaggttgtcctggcgtggtttaggtagtgtga |
| | gaggggtacccggggatcttgctaccagtggaacagccactaagga |
| | ttctgcagtgagagcagagggccagctaagtggtactctcccagag |
| | actgtctgactcacgccaccccctccaccttggacacaggacgctg |
| | tggtttctgagccaggtacaatgactcctttcggtaagtgcagtgg |
| | aagctgtacactgcccaggcaaagcgtccgggcagcgtaggcgggc |
| | gactcagatcccagccagtggacttagcccctgtttgctcctccga |
| | taactggggtgaccttggttaatattcaccagcagcctcccccgtt |
| | gcccctctggatccactgcttaaatacggacgaggacagggccctg |
| | tctcctcagcttcaggcaccaccactgacctgggacagtcaggtaa |
| | gtatcaaggttacaagacaggtttaaggagaccaatagaaactggg |
| | cttgtcgagacagagaagactcttgcgtttctgataggcacctatt |
| | ggtcttactgacatccactttgcctttctctccacaggttgaattc |
| | gccaccatgtacagaatgcagctgctgctgctcattgccctgtctc |
| | tggccctggtcaccaattctgaggacatccagatgacacagagccc |
| | ctccacactgtctgcatctgtgggagacagagtgaccatcacctgt |
| | agagccagccagtctatctctagctggctggcctggtatcagcaaa |
| | agcctggcaaggcccctaagctgctgatctacaaggccagcacact |
| | tgagtcaggggtgccctccagattttctggctctggatctggcaca |
| | gagttcaccctgaccatcagctccctgcagccagatgactttgcca |
| | cctactactgccagcagtacaatacctactggacctttggccaggg |
| | aacaaaggtggaaatcaagggtggaggcggttcaggcggaggtggc |
| | agcggcggtggagggtcggaggtccagctgcttgagagtggtggtg |
| | gactggttcagcctggtggcagcctgagactgtcttgtgctgcctc |
| | tggcttcaccttcagccactatatcatgatgtgggtcagacaggcc |
| | cctggcaaaggcctggaatgggtgtcaggcatctacagcagtggtg |
| | gcatcacagtgtatgctgactctgtgaagggcagattcaccatcag |
| | cagagacaacagcaagaacaccctgtacctgcagatgaactccctg |
| | agagctgaggacacagcagtgtactactgtgcctatagaagaattg |
| | gggtgcccagaagggatgagtttgacatctggggccagggcaccat |
| | ggttacagtgtcctctggcggaggaggcggaggtggaggaggagaa |
| | cccaagagctgtgacaagacccacacctgtcctccatgtcctgctc |
| | cagaactgcttggaggcccttctgtgttcctgtttcctccaaagcc |
| | taaggacaccctgatgatcagcagaacccctgaagtgacctgtgtg |
| | gtggttgatgtgtcccatgaggacccagaagtgaagttcaattggt |
| | atgtggatggggttgaagtgcacaatgccaagaccaagcctagaga |
| | ggaacagtacaacagcacctacagagtggtttctgtgctgacagtg |
| | ctgcaccaggactggctgaatggcaaagagtacaagtgcaaggtgt |
| | ccaacaaggccctgcctgctcctattgagaaaaccatctccaaggc |
| | caagggccagccaagagaaccccaggtttacaccctgccacctagc |
| | agagaagagatgaccaagaaccaggtgtccctgacctgcctggtta |
| | agggcttctacccctctgacattgctgtggaatgggagagcaatgg |
| | ccagcctgaaaacaactacaagacaacccctcctgtgctggactct |
| | gatggctcattcttcctgtacagcaagctgactgtggacaagtcca |
| | gatggcagcagggaaatgtgttcagctgctctgtgatgcatgaggc |
| | cctgcacaaccactacacccagaaaagtctgtctctgtcccctggc |
| | taatgactcgaggacggggtgaactacgcctgaggatccgatcttt |
| | ttccctctgccaaaaattatggggacatcatgaagccccttgagca |
| | tctgacttctggctaataaaggaaatttattttcattgcaatagtg |
| | tgttggaattttttgtgtctctcactcggcggccgcccgagtttaa |
| | ttggtttatagaactcttcaagctagcgaagcaattcgttgatctg |
| | aatttcgaccacccataatacccattaccctggtagataagtagca |
| | tggcgggttaatcattaactacaaggaacccctagtgatggagttg |
| | gccactccctctctgcgcgctcgctcgctcactgaggccgggcgac |
| | caaaggtcgcccgacgcccgggctttgcccgggcggcctcagtgag |
| | cgagcgagcgcgcag |
| LMTP6.LH-scFv-Fc | ctgcgcgctcgctcgctcactgaggccgcccgggcaaagcccgggc |
| 5′ITR-LMTP6 promoter- | gtcgggcgacctttggtcgcccggcctcagtgagcgagcgagcgcg |
| chimeric intron - leader- | cagagagggagtggccaactccatcactaggggttccttgtagtta |
| VL-4GSx3 linker-VH- | atgattaacccgccatgctacttatctaccagggtaatggggatcc |
| 9G linker-Fc-polyA- | tctagaaggctcagaggcacacaggagtttctgggctcaccctgcc |
| 3′ITR (SEQ ID NO: 333) | cccttccaacccctcagttcccatcctccagcagctgtttgtgtgc |
| 3862 nucleotides | tgcctctgaagtccacactgaacaaacttcagcctactcatgtccc |
| | taaaatgggcaaacattgcaagcagcaaacagcaaacacacagccc |
| | tccctgcctgctgaccttggagctggggcagaggtcagagacctct |
| | ctgggcccatgccacctccaacatccactcgaccccttggaatttc |
| | ggtggagaggagcagaggttgtcctggcgtggtttaggtagtgtga |
| | gagggccactacgggtttaggctgcccatgtaaggaggcaaggcct |
| | ggggacacccgagatgcctggttataattaacccagacatgtggct |
| | gccccccccccccccaacacctgctgcctctaaaaataaccctgtc |
| | cctggtggatcccactacgggtttaggctgcccatgtaaggaggca |
| | aggcctggggacacccgagatgcctggttataattaacccagacat |
| | gtggctgccccccccccccccaacacctgctgcctctaaaaataac |
| | cctgtccctggtggatcccactacgggtttaggctgcccatgtaag |
| | gaggcaaggcctggggacacccgagatgcctggttataattaaccc |
| | agacatgtggctgccccccccccccccaacacctgctgcctctaaa |
| | aataaccctgtccctggtggatcccctgcatgcgaagatcttcgaa |
| | caaggctgtgggggactgagggcaggctgtaacaggcttgggggcc |
| | agggcttatacgtgcctgggactcccaaagtattactgttccatgt |
| | tcccggcgaagggccagctgtcccccgccagctagactcagcactt |
| | agtttaggaaccagtgagcaagtcagcccttggggcagcccataca |
| | aggccatggggctgggcaagctgcacgcctgggtccggggtgggca |
| | cggtgcccgggcaacgagctgaaagctcatctgctctcaggggccc |
| | ctccctggggacagcccctcctggctagtcacaccctgtaggctcc |
| | tctatataacccaggggcacaggggctgccctcattctaccaccac |
| | ctccacagcacagacagacactcaggagccagccagcgtcgagatc |
| | ttgctaccagtggaacagccactaaggattctgcagtgagagcaga |
| | gggccagctaagtggtactctcccagagactgtctgactcacgcca |
| | ccccctccaccttggacacaggacgctgtggtttctgagccaggta |
| | cagtgactcctttcggtaagtgcagtggaagctgtacactgcccag |
| | gcaaagcgtccgggcagcgtaggcgggcgactcagatcccagccag |
| | tggacttagcccctgtttgctcctccgataactggggtgaccttgg |
| | ttaatattcaccagcagcctcccccgttgcccctctggatccactg |
| | cttaaatacggacgaggacagggccctgtctcctcagcttcaggca |
| | ccaccactgacctgggacagtcaggtaagtatcaaggttacaagac |
| | aggtttaaggagaccaatagaaactgggcttgtcgagacagagaag |
| | actcttgcgtttctgataggcacctattggtcttactgacatccac |
| | tttgcctttctctccacaggttcgaattcgccaccatgtacagaat |
| | gcagctgctgctgctcattgccctgtctctggccctggtcaccaat |
| | tctgaggacatccagatgacacagagcccctccacactgtctgcat |
| | ctgtgggagacagagtgaccatcacctgtagagccagccagtctat |
| | ctctagctggctggcctggtatcagcaaaagcctggcaaggcccct |
| | aagctgctgatctacaaggccagcacacttgagtcaggggtgccct |
| | ccagattttctggctctggatctggcacagagttcaccctgaccat |
| | cagctccctgcagccagatgactttgccacctactactgccagcag |
| | tacaatacctactggacctttggccagggaacaaaggtggaaatca |
| | agggtggaggcggttcaggcggaggtggcagcggcggtggagggtc |
| | ggaggtccagctgcttgagagtggtggtggactggttcagcctggt |
| | ggcagcctgagactgtcttgtgctgcctctggcttcaccttcagcc |
| | actatatcatgatgtgggtcagacaggcccctggcaaaggcctgga |
| | atgggtgtcaggcatctacagcagtggtggcatcacagtgtatgct |
| | gactctgtgaagggcagattcaccatcagcagagacaacagcaaga |
| | acaccctgtacctgcagatgaactccctgagagctgaggacacagc |
| | agtgtactactgtgcctatagaagaattggggtgcccagaagggat |
| | gagtttgacatctggggccagggcaccatggttacagtgtcctctg |
| | gcggaggaggcggaggtggaggaggagaacccaagagctgtgacaa |
| | gacccacacctgtcctccatgtcctgctccagaactgcttggaggc |
| | ccttctgtgttcctgtttcctccaaagcctaaggacaccctgatga |
| | tcagcagaacccctgaagtgacctgtgtggtggttgatgtgtccca |
| | tgaggacccagaagtgaagttcaattggtatgtggatggggttgaa |
| | gtgcacaatgccaagaccaagcctagagaggaacagtacaacagca |
| | cctacagagtggtttctgtgctgacagtgctgcaccaggactggct |
| | gaatggcaaagagtacaagtgcaaggtgtccaacaaggccctgcct |
| | gctcctattgagaaaaccatctccaaggccaagggccagccaagag |
| | aaccccaggtttacaccctgccacctagcagagaagagatgaccaa |
| | gaaccaggtgtccctgacctgcctggttaagggcttctacccctct |
| | gacattgctgtggaatgggagagcaatggccagcctgaaaacaact |
| | acaagacaacccctcctgtgctggactctgatggctcattcttcct |
| | gtacagcaagctgactgtggacaagtccagatggcagcagggaaat |
| | gtgttcagctgctctgtgatgcatgaggccctgcacaaccactaca |
| | cccagaaaagtctgtctctgtcccctggctaatgactcgaggacgg |
| | ggtgaactacgcctgaggatccgatctttttccctctgccaaaaat |
| | tatggggacatcatgaagccccttgagcatctgacttctggctaat |
| | aaaggaaatttattttcattgcaatagtgtgttggaattttttgtg |
| | tctctcactcggcggccgcccgagtttaattggtttatagaactct |
| | tcaagctagcgaagcaattcgttgatctgaatttcgaccacccata |
| | atacccattaccctggtagataagtagcatggcgggttaatcatta |
| | actacaaggaacccctagtgatggagttggccactccctctctgcg |
| | cgctcgctcgctcactgaggccgggcgaccaaaggtcgcccgacgc |
| | ccgggctttgcccgggcggcctcagtgagcgagcgagcgcgcag |

Expression of both the scFv-Fc protein constructs were confirmed by SDS-PAGE and LC-MS.

The binding affinities of Lanadelumab antibodies and scFv-Fc proteins is shown in FIG. 21A (lanadelumab full length antibodies) and FIG. 21B (scFv-Fcs). This study was performed to measure the binding affinity of antibodies to human kallikrein using Biacore T200. The assay was performed at 25° C. and the running buffer was HBS-EP+. Diluted antibodies were captured on the sensor chip through Fc capture method. Human kallikrein was used as the analyte, followed by injecting running buffer as dissociation phase. All the data were processed using the Biacore T200 evaluation software version 3.1. Flow cell 1 and blank injection of buffer in each cycle were used as double reference for Response Units subtraction. The binding kinetic data i shown in the bottom tables of FIGS. 21A and B and the binding sensor-grams are shown in the graphs of FIGS. 21A and B. The binding parameters for the Lanadelumab (LAN-) VH-VL-Fc and VL-VH-Fc proteins (SEQ ID NO: 324 and SEQ ID NO: 393, respectively) were comparable to the full length antibody. The full length antibody had a K_{D} of 1.74×10⁻⁹ M, while the LAN-VH-VL-Fc scFv-Fc had a K_{D} of 1.81×10⁻⁹ M and the LAN-VL-VH-Fc construct scFv-Fc had a K_{D} of 1.39×10⁻⁹ M.

FIG. 22 shows the results of the relative production levels of various Lanadelumab scFv-Fc constructs. Huh7 cells were seeded at 5×10⁵ cells/well in 6-well plate the day prior. Plasmids were transfected at 2.5 ug/well with Lipofectamine 3000 system. The media was changed to Opti-MEM (serum-free). On day 4 the supernatant and cells were harvested. 2 mL of supernatant was retained. Cell lysate was collected with 2 mL/well M-PER+protease inhibitor (1×)+5 mM EDTA lysed on ice for 10 mins. ELISAs were performed using the supernatant and cell lysates. Wells were coated w 1 ug/mL human kallikrein (activated, purified from human plasma). 1:10,000 dilution of HRP-conjugated AffiniPure Goat Anti-Human IgG, Fc Fragment Specific (min X Bovine, Horse, and Mouse Serum Proteins) from Jackson ImmunoResearch was used for detection.

FIG. 22 shows the results of the production of the four scFv-Fc constructs ApoE.hAAT.HL-scFv-Fc, LMTP6.HL-scFv-Fc, ApoE.hAAT.LH-scFv-Fc and LMTP6.LH-scFv-Fc. The data show that ApoE.hAAT.HL-scFv-FcRGX2281 is in both the supernatant and cell lysate fractions.

### Example 15: Mouse Study Protocol

Purpose: The objective of this study is to evaluate different AAV-HAE vectors for gene expression and biodistribution.

Study Synopsis: Thirty-three (33) C57BL/6 Female mice will be assigned to the study. The mice will be distributed into Seven (7) groups. Control Vehicle at 100 ul will be administered to Group 1 mice via intravenous route. VC-119 at 100 ul will be administered to Group 2 mice via intravenous route. VC-120 at 100 ul will be administered to Group 3 mice via intravenous route. VC-121 at 100 ul will be administered to Group 4 mice via intravenous route. VC-122 at 100 ul will be administered to Group 5 mice via intravenous route. HAE012 at 100 ul will be administered to Group 6 mice via intravenous route. HAE014 at 100 ul will be administered to Group 7 mice via intravenous route. Groups 2-7 mice will be dosed at 1×10¹² GC/kg body weight on study day 0. Blood will be collected on study day −7, 7 and 14 and 28 via retro-orbital collections and collected into SSTs for serum processing. Mice will be humanely euthanized on Day 28 post dosing. All animals will be perfused with sterile 1× cold PBS (free of DNAse and RNAse) to clear blood from tissues. For all the snap frozen samples, 2 ml round bottom microcentrifuge tubes will be used and frozen on dry ice. Liver: Left lateral lobe of the liver will be collected as follows: Carefully cut out pieces 1,2,3. Place into 3 tubes-need approximately 50 mg tissue piece in each tube and then snap freeze. Formalin Fixation of the Left lateral lobe of the liver-Please drop the rest of the tissue (after cutting small pieces for sampling in 1) in 10% Formalin. Samples to be paraffin embedded within 48 hours of collection. Right lateral lobe of the livery, brain, biceps, gastrocnemius muscle, and heart will be sampled and frozen for analysis.

### Materials and Methods

TEST ARTICLES—The names and concentration of the constructs to be tested are: Vehicle, VC-119 (1×10¹² vg/kg body wt- or 2×10¹⁰ per mouse for a 20 g mouse), VC-120 (1×10¹² vg/kg body wt- or 2×10¹⁰ per mouse for a 20 g mouse), VC-121 (1×10¹² vg/kg body wt- or 2×10¹⁰ per mouse for a 20 g mouse), VC-122 (1×10¹² vg/kg body wt- or 2×10¹⁰ per mouse for a 20 g mouse), HAE012 (1×10¹² vg/kg body wt- or 2×10¹⁰ per mouse for a 20 g mouse), HAE014 (1×10¹² vg/kg body wt- or 2×10¹⁰ per mouse for a 20 g mouse).

ANIMALS: C57BL/6 mice will be used. The 33 mice will be 8-10 weeks and female. Animals will be acclimated for between five to seven (5-7) days prior to study initiation. During the acclimation period, the health status of animals will be evaluated daily by technical staff for clinical presentation and behavioral signs indicating normality or illness. Only clinically healthy animals will be selected for the study. Animals will be housed in their respective groups.

The Study Schedule is presented in Table 15.

TABLE 15
Schedule of Experimental Procedures

| Event | Study Day |
|---|---|
| Animal Arrival | Day −14 |
| Dosing with test articles | Day 0 |
| Pre-bleed | Day −7 |
| Blood collection | Day 7 |
| Blood Collection | Day 14 |
| Blood Collection | Day 28 |
| Necropsy | Day 28 |

Group Designation and Dose Description: Thirty-three (33) C57BL/6 mice Female will be assigned to Seven (7) groups for the study (Table 16). Control and Test articles will be administered intravenously via the tail vein to designated groups on Study Day 0 as outlined in Table 16.

TABLE 16
Dosing Groups and Study Schedule

| | | Vector GC/ | | Dose | # of |
|---|---|---|---|---|---|
| Group | Treatment | kg body | Route | Volum | femal |
| 1 | Vehicle | — | IV (tail vain) | 100 uL | 3 |
| 2 | VC-119 | 1 × 10¹²- | IV (tail vain) | 100 ul | 5 |
| | AAV8-ApoE.hAAT.HL- | | | | |
| | scFv-Fc | | | | |
| 3 | VC-120 | 1 × 10¹² | IV (tail vain) | 100 uL | 5 |
| | AAV8-LMTP6.HL-scFv- | | | | |
| | Fc | | | | |
| 4 | VC-121 | 1 × 10¹² | IV (tail vain) | 100 ul | 5 |
| | AAV8-ApoE.hAAT.LH- | | | | |
| | scFv-Fc | | | | |
| 5 | VC-122 | 1 × 10¹² | IV (tail vain) | 100 uL | 5 |
| | AAV8-LMTP6.LH- | | | | |
| | scFv-Fc | | | | |
| 6 | HAE012 | 1 × 10¹² | IV (tail vain) | 100 ul | 5 |
| | AAV8-LMTP6-LANA | | | | |
| 7 | HAE014 | 1 × 10¹² | IV (tail vain) | 100 uL | 5 |
| | AAV9-LMTP6-LANA | | | | |

Cage side Observations: These observations will confirm the general health and viability of the animal. Any evidence of morbidity, hunched posture, ruffled fur, lethargy, diarrhea, and/or loss of >20% body weight will be documented, and the sponsor will be notified as soon as possible.

BLOOD COLLECTION: Blood will be collected for serum prior to dosing on Week 0 (i.e. pre-bleed) day −7, and Days 7, 14, and 28 (i.e. terminal blood collection). Antibody serum levels may be determined by ELISA using kallikrein as antigen.

Euthanasia/necropsy: Animals will be perfused at Necropsy with 1× cold PBS (DNAse and RNAse free) to remove blood from organs before the sample collection. All animals will be euthanized under a surgical plane of anesthesia. Liver, biceps-left forelimb, gastrocnemius muscle from hind limb, heart and brain will be harvested for analysis, such as vector copy number and mRNA transcripts of the injected vectors.

### Example 16: Mouse Serum Levels of Anti-pKal scFv-Fc Constructs

An experiment to assess mouse serum levels of AAV vectors encoding scFv-Fc constructs and full length lanadelumab antibodies for comparison was carried out as described in Example 15 above.

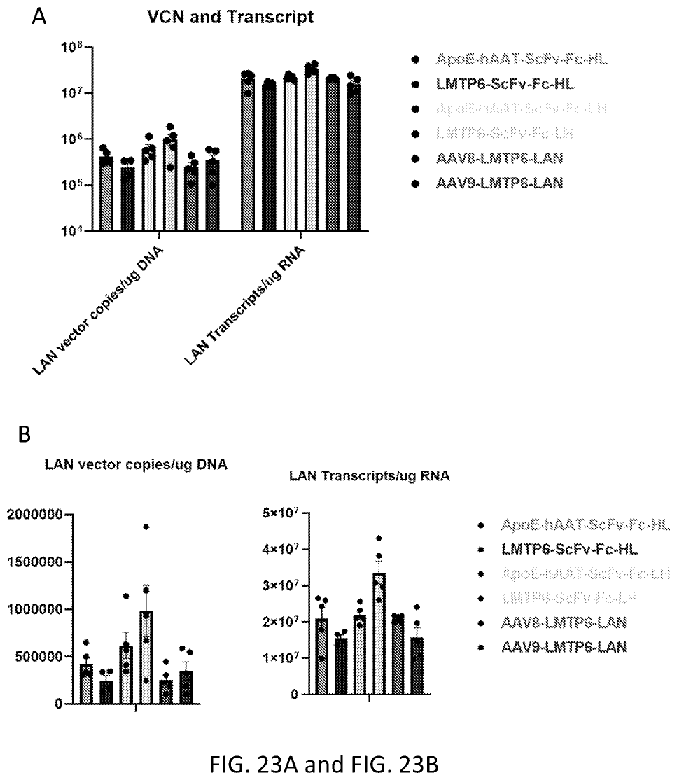

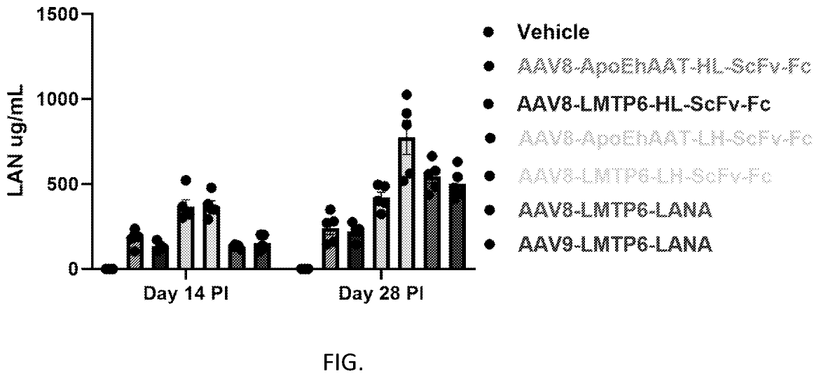

Results: All the vector constructs had similar vector copy numbers per μg DNA and transcript levels per μg/RNG in the left lateral lobe of the liver (FIGS. 23A and 23B). FIGS. 23A and B show vector copy number/ug gDNA and LAN transcripts/ug RNA in the left lateral lobe of the liver from treated mice at day 28 sacrifice. FIG. 23B presents the data at different scale of copy number and transcript. The data (numberical mean) is present in Table 17 below.

TABLE 17

| | ApoE-hAAT- | LMTP6- | ApoE-hAAT- | LMTP6- | AAV8- | AAV9- |
|---|---|---|---|---|---|---|
| | ScFv-Fc-HL | ScFv-Fc-HL | ScFv-Fc-LH | ScFv-Fc-LH | LMTP6-LAN | LMTP6-LAN |
| LAN vector | 4.20E+05 | 2.42E+05 | 6.19E+05 | 9.83E+05 | 2.55E+05 | 3.50E+05 |
| copies/μg | | | | | | |
| DNA | | | | | | |
| LAN | 2.09E+07 | 1.55E+07 | 2.19E+07 | 3.36E+07 | 2.09E+07 | 1.57E+07 |
| Transcripts/ | | | | | | |
| μg RNA | | | | | | |

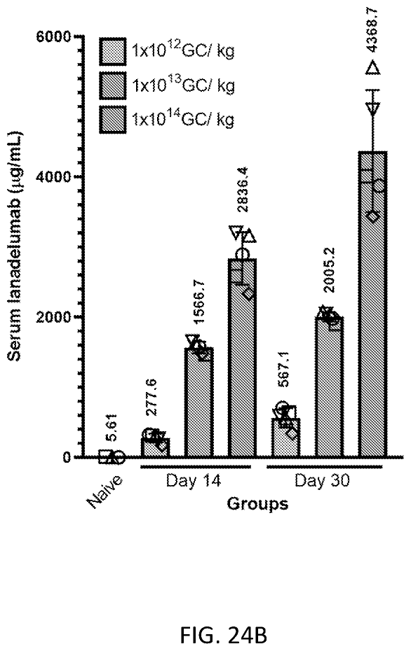

FIG. 24A shows serum LAN levels (or scFv-Fc levels) at 14 days and 28 days post infection in μg/ml. LMTP6-ScFv-Fc-LH construct generated the highest serum of kallikrein antigen binding level and that was statistically significant when compared to LMTP6-ScFv-Fc-HL construct. 24B, by way of example, shows LAN serum levels in mice injected with 1×10¹² GC/kg, 1×10¹³ GC/kg or 1×10¹⁴ GC/kg of AAV8-ApolEhAAT-LANA particles at day 14 and 30. All of the constructs showed an increase in LANA serum level compared to vehicle.

### EQUIVALENTS

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

1-29. (canceled)

30. A method of treating hereditary angioedema, diabetic retinopathy or diabetic edema in a human subject in need thereof, comprising intravenously or intramuscularly administering to the human subject a dose of a composition comprising a recombinant adeno-associated virus (AAV) vector comprising:
(a) a viral capsid that has a tropism for liver and/or muscle cells; and
(b) an artificial genome comprising an expression cassette flanked by AAV inverted terminal repeats (ITRs), wherein the expression cassette comprises a transgene encoding (i) lanadelumab or (ii) an antigen binding protein or (iii) an antigen binding fragment thereof, wherein (i), (ii) and (iii) comprise a heavy chain variable region and a light chain variable region, and wherein the transgene is operably linked to one or more regulatory sequences that control expression of the transgene in liver or muscle cells, in an amount sufficient to result in expression from the transgene and secretion of (i) lanadelumab, or (ii) the antigen binding protein or (iii) the antigen binding fragment thereof into the bloodstream of the human subject to produce (i) lanadelumab or (ii) the antigen binding protein or (iii) the antigen binding fragment thereof, plasma levels of at least 1.5 μg/ml to 35 μg/ml (i) lanadelumab or (ii) the antigen binding protein or (iii) the antigen binding fragment thereof, in the human subject within at least 60 days of said administering.

31. The method of claim 30 wherein the transgene has the nucleotide sequence of one of SEQ ID NO: 148-159.

32. The method of claim 30, wherein the viral capsid has a capsid protein that is at least 95% identical to the amino acid sequence of an AAV3B, AAV5, AAV7 (SEQ ID NO:1), AAV8 (SEQ ID NO:2), AAV9 (SEQ ID NO:3), AAVrh10 (SEQ ID NO:4), AAVrh46 (SEQ ID NO:5), AAVrh73 (SEQ ID NO:6), AAVS3 (SEQ ID NO: 8), AAV-LK03 (SEQ ID NO:7), AAVrh8, AAV64R1, or AAVhu37.

33. The method of claim 30, wherein the viral capsid is AAV8 or AAVS3 (SEQ ID NO:8).

34. The method of claim 30, wherein the regulatory sequence comprises a LSPX1 promoter (SEQ ID NO: 9), a LSPX2 promoter (SEQ ID NO: 10), a LTP1 promoter (SEQ ID NO: 11), a LTP2 promoter (SEQ ID NO: 12), a LTP3 promoter (SEQ ID NO:13), a LMTP6 promoter (SEQ ID NO: 14), a LMTP13 promoter (SEQ ID NO: 15), a LMTP14 promoter (SEQ ID NO: 16), a LMTP15 promoter (SEQ ID NO: 17), a LMTP18 promoter (SEQ ID NO: 18), a LMTP19 promoter (SEQ ID NO: 19), a LMTP20 promoter (SEQ ID NO: 20) or an ApoE.hAAT (SEQ ID NO:21) regulatory sequence.

35. The method of claim 30, wherein the regulatory sequence comprises an ApoE.hAAT (SEQ ID NO:21) regulatory sequence, a LSPX1 promoter (SEQ ID NO:9), a LSPX2 promoter (SEQ ID NO:10), a LTP1 promoter (SEQ ID NO:11), a LTP2 (SEQ ID NO:12) promoter, a LMTP6 promoter (SEQ ID NO: 14), CRE having a nucleotide sequence of one of SEQ ID NO: 163-293, CRE.hAAT, a LTP3 (SEQ ID NO:13) promoter, or a dual liver- and muscle-specific promoter.

36. The method of claim 30, wherein the transgene comprises a nucleotide sequence coding for a Furin/2A linker between the nucleotide sequence coding for the heavy chain variable region and the nucleotide sequence coding for the light chain variable region.

37. The method of claim 36, wherein the Furin 2A linker is a Furin/T2A linker having the amino acid sequence
(SEQ ID NO: 103)
RKRRAPVKQTLNFDLLKLAGDVESNPGP
or
(SEQ ID NO: 104)
RKRRGSGAPVKQTLNFDLLKLAGDVESNPGP

38. The method of claim 30, wherein the transgene encodes an scFv or scFv-Fc having the heavy chain variable region and light chain variable region.

39. The method of claim 30, wherein the transgene encodes a signal sequence at the N-terminus of the heavy chain variable region and the light chain variable region or at the N-terminus of an scFv or scFv-Fc, wherein the signal sequence directs secretion and post translational modification in said human liver or muscle cells.

40. The method of claim 39, wherein the signal sequence is
(SEQ ID NO: 50)
MYRMQLLLLIALSLALVTNS,
(SEQ ID NO: 52)
MKWVTFISLLFLFSSAYS,
(SEQ ID NO: 53)
MPSSVSWGILLLAGLCCLVPVSLA,
(SEQ ID NO: 54)
MKAAVLTLAVLFLTGSQA,
(SEQ ID NO: 55)
MKLLAATVLLLTICSLEG,
(SEQ ID NO: 56)
MDPPRPALLALLALPALLLLLLAGARA,
(SEQ ID NO: 57)
MQRVNMIMAESPGLITICLLGYLLSAEC,
(SEQ ID NO: 58)
MGPLMVLFCLLFLYPGLADS,
(SEQ ID NO: 59)
MWLLVSVILISRISSVGG,
(SEQ ID NO: 60)
MLLLFSVILISWVSTVGG,
(SEQ ID NO: 61)
MFSMRIVCLVLSVVGTAWT,
(SEQ ID NO: 62)
MKRMVSWSFHKLKTMKHLLLLLLCVFLVKS,
(SEQ ID NO: 63)
MSWSLHPRNLILYFYALLFLSSTCVA,
(SEQ ID NO: 64)
MKSLVLLLCLAQLWGCHS,
(SEQ ID NO: 65)
MARVLGAPVALGLWSLCWSLAIA,
(SEQ ID NO: 66)
MKLITILFLCSRLLLSLT,
(SEQ ID NO: 67)
MSLFPSLPLLLLSMVAASYS,
(SEQ ID NO: 68)
MEHKEVVLLLLLFLKSGQG,
(SEQ ID NO: 69)
MAHVRGLQLPGCLALAALCSLVHS,
(SEQ ID NO: 70)
MISRMEKMTMMMKILIMFALGMNYWSCSG,
(SEQ ID NO: 71)
MYSNVIGTVTSGKRKVYLLSLLLIGFWDCVTC
or
(SEQ ID NO: 71)
MRLAVGALLVCAVLGLCLA
or a signal sequence from Tables 2 or 3.

41. The method of claim 30, wherein the transgene has the structure: Signal sequence—Heavy chain—Furin site—2A site—Signal sequence—Light chain—PolyA.

42. (canceled)

43. The method of claim 30, wherein the vector is administered at a dosage of 1E11 to 1E14 vg/kg.

44. The method of claim 30, wherein said administering results in a vector genome concentration of 10-100 vg/dg as measured in the liver at 100 days after administration.

45. The method of claim 30, wherein the anti-pKal antibody plasma levels are maintained for at least 3 months.

46. The method of claim 30, wherein the anti-pKal antibody secreted into the plasma exhibits greater a greater than at least 40%, 45%, 50%, 55%, 60%, 65% or 70% reduction in pKal activity as measured by a kinetic enzymatic functional assay.

47. The method of claim 46 wherein the activity of the lanadelumab antibody is measured at 12 weeks after said administering.

48-53. (canceled)

54. The method of claim 30, wherein the human subject has plasma levels of at least 5 μg/ml to 20 μg/ml (i) lanadelumab or (ii) the antigen binding protein or (iii) the antigen binding fragment thereof, in the human subject within at least 60 days of said administering.

55. The method of claim 30, wherein the human subject has plasma levels of at least 5 μg/ml to 20 μg/ml (i) lanadelumab or (ii) the antigen binding protein or (iii) the antigen binding fragment thereof, in the human subject within at least 30 days of said administering.


SEQUENCE LISTING (393 sequences)

SEQ ID NO: 1 - protein, 737 aa, Artificial Sequence
  synthetic construct; AAV7 capsid
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 2 - protein, 738 aa, Artificial Sequence
  synthetic construct; AAV8 capsid
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 3 - protein, 736 aa, Artificial Sequence
  synthetic construct; AAV9 capsid
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 4 - protein, 738 aa, Artificial Sequence
  synthetic construct; AAVrh10 capsid
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 5 - protein, 738 aa, Artificial Sequence
  synthetic construct; AAVrh46 capsid
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKATNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 6 - protein, 738 aa, Artificial Sequence
  synthetic construct; AAVrh73 capsid
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQRQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 7 - protein, 736 aa, Artificial Sequence
  synthetic construct; AAVLK03 capsid
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 8 - protein, 736 aa, Artificial Sequence
  synthetic construct; AAVS3 capsid
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 9 - DNA, 968 bp, Artificial Sequence
  synthetic construct; LSPX1 promoter
ggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgtctagaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 10 - DNA, 1050 bp, Artificial Sequence
  synthetic construct; LSPX2 promoter
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtctagaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 11 - DNA, 1089 bp, Artificial Sequence
  synthetic construct; LTP1 promoter
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaggatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 12 - DNA, 1430 bp, Artificial Sequence
  synthetic construct; LTP2 promoter
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtctagagcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaggatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 13 - DNA, 1318 bp, Artificial Sequence
  synthetic construct; LTP3 promter
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaggatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtaaaacaggtaagtccgctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctggcctctactaaccatgttcatgttttctttttttttctacaggtcctgggtgacgaacag

SEQ ID NO: 14 - DNA, 1579 bp, Artificial Sequence
  synthetic construct; LMTP6 promoter
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 15 - DNA, 1080 bp, Artificial Sequence
  synthetic construct; LMTP13 promoter
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtctagaggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcgtgagtatcgatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 16 - DNA, 568 bp, Artificial Sequence
  synthetic construct; LMTP14 promoter
gaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcgatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 17 - DNA, 895 bp, Artificial Sequence
  synthetic construct; LMTP15 promoter
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtctagagaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcgatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 18 - DNA, 1291 bp, Artificial Sequence
  synthetic construct; LMTP18 promoter
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 19 - DNA, 1147 bp, Artificial Sequence
  synthetic construct; LMTP19 promoter
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 20 - DNA, 1490 bp, Artificial Sequence
  synthetic construct; LMTP20 promoter
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggcccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgaagctttggcccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 21 - DNA, 723 bp, Artificial Sequence
  synthetic construct; ApoE.hAAT promoter
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 22 - protein, 4 aa, Artificial Sequence
  synthetic construct; linker sequence
  MISC_FEATURE (2)..(2): Xaa can be any amino acid
RXKR

SEQ ID NO: 23 - protein, 4 aa, Artificial Sequence
  synthetic construct; linker sequence
  MISC_FEATURE (2)..(2): Xaa can be any amino acid
RXRR

SEQ ID NO: 24 - protein, 738 aa, Artificial Sequence
  synthetic construct; rh64R1 capsid
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPIGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIVWELQKENSKRWNPEIQYTSNYYKSTNVDFAVNTEGVYSEPRPIGTRYLTRNL

SEQ ID NO: 25 - protein, 204 aa, Artificial Sequence
  synthetic construct; lanadelumab Fc domain
FPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - DNA, 100 bp, Artificial Sequence
  synthetic construct; Alpha-Mic/Bik Enhancer
aggttaatttttaaaaagcagtcaaaagtccaagtggcccttggcagcatttactctctctgtttgctctggttaataatctcaggagcacaaacattcc

SEQ ID NO: 27 - DNA, 200 bp, Artificial Sequence
  synthetic construct; Tandem (2) alpha-Mic/Bik Enhancers
aggttaatttttaaaaagcagtcaaaagtccaagtggcccttggcagcatttactctctctgtttgctctggttaataatctcaggagcacaaacattccaggttaatttttaaaaagcagtcaaaagtccaagtggcccttggcagcatttactctctctgtttgctctggttaataatctcaggagcacaaacattcc

SEQ ID NO: 28 - DNA, 321 bp, Artificial Sequence
  synthetic construct; ApoE Hepatic Control Region containing ApoE Enhancer
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 29 - DNA, 554 bp, Artificial Sequence
  synthetic construct; Tandem (2) ApoE Enhancers
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtctagaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctg

SEQ ID NO: 30 - DNA, 393 bp, Artificial Sequence
  synthetic construct; hAAT Promoter
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 31 - DNA, 393 bp, Artificial Sequence
  synthetic construct; hAAT(?ATG) Promoter
gatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 32 - DNA, 144 bp, Artificial Sequence
  synthetic construct; Mck Enhancer
ccactacgggtttaggctgcccatgtaaggaggcaaggcctggggacacccgagatgcctggttataattaacccagacatgtggctgccccccccccccccaacacctgctgcctctaaaaataaccctgtccctggtggatc

SEQ ID NO: 33 - DNA, 288 bp, Artificial Sequence
  synthetic construct; Tandem (2) Mck Enhancers
ccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatc

SEQ ID NO: 34 - DNA, 432 bp, Artificial Sequence
  synthetic construct; Tandem Mck (3) Enhancers
ccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatc

SEQ ID NO: 35 - DNA, 199 bp, Artificial Sequence
  synthetic construct; Myosin heavy chain enhancer
cccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgaagctttggc

SEQ ID NO: 36 - DNA, 1725 bp, Artificial Sequence
  synthetic construct; CAG/CB7
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaag

SEQ ID NO: 37 - DNA, 865 bp, Artificial Sequence
  synthetic construct; CK8 promoter
ccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcga

SEQ ID NO: 38 - DNA, 251 bp, Artificial Sequence
  synthetic construct; mU1a
atggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcatact

SEQ ID NO: 39 - DNA, 212 bp, Artificial Sequence
  synthetic construct; EF-1alpha
gggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 40 - DNA, 460 bp, Artificial Sequence
  synthetic construct; TBG
gggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagat

SEQ ID NO: 41 - DNA, 133 bp, Artificial Sequence
  synthetic construct; chimeric intron
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 42 - DNA, 82 bp, Artificial Sequence
  synthetic construct; VH4 intron
gtgagtatctcagggatccagacatggggatatgggaggtgcctctgatcccagggctcactgtgggtctctctgttcacag

SEQ ID NO: 43 - DNA, 97 bp, Artificial Sequence
  synthetic construct; SV40 intron
gtaagtttagtctttttgtcttttatttcaggtcccggatccggtggtggtgcaaatcaaagaactgctcctcagtggatgttgcctttacttctag

SEQ ID NO: 44 - DNA, 55 bp, Artificial Sequence
  synthetic construct; beta-globin PolyA signal
ataaaggaaatttattttcattgcaatagtgtgttggaattttttgtgtctctca

SEQ ID NO: 45 - DNA, 127 bp, Artificial Sequence
  synthetic construct; Rabbit beta-globin polyA
gatctttttccctctgccaaaaattatggggacatcatgaagccccttgagcatctgacttctggctaataaaggaaatttattttcattgcaatagtgtgttggaattttttgtgtctctcactcg

SEQ ID NO: 46 - DNA, 130 bp, Artificial Sequence
  synthetic construct; 5′ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 47 - DNA, 106 bp, Artificial Sequence
  synthetic construct; 5′-ITR
ctgcgcgctcgctcgctcactgaggccgcccgggcaaagcccgggcgtcgggcgacctttggtcgcccggcctcagtgagcgagcgagcgcgcagagagggagtgg

SEQ ID NO: 48 - DNA, 143 bp, Artificial Sequence
  synthetic construct; 3′-ITR AAV
gaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 49 - DNA, 110 bp, Artificial Sequence
  synthetic construct; 3′-ITR
ttggccactccctctctgcgcgctcgctcgctcactgaggccgggcgaccaaaggtcgcccgacgcccgggctttgcccgggcggcctcagtgagcgagcgagcgcgcag

SEQ ID NO: 50 - protein, 20 aa, Artificial Sequence
  synthetic construct; Mutant interleukin 2 signal peptide
MYRMQLLLLIALSLALVTNS

SEQ ID NO: 51 - DNA, 60 bp, Artificial Sequence
  Synthetic construct; Mutant interleukin 2 signal peptide coding sequence
atgtataggatgcaactgctcctcctgattgctctgagcctggctcttgtgaccaactct

SEQ ID NO: 52 - protein, 18 aa, Artificial Sequence
  Synthetic construct; Human Serum albumin
MKWVTFISLLFLFSSAYS

SEQ ID NO: 53 - protein, 24 aa, Artificial Sequence
  Synthetic construct; Human alpha-1 Antitrypsin
MPSSVSWGILLLAGLCCLVPVSLA

SEQ ID NO: 54 - protein, 18 aa, Artificial Sequence
  Synthetic construct; Human Apolipoprotein A-1
MKAAVLTLAVLFLTGSQA

SEQ ID NO: 55 - protein, 18 aa, Artificial Sequence
  Synthetic construct; Human Apolipoprotein A-2
MKLLAATVLLLTICSLEG

SEQ ID NO: 56 - protein, 27 aa, Artificial Sequence
  Synthetic construct; Human Apolipoprotein B-100
MDPPRPALLALLALPALLLLLLAGARA

SEQ ID NO: 57 - protein, 28 aa, Artificial Sequence
  Synthetic construct; Human Coagulation Factor IX
MQRVNMIMAESPGLITICLLGYLLSAEC

SEQ ID NO: 58 - protein, 20 aa, Artificial Sequence
  Synthetic construct; Human Complement C2
MGPLMVLFCLLFLYPGLADS

SEQ ID NO: 59 - protein, 18 aa, Artificial Sequence
  Synthetic construct; Human Complement Factor H-related Protein 2
MWLLVSVILISRISSVGG

SEQ ID NO: 60 - protein, 18 aa, Artificial Sequence
  Synthetic construct; Human Complement Factor H-related Protein 5
MLLLFSVILISWVSTVGG

SEQ ID NO: 61 - protein, 19 aa, Artificial Sequence
  Synthetic construct; Human Fibrinogen alpha-chain
MFSMRIVCLVLSVVGTAWT

SEQ ID NO: 62 - protein, 30 aa, Artificial Sequence
  Synthetic construct; Human Fibrinogen beta-chain
MKRMVSWSFHKLKTMKHLLLLLLCVFLVKS

SEQ ID NO: 63 - protein, 26 aa, Artificial Sequence
  Synthetic construct;
MSWSLHPRNLILYFYALLFLSSTCVA

SEQ ID NO: 64 - protein, 18 aa, Artificial Sequence
  Synthetic construct; Human ?-2-HS-Glycoprotein
MKSLVLLLCLAQLWGCHS

SEQ ID NO: 65 - protein, 23 aa, Artificial Sequence
  Synthetic construct; Human Hemopexin
MARVLGAPVALGLWSLCWSLAIA

SEQ ID NO: 66 - protein, 18 aa, Artificial Sequence
  Synthetic construct; Human Kininogen-1
MKLITILFLCSRLLLSLT

SEQ ID NO: 67 - protein, 20 aa, Artificial Sequence
  Synthetic construct; Human Mannose-binding protein C
MSLFPSLPLLLLSMVAASYS

SEQ ID NO: 68 - protein, 19 aa, Artificial Sequence
  Synthetic construct;
MEHKEVVLLLLLFLKSGQG

SEQ ID NO: 69 - protein, 24 aa, Artificial Sequence
  Synthetic construct; Human Prothrombin
MAHVRGLQLPGCLALAALCSLVHS

SEQ ID NO: 70 - protein, 29 aa, Artificial Sequence
  Synthetic construct; Human Secreted Phosphoprotein 24
MISRMEKMTMMMKILIMFALGMNYWSCSG

SEQ ID NO: 71 - protein, 32 aa, Artificial Sequence
  Synthetic construct; Human Anti-thrombin-III
MYSNVIGTVTSGKRKVYLLSLLLIGFWDCVTC

SEQ ID NO: 72 - protein, 19 aa, Artificial Sequence
  Synthetic construct; Human Serotransferrin
MRLAVGALLVCAVLGLCLA

SEQ ID NO: 73 - protein, 17 aa, Artificial Sequence
  Synthetic construct; Human SPARC
MRAWIFFLLCLAGRALA

SEQ ID NO: 74 - protein, 22 aa, Artificial Sequence
  Synthetic construct; Human Collagen alpha-1(I) chain
MFSFVDLRLLLLLAATALLTHG

SEQ ID NO: 75 - protein, 19 aa, Artificial Sequence
  Synthetic construct; Human Lactotransferrin
MKLVFLVLLFLGALGLCLA

SEQ ID NO: 76 - protein, 22 aa, Artificial Sequence
  Synthetic construct; Human Complement C3
MGPTSGPSLLLLLLTHLPLALG

SEQ ID NO: 77 - protein, 18 aa, Artificial Sequence
  Synthetic construct; Human Lumican
MSLSAFTLFLALIGGTSG

SEQ ID NO: 78 - protein, 27 aa, Artificial Sequence
  Synthetic construct; Human Gelsolin isoform 1
MAPHRPAPALLCALSLALCALSLPVRA

SEQ ID NO: 79 - protein, 22 aa, Artificial Sequence
  Synthetic construct; Human Pro-cathepsin H
MWATLPLLCAGAWLLGVPVCGA

SEQ ID NO: 80 - protein, 19 aa, Artificial Sequence
  Synthetic construct; Human SERPINF1
MQALVLLLCIGALLGHSSC

SEQ ID NO: 81 - protein, 23 aa, Artificial Sequence
  Synthetic construct; Human SERPINE1
MQMSPALTCLVLGLALVFGEGSA

SEQ ID NO: 82 - protein, 20 aa, Artificial Sequence
  Synthetic construct; Human Cathepsin D
MQPSSLLPLALCLLAAPASA

SEQ ID NO: 83 - protein, 23 aa, Artificial Sequence
  Synthetic construct; Human TIMP1
MAPFEPLASGILLLLWLIAPSRA

SEQ ID NO: 84 - protein, 31 aa, Artificial Sequence
  Synthetic construct; Human Fibronectin
MLRGPGPGLLLLAVQCLGTAVPSTGASKSKR

SEQ ID NO: 85 - protein, 15 aa, Artificial Sequence
  Synthetic construct; Human Complement C1s subcomponent
MWCIVLFSLLAWVYA

SEQ ID NO: 86 - protein, 17 aa, Artificial Sequence
  Synthetic construct;
MNPTLILAAFCLGIASA

SEQ ID NO: 87 - protein, 17 aa, Artificial Sequence
  Synthetic construct; Human Cathepsin B
MWQLWASLCCLLVLANA

SEQ ID NO: 88 - protein, 16 aa, Artificial Sequence
  Synthetic construct; Human Salivary acidic proline-rich phosphoprotein
MLLILLSVALLAFSSA

SEQ ID NO: 89 - protein, 20 aa, Artificial Sequence
  Synthetic construct; Human Follistatin-related protein 1
MWKRWLALALALVAVAWVRA

SEQ ID NO: 90 - protein, 3 aa, Artificial Sequence
  Synthetic construct; linker
GSG

SEQ ID NO: 91 - protein, 4 aa, Artificial Sequence
  Synthetic construct; furin linker
RKRR

SEQ ID NO: 92 - protein, 4 aa, Artificial Sequence
  Synthetic construct; linker
RRRR

SEQ ID NO: 93 - protein, 4 aa, Artificial Sequence
  Synthetic construct; linker
RRKR

SEQ ID NO: 94 - protein, 4 aa, Artificial Sequence
  Synthetic construct; linker
RKKR

SEQ ID NO: 95 - protein, 18 aa, Artificial Sequence
  Synthetic construct; linker
EGRGSLLTCGDVEENPGP

SEQ ID NO: 96 - protein, 21 aa, Artificial Sequence
  Synthetic construct; linker
GSGEGRGSLLTCGDVEENPGP

SEQ ID NO: 97 - protein, 19 aa, Artificial Sequence
  Synthetic construct; linker
ATNFSLLKQAGDVEENPGP

SEQ ID NO: 98 - protein, 22 aa, Artificial Sequence
  Synthetic construct; linker
GSGATNFSLLKQAGDVEENPGP

SEQ ID NO: 99 - protein, 20 aa, Artificial Sequence
  Synthetic construct; linker
QCTNYALLKLAGDVESNPGP

SEQ ID NO: 100 - protein, 23 aa, Artificial Sequence
  Synthetic construct; linker
GSGQCTNYALLKLAGDVESNPGP

SEQ ID NO: 101 - protein, 24 aa, Artificial Sequence
  Synthetic construct; linker
APVKQTLNFDLLKLAGDVESNPGP

SEQ ID NO: 102 - protein, 27 aa, Artificial Sequence
  Synthetic construct; linker
GSGAPVKQTLNFDLLKLAGDVESNPGP

SEQ ID NO: 103 - protein, 22 aa, Artificial Sequence
  Synthetic construct; linker
RKRREGRGSLLTCGDVEENPGP

SEQ ID NO: 104 - protein, 25 aa, Artificial Sequence
  Synthetic construct;
RKRRGSGEGRGSLLTCGDVEENPGP

SEQ ID NO: 105 - protein, 28 aa, Artificial Sequence
  Synthetic construct; linker
RKRRAPVKQTLNFDLLKLAGDVESNPGP

SEQ ID NO: 106 - protein, 31 aa, Artificial Sequence
  Synthetic construct; linker
RKRRGSGAPVKQTLNFDLLKLAGDVESNPGP

SEQ ID NO: 107 - DNA, 75 bp, Artificial Sequence
  Synthetic construct; linker
agaaagagaagaggctctggagaaggcagaggctccctgctgacatgtggggatgttgaagagaatcctgggcct

SEQ ID NO: 108 - DNA, 12 bp, Artificial Sequence
  Synthetic construct; linker
agaaagagaaga

SEQ ID NO: 109 - DNA, 21 bp, Artificial Sequence
  Synthetic construct; linker
agaaagagaagaggctctgga

SEQ ID NO: 110 - DNA, 9 bp, Artificial Sequence
  Synthetic construct; linker
ggctctgga

SEQ ID NO: 111 - protein, 54 aa, Artificial Sequence
  Synthetic construct; linker
GAAGGCAGAGGCTCCCTGCTGACATGTGGGGATGTTGAAGAGAATCCTGGGCCT

SEQ ID NO: 112 - protein, 5 aa, Artificial Sequence
  Synthetic construct; hinge region
CPPCP

SEQ ID NO: 113 - protein, 6 aa, Artificial Sequence
  Synthetic construct; hinge region
CPPCPA

SEQ ID NO: 114 - protein, 5 aa, Artificial Sequence
  Synthetic construct; hinge region
CPPCA

SEQ ID NO: 115 - protein, 22 aa, Artificial Sequence
  Synthetic construct; hinge region
EPKSCDKTHTCPPCPAPELLGG

SEQ ID NO: 116 - protein, 22 aa, Artificial Sequence
  Synthetic construct; hinge region
EPKSCDKTHLCPPCPAPELLGG

SEQ ID NO: 117 - protein, 10 aa, Artificial Sequence
  Synthetic construct; hinge region
EPKSCDKTHL

SEQ ID NO: 118 - protein, 10 aa, Artificial Sequence
  Synthetic construct; hinge region
EPKSCDKTHT

SEQ ID NO: 119 - protein, 16 aa, Artificial Sequence
  Synthetic construct; hinge region
EPKSCDKTHTCPPCPA

SEQ ID NO: 120 - protein, 16 aa, Artificial Sequence
  Synthetic construct; hinge region
EPKSCDKTHLCPPCPA

SEQ ID NO: 121 - protein, 27 aa, Artificial Sequence
  Synthetic construct; hinge region
EPKSCDKTHTCPPCPAPELLGGPSVFL

SEQ ID NO: 122 - protein, 27 aa, Artificial Sequence
  Synthetic construct; hinge region
EPKSCDKTHLCPPCPAPELLGGPSVFL

SEQ ID NO: 123 - protein, 22 aa, Artificial Sequence
  Synthetic construct; hinge region
EPKSCDKTHTCPPCPAPEAAGG

SEQ ID NO: 124 - protein, 27 aa, Artificial Sequence
  Synthetic construct; hinge region
EPKSCDKTHTCPPCPAPEAAGGPSVFL

SEQ ID NO: 125 - protein, 27 aa, Artificial Sequence
  Synthetic construct; hinge region
EPKSCDKTHLCPPCPAPEAAGGPSVFL

SEQ ID NO: 126 - protein, 18 aa, Artificial Sequence
  Synthetic construct; hinge region
ERKSCVECPPCPAPPVAG

SEQ ID NO: 127 - protein, 13 aa, Artificial Sequence
  Synthetic construct; hinge region
ERKSCVECPPCPA

SEQ ID NO: 128 - protein, 19 aa, Artificial Sequence
  Synthetic construct; hinge region
ESKYGPPCPPCPAPEFLGG

SEQ ID NO: 129 - protein, 13 aa, Artificial Sequence
  Synthetic construct; hinge region
ESKYGPPCPPCPA

SEQ ID NO: 130 - protein, 13 aa, Artificial Sequence
  Synthetic construct; hinge region
ESKYGPPCPSCPA

SEQ ID NO: 131 - protein, 24 aa, Artificial Sequence
  Synthetic construct; hinge region
ESKYGPPCPSCPAPEFLGGPSVFL

SEQ ID NO: 132 - protein, 24 aa, Artificial Sequence
  Synthetic construct;
ESKYGPPCPPCPAPEFLGGPSVFL

SEQ ID NO: 133 - protein, 18 aa, Artificial Sequence
  Synthetic construct; hinge region
ERKCCVECPPCPAPPVAG

SEQ ID NO: 134 - protein, 13 aa, Artificial Sequence
  Synthetic construct; hinge region
ERKCCVECPPCPA

SEQ ID NO: 135 - protein, 22 aa, Artificial Sequence
  Synthetic construct; hinge region
EPKSCDKTHTCPPCPAPELAGA

SEQ ID NO: 136 - protein, 27 aa, Artificial Sequence
  Synthetic construct; hinge region
EPKSCDKTHTCPPCPAPELAGAPSVFL

SEQ ID NO: 137 - protein, 27 aa, Artificial Sequence
  Synthetic construct; hinge region
EPKSCDKTHLCPPCPAPELAGAPSVFL

SEQ ID NO: 138 - protein, 22 aa, Artificial Sequence
  Synthetic construct; hinge region
EPKSCDKTHTCPPCPAPEFEGG

SEQ ID NO: 139 - protein, 27 aa, Artificial Sequence
  Synthetic construct; hinge region
EPKSCDKTHTCPPCPAPEFEGGPSVFL

SEQ ID NO: 140 - protein, 27 aa, Artificial Sequence
  Synthetic construct; hinge region
EPKSCDKTHLCPPCPAPEFEGGPSVFL

SEQ ID NO: 141 - protein, 232 aa, Artificial Sequence
  Synthetic construct; IgG1
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 142 - protein, 228 aa, Artificial Sequence
  Synthetic construct; IgG2
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 143 - protein, 229 aa, Artificial Sequence
  Synthetic construct; IgG4
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 144 - protein, 247 aa, Artificial Sequence
  Synthetic construct; Lanadelumab Heavy
  MISC_FEATURE (227)..(230): wherein amino acids 227 to 230 are either present or absent
  MISC_FEATURE (227)..(227): wherein Xaa is K
  MISC_FEATURE (228)..(228): wherein Xaa is T
  MISC_FEATURE (229)..(229): wherein Xaa is H
  MISC_FEATURE (230)..(230): wherein Xaa is T or L
  MISC_FEATURE (231)..(236): wherein amino acids 231 to 236 are either present or absent
  MISC_FEATURE (237)..(247): wherein amino acids 237 to 247 are either present or absent
EVQLLESGGGLVQPGGSLRLSCAASGFTFSHYIMMWVRQAPGKGLEWVSGIYSSGGITVYADSVKGRFTISRDNSKNTLYLQMNSLRAEDTAVYYCAYRRIGVPRRDEFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDXXXXCPPCPAPELLGGPSVFL

SEQ ID NO: 145 - protein, 213 aa, Artificial Sequence
  Synthetic construct; Lanadelumab Light
DIQMTQSPSTLSASVGDRVTITCRASQSISSWLAWYQQKPGKAPKLLIYKASTLESGVPSRFSGSGSGTEFTLTISSLQPDDFATYYCQQYNTYWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 146 - protein, 741 aa, Artificial Sequence
  Synthetic construct; Lanadelumab Heavy
  MISC_FEATURE (678)..(690): wherein amino acids 678 to 690 are present or absent
  MISC_FEATURE (688)..(688): wherein Xaa is A or C
  MISC_FEATURE (689)..(689): wherein Xaa is C or T
  MISC_FEATURE (690)..(690): wherein Xaa is C or G
  MISC_FEATURE (691)..(708): wherein amino acids 691 to 708 are present or absent
  MISC_FEATURE (709)..(736): wherein amino acids 709 to 736 are present or absent
GAGGTGCAGCTGCTGGAGAGCGGCGGCGGCCTGGTGCAGCCCGGCGGCAGCCTGAGGCTGAGCTGCGCCGCCAGCGGCTTCACCTTCAGCCACTACATCATGATGTGGGTGAGGCAGGCCCCCGGCAAGGGCCTGGAGTGGGTGAGCGGCATCTACAGCAGCGGCGGCATCACCGTGTACGCCGACAGCGTGAAGGGCAGGTTCACCATCAGCAGGGACAACAGCAAGAACACCCTGTACCTGCAGATGAACAGCCTGAGGGCCGAGGACACCGCCGTGTACTACTGCGCCTACAGGAGGATCGGCGTGCCCAGGAGGGACGAGTTCGACATCTGGGGCCAGGGCACCATGGTGACCGTGAGCAGCGCCAGCACCAAGGGCCCCAGCGTGTTCCCCCTGGCCCCCAGCAGCAAGAGCACCAGCGGCGGCACCGCCGCCCTGGGCTGCCTGGTGAAGGACTACTTCCCCGAGCCCGTGACCGTGAGCTGGAACAGCGGCGCCCTGACCAGCGGCGTGCACACCTTCCCCGCCGTGCTGCAGAGCAGCGGCCTGTACAGCCTGAGCAGCGTGGTGACCGTGCCCAGCAGCAGCCTGGGCACCCAGACCTACATCTGCAACGTGAACCACAAGCCCAGCAACACCAAGGTGGACAAGAGGGTGGAGCCCAAGAGCTGCGACAAGACCCACXXXTGCCCCCCCTGCCCCGCCCCCGAGCTGCTGGGCGGCCCCAGCGTGTTCCTG

SEQ ID NO: 147 - protein, 639 aa, Artificial Sequence
  Synthetic construct; Lanadelumab Light
GACATCCAGATGACCCAGAGCCCCAGCACCCTGAGCGCCAGCGTGGGCGACAGGGTGACCATCACCTGCAGGGCCAGCCAGAGCATCAGCAGCTGGCTGGCCTGGTACCAGCAGAAGCCCGGCAAGGCCCCCAAGCTGCTGATCTACAAGGCCAGCACCCTGGAGAGCGGCGTGCCCAGCAGGTTCAGCGGCAGCGGCAGCGGCACCGAGTTCACCCTGACCATCAGCAGCCTGCAGCCCGACGACTTCGCCACCTACTACTGCCAGCAGTACAACACCTACTGGACCTTCGGCCAGGGCACCAAGGTGGAGATCAAGAGGACCGTGGCCGCCCCCAGCGTGTTCATCTTCCCCCCCAGCGACGAGCAGCTGAAGAGCGGCACCGCCAGCGTGGTGTGCCTGCTGAACAACTTCTACCCCAGGGAGGCCAAGGTGCAGTGGAAGGTGGACAACGCCCTGCAGAGCGGCAACAGCCAGGAGAGCGTGACCGAGCAGGACAGCAAGGACAGCACCTACAGCCTGAGCAGCACCCTGACCCTGAGCAAGGCCGACTACGAGAAGCACAAGGTGTACGCCTGCGAGGTGACCCACCAGGGCCTGAGCAGCCCCGTGACCAAGAGCTTCAACAGGGGCGAGTGC

SEQ ID NO: 148 - DNA, 2205 bp, Artificial Sequence
  Synthetic construct; L01
atgtaccggatgcagctgctgctgctcattgccctgtctctggccctggtcaccaatagcgaagtccagctgctggaatctggcggcggacttgttcaacctggcggctctctgagactgagctgtgccgcttccggcttcaccttcagccactatatcatgatgtgggtccgacaggcccctggcaaaggccttgaatgggtgtccggcatctatagcagcggcggcatcacagtgtacgccgactctgtgaagggcagattcaccatcagccgggacaacagcaagaacaccctgtacctgcagatgaacagcctgagagccgaggacaccgccgtgtactactgcgcctatagaagaatcggcgtgcccagacgggacgagttcgatatttggggccagggcaccatggtcaccgtgtctagcgcctctacaaagggccctagcgtgttccctctggctcctagcagcaagagcacaagcggaggaacagccgctctgggctgtctggtcaaggactactttcccgagcctgtgaccgtgtcctggaatagcggagcactgacaagcggcgtgcacacctttccagctgtgctgcaaagcagcggcctgtactctctgagcagcgtggtcacagtgccaagctctagcctgggcacccagacctacatctgcaatgtgaaccacaagcctagcaacaccaaggtggacaagagagtggaacccaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagccgggaagagatgaccaagaaccaggtgtccctgacctgcctcgtgaagggcttctacccttccgatatcgccgtggaatgggagagcaatggccagccagagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagagtctgagcctgtctccaggcctgcggaagagaagggctcctgtgaagcagaccctgaacttcgacctgctgaaactggccggcgacgtggaaagcaaccccggacctatgtatagaatgcagctcctcctgctgatcgcactgagcctggctctcgtgaccaacagcgacatccagatgacacagagccccagcacactgtctgccagcgtgggagacagagtgaccatcacctgtagagccagccagtccatctcctcttggctggcctggtatcagcaaaagcctggcaaggcccctaagctgctgatctacaaggcctccacactggaaagcggggtgccctccagattttctggcagcggatctggcaccgagttcaccctgaccatcagtagcctgcagcctgacgacttcgccacctactactgccagcagtacaatacctactggaccttcggccagggaacaaaggtggaaatcaagcggactgtggccgctccaagcgtgttcatctttccacctagcgacgagcagctgaagtccggcacagcctctgttgtgtgcctgctgaacaacttctaccccagagaagccaaggtgcagtggaaagtggacaatgccctgcagagcggcaactcccaagagagcgtgacagagcaggactccaaggattccacctacagcctgtctagcaccctgacactgagcaaggccgactacgagaagcacaaagtgtatgcctgcgaagtgacccaccagggccttagctctccagtgaccaagagcttcaaccggggcgagtgttgataa

SEQ ID NO: 149 - DNA, 2193 bp, Artificial Sequence
  Synthetic construct; L02
atgtacagaatgcagctgctgctgctcattgccctgtctctggccctggtcaccaattctgaggtccagctgcttgagagtggtggtggactggttcagcctggtggcagcctgagactgtcttgtgctgcctctggcttcaccttcagccactatatcatgatgtgggtcagacaggcccctggcaaaggcctggaatgggtgtcaggcatctacagcagtggtggcatcacagtgtatgctgactctgtgaagggcagattcaccatcagcagagacaacagcaagaacaccctgtacctgcagatgaactccctgagagctgaggacacagcagtgtactactgtgcctatagaagaattggggtgcccagaagggatgagtttgacatctggggccagggcaccatggttacagtgtcctctgccagcacaaagggcccctctgtttttccactggctcccagcagcaagagcaccagtggtggaacagctgccctgggctgtctggtcaaggattacttccctgagcctgtgactgtgtcctggaactctggggctctgacttctggggtgcacacctttccagctgtgctgcagtcctctggcctgtactctctgtcctctgtggtcacagtgcctagctctagcctgggcacacagacctacatctgcaatgtgaaccacaagcctagcaacaccaaggtggacaagagagtggaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgccaagaccaagcctagagaggaacagtacaacagcacctacagagtggtttctgtgctgacagtgctgcaccaggactggctgaatggcaaagagtacaagtgcaaggtgtccaacaaggccctgcctgctcctattgagaaaaccatctccaaggccaagggccagccaagagaaccccaggtttacaccctgccacctagcagagaagagatgaccaagaaccaggtgtccctgacctgcctggttaagggcttctacccctctgacattgctgtggaatgggagagcaatggccagcctgaaaacaactacaagacaacccctcctgtgctggactctgatggctcattcttcctgtacagcaagctgactgtggacaagtccagatggcagcagggaaatgtgttcagctgctctgtgatgcatgaggccctgcacaaccactacacccagaaaagtctgtctctgtcccctggcagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagcccctccacactgtctgcatctgtgggagacagagtgaccatcacctgtagagccagccagtctatctctagctggctggcctggtatcagcaaaagcctggcaaggcccctaagctgctgatctacaaggccagcacacttgagtcaggggtgccctccagattttctggctctggatctggcacagagttcaccctgaccatcagctccctgcagccagatgactttgccacctactactgccagcagtacaatacctactggacctttggccagggaacaaaggtggaaatcaagagaacagtggctgccccatctgtgttcatcttcccaccatctgatgaacagctgaagtctggcactgcctctgttgtgtgcctgctgaacaacttttaccccagagaagccaaggtgcagtggaaagtggataatgccctgcagtctggcaatagccaagaatctgtgacagagcaggactccaaggattccacctacagcctgagcagcaccctgacactgagcaaggctgactatgagaagcacaaagtgtatgcctgtgaagtgacacaccagggactgagcagcccagtgaccaagagcttcaacaggggagagtgctgataa

SEQ ID NO: 150 - DNA, 2211 bp, Artificial Sequence
  Synthetic construct; L03
atgtacagaatgcagctgctgctgctcattgccctgtctctggccctggtcaccaattctgaggtccagctgcttgagagtggtggtggactggttcagcctggtggcagcctgagactgtcttgtgctgcctctggcttcaccttcagccactatatcatgatgtgggtcagacaggcccctggcaaaggcctggaatgggtgtcaggcatctacagcagtggtggcatcacagtgtatgctgactctgtgaagggcagattcaccatcagcagagacaacagcaagaacaccctgtacctgcagatgaactccctgagagctgaggacacagcagtgtactactgtgcctatagaagaattggggtgcccagaagggatgagtttgacatctggggccagggcaccatggttacagtgtcctctgccagcacaaagggcccctctgtttttccactggctcccagcagcaagagcaccagtggtggaacagctgccctgggctgtctggtcaaggattacttccctgagcctgtgactgtgtcctggaactctggggctctgacctctggggtgcacacctttccagctgtgctgcagtcctctggcctgtactctctgtcctctgtggtcacagtgcctagctctagcctgggcacacagacctacatctgcaatgtgaaccacaagcctagcaacaccaaggtggacaagagagtggaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgccaagaccaagcctagagaggaacagtacaacagcacctacagagtggtttctgtgctgacagtgctgcaccaggactggctgaatggcaaagagtacaagtgcaaggtgtccaacaaggccctgcctgctcctattgagaaaaccatctccaaggccaagggccagccaagagaaccccaggtttacaccctgccacctagcagagaagagatgaccaagaaccaggtgtccctgacctgcctggttaagggcttctacccctctgacattgctgtggaatgggagagcaatggccagcctgaaaacaactacaagacaacccctcctgtgctggactctgatggctcattcttcctgtacagcaagctgactgtggacaagtccagatggcagcagggaaatgtgttcagctgctctgtgatgcatgaggccctgcacaaccactacacccagaaaagtctgtctctgtcccctggcagaaagagaagaggatcaggggccccagtgaagcagaccctgaactttgatctgctgaagctggctggggatgttgagagcaaccctggacctatgtataggatgcaactgctcctcctgattgctctgagcctggctcttgtgaccaactctgacatccagatgacacagagcccctccacactgtctgcatctgtgggagacagagtgaccatcacctgtagagccagccagtctatctcttcctggctggcctggtatcagcaaaagcctggcaaggcccctaagctgctgatctacaaggccagcacacttgagtcaggggtgccctccagattttctggctctggatctggcacagagttcaccctgaccatcagctccctgcagccagatgactttgccacctactactgccagcagtacaatacctactggacctttggccagggaacaaaggtggaaatcaagagaacagtggctgccccatctgtgttcatcttcccaccatctgatgaacagctgaagtctggcactgcctctgttgtgtgcctgctgaacaacttttaccctagagaagccaaggtgcagtggaaagtggataatgccctgcagtctggcaatagccaagaatctgtgacagagcaggactccaaggattccacctacagcctgagcagcaccctgacactgagcaaggctgactatgagaagcacaaagtgtatgcctgtgaagtgacacaccagggactgagcagcccagtgaccaagagcttcaacaggggagagtgctgataa

SEQ ID NO: 151 - DNA, 4196 bp, Artificial Sequence
  Synthetic construct; CAG.LAN.F2A
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgggcactttgcactggaacttacaacacccgagcaaggacgcgactctccaccatgtaccggatgcagctgctgctgctcattgccctgtctctggccctggtcaccaatagcgaagtccagctgctggaatctggcggcggacttgttcaacctggcggctctctgagactgagctgtgccgcttccggcttcaccttcagccactatatcatgatgtgggtccgacaggcccctggcaaaggccttgaatgggtgtccggcatctatagcagcggcggcatcacagtgtacgccgactctgtgaagggcagattcaccatcagccgggacaacagcaagaacaccctgtacctgcagatgaacagcctgagagccgaggacaccgccgtgtactactgcgcctatagaagaatcggcgtgcccagacgggacgagttcgatatttggggccagggcaccatggtcaccgtgtctagcgcctctacaaagggccctagcgtgttccctctggctcctagcagcaagagcacaagcggaggaacagccgctctgggctgtctggtcaaggactactttcccgagcctgtgaccgtgtcctggaatagcggagcactgacaagcggcgtgcacacctttccagctgtgctgcaaagcagcggcctgtactctctgagcagcgtggtcacagtgccaagctctagcctgggcacccagacctacatctgcaatgtgaaccacaagcctagcaacaccaaggtggacaagagagtggaacccaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagccgggaagagatgaccaagaaccaggtgtccctgacctgcctcgtgaagggcttctacccttccgatatcgccgtggaatgggagagcaatggccagccagagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagagtctgagcctgtctccaggcctgcggaagagaagggctcctgtgaagcagaccctgaacttcgacctgctgaaactggccggcgacgtggaaagcaaccccggacctatgtatagaatgcagctcctcctgctgatcgcactgagcctggctctcgtgaccaacagcgacatccagatgacacagagccccagcacactgtctgccagcgtgggagacagagtgaccatcacctgtagagccagccagtccatctcctcttggctggcctggtatcagcaaaagcctggcaaggcccctaagctgctgatctacaaggcctccacactggaaagcggggtgccctccagattttctggcagcggatctggcaccgagttcaccctgaccatcagtagcctgcagcctgacgacttcgccacctactactgccagcagtacaatacctactggaccttcggccagggaacaaaggtggaaatcaagcggactgtggccgctccaagcgtgttcatctttccacctagcgacgagcagctgaagtccggcacagcctctgttgtgtgcctgctgaacaacttctaccccagagaagccaaggtgcagtggaaagtggacaatgccctgcagagcggcaactcccaagagagcgtgacagagcaggactccaaggattccacctacagcctgtctagcaccctgacactgagcaaggccgactacgagaagcacaaagtgtatgcctgcgaagtgacccaccagggccttagctctccagtgaccaagagcttcaaccggggcgagtgttgataaagcggccgcggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 152 - DNA, 4106 bp, Artificial Sequence
  Synthetic construct; CAG.LANv2.F2A
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccgccaccatgtacagaatgcagctgctgctgctcattgccctgtctctggccctggtcaccaattctgaggtccagctgcttgagagtggtggtggactggttcagcctggtggcagcctgagactgtcttgtgctgcctctggcttcaccttcagccactatatcatgatgtgggtcagacaggcccctggcaaaggcctggaatgggtgtcaggcatctacagcagtggtggcatcacagtgtatgctgactctgtgaagggcagattcaccatcagcagagacaacagcaagaacaccctgtacctgcagatgaactccctgagagctgaggacacagcagtgtactactgtgcctatagaagaattggggtgcccagaagggatgagtttgacatctggggccagggcaccatggttacagtgtcctctgccagcacaaagggcccctctgtttttccactggctcccagcagcaagagcaccagtggtggaacagctgccctgggctgtctggtcaaggattacttccctgagcctgtgactgtgtcctggaactctggggctctgacctctggggtgcacacctttccagctgtgctgcagtcctctggcctgtactctctgtcctctgtggtcacagtgcctagctctagcctgggcacacagacctacatctgcaatgtgaaccacaagcctagcaacaccaaggtggacaagagagtggaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgccaagaccaagcctagagaggaacagtacaacagcacctacagagtggtttctgtgctgacagtgctgcaccaggactggctgaatggcaaagagtacaagtgcaaggtgtccaacaaggccctgcctgctcctattgagaaaaccatctccaaggccaagggccagccaagagaaccccaggtttacaccctgccacctagcagagaagagatgaccaagaaccaggtgtccctgacctgcctggttaagggcttctacccctctgacattgctgtggaatgggagagcaatggccagcctgaaaacaactacaagacaacccctcctgtgctggactctgatggctcattcttcctgtacagcaagctgactgtggacaagtccagatggcagcagggaaatgtgttcagctgctctgtgatgcatgaggccctgcacaaccactacacccagaaaagtctgtctctgtcccctggcagaaagagaagaggatcaggggccccagtgaagcagaccctgaactttgatctgctgaagctggctggggatgttgagagcaaccctggacctatgtataggatgcaactgctcctcctgattgctctgagcctggctcttgtgaccaactctgacatccagatgacacagagcccctccacactgtctgcatctgtgggagacagagtgaccatcacctgtagagccagccagtctatctcttcctggctggcctggtatcagcaaaagcctggcaaggcccctaagctgctgatctacaaggccagcacacttgagtcaggggtgccctccagattttctggctctggatctggcacagagttcaccctgaccatcagctccctgcagccagatgactttgccacctactactgccagcagtacaatacctactggacctttggccagggaacaaaggtggaaatcaagagaacagtggctgccccatctgtgttcatcttcccaccatctgatgaacagctgaagtctggcactgcctctgttgtgtgcctgctgaacaacttttaccctagagaagccaaggtgcagtggaaagtggataatgccctgcagtctggcaatagccaagaatctgtgacagagcaggactccaaggattccacctacagcctgagcagcaccctgacactgagcaaggctgactatgagaagcacaaagtgtatgcctgtgaagtgacacaccagggactgagcagcccagtgaccaagagcttcaacaggggagagtgctgataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctca

SEQ ID NO: 153 - DNA, 4092 bp, Artificial Sequence
  Synthetic construct; CAG.LANv2.T2A
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccgccaccatgtacagaatgcagctgctgctgctcattgccctgtctctggccctggtcaccaattctgaggtccagctgcttgagagtggtggtggactggttcagcctggtggcagcctgagactgtcttgtgctgcctctggcttcaccttcagccactatatcatgatgtgggtcagacaggcccctggcaaaggcctggaatgggtgtcaggcatctacagcagtggtggcatcacagtgtatgctgactctgtgaagggcagattcaccatcagcagagacaacagcaagaacaccctgtacctgcagatgaactccctgagagctgaggacacagcagtgtactactgtgcctatagaagaattggggtgcccagaagggatgagtttgacatctggggccagggcaccatggttacagtgtcctctgccagcacaaagggcccctctgtttttccactggctcccagcagcaagagcaccagtggtggaacagctgccctgggctgtctggtcaaggattacttccctgagcctgtgactgtgtcctggaactctggggctctgacttctggggtgcacacctttccagctgtgctgcagtcctctggcctgtactctctgtcctctgtggtcacagtgcctagctctagcctgggcacacagacctacatctgcaatgtgaaccacaagcctagcaacaccaaggtggacaagagagtggaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgccaagaccaagcctagagaggaacagtacaacagcacctacagagtggtttctgtgctgacagtgctgcaccaggactggctgaatggcaaagagtacaagtgcaaggtgtccaacaaggccctgcctgctcctattgagaaaaccatctccaaggccaagggccagccaagagaaccccaggtttacaccctgccacctagcagagaagagatgaccaagaaccaggtgtccctgacctgcctggttaagggcttctacccctctgacattgctgtggaatgggagagcaatggccagcctgaaaacaactacaagacaacccctcctgtgctggactctgatggctcattcttcctgtacagcaagctgactgtggacaagtccagatggcagcagggaaatgtgttcagctgctctgtgatgcatgaggccctgcacaaccactacacccagaaaagtctgtctctgtcccctggcagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagcccctccacactgtctgcatctgtgggagacagagtgaccatcacctgtagagccagccagtctatctctagctggctggcctggtatcagcaaaagcctggcaaggcccctaagctgctgatctacaaggccagcacacttgagtcaggggtgccctccagattttctggctctggatctggcacagagttcaccctgaccatcagctccctgcagccagatgactttgccacctactactgccagcagtacaatacctactggacctttggccagggaacaaaggtggaaatcaagagaacagtggctgccccatctgtgttcatcttcccaccatctgatgaacagctgaagtctggcactgcctctgttgtgtgcctgctgaacaacttttaccccagagaagccaaggtgcagtggaaagtggataatgccctgcagtctggcaatagccaagaatctgtgacagagcaggactccaaggattccacctacagcctgagcagcaccctgacactgagcaaggctgactatgagaagcacaaagtgtatgcctgtgaagtgacacaccagggactgagcagcccagtgaccaagagcttcaacaggggagagtgctgataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 154 - DNA, 3397 bp, Artificial Sequence
  Synthetic construct; TBG.LANv2.T2A
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagacagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcacgcgtgccgccaccatgtacagaatgcagctgctgctgctcattgccctgtctctggccctggtcaccaattctgaggtccagctgcttgagagtggtggtggactggttcagcctggtggcagcctgagactgtcttgtgctgcctctggcttcaccttcagccactatatcatgatgtgggtcagacaggcccctggcaaaggcctggaatgggtgtcaggcatctacagcagtggtggcatcacagtgtatgctgactctgtgaagggcagattcaccatcagcagagacaacagcaagaacaccctgtacctgcagatgaactccctgagagctgaggacacagcagtgtactactgtgcctatagaagaattggggtgcccagaagggatgagtttgacatctggggccagggcaccatggttacagtgtcctctgccagcacaaagggcccctctgtttttccactggctcccagcagcaagagcaccagtggtggaacagctgccctgggctgtctggtcaaggattacttccctgagcctgtgactgtgtcctggaactctggggctctgacttctggggtgcacacctttccagctgtgctgcagtcctctggcctgtactctctgtcctctgtggtcacagtgcctagctctagcctgggcacacagacctacatctgcaatgtgaaccacaagcctagcaacaccaaggtggacaagagagtggaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgccaagaccaagcctagagaggaacagtacaacagcacctacagagtggtttctgtgctgacagtgctgcaccaggactggctgaatggcaaagagtacaagtgcaaggtgtccaacaaggccctgcctgctcctattgagaaaaccatctccaaggccaagggccagccaagagaaccccaggtttacaccctgccacctagcagagaagagatgaccaagaaccaggtgtccctgacctgcctggttaagggcttctacccctctgacattgctgtggaatgggagagcaatggccagcctgaaaacaactacaagacaacccctcctgtgctggactctgatggctcattcttcctgtacagcaagctgactgtggacaagtccagatggcagcagggaaatgtgttcagctgctctgtgatgcatgaggccctgcacaaccactacacccagaaaagtctgtctctgtcccctggcagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagcccctccacactgtctgcatctgtgggagacagagtgaccatcacctgtagagccagccagtctatctctagctggctggcctggtatcagcaaaagcctggcaaggcccctaagctgctgatctacaaggccagcacacttgagtcaggggtgccctccagattttctggctctggatctggcacagagttcaccctgaccatcagctccctgcagccagatgactttgccacctactactgccagcagtacaatacctactggacctttggccagggaacaaaggtggaaatcaagagaacagtggctgccccatctgtgttcatcttcccaccatctgatgaacagctgaagtctggcactgcctctgttgtgtgcctgctgaacaacttttaccccagagaagccaaggtgcagtggaaagtggataatgccctgcagtctggcaatagccaagaatctgtgacagagcaggactccaaggattccacctacagcctgagcagcaccctgacactgagcaaggctgactatgagaagcacaaagtgtatgcctgtgaagtgacacaccagggactgagcagcccagtgaccaagagcttcaacaggggagagtgctgataagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctca

SEQ ID NO: 155 - DNA, 3226 bp, Artificial Sequence
  Synthetic construct; ApoE.hAAT.LANv2.T2A
ggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcaggtaagtatcaaggttacaagacaggtttaaggagaccaatagaaactgggcttgtcgagacagagaagactcttgcgtttctgataggcacctattggtcttactgacatccactttgcctttctctccacaggttcgaattcgccgccaccatgtacagaatgcagctgctgctgctcattgccctgtctctggccctggtcaccaattctgaggtccagctgcttgagagtggtggtggactggttcagcctggtggcagcctgagactgtcttgtgctgcctctggcttcaccttcagccactatatcatgatgtgggtcagacaggcccctggcaaaggcctggaatgggtgtcaggcatctacagcagtggtggcatcacagtgtatgctgactctgtgaagggcagattcaccatcagcagagacaacagcaagaacaccctgtacctgcagatgaactccctgagagctgaggacacagcagtgtactactgtgcctatagaagaattggggtgcccagaagggatgagtttgacatctggggccagggcaccatggttacagtgtcctctgccagcacaaagggcccctctgtttttccactggctcccagcagcaagagcaccagtggtggaacagctgccctgggctgtctggtcaaggattacttccctgagcctgtgactgtgtcctggaactctggggctctgacttctggggtgcacacctttccagctgtgctgcagtcctctggcctgtactctctgtcctctgtggtcacagtgcctagctctagcctgggcacacagacctacatctgcaatgtgaaccacaagcctagcaacaccaaggtggacaagagagtggaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgccaagaccaagcctagagaggaacagtacaacagcacctacagagtggtttctgtgctgacagtgctgcaccaggactggctgaatggcaaagagtacaagtgcaaggtgtccaacaaggccctgcctgctcctattgagaaaaccatctccaaggccaagggccagccaagagaaccccaggtttacaccctgccacctagcagagaagagatgaccaagaaccaggtgtccctgacctgcctggttaagggcttctacccctctgacattgctgtggaatgggagagcaatggccagcctgaaaacaactacaagacaacccctcctgtgctggactctgatggctcattcttcctgtacagcaagctgactgtggacaagtccagatggcagcagggaaatgtgttcagctgctctgtgatgcatgaggccctgcacaaccactacacccagaaaagtctgtctctgtcccctggcagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagcccctccacactgtctgcatctgtgggagacagagtgaccatcacctgtagagccagccagtctatctctagctggctggcctggtatcagcaaaagcctggcaaggcccctaagctgctgatctacaaggccagcacacttgagtcaggggtgccctccagattttctggctctggatctggcacagagttcaccctgaccatcagctccctgcagccagatgactttgccacctactactgccagcagtacaatacctactggacctttggccagggaacaaaggtggaaatcaagagaacagtggctgccccatctgtgttcatcttcccaccatctgatgaacagctgaagtctggcactgcctctgttgtgtgcctgctgaacaacttttaccccagagaagccaaggtgcagtggaaagtggataatgccctgcagtctggcaatagccaagaatctgtgacagagcaggactccaaggattccacctacagcctgagcagcaccctgacactgagcaaggctgactatgagaagcacaaagtgtatgcctgtgaagtgacacaccagggactgagcagcccagtgaccaagagcttcaacaggggagagtgctgataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctca

SEQ ID NO: 156 - DNA, 3472 bp, Artificial Sequence
  Synthetic construct; LSPX1.LANv2.T2A
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgtctagaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcaggtaagtatcaaggttacaagacaggtttaaggagaccaatagaaactgggcttgtcgagacagagaagactcttgcgtttctgataggcacctattggtcttactgacatccactttgcctttctctccacaggttgaattcgccgccaccatgtacagaatgcagctgctgctgctcattgccctgtctctggccctggtcaccaattctgaggtccagctgcttgagagtggtggtggactggttcagcctggtggcagcctgagactgtcttgtgctgcctctggcttcaccttcagccactatatcatgatgtgggtcagacaggcccctggcaaaggcctggaatgggtgtcaggcatctacagcagtggtggcatcacagtgtatgctgactctgtgaagggcagattcaccatcagcagagacaacagcaagaacaccctgtacctgcagatgaactccctgagagctgaggacacagcagtgtactactgtgcctatagaagaattggggtgcccagaagggatgagtttgacatctggggccagggcaccatggttacagtgtcctctgccagcacaaagggcccctctgtttttccactggctcccagcagcaagagcaccagtggtggaacagctgccctgggctgtctggtcaaggattacttccctgagcctgtgactgtgtcctggaactctggggctctgacttctggggtgcacacctttccagctgtgctgcagtcctctggcctgtactctctgtcctctgtggtcacagtgcctagctctagcctgggcacacagacctacatctgcaatgtgaaccacaagcctagcaacaccaaggtggacaagagagtggaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgccaagaccaagcctagagaggaacagtacaacagcacctacagagtggtttctgtgctgacagtgctgcaccaggactggctgaatggcaaagagtacaagtgcaaggtgtccaacaaggccctgcctgctcctattgagaaaaccatctccaaggccaagggccagccaagagaaccccaggtttacaccctgccacctagcagagaagagatgaccaagaaccaggtgtccctgacctgcctggttaagggcttctacccctctgacattgctgtggaatgggagagcaatggccagcctgaaaacaactacaagacaacccctcctgtgctggactctgatggctcattcttcctgtacagcaagctgactgtggacaagtccagatggcagcagggaaatgtgttcagctgctctgtgatgcatgaggccctgcacaaccactacacccagaaaagtctgtctctgtcccctggcagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagcccctccacactgtctgcatctgtgggagacagagtgaccatcacctgtagagccagccagtctatctctagctggctggcctggtatcagcaaaagcctggcaaggcccctaagctgctgatctacaaggccagcacacttgagtcaggggtgccctccagattttctggctctggatctggcacagagttcaccctgaccatcagctccctgcagccagatgactttgccacctactactgccagcagtacaatacctactggacctttggccagggaacaaaggtggaaatcaagagaacagtggctgccccatctgtgttcatcttcccaccatctgatgaacagctgaagtctggcactgcctctgttgtgtgcctgctgaacaacttttaccccagagaagccaaggtgcagtggaaagtggataatgccctgcagtctggcaatagccaagaatctgtgacagagcaggactccaaggattccacctacagcctgagcagcaccctgacactgagcaaggctgactatgagaagcacaaagtgtatgcctgtgaagtgacacaccagggactgagcagcccagtgaccaagagcttcaacaggggagagtgctgataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctca

SEQ ID NO: 157 - DNA, 3553 bp, Artificial Sequence
  Synthetic construct; LSPX2.LANv2.T2A
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtctagaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcaggtaagtatcaaggttacaagacaggtttaaggagaccaatagaaactgggcttgtcgagacagagaagactcttgcgtttctgataggcacctattggtcttactgacatccactttgcctttctctccacaggttgaattcgccgccaccatgtacagaatgcagctgctgctgctcattgccctgtctctggccctggtcaccaattctgaggtccagctgcttgagagtggtggtggactggttcagcctggtggcagcctgagactgtcttgtgctgcctctggcttcaccttcagccactatatcatgatgtgggtcagacaggcccctggcaaaggcctggaatgggtgtcaggcatctacagcagtggtggcatcacagtgtatgctgactctgtgaagggcagattcaccatcagcagagacaacagcaagaacaccctgtacctgcagatgaactccctgagagctgaggacacagcagtgtactactgtgcctatagaagaattggggtgcccagaagggatgagtttgacatctggggccagggcaccatggttacagtgtcctctgccagcacaaagggcccctctgtttttccactggctcccagcagcaagagcaccagtggtggaacagctgccctgggctgtctggtcaaggattacttccctgagcctgtgactgtgtcctggaactctggggctctgacttctggggtgcacacctttccagctgtgctgcagtcctctggcctgtactctctgtcctctgtggtcacagtgcctagctctagcctgggcacacagacctacatctgcaatgtgaaccacaagcctagcaacaccaaggtggacaagagagtggaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgccaagaccaagcctagagaggaacagtacaacagcacctacagagtggtttctgtgctgacagtgctgcaccaggactggctgaatggcaaagagtacaagtgcaaggtgtccaacaaggccctgcctgctcctattgagaaaaccatctccaaggccaagggccagccaagagaaccccaggtttacaccctgccacctagcagagaagagatgaccaagaaccaggtgtccctgacctgcctggttaagggcttctacccctctgacattgctgtggaatgggagagcaatggccagcctgaaaacaactacaagacaacccctcctgtgctggactctgatggctcattcttcctgtacagcaagctgactgtggacaagtccagatggcagcagggaaatgtgttcagctgctctgtgatgcatgaggccctgcacaaccactacacccagaaaagtctgtctctgtcccctggcagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagcccctccacactgtctgcatctgtgggagacagagtgaccatcacctgtagagccagccagtctatctctagctggctggcctggtatcagcaaaagcctggcaaggcccctaagctgctgatctacaaggccagcacacttgagtcaggggtgccctccagattttctggctctggatctggcacagagttcaccctgaccatcagctccctgcagccagatgactttgccacctactactgccagcagtacaatacctactggacctttggccagggaacaaaggtggaaatcaagagaacagtggctgccccatctgtgttcatcttcccaccatctgatgaacagctgaagtctggcactgcctctgttgtgtgcctgctgaacaacttttaccccagagaagccaaggtgcagtggaaagtggataatgccctgcagtctggcaatagccaagaatctgtgacagagcaggactccaaggattccacctacagcctgagcagcaccctgacactgagcaaggctgactatgagaagcacaaagtgtatgcctgtgaagtgacacaccagggactgagcagcccagtgaccaagagcttcaacaggggagagtgctgataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctca

SEQ ID NO: 158 - DNA, 3592 bp, Artificial Sequence
  Synthetic construct; LTP1.LANv2.T2A
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaggatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcaggtaagtatcaaggttacaagacaggtttaaggagaccaatagaaactgggcttgtcgagacagagaagactcttgcgtttctgataggcacctattggtcttactgacatccactttgcctttctctccacaggttgaattcgccgccaccatgtacagaatgcagctgctgctgctcattgccctgtctctggccctggtcaccaattctgaggtccagctgcttgagagtggtggtggactggttcagcctggtggcagcctgagactgtcttgtgctgcctctggcttcaccttcagccactatatcatgatgtgggtcagacaggcccctggcaaaggcctggaatgggtgtcaggcatctacagcagtggtggcatcacagtgtatgctgactctgtgaagggcagattcaccatcagcagagacaacagcaagaacaccctgtacctgcagatgaactccctgagagctgaggacacagcagtgtactactgtgcctatagaagaattggggtgcccagaagggatgagtttgacatctggggccagggcaccatggttacagtgtcctctgccagcacaaagggcccctctgtttttccactggctcccagcagcaagagcaccagtggtggaacagctgccctgggctgtctggtcaaggattacttccctgagcctgtgactgtgtcctggaactctggggctctgacttctggggtgcacacctttccagctgtgctgcagtcctctggcctgtactctctgtcctctgtggtcacagtgcctagctctagcctgggcacacagacctacatctgcaatgtgaaccacaagcctagcaacaccaaggtggacaagagagtggaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgccaagaccaagcctagagaggaacagtacaacagcacctacagagtggtttctgtgctgacagtgctgcaccaggactggctgaatggcaaagagtacaagtgcaaggtgtccaacaaggccctgcctgctcctattgagaaaaccatctccaaggccaagggccagccaagagaaccccaggtttacaccctgccacctagcagagaagagatgaccaagaaccaggtgtccctgacctgcctggttaagggcttctacccctctgacattgctgtggaatgggagagcaatggccagcctgaaaacaactacaagacaacccctcctgtgctggactctgatggctcattcttcctgtacagcaagctgactgtggacaagtccagatggcagcagggaaatgtgttcagctgctctgtgatgcatgaggccctgcacaaccactacacccagaaaagtctgtctctgtcccctggcagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagcccctccacactgtctgcatctgtgggagacagagtgaccatcacctgtagagccagccagtctatctctagctggctggcctggtatcagcaaaagcctggcaaggcccctaagctgctgatctacaaggccagcacacttgagtcaggggtgccctccagattttctggctctggatctggcacagagttcaccctgaccatcagctccctgcagccagatgactttgccacctactactgccagcagtacaatacctactggacctttggccagggaacaaaggtggaaatcaagagaacagtggctgccccatctgtgttcatcttcccaccatctgatgaacagctgaagtctggcactgcctctgttgtgtgcctgctgaacaacttttaccccagagaagccaaggtgcagtggaaagtggataatgccctgcagtctggcaatagccaagaatctgtgacagagcaggactccaaggattccacctacagcctgagcagcaccctgacactgagcaaggctgactatgagaagcacaaagtgtatgcctgtgaagtgacacaccagggactgagcagcccagtgaccaagagcttcaacaggggagagtgctgataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctca

SEQ ID NO: 159 - DNA, 4083 bp, Artificial Sequence
  Synthetic construct; LMTP6.LANv2.T2A
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcaggtaagtatcaaggttacaagacaggtttaaggagaccaatagaaactgggcttgtcgagacagagaagactcttgcgtttctgataggcacctattggtcttactgacatccactttgcctttctctccacaggttcgaattcgccgccaccatgtacagaatgcagctgctgctgctcattgccctgtctctggccctggtcaccaattctgaggtccagctgcttgagagtggtggtggactggttcagcctggtggcagcctgagactgtcttgtgctgcctctggcttcaccttcagccactatatcatgatgtgggtcagacaggcccctggcaaaggcctggaatgggtgtcaggcatctacagcagtggtggcatcacagtgtatgctgactctgtgaagggcagattcaccatcagcagagacaacagcaagaacaccctgtacctgcagatgaactccctgagagctgaggacacagcagtgtactactgtgcctatagaagaattggggtgcccagaagggatgagtttgacatctggggccagggcaccatggttacagtgtcctctgccagcacaaagggcccctctgtttttccactggctcccagcagcaagagcaccagtggtggaacagctgccctgggctgtctggtcaaggattacttccctgagcctgtgactgtgtcctggaactctggggctctgacttctggggtgcacacctttccagctgtgctgcagtcctctggcctgtactctctgtcctctgtggtcacagtgcctagctctagcctgggcacacagacctacatctgcaatgtgaaccacaagcctagcaacaccaaggtggacaagagagtggaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgccaagaccaagcctagagaggaacagtacaacagcacctacagagtggtttctgtgctgacagtgctgcaccaggactggctgaatggcaaagagtacaagtgcaaggtgtccaacaaggccctgcctgctcctattgagaaaaccatctccaaggccaagggccagccaagagaaccccaggtttacaccctgccacctagcagagaagagatgaccaagaaccaggtgtccctgacctgcctggttaagggcttctacccctctgacattgctgtggaatgggagagcaatggccagcctgaaaacaactacaagacaacccctcctgtgctggactctgatggctcattcttcctgtacagcaagctgactgtggacaagtccagatggcagcagggaaatgtgttcagctgctctgtgatgcatgaggccctgcacaaccactacacccagaaaagtctgtctctgtcccctggcagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagcccctccacactgtctgcatctgtgggagacagagtgaccatcacctgtagagccagccagtctatctctagctggctggcctggtatcagcaaaagcctggcaaggcccctaagctgctgatctacaaggccagcacacttgagtcaggggtgccctccagattttctggctctggatctggcacagagttcaccctgaccatcagctccctgcagccagatgactttgccacctactactgccagcagtacaatacctactggacctttggccagggaacaaaggtggaaatcaagagaacagtggctgccccatctgtgttcatcttcccaccatctgatgaacagctgaagtctggcactgcctctgttgtgtgcctgctgaacaacttttaccccagagaagccaaggtgcagtggaaagtggataatgccctgcagtctggcaatagccaagaatctgtgacagagcaggactccaaggattccacctacagcctgagcagcaccctgacactgagcaaggctgactatgagaagcacaaagtgtatgcctgtgaagtgacacaccagggactgagcagcccagtgaccaagagcttcaacaggggagagtgctgataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctca

SEQ ID NO: 160 - protein, 7 aa, Artificial Sequence
  synthetic construct; Y-sulfation site
EDTAVYY

SEQ ID NO: 161 - protein, 7 aa, Artificial Sequence
  synthetic construct; Y-sulfation site
DDFATYY

SEQ ID NO: 162 - protein, 22 aa, Artificial Sequence
  synthetic construct; hinge region
EPKSCDKTHLCPPCPAPELLGG

SEQ ID NO: 163 - DNA, 349 bp, Artificial Sequence
  Synthetic construct; Liver-specific CRE sequences
ggatataccctcctatgctcctcttttcattctcaaagccaccctgttcctggaatgtgagatctcctaattgttgactggagaatcaaatgctacctttgcaacagcttatcggaagcaaacaagctgaggggaattgagcaagaatttctgggataccaacagcataggaggaacaaaggacgtagagggagggttgactgtctacacaggacaaagccaatgattaaccaaacctcttgcagatttaaataggatgggaactaggagtggcagcaatcctttctttcagctggagtgctcctcaggagccagccccacccttagaaaagatgttttccatgaggat

SEQ ID NO: 164 - DNA, 349 bp, Artificial Sequence
  Synthetic construct; Liver-specific CRE sequences
ggtagacaggaaaaaaggcaaaggctaccatctgctattactgatgatattactgaggaattgatacatttctatctatagagatttattaatgtacaactggttctacaactttatttgttcagtctacataaccaaaatgaggcttcaagtccggaagttccctgttacccaaggcaaacgtattagggtctgttctgccatctggtgttctttgaaggaaacatccacagaggcattatgagataaatagccagtgttgcctatgttggtggtgggtgttacaacctcgatgaaaagcaattccagaatctctgccaaattccaataggagataatttgagaatat

SEQ ID NO: 165 - DNA, 349 bp, Artificial Sequence
  Synthetic construct; Liver-specific CRE sequences
caaccccggaaacaacaagtttctgtcaaaagcaataatgaactgttttgtgccattaacaaaaacgttatgaagacagaaaccatctcccaaagatttcataacagagccacataagtggaaagtaaatgattaaagaatgtgggtctcagagttccattcaaatcatgatactttatcttctatttacaaagataaaagtacaccagaaaatggttaatgtttaagcgctttcatatttggctctgtctttttagcagacgaaaaccactttggtagtgccagtgtgactcatccacaatgatttctccagtgctcatcttgttctcgagttttctctgccatgttg

SEQ ID NO: 166 - DNA, 329 bp, Artificial Sequence
  Synthetic construct; Liver-specific CRE sequences
aagccctgtgacatgtttataccccttataaatgcatatctagttcctgacatattttatcataaagagaaggcatttgaatcttttctctgaaatgtggttggtagatattgactagaattccaactcagaaggagacaacattgaaagtaggtttttaaacaaataatctaaactgttgctaaatgtttaatatcatggtcaaagtctctgcttgggaagtaaataagaaagtttttgaacatgtgatgcaatgtggtttcaggaatacaaaaaccttggactctgaatccaggtctctatagtttaatttacaaattagcgtaaga

SEQ ID NO: 167 - DNA, 237 bp, Artificial Sequence
  Synthetic construct; Liver-specific CRE sequences
agtgcatagttttccaattgaagtataatcaagtaaacacacagatcttaaatatttggcttgatggcttttgaaaattgcatgtagccaagtaacaaccacctaaaatagaatataaactatttcgacacccctaccccaagtttcctaatgcaagggagcccagccaccaggaaacatcattaaaaacccttctccttgtcctctctcaatatcaggcactcagtttgtagctgt

SEQ ID NO: 168 - DNA, 261 bp, Artificial Sequence
  Synthetic construct; Liver-specific CRE sequences
agcccacaaaagccgtcaaactaagtttgatttagtggtttctccattttcctgtcactgttattttgcataatttttcaaagctcagtagctggcaaataggagctgagtgtcaatgatggcggtggggtgggggccaagcttgtattcctctttagggggcgatgcaaataaaatttgaggcctcgtatgatccagtttaaaaatcacatctcaatagaaggactttcaaggaaaccacaaaaagcgtccttccttcct

SEQ ID NO: 169 - DNA, 301 bp, Artificial Sequence
  Synthetic construct; Liver-specific CRE sequences
agacccttccattcaagtgtcaccaaatgccccttcgggcccacactgtgcacccagttctcatgtccacctgggttccctaagtctttctttgcctttcatggcttttgcccttttgaagatgacagggcagtcattttgcataacgtccttcaatttgtgtgtgtacagtgccttctcatgacacaatccaggttactaagcttttggcaggaatatcacagaagtgacgctgtgtccttctcattgcatcctcttggtggaggacatacatgccaatttatcccattagtggtgacat

SEQ ID NO: 170 - DNA, 329 bp, Artificial Sequence
  Synthetic construct; Liver-specific CRE sequences
ggggctcatccatgtaagtctcatctgcaatcactgggctgatcccgtggccctgtctccagggcgccatcagagagggcttcaatcctcaggttacctgtggcccaccctgccctcagaggtgccatctctacattggccacgagatggcagcacatactcatagactgcattaatttcccagcaactcctggtgggttttccctcttatcaggatgtttgccttgctcagagagcaaatctgagagcagtgacacctaacttaactttcagcaaaatattttgagaagggtgcccctttacacatctgtgcagtccaggtgatgcat

SEQ ID NO: 171 - DNA, 253 bp, Artificial Sequence
  Synthetic construct; Liver-specific CRE sequences
tctgggtgcccactccagggcctggcttggctgccaactccagtcagggactttcagccacccctccccccaggttatttcaggagcacctgcctgggcctgggatggcttctctggtgaaagaaacaccaggattgcatcagggaggaggaggctgggatgtccagggtctgagcatctgagcagggacagatgaggttgaggttggcccacggccaggtgagaggcttccaaggcaggatacttgtgtctc

SEQ ID NO: 172 - DNA, 350 bp, Artificial Sequence
  Synthetic construct; Liver-specific CRE sequences
gagatcctcacaccttggccttccaaagcactgggattacaggcgtgagccactgtgctccctccgcctagactgtgatcggcctaaatctgatggtttctttaagaccagtttttacagagaaggcagcgggcttgcattcttctgtgccactggcaaggactgccaccagagggcgctctaccatcccttctggtgctccctgggccctgatcttgctttccactcatgccgcaaaaagccagctactcttcaagattaaactctccagggctttcttttatagataggatgagaagcaaagaacttctttgattttacagcctggatcttttcagttttggttgtat

SEQ ID NO: 173 - DNA, 283 bp, Artificial Sequence
  Synthetic construct; Liver-specific CRE sequences
ccttctcttcaagttggttgccccggtgacctttcgccaccaccgctatgatgatcttgtgcggacgctgtacaaggtgcaaaacgaatgccccggcatcacgcgggtctacagcattgggcgcagcgtggaggggagacacctctacgtgctggagttcagcgaccaccctggaatccacgagccctgtaagttctgaacagctcttgagggtgtgtggggcagggcctttcccttgcaaatccagtaattggagtggtttctggggtggtccgtggacgcc

SEQ ID NO: 174 - DNA, 285 bp, Artificial Sequence
  Synthetic construct; Liver-specific CRE sequences
ctaggattacaggtatgagccaccatgcctggcctcttttctctcttaaggtcaccctggcatgtagtctggcctgactctgcctgtgtaaacacaaaggagcaagggtcaagtgcaccatgtaatctgcccacatactctcaggatgaccctggtctttgtaaactttgctttgagaaccagtgatatgagcatcacttacagagggactgtgtgggaggggggaacagtgctctttgggagagtcaatcaggccagtgcagtgggttcaggcttcaagaaaga

SEQ ID NO: 175 - DNA, 350 bp, Artificial Sequence
  Synthetic construct; Liver-specific CRE sequences
caccttatgccaaagtctgttctatcctggaccacattctgtggtagtgtttgtttaacttttcaatttctctttcaaccctgactagtctgtaagttacttaagggtagtgactgtatcttcctcatgtttttacccccatagagcccagcatataatactagctcaacacatgtttgcccgatcaagtcaccaataaacaaaagaatgagccagcttcctcccggttccttggcaagtgtgaataactaagtcatttgtagttgacactcagctcagaggctgagggttctaaatgtatgtcccccaaagggattcatcttatgcacttcattaccagtcaagctatc

SEQ ID NO: 176 - DNA, 193 bp, Artificial Sequence
  Synthetic construct; Liver-specific CRE sequences
tttcttcacgtgaattcagattctactgaattcagctctgcaccaagtcccatcctctgctgccctctgtttcagcaaacactgaggcctgttgtttctcctcctctaccggctctgatgcggtcaaggtccagagagctgagtagttggcaagcggaccaccagcaacagacaacatcttcattcggctctc

SEQ ID NO: 177 - DNA, 346 bp, Artificial Sequence
  Synthetic construct; Liver-specific CRE sequences
gccagaagggccaaggggaggatggggcggacccgggcaggggtcctggctggaggggagcagaagaggagctctgggattaaccccggtgggtgccacttagggccagagcctctcccaggtgggacctgcgtctcatggtggctgcgcggccatcgctggggccaaggcgccgcctggtggtgaggccgggccctgcagccacacaggcgtgccccaggcaggggacaggtgggcagggtctgtggggtgcggtgccagccgccctgctcacatgggtcctgggacacatgggaggggtctgctgcctgacagtgtgggtgtcaggcttccaggaagccacgtg

SEQ ID NO: 178 - DNA, 314 bp, Artificial Sequence
  Synthetic construct; Liver-specific CRE sequences
actaggaccagtgcaggccgaggccagagagacagctgggcttccaggtagggtcaaagtgaggtgggcagcaggtgtgggggccaggggactcggggacttcctctccggctgggcccgcctgacgtgggaggcagccagggttaatcatttccacgaagccttgaccccacctgccttggccgctctgctcccgcctcccactgcccctcaggccagctcaggagccatggggcgctgggcctgggtccccagcccctggcccccaccggggctgggccccttcctcctcctcctcctgctgctgctgctgc

SEQ ID NO: 179 - DNA, 202 bp, Artificial Sequence
  Synthetic construct; Liver-specific CRE sequences
aaactgacgtcagggtttaagcttggtgggtagcagtgcctgggcctgggcagtgaccctgagcaggtgttaactaggggccgagggcggggaaggtgtcagggcctggctgaggcctcaggaatgtcgtgtgtcctcctggaacctgctccgagatctgggggccccagtatggaaacaggctcagggctgtgacctcctg

SEQ ID NO: 180 - DNA, 197 bp, Artificial Sequence
  Synthetic construct; Liver-specific CRE sequences
cctccccacagcactcaccgaggacgggaggccctgcaggttccccttccgctacgggggccgcatgctgcatgcctgcacttcggagggcagtgcacacaggaagtggtgggtccgggcagccggggcacccgagctggggtcacctgccccacgctgcctgcttttctgggagccgggcacacagtaggcgctca

SEQ ID NO: 181 - DNA, 192 bp, Artificial Sequence
  Synthetic construct; Liver-specific CRE sequences
gccaccctgaaaggaaggacacaaggttggctggtttcaccacctcctgattgtagaaccctaggacctttagcaaatataggtggtaacaaggaaatgcttaccttgggccttggccaagacccagtgctatgctgtcttcaggtctgacccaggacagtcctagtggtagtggcctcagggagtttttgt

SEQ ID NO: 182 - DNA, 184 bp, Artificial Sequence
  Synthetic construct; Liver-specific CRE sequences
tgggagcccactgccctaaagggtgagtcccaggtctggaagcattcaccacaagctgaccaaacagcccttggaccataagttaataatcaccctggaagtattccacatgggcctgtggcggtattggacacacagacagattcctcttctggtggaaaggggagagaagagtggaaaggat

SEQ ID NO: 183 - DNA, 350 bp, Artificial Sequence
  Synthetic construct; Liver-specific CRE sequences
ttccagccggctccgctccgggtcccccagtgctcgctggctccccgcttgagccagcccgcccgcccagcgctaggggccaaggagcgaaccgcacctgcgatccgattgtccggggcacgccgtagaggatgggaggacccatgaggcgctagcctgcgaaggtggcggtgctgctgctccggctgctcttggagttgcatgtacacgacgtggcagactttccaatgggaggattgtggattctgatgacataggaaacttcatcttccagacattgccacagaaagcacacagactttggaaaccagcagacctgaagtcaaatgtcgactttcagcttgctcctg

SEQ ID NO: 184 - DNA, 338 bp, Artificial Sequence
  Synthetic construct; Liver-specific CRE sequences
ctcgcgagcccgcgtgagtgcccaggggaaggggtgtaggcgaagggaggagacagctgggccatgccatgatgacctgcctctgctgcctcaacctctgtggccgctgctgggacagaggaaaggagcggtgctagctctgtctgcagatcccggccatcctgggctctttagcgccctctgcctgcagcccccgccttgacaactccgtagctgttgcccccttgctcactgaggcgcgggacctgggatcaatcgggaggacgcccgctgcagtccccagaatcaaaggatgatgtggcgcatctatgtttctttggagagtgttgtaggtctgg

SEQ ID NO: 185 - DNA, 344 bp, Artificial Sequence
  Synthetic construct; Liver-specific CRE sequences
acacagtgactctgggtagttgtgtaagagaggggcccttggcagacaaacaggttcttctctgttggtgggccagccagcaggtcagtgggaaggttaaaggtcatggggtttgggagaaactgggtgaggagttcagccccatcccccgtaaagctcctgggaagcacttctctactggggcagcccctgataccagggcactcattaaccctctgggtgccagggaaagggcaggaggtgagtgctgggaggcagctgaggtcaacttcttttgaacttccacgtggtatttactcagagcaattggtgccagaggctcagggccctggagtataaagcag

SEQ ID NO: 186 - DNA, 338 bp, Artificial Sequence
  Synthetic construct; Liver-specific CRE sequences
ggttataggaggagctggggagtatgggaagcctcccacttggtctttggttttccagaaactccaccatcacaagcaggatgttaatcagtaaccgtcccacaggggatcatactttggaatagcaaatatttgctgaaggttctgggctgcaaagctgaagctttggtttctgctctaaatgaaggacttttccaggacccaaggccacacactggtaagaggcagtgggttacaggagaccttcaatgagtctaatcagggagggaccgggaaggatggtatcatccctgggcgggctccaacgtgagggctgtgtggctgagcagtgcaaagac

SEQ ID NO: 187 - DNA, 184 bp, Artificial Sequence
  Synthetic construct; Liver-specific CRE sequences
ccagggctgagaagtggcaggtgagctggtgattccttactgcccaggttctgtctaggaaggtgcgtcctcaccatgctggatggtgtcctagtccaggagcaccccctgagctcctggcctagactccaaagggttgggtagatgagcaaagactttacaaagaccttaggcgatatatgtc

SEQ ID NO: 188 - DNA, 292 bp, Artificial Sequence
  Synthetic construct; Liver-specific CRE sequences
tgatgccacgtaatcgttttagctcaggctcctctaacaaaacaccacaggctgggtggctccaacagccattgatttttcacagttttggaggctgaaagtccgagtcagggtgccagcgtggccggattctggtagggctgtcttcttggcttgcagatggccaccttcgcaccgtgtcctcccatggagaggaggtgcggagggggactctgctctcttcttatgacagcactagtgctatcacaggggccttgccctcacgacctcatctaaacctaatcacctccca

SEQ ID NO: 189 - DNA, 256 bp, Artificial Sequence
  Synthetic construct; Liver-specific CRE sequences
tctgctctcccaaggccacagggccctacgattgtctctccctttcctttcgttaggccagcatatgattaacgctacgtgattttctatgaatgtgttttcacgtttcaaaaacagattgatacacatattgaacagtgccagacgctgtcatttgaggcccttccctggtatcctatgtgcttgtagtccttattattttcagagcactctacatagctcccctctgacacttagaagcatagggtctttccaa

SEQ ID NO: 190 - DNA, 340 bp, Artificial Sequence
  Synthetic construct; Liver-specific CRE sequences
ggtgtgaatgagacaaagtggcagtcagaggtgccaggcaaaggcttttctctaagctggagccaactatgagggaacgactgtgaattccgttcaggtccaggacaatgagaggagccagggattgttaggaaacatttccctgctttcgtgtgcgattcccaatagggcctgcgagtggagctgcattttgctagctgggctagaggacggggaaaattttggggaaatttattttgcctgcctgagctgtggaaaagccaacccaattagggaacgcctttcctagttggaacgagaagacgagaagtgagagaagtgagatagaaggctccctctc

SEQ ID NO: 191 - DNA, 227 bp, Artificial Sequence
  Synthetic construct; Liver-specific CRE sequences
gaagttttctttccgaagttcaagctagatcagaagtatgagatgcatgagctgcttaggcagatgggaatcagaagaatcttctcaccctttgctgaccttagtgaactctcagctactggaagaaatctccaagtatccagggtaagtcaggatctttcatcagagcccaacctcagcatgaaatgtcaccaaaacaaatgcttttacaaaccatttaactttga

SEQ ID NO: 192 - DNA, 200 bp, Artificial Sequence
  Synthetic construct; Liver-specific CRE sequences
gccactttcgcagagctggcaccagacttacaaagccccggcaggggaagccactttacaaccagccaggccacacccccagggcagacgtttatgtagagagtaatgtacctgcctgctagtagcctctgcattgtggggccttctctcagaaccacactaaacagtgggtgggtgagaagtgtcactcctgccacctt

SEQ ID NO: 193 - DNA, 351 bp, Artificial Sequence
  Synthetic construct; Liver-specific CRE sequences
gacagggacatttatgtctacagaagtttctgctgccttttgttcagctatgccctgcccccagaggtggagtttacagaagcagcaggccttgctgagctgcagtggtctctgcccagttcaagcttttccagctgctttgtttacctactcaagcctcagcattggcggacgcccctccccctgccaggctgctgcctcacaggtcgatctcagactgccacactagcagtgagcaaggctctgtgggcgtggtaactgccaagccaggcacaggatacagtctcctggtgtgtcctggagactggacacaccactaagaccactggaaaagtgcagtatttgtgtgtg

SEQ ID NO: 194 - DNA, 182 bp, Artificial Sequence
  Synthetic construct; Liver-specific CRE sequences
atttgaaggggtatcacaatattcatctattttctggaaatattttccaacatgcaattagcaggaaaattgaatgaaattaattctctccgttctctagtttgctaagggtaaatggtcaccaaatagataatgtaaagagaaatcaaaggagagaaaatctgctataataaagatgacaa

SEQ ID NO: 195 - DNA, 347 bp, Artificial Sequence
  Synthetic construct; Liver-specific CRE sequences
gcccagggacgtgtctttccctaaacaaaccaacctctcctcacatcccccactctgtactttcactgactatgcgttggcagattctgccaagttctacctgtaactggcttcattttcaagtcagacgtttggctgctgctctgtcccctgcaacaaggagccatgccagctggacacacacttcttccagggcctctggcagccaggacagagttgagaccacagctgttgagaccctgagccctgagtctgtattgctcaagaagggccttccccagcaatgacctcctcattgcttctggcctttctcctcctggctccaaccacagtggccactcccagag

SEQ ID NO: 196 - DNA, 344 bp, Artificial Sequence
  Synthetic construct; Liver-specific CRE sequences
tcccgacaacccccacatccctctggggtcactctggtagctcagacctgaccaataggcagctggaaagctggtaaatttcacttcttttgagattggctgccgtcatctcctccggctcctctctctgcaggccccacaaacccacaggctgtggtgtgagcccatccacttcctgggcctgtacacagcgactgggaggtgggagtctctcctttctctggccccaggatctagcccctcccttcccctctcctccagatgcctcatttgagtaaaagaagaaaaaataaaacaccaggactgcaatacctcaccatatgtcatgttcccatatatacctc

SEQ ID NO: 197 - DNA, 320 bp, Artificial Sequence
  Synthetic construct; Liver-specific CRE sequences
ataatattgccttatagcctcaaactcctggcctcaagggatcctcctgcttcagcgtcctgagtagctggggctataggcatgtgttcccatgaccagctaacaaaaggttttatagctgaccctttatttgttttgatcttcactctgagtccctgtattactggaaatgccctggatttgatttttgccctgaggtaatttcttactttaagaaccttttgtcagagagaaagaatgaaaaagaatttttgtcattatttttcttttctttcctctacatgtacaacatgaagtaaaaggttttgttttgttttgtt

SEQ ID NO: 198 - DNA, 341 bp, Artificial Sequence
  Synthetic construct; Liver-specific CRE sequences
tgagctctatcacctgagctcccctgccctctggcttcctgctgaggtcagggcatttcttatccctgttccctctctgtctaggtgtcatggttctgtgtaactgtggctattctgtgtccctacactacctggctacccccttccatggccccagctctgcctacattctgatataactgcttcaacactagggggtcctaaaggctttctatcttgctagtccctggggcctcaacatctcatactggttcccttaactctgcctatacctctgtaaataattccttcactaagttctcttgatgaagcaaaaacagacagctgaaaagtcctctatc

SEQ ID NO: 199 - DNA, 339 bp, Artificial Sequence
  Synthetic construct; Liver-specific CRE sequences
atttgtagtcttatagaaattgacaaagactacaaatcagggcttttgatttttttccctggagaccagttattaaacatttaccagcataccactgtctgcgtcctaaaaatacaagttctctacctcccacgcacttaacatacaatgctgagacagggacaggataaacataatagatattcctgttcaaaatggggtatgggagaataggagacacagagcagtcactgatccataaaacttcttatattcagccaggcaaatatctaggtccactccaaacccagaattcccttggttagttagggcctggttctgctccctggtatttcccat

SEQ ID NO: 200 - DNA, 199 bp, Artificial Sequence
  Synthetic construct; Liver-specific CRE sequences
catcctgggtggagctgcccataggccttttgtggcagcccgggtgagagttgggggcaaacaccagattcaccgacgaggcatcgactgccaaggagggtccaggatgtgctgtcgacaagagttttttgtggacttccgtgagattggctggcacgactggatcatccagcctgagggctacgccatgaacttctgc

SEQ ID NO: 201 - DNA, 210 bp, Artificial Sequence
  Synthetic construct; Liver-specific CRE sequences
agaaacagtgactggaaaagaattatcacattgcacaaggatggctctgaaatggactacagttctgctgatacaactcagtttttactttagctctgggagttgtggaaaggtgctggtatgggccgcagaatacagcctttggatgaatatgaagacaatcctgaaagaacttgttcagagaggtcatgaggtgactgtactggcatc

SEQ ID NO: 202 - DNA, 336 bp, Artificial Sequence
  Synthetic construct; Liver-specific CRE sequences
tcttcttttctttattcctatcaggaagacccactacattatctgagacaatgaggaaagctgacatatggcttatgcgaaactcctggaattttaaatttcctcatccattcttaccaaatgttgattttgttggaggactccactgcaaacctgccaaacccctacctaaggtaaacatactttcgttggttttattttgttggcttcaaatttcagtagaaatgactgtatagtcttcattcagagtgtttgactaacactgaaaaagatgggaagtaggtggggtaaagcagataccaattagaaactcatgtacatgttaataccatcaca

SEQ ID NO: 203 - DNA, 337 bp, Artificial Sequence
  Synthetic construct; Liver-specific CRE sequences
aaaaatacgagactgattatggtcgagtacagatctctatttcaataatttctcaaaaatttctagctataatttacaaatatttttacttaatattattaagatcttagcttggatccaaatgagtagttggtacaatgatttcagccatactcacaaaatagtccacagttaacttgaagaaccaaagataaaaggattagcttaatgagttgtgtaaactagactatttgttagaaaattgtttctatggttactgtagaattaattgattatggagctcaaagatttgtttaaatatctagaggctactattgaagctttaaagagaaaataa

SEQ ID NO: 204 - DNA, 348 bp, Artificial Sequence
  Synthetic construct; Liver-specific CRE sequences
ctcacattcagtacttctttagatcctcataagaaatctgggaattaagtattattcccattttctagataatgaggatcagacttagacaggggcagttaggccacatagctagaaagtgagtcacatttacttagttttaccatgtgacaggtctttcccctatatcatctcccttaatcctcacaataatcctggagggtgtgatgactgttctccctttacagatgaggaaggaggctccgtgtgtcatgtgccaaagatcacacagtcggaagcagggtagctggggtttgaagccaggttttccggctccagattcaatcctccttgtgcttcatctactac

SEQ ID NO: 205 - DNA, 299 bp, Artificial Sequence
  Synthetic construct; Liver-specific CRE sequences
tcaaatctctttaaaaatataaaagtcattcctggctcaaaggccatacaaaaataagctgtgggttggacttggacccaggctgcaatttgctaccatcctccacagttatctcatctctccattagaggaacctctgtcatagatactattccctcccattaaaaagattaggaaacaggctctgaaaaatcaagtgaaaatatttgaggtcatatagctcagaggtggcaatgcttggatttcagacctttggacttcgtatccagttctcacttctctctatcctagcacctcca

SEQ ID NO: 206 - DNA, 345 bp, Artificial Sequence
  Synthetic construct; Liver-specific CRE sequences
cttccagtaagtggttttctccccttggctggggaccctaccagtcctgatattacctgcctgctgacctcaaccgcaacccctgaccacaggccaaagtctgtttgccacaggtctttctgtacacatttccaggaactcagacaaagctattgccagtgccctcagttgacttctgtttccagggaagtgcgactaccgtaatgtggctcagtcctactgctgacttgctctggcacagtcctactgctgacttgctctgtgaccttggcaaatcacttcccctttgggggcctcaggttcctctccttaaaatgagagatctggagaagtgccttcctttta

SEQ ID NO: 207 - DNA, 325 bp, Artificial Sequence
  Synthetic construct; Liver-specific CRE sequences
ccacccacagtggggcaaggaggtgttactggcatccaggaggcagaggcctgggatgctgctaaacatccttccatgcacagaacagaccccacagcaaagaaatatctggcctcaagtgtcaatactgccaaaattgagaaaccctgccttaaatgcttaacctcacaaagtgtgaaaaattctattttaaccacaaaggaagtgattgttctagcagttgccaaactatttctgtccatatgtggtgtagtccatttaagagactgcatttcagactttgaagcaaaagatcatcattttctcatccattttctccccaccc

SEQ ID NO: 208 - DNA, 340 bp, Artificial Sequence
  Synthetic construct; Liver-specific CRE sequences
tcgaggcaattgtttcccccaaggaatatttggcaatgtcaggagacagtcttggttttcacaactggaagggggctgctggcatgtagtgggtagaggccagagactctgctaaacagcctacaatacaaggacagcccactcctgccagtaaagaattatccaacttgatacgcaatagtgccgaagttgaaaaaccctgaactacggtcatgtctggaatgggttgcatcagttgtaaataactcccacccagccacatgtagctgccacagttacaccattagggaaattgctcagcaatgacaacaacactacctgtcaaggattttattaattc

SEQ ID NO: 209 - DNA, 204 bp, Artificial Sequence
  Synthetic construct; Liver-specific CRE sequences
cacagatgccttccctgaccctgctgccctgtgtcagagtcctcctcgcacactacaataattatctgtttccttgtccatttccccacacttgactctcagctcatagaggtcaagcactctcctgagtcatctttatttctcaactagctctaacacagtaaaacgtcattaggtacttatcaaagttgtctcaaatgttct

SEQ ID NO: 210 - DNA, 346 bp, Artificial Sequence
  Synthetic construct; Liver-specific CRE sequences
atagctttgaaagtgagtaaattgggttttaaaaataattaagggagtattatttttatatctatttctaaatgtaatgttgagtcagtttgacccattgattgggaattgtaactgggagaattgttaatgtttaaactgaaaagcatgagattgttctgaaacttaagtacaggcacagagccaggaacaagcagagtgtgctggctggtgacactcaaagaggtgaacctcagagacttgcatgtcggaagaagtgtctaagccagggatggcaagtggtcttaatagcaaatgctacctagactgctgagttgaagattggaacacaagatgcgggctcagc

SEQ ID NO: 211 - DNA, 318 bp, Artificial Sequence
  Synthetic construct; Liver-specific CRE sequences
gtaccttatctgtacgtacctccaacaagctgcagtcgaccagaggaggggaggccagttcctctcccaagggtgccacacacccctccctgttcatcaccagacaggcccttccttcttagccatatgctaaccttctcctccctgggaaatttcctctgcaggagccaaagcagatgggagctggagttgctggagctcctggtctgtatgcagagcaggcatccaggaaaggagaagagagtgtgacaatccagcacctcagaatggaggggcctcgtgtccagggcggaaagtacagacgcaggcttgctgagg

SEQ ID NO: 212 - DNA, 246 bp, Artificial Sequence
  Synthetic construct; Liver-specific CRE sequences
ttttgcatcatattcacattagttatattctcaaggtgtctacattgcccaatatgcgatatgctggggatggtacacagtaggtgcttactacattttcctctcagatggcagaattcttttgtctgaggcaataactgagattagtaaaataccacaggcttctaaacataacagacttgagcgtgaatctcagcttcaccattttttaactgtatggccttaggcaatataattaaccttcat

SEQ ID NO: 213 - DNA, 313 bp, Artificial Sequence
  Synthetic construct; Liver-specific CRE sequences
aagtctggatgggaaatccaaggtattacaattgactccagaaaacaaagggttgaagcacagggtggagggggctgaagccatgtggctcaatggccatgtgaaagaaacaggccaaccattagtcccagttcactcagtgggagcctgatacagacggccccacagtgaaagacgggttaacaaaagtcaagagctcaggataagattcttctctggcgtaccaagatttttgttcttggtgccacacctcaccggatactgacaaagcaaagcaggcaatccagagaagtgggaaggctcaaacctatgt

SEQ ID NO: 214 - DNA, 347 bp, Artificial Sequence
  Synthetic construct; Liver-specific CRE sequences
taaaggaatcatgtctcaaaatacaaactcccatttacaacttccagatgactcctttagctaccgaaaaggttgaactctgtagcatgggcaatattcctccagtctgtttatctagttttatcttgccaggcatacctagggttggaaacccagctctgtttatttacatagcaatgtggctttactgctttgagaattattttcctcatctacaaaatgcaggttcctcagtgggtcctcagatgtagacaaatacagagatggctaacgtgggcctgacagtctttacggagtctgaagggcaggtaaactttccatggtgatccatccatcttctagcaaga

SEQ ID NO: 215 - DNA, 203 bp, Artificial Sequence
  Synthetic construct; Liver-specific CRE sequences
taaatatcttcctttccttaaccacacggatattgtttttcaaaaggtctaaagggcagtggcctttggtgcctggaacacttttagagtatttaggtaatcaataccctggttactatttaaccatatttgaaagagaatcaagtgagttctgtgcaaatcaatgtgtatctgggtgagtttccaacatcagatagatctta

SEQ ID NO: 216 - DNA, 345 bp, Artificial Sequence
  Synthetic construct; Liver-specific CRE sequences
gctttattccttcaaggtaatatagtgcggtggcttctggctgagatgtttgctgttgttttcttcatcttgtctttgatgacttgtcagcctggggtaactgcacaggagaaggtgaaccagtaagtgggccatatgtctctgcaaaacttgcacgtaggaatcacctgctggggaactaagacacttttatgtttgcagcagaggctgtgttagaatggaaaataacctcttttcatgttattactactcctgtggaggaggatgggacagtgaaaagaccgtgcacttggagtcaaattggtctatgtgtaaatcacagctctaatgcttagaagctgaata

SEQ ID NO: 217 - DNA, 350 bp, Artificial Sequence
  Synthetic construct; Liver-specific CRE sequences
ccacctctgtgaaacccgctctgatattgctccaccccttctcccatagtttcatctgtttatacattcattcattcaaccatgtccacatgagtatcacatgtgaaaagaacaaaatcagtatggtcactgttcttgcaaagcttacgatccagtggaggaaacaaaataaacagccaaagtccaaatatcatacttggtcctcttttacttcctggccccatcagcgagatatattgactttacttctaaagcatatgcatcacctgttctccctctattactgtctcacttgtccaagccaccattgtctctcacctggatactgcaagagtatggtggtctcttat

SEQ ID NO: 218 - DNA, 345 bp, Artificial Sequence
  Synthetic construct; Liver-specific CRE sequences
catgtctcatacttttgttttcactccaaactgcccagtgaagtaacattatcccaattcttcctatgaaacagtgaaagccctaagagtttttgaaactttacctggtttactcaatttgggaatggcagagcagaattcagtccttgaatatcctcccactgcaggttcatgctctttgatctaggtgtaacatttactctgagtaaactaggactctgggctaacagagatgaagcaagacaggctggatattaggagaatctaagagcaatctaacgaccattataataaaatcatgagttctagacttaaaaaaagggaaaaacctgtttttttgcttat

SEQ ID NO: 219 - DNA, 291 bp, Artificial Sequence
  Synthetic construct; Liver-specific CRE sequences
cttaatttcaattcatttttctgtgtctacttacaactttggcactagaattcacaatttttttttagaggtatatctccttaaagggaagggttctgacactgttacatgttctcaattgtttgcaaataggttaataattattccagtgtctctaagtacatatcaaccatgccagtgttcagcctccataattttattagcttctgtgcttattttggaaaaacatttcccattaccatgaaagacctcagtttaggatggtttggtatgttagcctgatttctgcat

SEQ ID NO: 220 - DNA, 177 bp, Artificial Sequence
  Synthetic construct; Liver-specific CRE sequences
tcatgaggaagatgaaaagacagacccaggcagggatggaaggactgacgagaaccaacttacaaagagaagttttgtttttactacatttctatgtgatcaagttcccaggttaatatttgactaaactgctaggaatccactgtgactataatgctggaaatgacttagtagggc

SEQ ID NO: 221 - DNA, 246 bp, Artificial Sequence
  Synthetic construct; Liver-specific CRE sequences
tatgggtctgactccatcactgatttcaggagccctgccctttcctccacaccatgcccccttgctttcagaaaaaaaggcttgttgactgaatggttgtatgcacagttcaaagcagaaacacacgatgacatcttttgagatactctaacagtgagaacttgaaaatgaagttaaaaattaagcggcaaaaccaagccgaggctttctgagaaagtggggccaaacctgttgccgtctgactgc

SEQ ID NO: 222 - DNA, 343 bp, Artificial Sequence
  Synthetic construct; Liver-specific CRE sequences
agaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagatgcaggtggattct

SEQ ID NO: 223 - DNA, 210 bp, Artificial Sequence
  Synthetic construct; Liver-specific CRE sequences
tctcttttctttgtattttaagcccagtgggcaaatccttttgatccatccaagacagaagacagttccagcttcttaatagacaagaccaccactgttcaagtgcccatgatgcaccagatggaacaatactatcacctagtggatatggaattgaactgcacagttctgcaaatggactacagcaagaatgctctggcactctttgtt

SEQ ID NO: 224 - DNA, 346 bp, Artificial Sequence
  Synthetic construct; Liver-specific CRE sequences
ggaattgaaagtaagccacagatgagctagagaaggctgctgataagaatatgttgcacaaattatctggcttatccaggtgctctgccgtgcgtctaacccgttgagcctgtgtgttatctccacgtgacttaatctgtgagatcttgccacaggttaacttgctcgtgtgtacaagaggcggacattcctttcagtgcagtcggaactactgttgatcaagcagagagtggcagttactgaagtgtccctgcaaagtggaaacacacctgttccttatacagttggctctcctaacctcagatttcgcatctgcagattcaaccagccacagatggaaaatatg

SEQ ID NO: 225 - DNA, 197 bp, Artificial Sequence
  Synthetic construct; Liver-specific CRE sequences
taaccatttaccttttggtgataactatttaccttagtcatttctgaggactgacccaatgctgccctctagtgataaatcttaaaagggctgccatgagctattgaaatgtgatgactggtgactcttggagggctgtgtaataaggcgtcatccatgtcccatcagcttaaaatgaggggattctgaatctcttc

SEQ ID NO: 226 - DNA, 333 bp, Artificial Sequence
  Synthetic construct; Liver-specific CRE sequences
ttttgatatttgtcaacttcataggttaaaaaaaaaataggtattttggcacggttttaatttgcctttctcttattgagtcagattgcacatttttttcatacattgaaaggccatatatatgtttttcttcgcgaactattcatttcctttgtccattgttctattgagtctcctgtctttctcttacatatttctatgatcacttcataaattagtctttgtctctgatatgaattacacatatttttcccagtttgtcattcttcctttgactttatgtggtatgttttagtggagttactttcatatttaatttattcttcacttttt

SEQ ID NO: 227 - DNA, 314 bp, Artificial Sequence
  Synthetic construct; Liver-specific CRE sequences
ggcatgtcctggataactttctgagtcaatatgtgggaacttcctcattgtcatagctgattaattcttactgatgaacatttgggtgtttccaatctttcgcactagaaataatgttgcaatgaatatccttgtttatatatcattttgtaagtggttaggtgtacctgaggattaattcccagagtgatctgagtcaaaagataatcactttgtgacagtgctaaaattgccctatttgtattattttgcactttcaccagcagagtaagagtacttgtttctcacagccttgcgacaggaaccacaggcca

SEQ ID NO: 228 - DNA, 343 bp, Artificial Sequence
  Synthetic construct; Liver-specific CRE sequences
gttagctataatagattctggattgtcacaacagtgaagagaaagtggaaatccatgaaataggaccgtgagtaaattaccgtctgatctatttcacaccttcttctgtctcaaacccacacacactccctttccgggagtgtaaggaagactagtagataagacctgtaccagaacaacctcagctgacatccactataaaatcactcattagtgcactattggttcatgccttttacccttggctgcctcttgttttttactcagtcatatttgacctcaccaagacacgactgtgtggactgctctaggctggcacaggcccctctggagagctgttgca

SEQ ID NO: 229 - DNA, 220 bp, Artificial Sequence
  Synthetic construct; Liver-specific CRE sequences
taaagtaccctataagcaacatgacgtacttaaaataataacaatagtagtaatagcaaggtaggaaaagcctgagatgtggagccagacatagcaagtacaaatgtgcagttactcgtccctggctgacgctggacaaatgatgtaatctcaatgtgtctccatttcctcagctgtaaaacagattattttcaggattaaatgagattgcatatataaa

SEQ ID NO: 230 - DNA, 251 bp, Artificial Sequence
  Synthetic construct; Liver-specific CRE sequences
gacctgtcctcccagatggtggagatggacaggaggactatctacccacccgtccccacggccctgaccctctgacctcaccctctccgctgatttcttcatgttagttcaacattaacccagaggggtcaggacagacaattcctcagtgacccaggagctgacacactatggcgcacgtccgaggcttgcagctgcctggctgcctggccctggctgccctgtgtagccttgtgcacagccagcatggt

SEQ ID NO: 231 - DNA, 158 bp, Artificial Sequence
  Synthetic construct; Liver-specific CRE sequences
tgaggccgctgtcccatgacccccccaccgccttacagtgttcctggctcctcagcaagcacggtcgctgctccagcgggtccggcgagccaacaccttcttggaggaggtgcgcaagggcaacctggagcgagagtgcgtggaggagacgtgcagct

SEQ ID NO: 232 - DNA, 157 bp, Artificial Sequence
  Synthetic construct; Liver-specific CRE sequences
cacagagaagcaagcgaggaacgccacagccccttcgctgctcacagcctcatttcaactctgagcccctcctcacagggctggcaagaggagcggcctcagcctttcctgggggtctctgtgcctggactgtgtccctgtgcagctccatgacatg

SEQ ID NO: 233 - DNA, 351 bp, Artificial Sequence
  Synthetic construct; Liver-specific CRE sequences
ttttatctaattttacgtagcaattacgttttgtgcattggttgacaagcctctgtattatcctcagaacagaaaatactgtttaagggaaattaagagcccgcagttactaaagtgactgcgccaccaagtggacaagtgtaaagccactgtctggagatggaaggattcagctttgctttataaatgggaatttgacctttaaaaatgtcccttttggcacgcacgcgcgcgcgcgcgcgcgaacacacacacacacacacacacacacacacacacacacacacacggctgctgccctgcagatttgcttgttcttgtcataaagctttcattgtttctctagctcta

SEQ ID NO: 234 - DNA, 159 bp, Artificial Sequence
  Synthetic construct; Liver-specific CRE sequences
aaaagaaggattaaatattcatttatccttttgcttctgacttgttatgtgggcaagtgccacatgagggagtgctgggacctcatatcaagaaaaattaaaacctacctaatgcgttccaggaatgttcagcatattagcaaattcttattaaactgt

SEQ ID NO: 235 - DNA, 213 bp, Artificial Sequence
  Synthetic construct; Liver-specific CRE sequences
taatgccttccaaggctggcatgccaatggctgctattaagatcgttttctctcattctaataacacacttagagatgattggtaataaaaactctcttcaaggcttctgcttctcccccttcaaaatggagatcaaagaatcatgctgtgagggtccgtcaagaagaaaagactttcagcaacagagcatgtggtgtggcataaaataatga

SEQ ID NO: 236 - DNA, 330 bp, Artificial Sequence
  Synthetic construct; Liver-specific CRE sequences
ttaaaagtaattgcctgaagctgctgtttatcacataaaaagaagacaaactagacatagcatatcttcttaaactctaatctaaactctatgcatttgtataccatcttgattttcaagattggggaagtgaaacgaaaactatgttcacacaagaacctgtacgtgaatgtttgtagtggctttatttagaatttccccccaaactgtaagtattcaaaatgtcttttagcttgggaatgactggacaaatgatagtacccctgtatgatggaatattattcatcaaccaaaaggaacaaactattgacacgtacaacaacatgagaa

SEQ ID NO: 237 - DNA, 285 bp, Artificial Sequence
  Synthetic construct; Liver-specific CRE sequences
tcttagtatgacagtaaatgcttatgttcatggtgtaggaaacagcattaaatgccagataaccatcttatccggatgaaccagactggattgttggctcaaatgttttcttcctgctggcttttcgtgttatcattcattttgattactgttgtctaaactttcactttagatttcaatttgtctatgcagcattaatctttcaactttgctgtttcatctctccttcaaagcacttcatctctcttcccaaattagttttcctttgactttcatatttcaaag

SEQ ID NO: 238 - DNA, 219 bp, Artificial Sequence
  Synthetic construct; Liver-specific CRE sequences
gttatgatagtttcttttttatagtaattgcacaggggaagagctaaaggatagaactctgtcattgtgaaccctgaaagctcagaaaaaggggagggaaatgagcctcaaggtgatgaccagtaaccaataaatcacgtcactctgccctttgacaaacatttctttatccatggctccctcagtcccctgaatgcgaaattgctttgaggagattgt

SEQ ID NO: 239 - DNA, 166 bp, Artificial Sequence
  Synthetic construct; Liver-specific CRE sequences
tagtcagcctttcctcctttgctcagctgctctgattcagagctactctgattttactctggaggacacatgcaaccaatgtctgaacagtcatactcaatccctgcattgaggtagaaagcactgattcctagaggctgatctgaatcatgcaactgtattgaag

SEQ ID NO: 240 - DNA, 312 bp, Artificial Sequence
  Synthetic construct; Liver-specific CRE sequences
acttaattgaataaacaagtaaaagccattcccaaaagtaaggtttacttgttaagattaacaaaaaataatgtgagaattctgagaaatataatctttaaatattggcaactggagtgaactcttaaaactaactaggttttatatgtttgactagagcaatgacataataaggtggttaatcatcactggacttgttttcaaaaagccaactactttaagaggaataaagggtggacttgttgcagttgctgtaggattctaaatccaggtaagaaccattgagattctctaatttttacatatatttta

SEQ ID NO: 241 - DNA, 350 bp, Artificial Sequence
  Synthetic construct; Liver-specific CRE sequences
ggctcaccttctctctgggcaaacaagttgggaacaagttcttcctgaccaatggtgaaataatgacctttgaaaaagtgaaggccttgtgtgtcaagttccaggcctctgtggccacccccaggaatgctgcagagaatggagccattcagaatctcatcaaggaggaagccttcctgggcatcactgatgagaagacagaagggcagtttgtggatctgacaggaaatagactgacctacacaaactggaacgagggtgaacccaacaatgctggttctgatgaagattgtgtattgctactgaaaaatggccagtggaatgacgtcccctgctccacctcccatctg

SEQ ID NO: 242 - DNA, 157 bp, Artificial Sequence
  Synthetic construct; Liver-specific CRE sequences
tctgtgagttccctatctgaagggtcatatcactcaggccctccttgtctttttactgcaacccacaggcccacagtatgcttgaaaagataaattatatcaatttcctcatatccagtattgttccttttgtgggcaatcactaaaaatgatcact

SEQ ID NO: 243 - DNA, 333 bp, Artificial Sequence
  Synthetic construct; Liver-specific CRE sequences
ggtcccatttgttctcactgccaccgaaagcatgtttatagtcttccagcagcaacgccaggtgtctaggcacagatgaacccctccttaggatccccactgctcatcatagtgcctacctttgttaaagtactagtcacgcagtgtcacaaggaatgtttacttttccaaatccccagctagaggccagggatgggtcatctatttctatatagcctgcacccagattgtaggacagagggcatgctcggtaaatatgtgttcattaactgagattaaccttccctgagttttctcacaccaaggtgaggaccatgtccctgtttccatcac

SEQ ID NO: 244 - DNA, 312 bp, Artificial Sequence
  Synthetic construct; Liver-specific CRE sequences
ctttaccttccagaggaaactgcctggggatatgaggagactgatgtcctatttgagtatatttttctcaactatactgtaactcaaaacagagattcagctctcattctctggcatctcagaattccacacagcagtttgtgactaatagttgtcttgccagcccaggaaagtggcccacaggtcaggccatcccgtgggacacaggatgaatttttcttctctgggtcattgtcatgtcagacccctattcacttcagtagggatggcaccaggttcaagaggccaaagaagagatggagtcagcaaaca

SEQ ID NO: 245 - DNA, 348 bp, Artificial Sequence
  Synthetic construct; Liver-specific CRE sequences
tgcctgattactacaaaccaagtgcacacagggtctcatgatctgggccttggttatcttctcaggtttatctcctcccctgccacattcactgtgtgccagccatacgaatctacatgaggttggagcacactgcttcctcatgtttgggctctgcatgctgctccctctgctggtaacaccctttcctcacttgtcaacctggaaaattcctgctgatttttcagctcttgggcccaatgcttcctctttggtgtgaaaccttccacaacttctctaggcagacttaggcactctgtctatattctcagtgcactctttacactacaccttggtagttgcatggct

SEQ ID NO: 246 - DNA, 271 bp, Artificial Sequence
  Synthetic construct; Liver-specific CRE sequences
gggttggtagtgatgccagtagtgatggtgatagtggtagtgatggtggtggtggagccactatgtggctttttaaggaagggaaatagagaagccacgtatggtctagaggtcacgtgagggaaggagaggaagtcattctggtgaaggcaactgtgtgtaattctgtgtgaatagtccctcattgttccccatgacccttaggacaaatctaccctctttagtcttacatacaagtctctccatggccaaatccctattggcccttcag

SEQ ID NO: 247 - DNA, 344 bp, Artificial Sequence
  Synthetic construct; Liver-specific CRE sequences
ctaaaatgttcaagtgggaaattacagttaaataccatggtaatgaataaaaggtacaaatcgtttaaactcttatgtaaaatttgataagatgttttacacaactttaatacattgacaaggtcttgtggagaaaacagttccagatggtaaatatacacaagggatttagtcaaacaattttttggcaagaatattatgaattttgtaatcggttggcagccaatgaaatacaaagatgagtctagttaataatctacaattattggttaaagaagtatattagtgctaatttccctccgtttgtcctagcttttctcttctgtcaaccccacacgcctttg

SEQ ID NO: 248 - DNA, 351 bp, Artificial Sequence
  Synthetic construct; Liver-specific CRE sequences
tctttgcccctattttagggataaggattctgaaatgtggagatggtaagtaaaattgcacaactgaagaatgagttacatgacttggctcaaatactggtcattgaactccagagcctgaatattcttaaccacttacatgatgcaagctcaccaaataaatagttcgaatgtattgtgacagagcggcattgatattcatctattcatgtggctttgagtaggaagaagaaaggatatcattctgaccagaggggtgaaaaacaacctgcatctgatcctgaggcataatactattaacacaattcttttatgtttcagttcgatgaatttaaacctcttgtggaagag

SEQ ID NO: 249 - DNA, 280 bp, Artificial Sequence
  Synthetic construct; Liver-specific CRE sequences
tgcaacattaatctaaaaacctcaatgacccaaatagaaaaggaaaaaagctttctgaacagccaaacagagattccaaagttcaggcaccaaagttcagaccctaacagttatttacaagggtcagttaaccttttgttctagtgggataggattcccatggtgagatttgaagaactgtggaaccactgtttcaggttctttttccactttgccacccgtttgaaattccatccattaggaattaactggcatgatgggagtcccttttataataaaa

SEQ ID NO: 250 - DNA, 198 bp, Artificial Sequence
  Synthetic construct; Liver-specific CRE sequences
gagttcaagaccagcctgggcaagtctctttaaaaaaaacaaaacaaacaaacaaaaaaattaggcatggtggcacatgcctgtagtcctagctacttaggaggctgacgtaggaggatcgtttggacctgagaggtcaaggctacagtgagccatgattgtgccactgcactccagcctgggtgacagagtgagact

SEQ ID NO: 251 - DNA, 223 bp, Artificial Sequence
  Synthetic construct; Liver-specific CRE sequences
ctctatttttaaacttctcccacaaagatgagaaaacatttaaaaatcattggggctatttttctgtttaccgagtaaagagaatctctaaaccatatttataactcttactctaaatatttgcatttaccctcatgccagagcccgttgatgactgactaaacagagtttcaaagtttgaagaacaggaaatttagaaatgactaacaattatgtaggttta

SEQ ID NO: 252 - DNA, 269 bp, Artificial Sequence
  Synthetic construct; Liver-specific CRE sequences
gtgaacaaaagaacaacccctgaatggacagatttgtggctgttagtttaatttggctaatcaagaagcaactgcaccaatgtagtaagtggccatgagcaagtttctctacaacaaaaaacattgcttcacagggtggttgagtcactggaaagattttcatatttaaactcctttagcatcccctttgtctactcacctaaaccaaatacaaaataatatcaatgggaattagtactggtttgggacttggatttgagtggggaggc

SEQ ID NO: 253 - DNA, 152 bp, Artificial Sequence
  Synthetic construct; Liver-specific CRE sequences
ctacactctcgtttcttcttttaagatttgccaatgatgatctgtcagaggtaatcactgtgcatgtgtttaaagatttcaccactttttatggtggtgatcactatagtgaaatactgaaacttgtttgtcaaattgcacagcaaggggcc

SEQ ID NO: 254 - DNA, 213 bp, Artificial Sequence
  Synthetic construct; Liver-specific CRE sequences
ctcagggcactgtgtgggagggtgagaaggagggaggcccacagaggcatgacctctgattgccactgtcacctgggccctgctctctgaagtctctgccaagcggggaggtggccgggggagggccctgctctgtgcagcctcccctcccccggcccgcagagttgagcacagagggacagaggcacggaacccccagaaatgtccctcctc

SEQ ID NO: 255 - DNA, 347 bp, Artificial Sequence
  Synthetic construct; Liver-specific CRE sequences
ggcaccattccatgtgcatcgtatgtagtaactcttaattctcacgataaccctgaggtagatattattaccccgttctacaaaaggagaaacagtcctggggagacaggataagtcaccggccaaggcacacagctagctacatgtggcccccgcgtgacggctggtctctgtaggcgaggctttgtccagatgcgtgggtagaaggtctggcccggaaagaggaactgacagcaaggctaagccaatgtctgcccctgggggcagaaagtcacctcctgctctccctccactgtccacagaggtagctcagacagggtgggggtcacaggagaacgaagggagaa

SEQ ID NO: 256 - DNA, 349 bp, Artificial Sequence
  Synthetic construct; Liver-specific CRE sequences
aagcagcaagagtgagatgagtcgtcaggtaaatcagccctcgataaaatagcaggaacctgttcacccaaaactgaaatgagtcgtcaggtaaatcagcccttgataaaacagcaggaacctgttcacccaaaaccaaaatgagttgtcaggtaaatcagaccttgataaaatagcagcgcctgttcacccgaaaccgaaatgagtcatcaggtaaatcagccctggataaaatagcaggaacctgttccgccgaaaccaaatgagtcgtcaggtaaatcagcccttgataaaataacagcacttgttgaccagaagccatgggaccagcaggcagtgagatgacaca

SEQ ID NO: 257 - DNA, 306 bp, Artificial Sequence
  Synthetic construct; Liver-specific CRE sequences
agatcaggattagggcgatggcccccgcgcgttgcctgcggagcgaggcgcactgagtcgcccaggaatccggcctctcggcgactgtgcgggagagtttatggggatgggcggggctgcttctgagcaggagtcgccgcccccacccccaccgttccgcctctgggcccgcaggctcctcccgggagcgctttcccctcctgttcaaccgccggggtacaggtggcttcgtccaccgaggtcccctcacccacgctgaggcgtcggaagctgcggacactgctcgcttcagggctttgctcagct

SEQ ID NO: 258 - DNA, 350 bp, Artificial Sequence
  Synthetic construct; Liver-specific CRE sequences
gcaggcgcctcccaggagtggtcagtggtcaatcagctaatgaagctgcataggacatgacccttgtttaccgcagaatgcccagagctggcaggatgtcttatatgcaggaagtacccaaaatgtatttattgaggaagtgatgatggataagaggaagacggagagcgagggagagaggggctaggggcccctgcggtgtaaagggggtgtggctgggagtgtgcaggggaacagggatcatttcaaggttcctatctgggagaaaataaaaaggtttacagttagttgagataagcgtgggaatatgcgaacatttttaaagaataaaaagtttagctttaaatttg

SEQ ID NO: 259 - DNA, 232 bp, Artificial Sequence
  Synthetic construct; Liver-specific CRE sequences
ataaagaacgccgtcagcacagagcagacgctgggtgttcgcgctcttgagcgtgcgctctgcggggcgcgggctggtggcgggcgggggtcgccggctccagctcaggttcccgcgccaggaccgcgtccagaaccttgtctccggaagcgggcaacggggtggttgtatcacaattagtggcatttggttttccttcttctgcattgtgggttttacttctctggggttg

SEQ ID NO: 260 - DNA, 348 bp, Artificial Sequence
  Synthetic construct; Liver-specific CRE sequences
gctggtgacctccagagagggagtctctgatgtcccgctggggtggatgtcctgagaccgggaagggggaagagacccactgaaatcctatctcccagcctcacctctgctgtctcctccacgcttcctgtctccagagccccgagttcagcataagcagaaagcggcctgttccctctctagggagaggagggttgcggtctggaggtctggctcgtctttatctgcgcattctcccagcctcctggcttcagacctcagcgaggcggcggctgccggccggctctcctcttcctgcctgcagacctggcctgctgcttctttctccttcctccctccctgcctgcc

SEQ ID NO: 261 - DNA, 173 bp, Artificial Sequence
  Synthetic construct; Liver-specific CRE sequences
tggcaccaatacctgccgcctacgactgttgtgaacactgaatgggccaacaaacctaaacgttaaatgaactgataacgccgtcagcacggagcaggcgctgggtgtttgcgctcttgcgcgtgcgctctgtggggcgcaggctgacggcgggcgggggtcgcctgctccag

SEQ ID NO: 262 - DNA, 218 bp, Artificial Sequence
  Synthetic construct; Liver-specific CRE sequences
ctcgccgcgcggcgggcctgacttctagccacgggtctccgcagttggcccagcgcttcgggccggtgttcacgctgtacgtgggctcgcagcgcatggtggtgatgcacggctacaaggcggtgaaggaagcgctgctggactacaaggacgagttctcgggcagaggcgacctccccgcgttccatgcgcacagggacaggggtgagtccgcgtcc

SEQ ID NO: 263 - DNA, 151 bp, Artificial Sequence
  Synthetic construct; Liver-specific CRE sequences
gagcagagcagctggaaccccccgagcgccctgcagacgcagcagcctcttgaggggagggtctcccccacctcgggctggacaaagacagcttttccccacgtccctctgggttctctagagcaacagcaatacccgcccggcaggggtg

SEQ ID NO: 264 - DNA, 339 bp, Artificial Sequence
  Synthetic construct; Liver-specific CRE sequences
gaagattagggctttcatcttcatttagacctgacccctgactgctttctatctaatccttcactaagcaactccttcaactggaaatatactatcctatatagcataatattcaaaactacattcttcactgggggtttccagatgaaagcccacattttgttaacatgactcactgagacagtctttgtttctcctagggcacagtcgtagtgccaactctggactctgttttgtatgacaaccaagaatttcctgatccagaaaagtttaagccagaacacttcctgaatgaaaatggaaagttcaagtacagtgactatttcaagccattttcca

SEQ ID NO: 265 - DNA, 328 bp, Artificial Sequence
  Synthetic construct; Liver-specific CRE sequences
ggagtgggctgcctggggttcatacgtcaccacacttgataatttacagaagcctttcacgcgttttttcctgtgtctcaaacggggtaccaggaggctcagggaaacagcgcaggcgcctccccggtggcttctctctggcgccctctggtgggcggggcccgggtgagcggggtcccctcggggcctcagggagccgcagccccagcggaaccaggagggcgccctccgcccagccgcgctgccacttccgggttgtttgctcccggagccataaccgcagctcagcgtgcgggcagagccggctgacaggagacaggacacagca

SEQ ID NO: 266 - DNA, 156 bp, Artificial Sequence
  Synthetic construct; Liver-specific CRE sequences
aaatcgttctttgtcattacccatcccaggttgtcctcctgtctccttgtggtgaacattggcctgtgaccctgttttatgattaacagagaggtgacatttgtgctaacgtggtacagacctttaatcccactgtagaagtttaactccctgtaa

SEQ ID NO: 267 - DNA, 333 bp, Artificial Sequence
  Synthetic construct; Liver-specific CRE sequences
ttatagaaggggatggtatccccagcaatattcagcaacattgctgtaaaaagaagaaaatctgagtgaaggtttgactggtttcccatgagaggcagacagggtcaaggtggggtcacatttactctaaccagtctcctctctcatatttgtcttctaggctaactgccaggcggccaaaggacagtatgttcacacgggttcttctggggctgcttccacccagtcgctcttcacagcctgctatacctactagggtccaatgcccgccagcctagctccagtgcttctagtaggagggctgaaagggagcaacttttcctccaatcctgg

SEQ ID NO: 268 - DNA, 329 bp, Artificial Sequence
  Synthetic construct; Liver-specific CRE sequences
gcctggggtcccggcttagaggacctctgagagctccggggccccttctgggtcgtggttgcctcatcgtggtcgggtgggtctccaggttctcccaggctcagtcccgcaggcgccaaatctgcgcaggagagcactagcaaccgatgacgtattgaggcccacacctctgggattggctgtcctgcttcgacagccttgaaagtgggtaagctgggtggggggctctgggagaggtcagtgctgagtaaggcaattcccagcagcttgagccccaccaggtcactccagtattcctccccattctttttttttttttttttttttct

SEQ ID NO: 269 - DNA, 161 bp, Artificial Sequence
  Synthetic construct; Liver-specific CRE sequences
cctgggagggacacccgcctacactctgcaagaaactcaaaaagggagatgaggggatcgtgggagggaggtagggagggaggagggtgccactgatcccctgaacccctgcctctgcctccagagtgcccctccggcctcgccatgaggctcttcctgtc

SEQ ID NO: 270 - DNA, 201 bp, Artificial Sequence
  Synthetic construct; Liver-specific CRE sequences
agagttgtgagcccttaaaaggaacagggattgctcactcggggagctcggctcttgagacaggaatcttgcccattccccgaacgaataaaccccttccttaactcagcgtctgaggaattttgtctgcggctcctcctgctacattctgagtggggaaagggactaaggtggtctgaggaccccacagagtcaggaaga

SEQ ID NO: 271 - DNA, 253 bp, Artificial Sequence
  Synthetic construct; Liver-specific CRE sequences
gagagtgctgaacggggaggggctttggggctaagggaagtgcccgggaccccacctgaccccaacgctcacgggacaggggcagaggagaaaaacgtgggtggacagagggaggcaggcggtcaggggaaggctcaggaggagggagatcaacatcaacctgccccgccccctccccagcctgataaaggtcctgcgggcaggacaggacctcccaaccaagccctccagcaaggattcaggttggtgctga

SEQ ID NO: 272 - DNA, 286 bp, Artificial Sequence
  Synthetic construct; Liver-specific CRE sequences
agggaggagaccacccctcatattgtcttatgcccaatttctgcctccaaagaaagaaaaagtaaaaactaaaaggcagaaatgaaatccacaagcagacagcccgcgccacaccctgggcctggtggttaaagattgacccctgacctaatccgttaggttatctatagattacagacattgtatagaaaagcactgtgaaaatccctattctgttttgttccgatctaattaccggtgcatgcagcccccagtcacgcatcccctgcttgttcaatcgatcacg

SEQ ID NO: 273 - DNA, 202 bp, Artificial Sequence
  Synthetic construct; Liver-specific CRE sequences
cggagtctcgctctgtcgccgaggctggagtgcagtggcgcgatctcggctcactgcaagctccgcctccctggttcacgccattctcctgcctcagcaggactacaggcgcccgccaccgcgcccggctaattttttgtattttcagtagagacagggtttcaccgtggtctcgatctcctgactttgtgatccgcctgcc

SEQ ID NO: 274 - DNA, 341 bp, Artificial Sequence
  Synthetic construct; Liver-specific CRE sequences
attttgaacccctgcccatcttcctggcaggcccagccccagcccaggggaccccagacgtctccagtgccttggataagctgaaggagtttggaaacacactggaggacaaggctcgggaactcatcagccgcatcaaacagagtgaactttctgccaagatgcggttagaacccttcccagggcacgggagagctggggtgtgtttttgggtggagccctggcagatggtccaagatgaacagattgaaaaaaaaacaagtcctggagaggctgacaacatccctctggtcacacagctagatctcaaggtgctcagacttcaaggacagtttccctga

SEQ ID NO: 275 - DNA, 245 bp, Artificial Sequence
  Synthetic construct; Liver-specific CRE sequences
tgagaaaaccgacctctaagagaacttctcagagaagctcaaaaggtgtatgtgaggagggacagggaaaaacaaaaacagaagtcaaaaccaatgttatctactttccagcaggtggctccaaacccatatgctactaaatgaggcttccagggagccagaaactataaaaggtcccaagcctcccaaacccagtttagagaaaccaaaccttcagctagaggacccaagtctacatttcccag

SEQ ID NO: 276 - DNA, 350 bp, Artificial Sequence
  Synthetic construct; Liver-specific CRE sequences
aattcttcttcaaaggttcagcttgcttaagtttccttgttctttgctccctgctttcaaggccagacttccttactctctgtgttctccctgcgctggtaaacaaccttcccgccagtcgttatctatagagcccacattccacatctgctacccactctgtgaattacccctcccatcacaatggcttctcccggcaaaactgatcttcctgccttcccagcagtgtaatcacattcctgcacttttcaagttagccaaccgggttcagcttagattgtacggtccaactctagccaatggagtcaggatacagtagcagggacaagctgcgttagacataaaaacct

SEQ ID NO: 277 - DNA, 241 bp, Artificial Sequence
  Synthetic construct; Liver-specific CRE sequences
aaattctttggcaaatggaagggatttatgtcctcaatcctcatgtctcttgccatcagaatagggttgcttattcttgtaggatgctgtgtcataccctgtgccctaggattaatacaaaagctcattgaaacagctctccccaaaacctccctcaatcctcctcctcctcgttccagtgagctttttcttttagaagacccaagtagaataacagagccaaattatgttaaaagggttt

SEQ ID NO: 278 - DNA, 351 bp, Artificial Sequence
  Synthetic construct; Liver-specific CRE sequences
ccctctgcagctggccccagaaacctgccagttattggtgccaggtctgtgccaggagggcgaggcctgtcatttctagtaatcctctgggcagtgtgactgtacctcttgcggcaactcaaagggagagggtgacttgtcccgggtcacagagctgaaagggcaggtacaacaggtgacatgccgggctgtctgagtttatgagggcccagtcttgtgtctgccgggcaatgagcaaggctccttcctgtccaagctccccgcccctccccagcctactgcctccacccgaagtctacttcctgggtgggcaggaactgggcactgtgcccagggcatgcactgcctcca

SEQ ID NO: 279 - DNA, 204 bp, Artificial Sequence
  Synthetic construct; Liver-specific CRE sequences
ccgaggaaggcctagccgctgctgctgccaggaattccaggttggaggggcggcaacctcctgccagccttcaggccactctcctgtgcctgccagaagagacagagcttgaggagagcttgaggagagcaggaaaggtgggacattgctgctgctgctcactcagttccacaggtgggagggacagcagggcttagagtgggg

SEQ ID NO: 280 - DNA, 303 bp, Artificial Sequence
  Synthetic construct; Liver-specific CRE sequences
acgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 281 - DNA, 330 bp, Artificial Sequence
  Synthetic construct; Liver-specific CRE sequences
tggccccaaatcacacaccatccataattcgggctcaggcacctggctccagtccccaaactcttgaacctggccctagtgtcactgtttctcttgggtctcaggcgctggatggggaacaggaaacctgggctggacttgaggcctctctgatgctcggtgacttcagacagttgctcaacctctctgttctcttgggcaaaacatgataacctttgacttctgtcccctcccctcaccccacccgaccttgatctctgaagtgttggaaggatttaatttttcctgcactgagttttggagacaggtcaaaaagatgaccaaggccaa

SEQ ID NO: 282 - DNA, 348 bp, Artificial Sequence
  Synthetic construct; Liver-specific CRE sequences
agagtgaggccccaggaccagaggattcttgctaaaggccaagtggatagtgatggccctgccagggctagaagccacaacctctggccctgaggccactcagcatatttagtgtccccaccctgcagaggcccaactccctcctgaccactgagccctgtaatgatgggggaatttccataagccatgaaggactgcacaaagttcagttgggaagtgaaagagaaattaaagggagatggaaatatacagcactaattttagcaccgtctttagttctaacaacactagctagctgaagaaaaatacaaacatgtattatgtaatgtgtggtctgttccatttgga

SEQ ID NO: 283 - DNA, 304 bp, Artificial Sequence
  Synthetic construct; Liver-specific CRE sequences
tggccagtttcctatagaacgcctctaaaagacctgcagcaatagcagcaagaactggtattctcgagaacttgctgcgcagcaggcacttcttggcattttatgtgtatttaatttcacaatagctctatgacaaagtccacctttctcatctccaggaaactgaggttcagagaggttaagtaacttgtccaaggtcacacagctaatagcaagttgacgtggagcaatctggcctcagagcctttaattttagccacagactgatgctcccctcttcatttagccaggctgcctctgaagt

SEQ ID NO: 284 - DNA, 259 bp, Artificial Sequence
  Synthetic construct; Liver-specific CRE sequences
ctcaatttttctatctataaaatagggctaataatttgcaccttatagggtaagctttgaggacagattagatgatacggtgcctgtaaaacaccaggtgttagtaagtgtggcaatgatggtgacgctgaggctgatgtttgcttagcatagggttaggcagctggcaggcagtaaacagttggataatttaatggaaaatttgccaaactcagatgctgttcactgctgagcaggagccccttcctgctgaaatggt

SEQ ID NO: 285 - DNA, 155 bp, Artificial Sequence
  Synthetic construct; Liver-specific CRE sequences
tgcagatgggaaaacaaaggcccagagaggggaagaaatgcccaggagctaccgagggcaggcgacctcaaccacagcccagtgctggagctgtgagtggatgtagagcagcggaatatccattcagccagctcaggggaaggacaggggccctg

SEQ ID NO: 286 - DNA, 258 bp, Artificial Sequence
  Synthetic construct; Liver-specific CRE sequences
gcagatggagaggctgcagctgagctcctattttcataataacagcagccatgagggttgtgtcctgtttcccagtcctgcccggtcccccctcggtacctcctggtggatacactggttcctgtaagcagaagtggatgagggtgtctaggtctgcagtcctggcaccccaggatgggggacaccagccaagatacagcaacagcaacaaagcgcagccatttctttctgtttgcacagctcctctgtctgtcgggg

SEQ ID NO: 287 - DNA, 337 bp, Artificial Sequence
  Synthetic construct; Liver-specific CRE sequences
aactcttcactgtgaaaggaggaaaccagagtaggtgatgtgacaccaggaaagatggatgggtgtgggggaatgtgctcctcccagctgtcaccccctcgccaccctccctgcaccagcctctccacctcctttgagcccagaattcccctgtctaggagggcacctgtctcatgcctagccatgggaattctccatctgttttgctacattgaacccagatgccattctaaccaagaatcctggctgggtgcaggggctctcgcctgtaaccccagcactttgggaggccaaggcaggcggatcaagaggtcaggagttcaagacctgcctggcc

SEQ ID NO: 288 - DNA, 317 bp, Artificial Sequence
  Synthetic construct; Liver-specific CRE sequences
ttatttctttagcctcaccagcttcctttcaaatgcgtggctaatggcttccctatcaggcagtgcagactgaagaacatttccatggttactgagagttctactcagcacttctgttgtgaaggattcaccttccccaagctgtgtgccacatttcacacagcaggtgcacagggatgccagctctcttttttccaccccttctctttactctcctcttctttgcatcactcccctctgattcacttctcttcctttcccccttcccttcctgtccccttcctacttcagaacagaacaggcaacctatttcacct

SEQ ID NO: 289 - DNA, 237 bp, Artificial Sequence
  Synthetic construct; Liver-specific CRE sequences
attttataaatataactttttataatatttagaccttttcctttgagcaaaattagtaaagtgttcttacagtagcataagtcagaaaagcaaaacaaaacaaaaacccttgtatctctagggggcataggagaccaggatctaaatacttcgaaaaggttttcaggttaacaatatgtccatttcgaagtcacatgttaactttaattctaaatttatgctagaatccattcctaa

SEQ ID NO: 290 - DNA, 351 bp, Artificial Sequence
  Synthetic construct; Liver-specific CRE sequences
gtctgcagggcttctgtgctgggcccatacaggttacaagcctccccccatctcagaccccaagagtacactccatttacaaggtggaggaggtagttaagccctgggcctcaggagagaatgtctattcctacgcatgccaaaaccacatctgtaggaacttaagcattttctgctgttgtgtcattctcattgttatcgtggtgattgttgtaaaaactgctatatgagccttggaaacatgaagctaagtcagaaaaaaaatcagatacaaaaagtcatatgttgtatgatttcatttatgtgaaaggtccagaataggcaaatctattagatgaactgctagcaggg

SEQ ID NO: 291 - DNA, 171 bp, Artificial Sequence
  Synthetic construct; Liver-specific CRE sequences
actgatttaaccttacacgtggctcccaaggtcaccgaagcagaggaggagagagctggaatgtcttaccaaggcttctgtgaccatatctggaaggaacatatatcactgctggcctcatctcattgcccaactggtcccatggccccagcctcacggcaaaggggctgg

SEQ ID NO: 292 - DNA, 333 bp, Artificial Sequence
  Synthetic construct; Liver-specific CRE sequences
aatatcatttgcattttgcaaagggagacactgagtttcagacagctgcttttgcacagtatgaggcagtgagttactgcacagccttgattccaaggtcccctcatttacaattgaatcccctttgggaaataggtcatttgcaagtcagggagtccctggggtgcagtggagtagaaaatggtgctgagttcaatgggataaaaagaataaaccagaaatagtttcattacaggcacttcattttacccaggggtagaacaatttttaaattgatttgcatcttccaaattttcgatcaaaagaaaatctgaagtgtatggaagctgtcat

SEQ ID NO: 293 - DNA, 219 bp, Artificial Sequence
  Synthetic construct; Liver-specific CRE sequences
cccacattgggagttcactggggctgtcattaagtgaacccacactgtgctggagaaagggaggacgtagccttgggggcgtgcccacagtactttgaccagcaaacgcatttcaacatagcagataatgagaccacattcgtccctcctgacacctggcctttgtcgtttggcaggctacttagggcaccaggggctacaaacccatttgcaaaaata

SEQ ID NO: 294 - DNA, 347 bp, Artificial Sequence
  Synthetic construct; Liver-specific CRE sequences
ggatcatacaaccacagacacaggcaacagccacaaccacaccaccacctgctgccagcatcagaatattctcaaagcgagcctgggcttaagtcacaatccaccatttgtttgtgggacaggcagtcagggcagatactttgaaaaaaaagagcccttcctgtttgctgtgcattgtcttcataccagttcccagaattgacctgacaaatattgaccaggttgctgtcatcttcaaacaccattttcctgtgggtagaggtgatgcagtgctgaagacctgggcccctgctcagtgcctttgctctgtaagtactttgcctgtgcccaaaatgagggcaggggca

SEQ ID NO: 295 - DNA, 335 bp, Artificial Sequence
  Synthetic construct; Liver-specific CRE sequences
ccctgcccagccccaaactcgaactaaaagtaggaaactccctgttcctagacaagcgactaaagcctcggcagcactatttggacagcatcaaggagctttatggagcttttgctttttctgccaacttcacagattctgttacaactgggaggcagattaatgactatttgagaaggcaaacatacgggcaagtcgtggactgcctcccggagttcagccaggacacgttcatggttcttgccaattacatcttcttcaaaggtgaggcttgctctgacagaattattgccactgcctctgcaagtaaatgctcttacagcagagagccagct

SEQ ID NO: 296 - DNA, 209 bp, Artificial Sequence
  Synthetic construct; Liver-specific CRE sequences
attctgggtgcttcaagagctttcgggagcccagctgttccccaactaaactccagcagcatccctccaccaggcattgagctgatccaaaatgtcacctctatctcctttacacacagccctccccctgcctttgtcaggagccagtgttacaatgcatgtaatatcctatcacttcataaacccttaataagtgagaatattatact

SEQ ID NO: 297 - DNA, 329 bp, Artificial Sequence
  Synthetic construct; Liver-specific CRE sequences
cttcaggttttcaatctccttttatggaaaacttcaagaattttgagagaagaatcccaaatattcctgccagttagacaagaaaatgaaacaatacaacccaaagcacaatgacaatgatgctgcagcaaatttactagaagctgtcctgtgaaaagggtgcttaggagctctggtctggaatctgagtcttgctcctgctctcaccaggcggcttgtcctaaccctgcatcagtcattccctgtgtacagtagggctaataatccctgtgctttgaggttctcataaggattaaattaaataagacaaagcaagtgtttctcaacaa

SEQ ID NO: 298 - DNA, 256 bp, Artificial Sequence
  Synthetic construct; Liver-specific CRE sequences
gaatggaacggtggaagagttggatttgaagggagaagagaaggctgccttccttactgagaaaatcaagatttgggagggtcccagtagcaatgtggtcatgagtcttctgcaagaacctgaagatgggtgtagggtcgggggatgggactgaaagggaaaggagagcatggaacattgggcagggtgagtagagacaaccctgtttgcactgcagttccccctccagctcaggctgcattcccagccctaaaac

SEQ ID NO: 299 - DNA, 169 bp, Artificial Sequence
  Synthetic construct; Liver-specific CRE sequences
gaaaataaataccttgatgacccaatttggaaaactacttctagataaggaaacactattacccaatatgttaatttttaaaatacccaaagaattgagcaaaaaggactttggcaaggcaggttgttggatttaaaaccgttctcccattaatgccaaagccagcaga

SEQ ID NO: 300 - DNA, 333 bp, Artificial Sequence
  Synthetic construct; Liver-specific CRE sequences
gaagatagttggaggccctcaaacttacctcctcaaggaaatctgggctggagtttttaaggggttaatggagggtgatggactgaaaaattggagtcatagattagtcaggataatgaagataaaatcagtaggagttggaaactgcattctttggtgagtcagctccttgtggggtccttgagagcagctggcatcagggaggtccctcagaccagctgagtcagtagtttcatcagtatgctggacctgaaggaatacctcaaagagaaaaccacatttcaaaatgtttaagttattatctatagaacagttaagatgaactttaatctt

SEQ ID NO: 301 - DNA, 346 bp, Artificial Sequence
  Synthetic construct; Liver-specific CRE sequences
ttaaattttacacaataattgataatttgtatgatatctgaattatacgtcaacaaatttacaaaagtttttgaaccacatttagctattttatctttagttctgtgtcaaagagactgcaggaacatgagcctctcactgctactcttatgcacactattctgaaatatattacatgagtcaggtgatgagtccatccaagatcattgatattagctgaattacagcacttaaaaatctagggctgggtgcggtggctcacgcctgtaatcccagcactttgggaggccaaggagggcagatcacaaggtcaggggttcgagaccatcctgccgaatacagtgaa

SEQ ID NO: 302 - DNA, 295 bp, Artificial Sequence
  Synthetic construct; Liver-specific CRE sequences
gaggggtcctgcaacatctcagtctgcgcagctggctgtgggggtaactctgtcttaggccaggcagccctgccttcagtttccccacctttcccagggcaggggagaggcctctggcctgacatcatccacaatgcaaagaccaaaacagccgtgacctccattcacatgggctgagtgccaactctgagccagggatctgaggacagcatcgcctcaagtgacgcagggactggccgggcgcagcagctcacgcctgtaattccagcactttgggaggccgaggctggctgat

SEQ ID NO: 303 - DNA, 349 bp, Artificial Sequence
  Synthetic construct; Liver-specific CRE sequences
aggctgttgttacctaggtccgactcctggctcccagagcagcctgtcctgcctgcctggaactctgagcaggctggagtcatggagtcgattcccagaatcccagagtcagggaggctgggggcaggggcaggtcactggacaaacagatcaaaggtgagaccagcgtagggctgcagaccaggccaggccagctggacgggcacaccatgaggtaggtgggcgcccacagcctccctgcagggtgtggggtgggagcacaggcctgggccctcaccgcccctgccctgcccataggctgctgaccctcctgggccttctgtgtggctcggtggccacccccttgggc

SEQ ID NO: 304 - DNA, 154 bp, Artificial Sequence
  Synthetic construct; Liver-specific CRE sequences
acacctgaccccacagctgagctcgggggccaaggtgctggccacgctgtgcgggcaggagagcacagacacggagcgggcccctggcaaggacactttctactcgctgggctccagcctggacattaccttccgctccgactactccaacgag

SEQ ID NO: 305 - DNA, 56 bp, Artificial Sequence
  synthetic construct; poly A
aataaaggaaatttattttcattgcaatagtgtgttggaattttttgtgtctctca

SEQ ID NO: 306 - DNA, 130 bp, Artificial Sequence; identical to SEQ ID NO: 46
  synthetic construct; 5′ ITR

SEQ ID NO: 307 - DNA, 130 bp, Artificial Sequence
  Synthetic construct; 3′ ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 308 - DNA, 3005 bp, Artificial Sequence
  Synthetic construct; 5′ITR-ApoE.hAAT promoter- chimeric intron - leader-VH-4GSx3 linker-VL-9G linker-Fc-polyA- 3′ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcaggtaagtatcaaggttacaagacaggtttaaggagaccaatagaaactgggcttgtcgagacagagaagactcttgcgtttctgataggcacctattggtcttactgacatccactttgcctttctctccacaggttgaattcgccaccatgtacagaatgcagctgctgctgctcattgccctgtctctggccctggtcaccaattctgaggaggtccagctgcttgagagtggtggtggactggttcagcctggtggcagcctgagactgtcttgtgctgcctctggcttcaccttcagccactatatcatgatgtgggtcagacaggcccctggcaaaggcctggaatgggtgtcaggcatctacagcagtggtggcatcacagtgtatgctgactctgtgaagggcagattcaccatcagcagagacaacagcaagaacaccctgtacctgcagatgaactccctgagagctgaggacacagcagtgtactactgtgcctatagaagaattggggtgcccagaagggatgagtttgacatctggggccagggcaccatggttacagtgtcctctggtggaggcggttcaggcggaggtggcagcggcggtggagggtcggacatccagatgacacagagcccctccacactgtctgcatctgtgggagacagagtgaccatcacctgtagagccagccagtctatctctagctggctggcctggtatcagcaaaagcctggcaaggcccctaagctgctgatctacaaggccagcacacttgagtcaggggtgccctccagattttctggctctggatctggcacagagttcaccctgaccatcagctccctgcagccagatgactttgccacctactactgccagcagtacaatacctactggacctttggccagggaacaaaggtggaaatcaagggcggaggaggcggaggtggaggaggagaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgccaagaccaagcctagagaggaacagtacaacagcacctacagagtggtttctgtgctgacagtgctgcaccaggactggctgaatggcaaagagtacaagtgcaaggtgtccaacaaggccctgcctgctcctattgagaaaaccatctccaaggccaagggccagccaagagaaccccaggtttacaccctgccacctagcagagaagagatgaccaagaaccaggtgtccctgacctgcctggttaagggcttctacccctctgacattgctgtggaatgggagagcaatggccagcctgaaaacaactacaagacaacccctcctgtgctggactctgatggctcattcttcctgtacagcaagctgactgtggacaagtccagatggcagcagggaaatgtgttcagctgctctgtgatgcatgaggccctgcacaaccactacacccagaaaagtctgtctctgtcccctggctaatgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggcggccgcccgagtttaattggtttatagaactcttcaagctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 309 - DNA, 723 bp, Artificial Sequence; identical to SEQ ID NO: 21
  Synthetic construct; ApoE.hAAT promoter

SEQ ID NO: 310 - DNA, 133 bp, Artificial Sequence; identical to SEQ ID NO: 41
  Synthetic construct; chimeric intron

SEQ ID NO: 311 - DNA, 63 bp, Artificial Sequence
  Synthetic construct; mIL2 leader
atgtacagaatgcagctgctgctgctcattgccctgtctctggccctggtcaccaattctgag

SEQ ID NO: 312 - protein, 21 aa, Artificial Sequence
  Synthetic construct; mIL2 leader
MYRMQLLLLIALSLALVTNSE

SEQ ID NO: 313 - DNA, 366 bp, Artificial Sequence
  Synthetic construct; Lanadelumab VH (codon-optimized and CpG-deleted)
gaggtccagctgcttgagagtggtggtggactggttcagcctggtggcagcctgagactgtcttgtgctgcctctggcttcaccttcagccactatatcatgatgtgggtcagacaggcccctggcaaaggcctggaatgggtgtcaggcatctacagcagtggtggcatcacagtgtatgctgactctgtgaagggcagattcaccatcagcagagacaacagcaagaacaccctgtacctgcagatgaactccctgagagctgaggacacagcagtgtactactgtgcctatagaagaattggggtgcccagaagggatgagtttgacatctggggccagggcaccatggttacagtgtcctct

SEQ ID NO: 314 - protein, 122 aa, Artificial Sequence
  Synthetic construct; Lanadelumab VH (codon- optimized and CpG-deleted)
EVQLLESGGGLVQPGGSLRLSCAASGFTFSHYIMMWVRQAPGKGLEWVSGIYSSGGITVYADSVKGRFTISRDNSKNTLYLQMNSLRAEDTAVYYCAYRRIGVPRRDEFDIWGQGTMVTVSS

SEQ ID NO: 315 - DNA, 45 bp, Artificial Sequence
  Synthetic construct; linker
ggtggaggcggttcaggcggaggtggcagcggcggtggagggtcg

SEQ ID NO: 316 - protein, 15 aa, Artificial Sequence
  Synthetic construct; linker
GGGGSGGGGSGGGGS

SEQ ID NO: 317 - DNA, 318 bp, Artificial Sequence
  Synthetic construct; Lanadelumab VL (codon-optimized and CpG-deleted)
gacatccagatgacacagagcccctccacactgtctgcatctgtgggagacagagtgaccatcacctgtagagccagccagtctatctctagctggctggcctggtatcagcaaaagcctggcaaggcccctaagctgctgatctacaaggccagcacacttgagtcaggggtgccctccagattttctggctctggatctggcacagagttcaccctgaccatcagctccctgcagccagatgactttgccacctactactgccagcagtacaatacctactggacctttggccagggaacaaaggtggaaatcaag

SEQ ID NO: 318 - protein, 106 aa, Artificial Sequence
  Synthetic construct; Lanadelumab VL (codon- optimized and CpG-deleted)
DIQMTQSPSTLSASVGDRVTITCRASQSISSWLAWYQQKPGKAPKLLIYKASTLESGVPSRFSGSGSGTEFTLTISSLQPDDFATYYCQQYNTYWTFGQGTKVEIK

SEQ ID NO: 319 - DNA, 27 bp, Artificial Sequence
  Synthetic construct; linker
ggcggaggaggcggaggtggaggagga

SEQ ID NO: 320 - protein, 9 aa, Artificial Sequence
  Synthetic construct; linker
GGGGGGGGG

SEQ ID NO: 321 - DNA, 693 bp, Artificial Sequence
  Synthetic construct; Lanadelumab Fc (codon- optimized and CpG-deleted)
gaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgccaagaccaagcctagagaggaacagtacaacagcacctacagagtggtttctgtgctgacagtgctgcaccaggactggctgaatggcaaagagtacaagtgcaaggtgtccaacaaggccctgcctgctcctattgagaaaaccatctccaaggccaagggccagccaagagaaccccaggtttacaccctgccacctagcagagaagagatgaccaagaaccaggtgtccctgacctgcctggttaagggcttctacccctctgacattgctgtggaatgggagagcaatggccagcctgaaaacaactacaagacaacccctcctgtgctggactctgatggctcattcttcctgtacagcaagctgactgtggacaagtccagatggcagcagggaaatgtgttcagctgctctgtgatgcatgaggccctgcacaaccactacacccagaaaagtctgtctctgtcccctggc

SEQ ID NO: 322 - protein, 231 aa, Artificial Sequence
  Synthetic construct; Lanadelumab Fc (codon- optimized and CpG-deleted)
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 323 - DNA, 1512 bp, Artificial Sequence
  Synthetic construct; VH-VL-Fc sequence (including leader and linkers)
atgtacagaatgcagctgctgctgctcattgccctgtctctggccctggtcaccaattctgaggaggtccagctgcttgagagtggtggtggactggttcagcctggtggcagcctgagactgtcttgtgctgcctctggcttcaccttcagccactatatcatgatgtgggtcagacaggcccctggcaaaggcctggaatgggtgtcaggcatctacagcagtggtggcatcacagtgtatgctgactctgtgaagggcagattcaccatcagcagagacaacagcaagaacaccctgtacctgcagatgaactccctgagagctgaggacacagcagtgtactactgtgcctatagaagaattggggtgcccagaagggatgagtttgacatctggggccagggcaccatggttacagtgtcctctggtggaggcggttcaggcggaggtggcagcggcggtggagggtcggacatccagatgacacagagcccctccacactgtctgcatctgtgggagacagagtgaccatcacctgtagagccagccagtctatctctagctggctggcctggtatcagcaaaagcctggcaaggcccctaagctgctgatctacaaggccagcacacttgagtcaggggtgccctccagattttctggctctggatctggcacagagttcaccctgaccatcagctccctgcagccagatgactttgccacctactactgccagcagtacaatacctactggacctttggccagggaacaaaggtggaaatcaagggcggaggaggcggaggtggaggaggagaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgccaagaccaagcctagagaggaacagtacaacagcacctacagagtggtttctgtgctgacagtgctgcaccaggactggctgaatggcaaagagtacaagtgcaaggtgtccaacaaggccctgcctgctcctattgagaaaaccatctccaaggccaagggccagccaagagaaccccaggtttacaccctgccacctagcagagaagagatgaccaagaaccaggtgtccctgacctgcctggttaagggcttctacccctctgacattgctgtggaatgggagagcaatggccagcctgaaaacaactacaagacaacccctcctgtgctggactctgatggctcattcttcctgtacagcaagctgactgtggacaagtccagatggcagcagggaaatgtgttcagctgctctgtgatgcatgaggccctgcacaaccactacacccagaaaagtctgtctctgtcccctggc

SEQ ID NO: 324 - protein, 504 aa, Artificial Sequence
  Synthetic construct; VH-VL-Fc sequence (including leader and linkers)
MYRMQLLLLIALSLALVTNSEEVQLLESGGGLVQPGGSLRLSCAASGFTFSHYIMMWVRQAPGKGLEWVSGIYSSGGITVYADSVKGRFTISRDNSKNTLYLQMNSLRAEDTAVYYCAYRRIGVPRRDEFDIWGQGTMVTVSSGGGGSGGGGSGGGGSDIQMTQSPSTLSASVGDRVTITCRASQSISSWLAWYQQKPGKAPKLLIYKASTLESGVPSRFSGSGSGTEFTLTISSLQPDDFATYYCQQYNTYWTFGQGTKVEIKGGGGGGGGGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 325 - DNA, 3862 bp, Artificial Sequence
  Synthetic construct; 5′ITR-LMTP6 promoter- chimeric intron - leader- VH- 4GSx3 linker- VL- 9G linker- Fc- polyA-3′ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcaggtaagtatcaaggttacaagacaggtttaaggagaccaatagaaactgggcttgtcgagacagagaagactcttgcgtttctgataggcacctattggtcttactgacatccactttgcctttctctccacaggttcgaattcgccaccatgtacagaatgcagctgctgctgctcattgccctgtctctggccctggtcaccaattctgaggaggtccagctgcttgagagtggtggtggactggttcagcctggtggcagcctgagactgtcttgtgctgcctctggcttcaccttcagccactatatcatgatgtgggtcagacaggcccctggcaaaggcctggaatgggtgtcaggcatctacagcagtggtggcatcacagtgtatgctgactctgtgaagggcagattcaccatcagcagagacaacagcaagaacaccctgtacctgcagatgaactccctgagagctgaggacacagcagtgtactactgtgcctatagaagaattggggtgcccagaagggatgagtttgacatctggggccagggcaccatggttacagtgtcctctggtggaggcggttcaggcggaggtggcagcggcggtggagggtcggacatccagatgacacagagcccctccacactgtctgcatctgtgggagacagagtgaccatcacctgtagagccagccagtctatctctagctggctggcctggtatcagcaaaagcctggcaaggcccctaagctgctgatctacaaggccagcacacttgagtcaggggtgccctccagattttctggctctggatctggcacagagttcaccctgaccatcagctccctgcagccagatgactttgccacctactactgccagcagtacaatacctactggacctttggccagggaacaaaggtggaaatcaagggcggaggaggcggaggtggaggaggagaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgccaagaccaagcctagagaggaacagtacaacagcacctacagagtggtttctgtgctgacagtgctgcaccaggactggctgaatggcaaagagtacaagtgcaaggtgtccaacaaggccctgcctgctcctattgagaaaaccatctccaaggccaagggccagccaagagaaccccaggtttacaccctgccacctagcagagaagagatgaccaagaaccaggtgtccctgacctgcctggttaagggcttctacccctctgacattgctgtggaatgggagagcaatggccagcctgaaaacaactacaagacaacccctcctgtgctggactctgatggctcattcttcctgtacagcaagctgactgtggacaagtccagatggcagcagggaaatgtgttcagctgctctgtgatgcatgaggccctgcacaaccactacacccagaaaagtctgtctctgtcccctggctaatgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggcggccgcccgagtttaattggtttatagaactcttcaagctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 326 - DNA, 1579 bp, Artificial Sequence; identical to SEQ ID NO: 14
  Synthetic construct; LMTP6 promoter

SEQ ID NO: 327 - DNA, 133 bp, Artificial Sequence; identical to SEQ ID NO: 41
  Synthetic construct; chimeric intron

SEQ ID NO: 328 - DNA, 63 bp, Artificial Sequence; identical to SEQ ID NO: 311
  Synthetic construct; mIL2 leader

SEQ ID NO: 329 - protein, 21 aa, Artificial Sequence
  Synthetic construct;
MYRMQLLLLIALSLALVTNSE

SEQ ID NO: 330 - DNA, 366 bp, Artificial Sequence; identical to SEQ ID NO: 313
  Synthetic construct; Lanadelumab VH (codon- optimized and CpG-deleted)

SEQ ID NO: 331 - protein, 122 aa, Artificial Sequence; identical to SEQ ID NO: 314
  Synthetic construct; Lanadelumab VH (codon- optimized and CpG-deleted)

SEQ ID NO: 332 - DNA, 3005 bp, Artificial Sequence
  Synthetic construct; 5′ITR-ApoE.hAAT promoter- chimeric intron - leader- VL- 4GSx3 linker- VH- 9G linker- Fc- polyA-3′ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcaggtaagtatcaaggttacaagacaggtttaaggagaccaatagaaactgggcttgtcgagacagagaagactcttgcgtttctgataggcacctattggtcttactgacatccactttgcctttctctccacaggttgaattcgccaccatgtacagaatgcagctgctgctgctcattgccctgtctctggccctggtcaccaattctgaggacatccagatgacacagagcccctccacactgtctgcatctgtgggagacagagtgaccatcacctgtagagccagccagtctatctctagctggctggcctggtatcagcaaaagcctggcaaggcccctaagctgctgatctacaaggccagcacacttgagtcaggggtgccctccagattttctggctctggatctggcacagagttcaccctgaccatcagctccctgcagccagatgactttgccacctactactgccagcagtacaatacctactggacctttggccagggaacaaaggtggaaatcaagggtggaggcggttcaggcggaggtggcagcggcggtggagggtcggaggtccagctgcttgagagtggtggtggactggttcagcctggtggcagcctgagactgtcttgtgctgcctctggcttcaccttcagccactatatcatgatgtgggtcagacaggcccctggcaaaggcctggaatgggtgtcaggcatctacagcagtggtggcatcacagtgtatgctgactctgtgaagggcagattcaccatcagcagagacaacagcaagaacaccctgtacctgcagatgaactccctgagagctgaggacacagcagtgtactactgtgcctatagaagaattggggtgcccagaagggatgagtttgacatctggggccagggcaccatggttacagtgtcctctggcggaggaggcggaggtggaggaggagaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgccaagaccaagcctagagaggaacagtacaacagcacctacagagtggtttctgtgctgacagtgctgcaccaggactggctgaatggcaaagagtacaagtgcaaggtgtccaacaaggccctgcctgctcctattgagaaaaccatctccaaggccaagggccagccaagagaaccccaggtttacaccctgccacctagcagagaagagatgaccaagaaccaggtgtccctgacctgcctggttaagggcttctacccctctgacattgctgtggaatgggagagcaatggccagcctgaaaacaactacaagacaacccctcctgtgctggactctgatggctcattcttcctgtacagcaagctgactgtggacaagtccagatggcagcagggaaatgtgttcagctgctctgtgatgcatgaggccctgcacaaccactacacccagaaaagtctgtctctgtcccctggctaatgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggcggccgcccgagtttaattggtttatagaactcttcaagctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 333 - DNA, 3862 bp, Artificial Sequence
  Synthetic construct; 5′ITR-LMTP6 promoter- chimeric intron - leader- VL- 4GSx3 linker- VH- 9G linker- Fc- polyA-3′ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcaggtaagtatcaaggttacaagacaggtttaaggagaccaatagaaactgggcttgtcgagacagagaagactcttgcgtttctgataggcacctattggtcttactgacatccactttgcctttctctccacaggttcgaattcgccaccatgtacagaatgcagctgctgctgctcattgccctgtctctggccctggtcaccaattctgaggacatccagatgacacagagcccctccacactgtctgcatctgtgggagacagagtgaccatcacctgtagagccagccagtctatctctagctggctggcctggtatcagcaaaagcctggcaaggcccctaagctgctgatctacaaggccagcacacttgagtcaggggtgccctccagattttctggctctggatctggcacagagttcaccctgaccatcagctccctgcagccagatgactttgccacctactactgccagcagtacaatacctactggacctttggccagggaacaaaggtggaaatcaagggtggaggcggttcaggcggaggtggcagcggcggtggagggtcggaggtccagctgcttgagagtggtggtggactggttcagcctggtggcagcctgagactgtcttgtgctgcctctggcttcaccttcagccactatatcatgatgtgggtcagacaggcccctggcaaaggcctggaatgggtgtcaggcatctacagcagtggtggcatcacagtgtatgctgactctgtgaagggcagattcaccatcagcagagacaacagcaagaacaccctgtacctgcagatgaactccctgagagctgaggacacagcagtgtactactgtgcctatagaagaattggggtgcccagaagggatgagtttgacatctggggccagggcaccatggttacagtgtcctctggcggaggaggcggaggtggaggaggagaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgccaagaccaagcctagagaggaacagtacaacagcacctacagagtggtttctgtgctgacagtgctgcaccaggactggctgaatggcaaagagtacaagtgcaaggtgtccaacaaggccctgcctgctcctattgagaaaaccatctccaaggccaagggccagccaagagaaccccaggtttacaccctgccacctagcagagaagagatgaccaagaaccaggtgtccctgacctgcctggttaagggcttctacccctctgacattgctgtggaatgggagagcaatggccagcctgaaaacaactacaagacaacccctcctgtgctggactctgatggctcattcttcctgtacagcaagctgactgtggacaagtccagatggcagcagggaaatgtgttcagctgctctgtgatgcatgaggccctgcacaaccactacacccagaaaagtctgtctctgtcccctggctaatgactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggcggccgcccgagtttaattggtttatagaactcttcaagctagcgaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 334 - protein, 735 aa, Artificial Sequence [sequence omitted]

SEQ ID NO: 335 - DNA, 5 bp, Artificial Sequence
  Synthetic construct; transcription start site
tctcc

SEQ ID NO: 336 - DNA, 19 bp, Artificial Sequence
  Synthetic construct; promoter sequence
ggtacagtgactcctttcg

SEQ ID NO: 337 - DNA, 19 bp, Artificial Sequence
  Synthetic construct; promoter sequence
ggtacaatgactcctttcg

SEQ ID NO: 338 - DNA, 10 bp, Artificial Sequence
  Synthetic construct; transcription start site
tcattctacc

SEQ ID NO: 339 - protein, 6 aa, Artificial Sequence
  synthetic construct; mutations in AAV capsids
STVDHP

SEQ ID NO: 340 - protein, 4 aa, Artificial Sequence
  synthetic construct; mutations in AAV capsids
ETVG

SEQ ID NO: 341 - protein, 4 aa, Artificial Sequence
  synthetic construct; mutations in AAV capsids
QFLK

SEQ ID NO: 342 - protein, 6 aa, Artificial Sequence
  synthetic construct; mutations in AAV capsids
PAERKK

SEQ ID NO: 343 - protein, 4 aa, Artificial Sequence
  synthetic construct; mutations in AAV capsids
QGGE

SEQ ID NO: 344 - protein, 6 aa, Artificial Sequence
  synthetic construct; mutations in AAV capsids
GIDDHF

SEQ ID NO: 345 - protein, 18 aa, Artificial Sequence
  synthetic construct; mutations in AAV capsids
KKQARTREKSVPEDETGA

SEQ ID NO: 346 - protein, 4 aa, Artificial Sequence
  synthetic construct; mutations in AAV capsids
ALIP

SEQ ID NO: 347 - protein, 4 aa, Artificial Sequence
  synthetic construct; mutations in AAV capsids
STSS

SEQ ID NO: 348 - protein, 21 aa, Artificial Sequence
  synthetic construct; mutations in AAV capsids
QLQQTSGTMASLDPNEVRAAA

SEQ ID NO: 349 - protein, 8 aa, Artificial Sequence
  synthetic construct; mutations in AAV capsids
GAMGEGGQ

SEQ ID NO: 350 - protein, 4 aa, Artificial Sequence
  synthetic construct; mutations in AAV capsids
MEGH

SEQ ID NO: 351 - protein, 6 aa, Artificial Sequence
  synthetic construct; mutations in AAV capsids
AQPATA

SEQ ID NO: 352 - protein, 14 aa, Artificial Sequence
  synthetic construct; mutations in AAV capsids
RRLGSGSQSDATQA

SEQ ID NO: 353 - protein, 7 aa, Artificial Sequence
  synthetic construct; mutations in AAV capsids
AATTEGL

SEQ ID NO: 354 - protein, 5 aa, Artificial Sequence
  synthetic construct; mutations in AAV capsids
RAMRV

SEQ ID NO: 355 - protein, 6 aa, Artificial Sequence
  synthetic construct; mutations in AAV capsids
VQDSTT

SEQ ID NO: 356 - protein, 5 aa, Artificial Sequence
  synthetic construct; mutations in AAV capsids
GNPTD

SEQ ID NO: 357 - protein, 5 aa, Artificial Sequence
  synthetic construct; mutations in AAV capsids
WGFNR

SEQ ID NO: 358 - protein, 5 aa, Artificial Sequence
  synthetic construct; mutations in AAV capsids
QTNTS

SEQ ID NO: 359 - protein, 7 aa, Artificial Sequence
  synthetic construct; mutations in AAV capsids
AGTKRTQ

SEQ ID NO: 360 - protein, 10 aa, Artificial Sequence
  synthetic construct; mutations in AAV capsids
TQGSAATFSN

SEQ ID NO: 361 - protein, 5 aa, Artificial Sequence
  synthetic construct; mutations in AAV capsids
NSTPT

SEQ ID NO: 362 - protein, 7 aa, Artificial Sequence
  synthetic construct; mutations in AAV capsids
TQNSDVN

SEQ ID NO: 363 - protein, 4 aa, Artificial Sequence
  synthetic construct; mutations in AAV capsids
NKNL

SEQ ID NO: 364 - protein, 5 aa, Artificial Sequence
  synthetic construct; mutations in AAV capsids
QGYRD

SEQ ID NO: 365 - protein, 4 aa, Artificial Sequence
  synthetic construct; mutations in AAV capsids
YRLR

SEQ ID NO: 366 - protein, 4 aa, Artificial Sequence
  synthetic construct; mutations in AAV capsids
CIKT

SEQ ID NO: 367 - protein, 8 aa, Artificial Sequence
  synthetic construct; mutations in AAV capsids
GWNLGSGV

SEQ ID NO: 368 - protein, 11 aa, Artificial Sequence
  synthetic construct; mutations in AAV capsids
DSLIKYETHST

SEQ ID NO: 369 - protein, 6 aa, Artificial Sequence
  synthetic construct; mutations in AAV capsids
ASYQVP

SEQ ID NO: 370 - protein, 8 aa, Artificial Sequence
  synthetic construct; mutations in AAV capsids
QTPGMTAG

SEQ ID NO: 371 - protein, 11 aa, Artificial Sequence
  synthetic construct; mutations in AAV capsids
NYTFATTNRME

SEQ ID NO: 372 - protein, 20 aa, Artificial Sequence
  synthetic construct; mutations in AAV capsids
LQGSNTYAMENTMFANPKQN

SEQ ID NO: 373 - protein, 8 aa, Artificial Sequence
  synthetic construct; mutations in AAV capsids
TNTATVPG

SEQ ID NO: 374 - protein, 4 aa, Artificial Sequence
  synthetic construct; mutations in AAV capsids
TLIF

SEQ ID NO: 375 - protein, 4 aa, Artificial Sequence
  synthetic construct; mutations in AAV capsids
TQPV

SEQ ID NO: 376 - protein, 7 aa, Artificial Sequence
  synthetic construct; mutations in AAV capsids
ATDYDMW

SEQ ID NO: 377 - protein, 6 aa, Artificial Sequence
  synthetic construct; mutations in AAV capsids
NLPGGD

SEQ ID NO: 378 - protein, 5 aa, Artificial Sequence
  synthetic construct; mutations in AAV capsids
PADEK

SEQ ID NO: 379 - protein, 5 aa, Artificial Sequence
  synthetic construct; mutations in AAV capsids
QHQLI

SEQ ID NO: 380 - protein, 4 aa, Artificial Sequence
  synthetic construct; mutations in AAV capsids
SESA

SEQ ID NO: 381 - protein, 8 aa, Artificial Sequence
  synthetic construct; mutations in AAV capsids
EASKAALE

SEQ ID NO: 382 - protein, 4 aa, Artificial Sequence
  synthetic construct; mutations in AAV capsids
NMLM

SEQ ID NO: 383 - protein, 4 aa, Artificial Sequence
  synthetic construct; mutations in AAV capsids
TMSN

SEQ ID NO: 384 - protein, 17 aa, Artificial Sequence
  synthetic construct; mutations in AAV capsids
RNSNLPTVDRLTALEAV

SEQ ID NO: 385 - protein, 5 aa, Artificial Sequence
  synthetic construct; mutations in AAV capsids
ASNTA

SEQ ID NO: 386 - protein, 8 aa, Artificial Sequence
  synthetic construct; mutations in AAV capsids
AIADYHTM

SEQ ID NO: 387 - protein, 5 aa, Artificial Sequence
  synthetic construct; mutations in AAV capsids
NDPQF

SEQ ID NO: 388 - protein, 5 aa, Artificial Sequence
  synthetic construct; mutations in AAV capsids
QGTRD

SEQ ID NO: 389 - protein, 4 aa, Artificial Sequence
  synthetic construct; mutations in AAV capsids
IAAE

SEQ ID NO: 390 - protein, 6 aa, Artificial Sequence
  synthetic construct; mutations in AAV capsids
SDVPVS

SEQ ID NO: 391 - protein, 13 aa, Artificial Sequence
  synthetic construct; mutations in AAV capsids
GQQVSLLWTPDAA

SEQ ID NO: 392 - DNA, 1512 bp, Artificial Sequence
  synthetic construct; VL-VH-Fc sequence
atgtacagaatgcagctgctgctgctcattgccctgtctctggccctggtcaccaattctgaggacatccagatgacacagagcccctccacactgtctgcatctgtgggagacagagtgaccatcacctgtagagccagccagtctatctctagctggctggcctggtatcagcaaaagcctggcaaggcccctaagctgctgatctacaaggccagcacacttgagtcaggggtgccctccagattttctggctctggatctggcacagagttcaccctgaccatcagctccctgcagccagatgactttgccacctactactgccagcagtacaatacctactggacctttggccagggaacaaaggtggaaatcaagggtggaggcggttcaggcggaggtggcagcggcggtggagggtcggaggtccagctgcttgagagtggtggtggactggttcagcctggtggcagcctgagactgtcttgtgctgcctctggcttcaccttcagccactatatcatgatgtgggtcagacaggcccctggcaaaggcctggaatgggtgtcaggcatctacagcagtggtggcatcacagtgtatgctgactctgtgaagggcagattcaccatcagcagagacaacagcaagaacaccctgtacctgcagatgaactccctgagagctgaggacacagcagtgtactactgtgcctatagaagaattggggtgcccagaagggatgagtttgacatctggggccagggcaccatggttacagtgtcctctggcggaggaggcggaggtggaggaggagaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgccaagaccaagcctagagaggaacagtacaacagcacctacagagtggtttctgtgctgacagtgctgcaccaggactggctgaatggcaaagagtacaagtgcaaggtgtccaacaaggccctgcctgctcctattgagaaaaccatctccaaggccaagggccagccaagagaaccccaggtttacaccctgccacctagcagagaagagatgaccaagaaccaggtgtccctgacctgcctggttaagggcttctacccctctgacattgctgtggaatgggagagcaatggccagcctgaaaacaactacaagacaacccctcctgtgctggactctgatggctcattcttcctgtacagcaagctgactgtggacaagtccagatggcagcagggaaatgtgttcagctgctctgtgatgcatgaggccctgcacaaccactacacccagaaaagtctgtctctgtcccctggc

SEQ ID NO: 393 - protein, 504 aa, Artificial Sequence
  synthetic construct; VH-VL-Fc sequence
MYRMQLLLLIALSLALVTNSEDIQMTQSPSTLSASVGDRVTITCRASQSISSWLAWYQQKPGKAPKLLIYKASTLESGVPSRFSGSGSGTEFTLTISSLQPDDFATYYCQQYNTYWTFGQGTKVEIKGGGGSGGGGSGGGGSEVQLLESGGGLVQPGGSLRLSCAASGFTFSHYIMMWVRQAPGKGLEWVSGIYSSGGITVYADSVKGRFTISRDNSKNTLYLQMNSLRAEDTAVYYCAYRRIGVPRRDEFDIWGQGTMVTVSSGGGGGGGGGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG